SEQUENCE LISTING (145 sequences)

SEQ ID NO: 1 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - protein, 227 aa, Mus musculus
MGPLHQFLLLLITALSQALNTTVLQGMAGQSLRVSCTYDALKHWGRRKAWCRQLGEEGPCQRVVSTHGVWLLAFLKKRNGSTVIADDTLAGTVTITLKNLQAGDAGLYQCQSLRGREAEVLQKVLVEVLEDPLDDQDAGDLWVPEESSSFEGAQVEHSTSRNQETSFPPTSILLLLACVLLSKFLAASILWAVARGRQKPGTPVVRGLDCGQDAGHQLQILTGPGGT

SEQ ID NO: 3 - protein, 228 aa, Rattus rattus
MEPLHVFVLLLVTELSQALNTTVLQGVAGQSLRVSCTYDALRHWGRRKAWCRQLAEEGPCQRVVSTHGVWLLAFLRKQNGSTVITDDTLAGTVTITLRNLQAGDAGLYQCQSLRGREAEVLQKVVVEVLEDPLDDQDAGDLWVPEESESFEGAQVEHSTSSQVSSCGSPLTYHLPPKEPIRKDLLPTHFHSSPPGLCPPEQASYSQHPLGCGQGQAEAGDTCGQWARL

SEQ ID NO: 4 - protein, 260 aa, Macaca mulatta
MPDPLFSAVQGKDKILHKALCICPWPGKGGMEPLRLLILLFATELSGAHNTTVFQGVEGQSLQVSCPYDSMKHWGRRKAWCRQLGEKGPCQRVVSTHNLWLLSFLRRRNGSTAITDDTLGGTLTITLRNLQPHDAGFYQCQSLHGSEADTLRKVLVEVLADPLDHRDAGDLWVPGESESFEDAHVEHSISRSLLEGEIPFPPTSVLLLLACIFLIKILAASALWAAAWHGQKPGTHPPSEPDCGHDPGHQLQTLPGLRDT

SEQ ID NO: 5 - protein, 230 aa, Macaca fascicularis
MEPLRLLILLFATELSGAHNTTVFQGVEGQSLQVSCPYDSMKHWGRRKAWCRQLGEKGPCQRVVSTHNLWLLSFLRRRNGSTAITDDTLGGTLTITLRNLQPHDAGFYQCQSLHGSEADTLRKVLVEVLADPLDHRDAGDLWVPGESESFEDAHVEHSISRSLLEGEIPFPPTSVLLLLACIFLIKILAASALWAAAWHGQKPGTHPPSEPDCGHDPGHQLQTLPGLRDT

SEQ ID NO: 6 - protein, 230 aa, Equus caballus
MEPLPLLILLSVAELSRGHNTTVFQGTAGRSLKVSCPYNSLMHWGRRKAWCRQLGEDGPCQQVVSTHSLWLLSFLKRRNGSTVITDDALGGILTITLRNLQAHDAGFYQCQSLHGGEADTLRKVLVEVLADPLDHQEPGDLWIPKESESFEDAQVEHSISRSLVEEEIPSLPTSILLLLACIFLSKLLAASAIWAAAWHGQKQETPPASEPDRGHDPGYQLHTLTGERDT

SEQ ID NO: 7 - protein, 233 aa, Sus scrofa
METLGLLLLLWVAELSRAHNTSVFQGTAGQSLRVSCSYNSLKHWGRRKAWCRQLSEEGLCQHVVSTHPTWLLSFLKRRNGSTAITDDALGGTLTITLRNLQAHDAGLYQCQSLHGSEADTLKKVLVEVLADPLESQSKSFQDVQMEHSISRNLSEESLFPPTSTLFLLACVFLSKLLVASALWAAAWHGHKQRTSPAGGLDCGRDPGDQDQTLTDELGESSDQDQTLTELRDT

SEQ ID NO: 8 - protein, 230 aa, Canis lupus familiaris
MEPLWLLILLAVTELSGAHNTTVFQGMAGRSLQVSCPYNSLKHWGRRKAWCRQVDKEGPCQRVVSTHRSWLLSFLKRWNGSTAIVDDALGGTLTITLRNLQAHDAGLYQCQSLYGDEADTLRKVLVEVLADPLDHLDPGDLWIPEESKGFEDAHVEPSVSRSLSEEEIPFPPTSILFLLACIFLSKFLAASALWAAAWRGQKLGTPQASELDCSCDPGYQLQTLTEPRDM

SEQ ID NO: 9 - protein, 26 aa, Artificial Sequence
  Synthetic Construct
QVQLVQSGAEVKKPGSSVKVSCKASG

SEQ ID NO: 10 - protein, 26 aa, Artificial Sequence
  Synthetic Construct
EVQLVQSGAEVKKPGSSVKVSCKASG

SEQ ID NO: 11 - protein, 26 aa, Artificial Sequence
  Synthetic Construct
QVQLVQSGAEVKKPGASVKVSCKASG

SEQ ID NO: 12 - protein, 14 aa, Artificial Sequence
  Synthetic Construct
WVRQAPGQGLEWMG

SEQ ID NO: 13 - protein, 14 aa, Artificial Sequence
  Synthetic Construct
WVRQAPGQRLEWIG

SEQ ID NO: 14 - protein, 30 aa, Artificial Sequence
  Synthetic Construct
RVTITADESTSTAYMELSSLRSEDTAVYYC

SEQ ID NO: 15 - protein, 30 aa, Artificial Sequence
  Synthetic Construct
RVTITADTSASTAYMELSSLRSEDTAVYYC

SEQ ID NO: 16 - protein, 11 aa, Artificial Sequence
  Synthetic Construct
WGQGTLVTVSS

SEQ ID NO: 17 - protein, 23 aa, Artificial Sequence
  Synthetic Construct
DVVMTQTPLSLSVTPGQPASISC

SEQ ID NO: 18 - protein, 23 aa, Artificial Sequence
  Synthetic Construct
GVVMTQTPLSLSVTPGQPASISC

SEQ ID NO: 19 - protein, 23 aa, Artificial Sequence
  Synthetic Construct
GVVMAQTPLSLSVTPGQPASISC

SEQ ID NO: 20 - protein, 23 aa, Artificial Sequence
  Synthetic Construct
DVVMTQSPDSLAVSLGERATINC

SEQ ID NO: 21 - protein, 15 aa, Artificial Sequence
  Synthetic Construct
WYLQKPGQSPQLLIY

SEQ ID NO: 22 - protein, 15 aa, Artificial Sequence
  Synthetic Construct
WYQQKPGQSPKLLIY

SEQ ID NO: 23 - protein, 32 aa, Artificial Sequence
  Synthetic Construct
GVPDRFSGSGSGTDFTLKISRVEAEDVGVYYC

SEQ ID NO: 24 - protein, 32 aa, Artificial Sequence
  Synthetic Construct
GVPDRFSGSGSGTDFTLTISSLQAEDVAVYYC

SEQ ID NO: 25 - protein, 10 aa, Artificial Sequence
  Synthetic Construct
FGQGTKLEIK

SEQ ID NO: 26 - protein, 10 aa, Artificial Sequence
  Synthetic Construct
FGGGTKVEIK

SEQ ID NO: 27 - protein, 123 aa, Artificial Sequence
  Synthetic Construct
QVQLVQSGAEVKKPGASVKVSCKASGYAFSSQWMNWVRQAPGQRLEWIGRIYPGGGDTNYAGKFQGRVTITADTSASTAYMELSSLRSEDTAVYYCARLLRNQPGESYAMDYWGQGTLVTVSS

SEQ ID NO: 28 - protein, 123 aa, Artificial Sequence
  Synthetic Construct
QVQLVQSGAEVKKPGSSVKVSCKASGYAFSSDWMNWVRQAPGQGLEWMGRIYPGEGDTNYARKFHGRVTITADESTSTAYMELSSLRSEDTAVYYCARLLRNKPGESYAMDYWGQGTLVTVSS

SEQ ID NO: 29 - protein, 112 aa, Artificial Sequence
  Synthetic Construct
DVVMTQTPLSLSVTPGQPASISCRTSQSLVHSNAYTYLHWYLQKPGQSPQLLIYKVSNRVSGVPDRFSGSGSGTDFTLKISRVEAEDVGVYYCSQSTRVPYTFGQGTKLEIK

SEQ ID NO: 30 - protein, 112 aa, Artificial Sequence
  Synthetic Construct
DVVMTQSPDSLAVSLGERATINCRSSQSLVHSNRYTYLHWYQQKPGQSPKLLIYKVSNRFSGVPDRFSGSGSGTDFTLKISRVEAEDVGVYYCSQSTRVPYTFGQGTKLEIK

SEQ ID NO: 31 - protein, 16 aa, Artificial Sequence
  Synthetic Construct
ARLLRNQPGESYAMDY

SEQ ID NO: 32 - protein, 9 aa, Artificial Sequence
  Synthetic Construct
SQSTRVPYT

SEQ ID NO: 33 - protein, 7 aa, Artificial Sequence
  Synthetic Construct
KVSNRFS

SEQ ID NO: 34 - protein, 9 aa, Artificial Sequence
  Synthetic Construct
YAFSSQWMN

SEQ ID NO: 35 - protein, 17 aa, Artificial Sequence
  Synthetic Construct
RIYPGGGDTNYAGKFQG

SEQ ID NO: 36 - protein, 9 aa, Artificial Sequence
  Synthetic Construct
YAFSSDWMN

SEQ ID NO: 37 - protein, 17 aa, Artificial Sequence
  Synthetic Construct
RIYPGEGDTNYARKFHG

SEQ ID NO: 38 - protein, 16 aa, Artificial Sequence
  Synthetic Construct
ARLLRNKPGESYAMDY

SEQ ID NO: 39 - protein, 16 aa, Artificial Sequence
  Synthetic Construct
RTSQSLVHSNAYTYLH

SEQ ID NO: 40 - protein, 7 aa, Artificial Sequence
  Synthetic Construct
KVSNRVS

SEQ ID NO: 41 - protein, 16 aa, Artificial Sequence
  Synthetic Construct
RSSQSLVHSNRYTYLH

SEQ ID NO: 42 - protein, 110 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCP

SEQ ID NO: 43 - protein, 453 aa, Artificial Sequence
  Synthetic Construct
QVQLVQSGAEVKKPGASVKVSCKASGYAFSSQWMNWVRQAPGQRLEWIGRIYPGGGDTNYAGKFQGRVTITADTSASTAYMELSSLRSEDTAVYYCARLLRNQPGESYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 44 - protein, 452 aa, Artificial Sequence
  Synthetic Construct
QVQLVQSGAEVKKPGASVKVSCKASGYAFSSQWMNWVRQAPGQRLEWIGRIYPGGGDTNYAGKFQGRVTITADTSASTAYMELSSLRSEDTAVYYCARLLRNQPGESYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 45 - protein, 453 aa, Artificial Sequence
  Synthetic Construct
QVQLVQSGAEVKKPGSSVKVSCKASGYAFSSDWMNWVRQAPGQGLEWMGRIYPGEGDTNYARKFHGRVTITADESTSTAYMELSSLRSEDTAVYYCARLLRNKPGESYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 452 aa, Artificial Sequence
  Synthetic Construct
QVQLVQSGAEVKKPGSSVKVSCKASGYAFSSDWMNWVRQAPGQGLEWMGRIYPGEGDTNYARKFHGRVTITADESTSTAYMELSSLRSEDTAVYYCARLLRNKPGESYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 219 aa, Artificial Sequence
  Synthetic Construct
DVVMTQSPDSLAVSLGERATINCRSSQSLVHSNRYTYLHWYQQKPGQSPKLLIYKVSNRFSGVPDRFSGSGSGTDFTLKISRVEAEDVGVYYCSQSTRVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 219 aa, Artificial Sequence
  Synthetic Construct
DVVMTQTPLSLSVTPGQPASISCRTSQSLVHSNAYTYLHWYLQKPGQSPQLLIYKVSNRVSGVPDRFSGSGSGTDFTLKISRVEAEDVGVYYCSQSTRVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 19 aa, Artificial Sequence
  Synthetic Construct
  VARIANT 1: Xaa = Asp or Glu
  VARIANT 2, 3: Xaa = Any Amino Acid, and up to two can be present or absent
  VARIANT 5, 6: Xaa = Any Amino Acid
  VARIANT 7: Xaa = Leu or Ile
  VARIANT 8, 9, 10, 11, 12, 13, 14, 15: Xaa = Any Amino Acid, and up to two can be present or absent
  VARIANT 17, 18: Xaa = Any Amino Acid
  VARIANT 19: Xaa = Leu or Ile
XXXYXXXXXXXXXXXYXXX

SEQ ID NO: 50 - protein, 11 aa, Artificial Sequence
  Synthetic Construct
GYSITSDYAWN

SEQ ID NO: 51 - protein, 16 aa, Artificial Sequence
  Synthetic Construct
YINYSGRTIYNPSLKS

SEQ ID NO: 52 - protein, 11 aa, Artificial Sequence
  Synthetic Construct
ARWNGNYGFAY

SEQ ID NO: 53 - protein, 16 aa, Artificial Sequence
  Synthetic Construct
RSSQSLVHINGNTYLH

SEQ ID NO: 54 - protein, 7 aa, Artificial Sequence
  Synthetic Construct
KVSNRFS

SEQ ID NO: 55 - protein, 9 aa, Artificial Sequence
  Synthetic Construct
SQTTHALFT

SEQ ID NO: 56 - protein, 118 aa, Artificial Sequence
  Synthetic Construct
DVQLQESGPGLVKPSQSLSLTCTVTGYSITSDYAWNWIRQFPGNKLEWMGYINYSGRTIYNPSLKSRISITRDTSKNHFFLQLISVTTEDTATYYCARWNGNYGFAYWGQGTLVTVSA

SEQ ID NO: 57 - protein, 111 aa, Artificial Sequence
  Synthetic Construct
DWMTQNPLSLPVSLGDQASISCRSSQSLVHINGNTYLHWYLQKPGQSPKLLIYKVSNRFSGVPDRFSGSGSGTDFTLKISRVEAEDLGVYFCSQTTHALFTFGSGTKLEIK

SEQ ID NO: 58 - protein, 7 aa, Artificial Sequence
  Synthetic Construct
GYTFTSY

SEQ ID NO: 59 - protein, 15 aa, Artificial Sequence
  Synthetic Construct
IGRSDPTTGGTNYNE

SEQ ID NO: 60 - protein, 9 aa, Artificial Sequence
  Synthetic Construct
VRTSGTGDY

SEQ ID NO: 61 - protein, 16 aa, Artificial Sequence
  Synthetic Construct
RSSQSLVHNNGNTFLH

SEQ ID NO: 62 - protein, 6 aa, Artificial Sequence
  Synthetic Construct
VSNRFS

SEQ ID NO: 63 - protein, 9 aa, Artificial Sequence
  Synthetic Construct
SQTTHVPPT

SEQ ID NO: 64 - protein, 140 aa, Artificial Sequence
  Synthetic Construct
QVQLQQPGAELVKPGASVKLSCKASGYTFTSYWMHWVKQSPGRGLEWIGRSDPTTGGTNYNEKFKTKATLTVDKPSSTAYMQLSSLTSDDSAVYYCVRTSGTGDYWGQGTSLTVSSAKTTAPSVYPLAPVCGGTTGSSVT

SEQ ID NO: 65 - protein, 140 aa, Artificial Sequence
  Synthetic Construct
DVVMTQTPLSLPVSLGDQASISCRSSQSLVHNNGNTFLHWYLQKPGQSPKLLIYKVSNRFSGVPDRFSGSGSGTDFTLKISRVEAEDLGVYFCSQTTHVPPTFGGGTKLEIKRADAAPTVSIFPPSSEQLTSGGASVVCF

SEQ ID NO: 66 - protein, 8 aa, Artificial Sequence
  Synthetic Construct
GFTFTDFY

SEQ ID NO: 67 - protein, 10 aa, Artificial Sequence
  Synthetic Construct
IRNKANGYTT

SEQ ID NO: 68 - protein, 15 aa, Artificial Sequence
  Synthetic Construct
ARIGINNGGSLDYWG

SEQ ID NO: 69 - protein, 12 aa, Artificial Sequence
  Synthetic Construct
QSLLYSENNQDY

SEQ ID NO: 70 - protein, 3 aa, Artificial Sequence
  Synthetic Construct
GAS

SEQ ID NO: 71 - protein, 9 aa, Artificial Sequence
  Synthetic Construct
EQTYSYPYT

SEQ ID NO: 72 - protein, 121 aa, Artificial Sequence
  Synthetic Construct
EVKLLESGGGLVQPGGSMRLSCAASGFTFTDFYMNWIRQPAGKAPEWLGLIRNKANGYTTEYNPSVKGRFTISRDNTQNMLYLQMNTLREDTATYYCARIGINNGGSLDYWGQGVMVTVSS

SEQ ID NO: 73 - protein, 113 aa, Artificial Sequence
  Synthetic Construct
DILINQSPASLTVSAGEKVTMSCKSSQSLLYSENNQDYLAWYQQKPGQFPKLLIYGASNRHTGVPDRFTGSGSGTDFTLTISSVQAEDLADYYCEQTYSYPYTFGAGTKLELK

SEQ ID NO: 74 - protein, 8 aa, Artificial Sequence
  Synthetic Construct
GFTFTDFY

SEQ ID NO: 75 - protein, 10 aa, Artificial Sequence
  Synthetic Construct
IRNKTKGYTT

SEQ ID NO: 76 - protein, 15 aa, Artificial Sequence
  Synthetic Construct
ARIGVNNGGSLDYWG

SEQ ID NO: 77 - protein, 12 aa, Artificial Sequence
  Synthetic Construct
QSLLYSENNQDY

SEQ ID NO: 78 - protein, 3 aa, Artificial Sequence
  Synthetic Construct
GAS

SEQ ID NO: 79 - protein, 9 aa, Artificial Sequence
  Synthetic Construct
EQTYSYPYT

SEQ ID NO: 80 - protein, 122 aa, Artificial Sequence
  Synthetic Construct
EVKLLEFGGGLVQPGGSMRLSCAASGFTFTDFYMNWIRQPAGRAPEWLGLIRNKTKGYTTEYNRSVKGRFTISRDNTQNMLYLQMNSLRPEDTATYYCARIGVNNGGSLDYWGQGVMVTVSS

SEQ ID NO: 81 - protein, 113 aa, Artificial Sequence
  Synthetic Construct
DILIIQSPASLTVSAGARVTMSCKSSQSLLYSENNQDYLAWYQQKPGQFPKLLIYGASNRHTGVPDRFTGSGSGTDFTLTISSVQAEDLADYYCEQTYSYPYTFGAGTKLELK

SEQ ID NO: 82 - protein, 8 aa, Artificial Sequence
  Synthetic Construct
GFTFTDFY

SEQ ID NO: 83 - protein, 10 aa, Artificial Sequence
  Synthetic Construct
IRNKANGYTT

SEQ ID NO: 84 - protein, 15 aa, Artificial Sequence
  Synthetic Construct
ARIGINNGGSLDYWG

SEQ ID NO: 85 - protein, 12 aa, Artificial Sequence
  Synthetic Construct
QSLLYSEKNQDY

SEQ ID NO: 86 - protein, 3 aa, Artificial Sequence
  Synthetic Construct
GAS

SEQ ID NO: 87 - protein, 9 aa, Artificial Sequence
  Synthetic Construct
EQTYSYPYT

SEQ ID NO: 88 - protein, 122 aa, Artificial Sequence
  Synthetic Construct
EVKLLESGGGLVQPGGSMRLSCAASGFTFTDFYMNWIRQPAGKAPEWLGLIRNKANGYTTVYNPSVKGRFTISRDNTQNMLYLQMNTLRGEDTATYYCARIGINNGGSLDYWGQGVMVTVSS

SEQ ID NO: 89 - protein, 113 aa, Artificial Sequence
  Synthetic Construct
DILINQSPASLTVSTGEKVTMSCRSSQSLLYSEKNQDYLAWYQQKPGQFPKLLIYGASYRHTGVPDRFTGSGSGTDFTLTISSVQAEDLADYYCEQTYSYPYTFGAGTKLELK

SEQ ID NO: 90 - protein, 8 aa, Artificial Sequence
  Synthetic Construct
GFTFTDFY

SEQ ID NO: 91 - protein, 10 aa, Artificial Sequence
  Synthetic Construct
IRNKANGYTT

SEQ ID NO: 92 - protein, 15 aa, Artificial Sequence
  Synthetic Construct
ARIGINNGGSLDYWG

SEQ ID NO: 93 - protein, 12 aa, Artificial Sequence
  Synthetic Construct
QSLLYSEKNQDY

SEQ ID NO: 94 - protein, 3 aa, Artificial Sequence
  Synthetic Construct
GAS

SEQ ID NO: 95 - protein, 9 aa, Artificial Sequence
  Synthetic Construct
EQTYSYPYT

SEQ ID NO: 96 - protein, 122 aa, Artificial Sequence
  Synthetic Construct
EVKLLESGGGLVQPGGSMRLSCAASGFTFTDFYMNWIRQPAGKAPEWLGLIRNKANGYTTQYNPSVKGRFTISRDNTQNMLYLQMNTLRGEDTATYYCARIGINNGGSLDYWGQGVMVTVSS

SEQ ID NO: 97 - protein, 113 aa, Artificial Sequence
  Synthetic Construct
DILINQSPASLTVSAGEKVTMSCKSSQSLLYSEKNQDYLAWYQQKPGQSPKLLMYGASYRHTGVPDRFTGSGSGTDFTLTISSVQAEDLADYYCEQTYSYPYTFGAGTKLELK

SEQ ID NO: 98 - protein, 8 aa, Artificial Sequence
  Synthetic Construct
GFTFTDFY

SEQ ID NO: 99 - protein, 10 aa, Artificial Sequence
  Synthetic Construct
IRNKTKGYTT

SEQ ID NO: 100 - protein, 15 aa, Artificial Sequence
  Synthetic Construct
ARIGVNNGGSLDYWG

SEQ ID NO: 101 - protein, 12 aa, Artificial Sequence
  Synthetic Construct
QSLLYSENNQDY

SEQ ID NO: 102 - protein, 3 aa, Artificial Sequence
  Synthetic Construct
GAS

SEQ ID NO: 103 - protein, 9 aa, Artificial Sequence
  Synthetic Construct
EQTYSYPYT

SEQ ID NO: 104 - protein, 122 aa, Artificial Sequence
  Synthetic Construct
EVKLLESGGGLVQPGGSMRLSCAASGFTFTDFYMNWIRQPAGKAPEWLGLIRNKTKGYTTEYNTSVKGRFTISRDNTQNMLYLQMNSLRPEDTATYYCARIGVNNGGSLDYWGQGVMVTVSS

SEQ ID NO: 105 - protein, 113 aa, Artificial Sequence; identical to SEQ ID NO: 81
  Synthetic Construct

SEQ ID NO: 106 - protein, 8 aa, Artificial Sequence
  Synthetic Construct
GFTFTDFY

SEQ ID NO: 107 - protein, 10 aa, Artificial Sequence
  Synthetic Construct
IRNKANGYTT

SEQ ID NO: 108 - protein, 15 aa, Artificial Sequence
  Synthetic Construct
ARIGINNGGSLDYWG

SEQ ID NO: 109 - protein, 12 aa, Artificial Sequence
  Synthetic Construct
QSLLYSEKNQDY

SEQ ID NO: 110 - protein, 3 aa, Artificial Sequence
  Synthetic Construct
GAS

SEQ ID NO: 111 - protein, 9 aa, Artificial Sequence
  Synthetic Construct
EQTYSYPYT

SEQ ID NO: 112 - protein, 122 aa, Artificial Sequence; identical to SEQ ID NO: 96
  Synthetic Construct

SEQ ID NO: 113 - protein, 113 aa, Artificial Sequence; identical to SEQ ID NO: 97
  Synthetic Construct

SEQ ID NO: 114 - protein, 8 aa, Artificial Sequence
  Synthetic Construct
GFTFTDFY

SEQ ID NO: 115 - protein, 10 aa, Artificial Sequence
  Synthetic Construct
IRNKTKGYTT

SEQ ID NO: 116 - protein, 15 aa, Artificial Sequence
  Synthetic Construct
ARIGTNNGGSLDYWG

SEQ ID NO: 117 - protein, 12 aa, Artificial Sequence
  Synthetic Construct
QSLLYSENNQDY

SEQ ID NO: 118 - protein, 3 aa, Artificial Sequence
  Synthetic Construct
GAS

SEQ ID NO: 119 - protein, 9 aa, Artificial Sequence
  Synthetic Construct
EQTYSYPYT

SEQ ID NO: 120 - protein, 122 aa, Artificial Sequence
  Synthetic Construct
EVKLLESGGGLVQPGGSMRLSCAASGFTFTDFYMNWIRQPAGETPEWLGLIRNKTKGYTTEYNPSVKGRFTISRDNTQNMLYLQMNSLRPEDTATYYCARIGTNNGGSLDYWGQGVMVTVSS

SEQ ID NO: 121 - protein, 113 aa, Artificial Sequence; identical to SEQ ID NO: 81
  Synthetic Construct

SEQ ID NO: 122 - protein, 8 aa, Artificial Sequence
  Synthetic Construct
GFTFTDFY

SEQ ID NO: 123 - protein, 10 aa, Artificial Sequence
  Synthetic Construct
IRNKVNGYRT

SEQ ID NO: 124 - protein, 15 aa, Artificial Sequence
  Synthetic Construct
ARIGINNGGSLDYWG

SEQ ID NO: 125 - protein, 12 aa, Artificial Sequence
  Synthetic Construct
QSLLYSENNQDY

SEQ ID NO: 126 - protein, 3 aa, Artificial Sequence
  Synthetic Construct
GAS

SEQ ID NO: 127 - protein, 9 aa, Artificial Sequence
  Synthetic Construct
EQTYSYPYT

SEQ ID NO: 128 - protein, 122 aa, Artificial Sequence
  Synthetic Construct
EVKLLESGGGLVQPGGSMRLSCVVSGFTFTDFYMNWIRQAAGKAPEWLGLIRNKVNGYRTEYNPSVKGRFTISRDNIQNMLYLQMNTLRAEDTATYYCARIGINNGGSLDYWGQGVMVTVSS

SEQ ID NO: 129 - protein, 113 aa, Artificial Sequence; identical to SEQ ID NO: 73
  Synthetic Construct

SEQ ID NO: 130 - protein, 8 aa, Artificial Sequence
  Synthetic Construct
GFTFTDFY

SEQ ID NO: 131 - protein, 10 aa, Artificial Sequence
  Synthetic Construct
IRNKAYGYTT

SEQ ID NO: 132 - protein, 15 aa, Artificial Sequence
  Synthetic Construct
ARIGINYGGSLDYWG

SEQ ID NO: 133 - protein, 12 aa, Artificial Sequence
  Synthetic Construct
QSLLYSESNQDY

SEQ ID NO: 134 - protein, 3 aa, Artificial Sequence
  Synthetic Construct
GAS

SEQ ID NO: 135 - protein, 9 aa, Artificial Sequence
  Synthetic Construct
EQTYSYPYT

SEQ ID NO: 136 - protein, 122 aa, Artificial Sequence
  Synthetic Construct
EVKLLESGGGLVQPGGSMRLSCAASGFTFTDFYMNWIRQPAGKAPEWLGLIRNKAYGYTTEYNPSVKGRFTISRDNTQDMLYLQMNTLRAEDTATYYCARIGINYGGSLDYWGQGVMVTVSS

SEQ ID NO: 137 - protein, 113 aa, Artificial Sequence
  Synthetic Construct
DILINQSPASLTVSAGEKVTVSCKSSQSLLYSESNQDYLAWYQQKPGQFPKLLIYGASYRHTGVPDRFTGSGSGTDFTLTISSVQAEDLAHYYCEQTYSYPYTFGAGTKLELK

SEQ ID NO: 138 - protein, 8 aa, Artificial Sequence
  Synthetic Construct
GFTFTDFY

SEQ ID NO: 139 - protein, 10 aa, Artificial Sequence
  Synthetic Construct
IRNKANGFTT

SEQ ID NO: 140 - protein, 15 aa, Artificial Sequence
  Synthetic Construct
ARIGINNGGSLDYWG

SEQ ID NO: 141 - protein, 12 aa, Artificial Sequence
  Synthetic Construct
QSLLYSENKQDY

SEQ ID NO: 142 - protein, 3 aa, Artificial Sequence
  Synthetic Construct
GAS

SEQ ID NO: 143 - protein, 9 aa, Artificial Sequence
  Synthetic Construct
EQTYSYPYT

SEQ ID NO: 144 - protein, 122 aa, Artificial Sequence
  Synthetic Construct
EVKLLESGGGLVQPGGSLRLSCVASGFTFTDFYMNWIRQPAGKAPEWLGLIRNKANGFTTEYNPSVKGRFTISRDNTQHMLYLQMNTLRAEDTATYYCARIGINNGGSLDYWGQGVMVTVSS

SEQ ID NO: 145 - protein, 113 aa, Artificial Sequence
  Synthetic Construct
DILINQSPASLTVSTGEKVTMSCKSSQSLLYSENKQDYLAWYQQKPGQFPKLLIYGASNRHTGVPDRFTGSGSGTDFTLTINIVQAEDLADYYCEQTYSYPYTFGAGTKLELK

(19) United States
(12) Patent Application Publication
(10) Pub. No.: US 2023/0183341 A1
(43) Pub. Date: Jun. 15, 2023

(54) METHODS OF USE OF ANTI-TREM2 ANTIBODIES

(71) Applicant: Alector LLC, South San Francisco, CA (US)

(72) Inventors: Robert PAUL, San Francisco, CA (US); Michael F. WARD, San Francisco, CA (US); Hua LONG, San Carlos, CA (US); Omer Rizwan SIDDIQUI, Redwood City, CA (US); Arnon ROSENTHAL, Woodside, CA (US); Felix Leejia YEH, Belmont, CA (US); Sam JACKSON, Oakland, CA (US)

(73) Assignee: Alector LLC, South San Francisco, CA (US)

(21) Appl. No.: 17/916,728

(22) PCT Filed: Apr. 2, 2021
(86) PCT No.: PCT/US2021/025626

§ 371 Date: Oct. 3, 2022

Related U.S. Application Data
(60) Provisional application No. 63/005,130, filed on Apr. 3, 2020.
(60) Provisional application No. 63/079,810, filed on Sep. 17, 2020.

(51) Int. Cl. C07K 16/28; A61P 25/28

(52) U.S. Cl. CPC C07K 16/2803; A61P 25/28; A61K 2039/505

(57) ABSTRACT

The present disclosure is generally directed to the use of compositions that include antibodies, e.g., monoclonal, chimeric, affinity-matured, humanized antibodies, antibody fragments, etc., that specifically bind one or more epitopes within a TREM2 protein, e.g., human TREM2, for use in treating and/or delaying progression of a disease or injury in an individual in need thereof.

### CROSS-REFERENCE TO RELATED APPLICATION

This application claims the benefit of U.S. Provisional Application 63/005,130, filed Apr. 3, 2020, and U.S. Provisional Application 63/079,810, filed Sep. 17, 2020, each of which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 735022003540SEQLIST.TXT, date recorded: Mar. 30, 2021, size: 88 KB).

### FIELD

The present disclosure relates to therapeutic uses of anti-TREM2 antibodies.

### BACKGROUND

Alzheimer's disease (AD) is a degenerative brain disease and is the most common cause of dementia in the United States, affecting approximately 5.5 million Americans. Worldwide, 50 million people are living with dementia, and this prevalence is expected to triple by 2050. Of the top 10 causes of death in the United States, AD is the only major cause of morbidity and mortality without suitable treatments for prevention, slowing, or cure (2017 Alzheimer's Association Report). Current therapies for AD such as acetylcholinesterase inhibitors (e.g. donepezil) and N-methyl-D-aspartate (NMDA) receptor antagonists (e.g. memantine) show modest and transient benefits to cognition and behavior parameters in AD patients, but do not slow or halt the progression of the disease (Cummings (2004) N Engl J Med, 351:56-67).

Recent studies have suggested that Triggering Receptor Expressed on Myeloid cells-2 (TREM2), an immunoglobulin-like receptor, may play a key role in AD. For example, heterozygous mutations in the TREM2 gene have been found to increase the risk of AD by up to 3-fold (Guerreiro et al (2013), N Engl J Med, 368:117-127; Jonsson et al (2013) N Engl J Med, 368:107-116), and increase the rate at which brain volume shrinks (Rajagopalan et al (2013) N Engl J Med, 369:1565-1567). Even individuals without AD who carry a heterozygous TREM2 mutation show impaired cognition compared to individuals with 2 normal TREM2 alleles. In the context of AD pathology, TREM2 expression impacts amyloid pathology, modulates neuritic dystrophy, tau hyperphosphorylation and aggregation, and affects synaptic and neuronal loss (Jay et al (2017) Mol Neurodegener, 12(1):56). In addition, it has been shown that TREM2 plays a key role in limiting the development of peri-plaque tau pathologies (Leyns et al (2019) Nat Neurosci, PMID: 31235932). Recent mouse genetic model studies also strongly support a key role for TREM2 in AD, with loss or deficiency of TREM2 being associated with increased pathology (Cheng-Hathaway et al (2018) Mol Neurodegener, 13(1):29; Wang et al (2015) Cell, 160:1061-1071; Wang et al (2016) J Exp Med, 213:667-675; Yuan et al (2016) Neuron, 90:724-739; Jay et al (2017) J Neurosci, 37:637-647).

TREM2 is expressed primarily on myeloid lineage cells, including microglia (Colonna and Wang (2016) Nat Rev Neurosci, 17:201-207). Microglia are resident macrophages of the central nervous system that, when activated appropriately, are thought to serve an important protective role in Alzheimer's disease through their housekeeping functions such as facilitating clearance of cellular debris through phagocytosis, as well as secretion of growth factors. It has been shown that TREM2 expression regulates microglial chemotaxis and phagocytosis, and enhances microglial cell survival, proliferation, and differentiation. In addition, it is well known that TREM2 is required to sustain microglial trophic function in the aging brain, and animal studies showed that an overlap exists between aged microglia phenotype and microglial molecular signatures found in models of AD, which include TREM2 pathways (Krasemann et al (2017) Immunity, 47(3):566-581). These findings suggest that activation of TREM2 may ameliorate AD pathology and result in improvements in cognitive function through activation of the innate immune system.

Accordingly, there is a need in the art for novel methods of treating AD and other neurodegenerative diseases through activation of the innate immune system (e.g., microglial activity), for example, using agonistic antibodies that target TREM2.

All references cited herein, including patents, patent applications and publications, are hereby incorporated by reference in their entirety.

### SUMMARY

The present disclosure is generally directed to methods of using compositions that include antibodies, e.g., monoclonal, chimeric, humanized antibodies, antibody fragments, etc., that specifically bind human TREM2.

In one aspect, provided herein is a method of treating and/or delaying the progression of a disease or injury in an individual, comprising administering to the individual an anti-TREM2 antibody at a dose of at least about 15 mg/kg intravenously, wherein the anti-TREM2 antibody is an agonist. In a further aspect, provided herein is a method of treating and/or delaying the progression of a disease or injury in an individual, comprising administering to the individual an anti-TREM2 antibody at a dose of at least about 15 mg/kg intravenously, wherein the antibody comprises a heavy chain variable region comprising an HVR-H1, HVR-H2, and HVR-H3 and a light chain variable region comprising an HVR-L1, HVR-L2, and HVR-L3, and wherein: (i) the HVR-H1 comprises the amino acid sequence YAFSSQWMN (SEQ ID NO: 34), the HVR-H2 comprises the amino acid sequence RIYPGGGDTNYAGKFQG (SEQ ID NO: 35), the HVR-H3 comprises the amino acid sequence ARLLRNQPGESYAMDY (SEQ ID NO: 31), the HVR-L1 comprises the amino acid sequence RSSQSLVHSNRYTYLH (SEQ ID NO: 40), the HVR-L2 comprises the amino acid sequence KVSNRFS (SEQ ID NO: 33), and the HVR-L3 comprises the amino acid sequence SQSTRVPYT (SEQ ID NO: 32); or (ii) the HVR-H1 comprises the amino acid sequence YAFSSDWMN (SEQ ID NO: 36), the HVR-H2 comprises the amino acid sequence RIYPGEGDTNYARKFHG (SEQ ID NO: 37), the HVR-H3 comprises the amino acid sequence ARLLRNKPGESYAMDY (SEQ ID NO: 38), the HVR-L1 comprises the amino acid sequence RTSQSLVHSNAYTYLH (SEQ ID NO: 39), the HVR-L2 comprises the amino acid sequence KVSNRVS (SEQ ID NO: 40), and the HVR-L3 comprises the amino acid sequence SQSTRVPYT (SEQ ID NO: 32).

In some embodiments, the dose is between about 15 mg/kg to about 60 mg/kg. In some embodiments, the dose is about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, or about 60 mg/kg. In some embodiments, the anti-TREM2 antibody is administered about once every four weeks at a dose of at least about 15 mg/kg. In some embodiments, the anti-TREM2 antibody is administered about once every week at a dose of at least about 15 mg/kg. In some embodiments, the anti-TREM2 antibody is administered about once every four weeks at a dose of about 15 mg/kg. In some embodiments, the anti-TREM2 antibody is administered about once every four weeks at a dose of about 20 mg/kg. In some embodiments, the anti-TREM2 antibody is administered about once every four weeks at a dose of about 25 mg/kg. In some embodiments, the anti-TREM2 antibody is administered about once every four weeks at a dose of about 30 mg/kg. In some embodiments, the anti-TREM2 antibody is administered about once every four weeks at a dose of about 35 mg/kg. In some embodiments, the anti-TREM2 antibody is administered about once every four weeks at a dose of about 40 mg/kg. In some embodiments, the anti-TREM2 antibody is administered about once every four weeks at a dose of about 45 mg/kg. In some embodiments, the anti-TREM2 antibody is administered about once every four weeks at a dose of about 50 mg/kg. In some embodiments, the anti-TREM2 antibody is administered about once every four weeks at a dose of about 55 mg/kg. In some embodiments, the anti-TREM2 antibody is administered about once every four weeks at a dose of about 60 mg/kg.

In some embodiments, the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSQWMN (SEQ ID NO: 34), the HVR-H2 comprises the amino acid sequence RIYPGGGDTNYAGKFQG (SEQ ID NO: 35), the HVR-H3 comprises the amino acid sequence ARLLRNQPGESYAMDY (SEQ ID NO: 31), the HVR-L1 comprises the amino acid sequence RSSQSLVHSNRYTYLH (SEQ ID NO: 41), the HVR-L2 comprises the amino acid sequence KVSNRFS (SEQ ID NO: 33), and the HVR-L3 comprises the amino acid sequence SQSTRVPYT (SEQ ID NO: 32). In some embodiments, the antibody comprises a heavy chain variable region comprising the amino acid sequence of SEQ ID NO: 27 and a light chain variable region comprising the amino acid sequence of SEQ ID NO: 30.

In some embodiments, the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSDWMN (SEQ ID NO: 36), the HVR-H2 comprises the amino acid sequence RIYPGEGDTNYARKFHG (SEQ ID NO: 37), the HVR-H3 comprises the amino acid sequence ARLLRNKPGESYAMDY (SEQ ID NO: 38), the HVR-L1 comprises the amino acid sequence RTSQSLVHSNAYTYLH (SEQ ID NO: 39), the HVR-L2 comprises the amino acid sequence KVSNRVS (SEQ ID NO: 40), and the HVR-L3 comprises the amino acid sequence SQSTRVPYT (SEQ ID NO: 32). In some embodiments, the antibody comprises a heavy chain variable region comprising the amino acid sequence of SEQ ID NO: 28 and a light chain variable region comprising the amino acid sequence of SEQ ID NO: 29.

In some embodiments, the antibody has a human IgG1 isotype.

In some embodiments, the antibody has a human IgG1 isotype and comprises amino acid substitutions in the Fc region at the residue positions P331S and E430G, wherein the numbering of the residues is according to EU numbering.

In some embodiments, the antibody comprises (a) a heavy chain comprising the amino acid sequence of SEQ ID NO: 43, and a light chain comprising the amino acid sequence of SEQ ID NO: 47; or (b) a heavy chain comprising the amino acid sequence of SEQ ID NO: 44, and a light chain comprising the amino acid sequence of SEQ ID NO: 47.

In some embodiments, the antibody comprises (a) a heavy chain comprising the amino acid sequence of SEQ ID NO: 45, and a light chain comprising the amino acid sequence of SEQ ID NO: 48; or (b) a heavy chain comprising the amino acid sequence of SEQ ID NO: 46, and a light chain comprising the amino acid sequence of SEQ ID NO: 48.

In some embodiments, the disease or injury is selected from dementia, frontotemporal dementia, Alzheimer's disease, Nasu-Hakola disease, cognitive deficit, memory loss, spinal cord injury, traumatic brain injury, a demyelination disorder, multiple sclerosis, Parkinson's disease, amyotrophic lateral sclerosis (ALS), Huntington's disease, adult-onset leukoencephalopathy with axonal spheroids and pigmented glia (ALSP), or a tauopathy disease.

In some embodiments, the disease or injury is Alzheimer's disease. In some embodiments, the individual has a Mini-Mental State Examination (MMSE) score of between about 16 points to about 28 points prior to administration of the anti-TREM2 antibody. In some embodiments, the individual has a Clinical Dementia Rating-Global Score (CDR-GS) of 0.5, 1.0, or 2.0 prior to administration of the anti-TREM2 antibody. In some embodiments, the individual has a positive amyloid-PET scan prior to administration of the anti-TREM2 antibody. In some embodiments, the individual is being administered a cholinesterase inhibitor and/or memantine therapy. In some embodiments, the individual has symptoms of Alzheimer's disease prior to administration of the anti-TREM2 antibody. In some embodiments, the symptoms are mild cognitive impairment and/or mild dementia. In some embodiments, the individual does not have symptoms of Alzheimer's disease prior to administration of the anti-TREM2 antibody.

In some embodiments, the individual is heterozygous or homozygous for a mutation in TREM2. In some embodiments, the individual comprises an amino acid substitution in a human TREM2 protein at residue position R47H, R62H, or both.

In some embodiments, the individual has a positive amyloid or tau blood test prior to administration of the anti-TREM2 antibody.

In some embodiments, administration of the anti-TREM2 antibody results in at least about a 30% decrease in the levels of soluble TREM2 in the cerebrospinal fluid of the individual, compared to the levels of soluble TREM2 in the cerebrospinal fluid of the individual prior to administration of the anti-TREM2 antibody. In some embodiments, administration of the anti-TREM2 antibody results in at least about a 40% decrease in the levels of soluble TREM2 in the cerebrospinal fluid of the individual, compared to the levels of soluble TREM2 in the cerebrospinal fluid of the individual prior to administration of the anti-TREM2 antibody. In some embodiments, the decrease in the levels of soluble TREM2 in the cerebrospinal fluid of the individual is present at about 2 days after administration of the anti-TREM2 antibody. In some embodiments, the levels of soluble TREM2 in the cerebrospinal fluid of the individual are measured in a sample of cerebrospinal fluid obtained from the individual using an electrochemiluminescent assay.

In some embodiments, administration of the anti-TREM2 antibody results in at least about a 5% increase in the levels of soluble CSF1R in the cerebrospinal fluid of the individual, compared to the levels of soluble CSF1R in the cerebrospinal fluid of the individual prior to administration of the anti-TREM2 antibody. In some embodiments, the increase in the levels of soluble CSF1R in the cerebrospinal fluid of the individual is present at about 2 days after administration of the anti-TREM2 antibody. In some embodiments, the levels of soluble CSF1R in the cerebrospinal fluid of the individual are measured in a sample of cerebrospinal fluid obtained from the individual using an ELISA assay.

In some embodiments, the methods provided herein also comprise measuring the levels of soluble TREM2 in a sample of blood, plasma, and/or cerebrospinal fluid from the individual before and after the individual has received one or more doses of the anti-TREM2 antibody. In some embodiments, the methods provided herein also comprise measuring the levels of soluble CSF1R in a sample of blood, plasma, and/or cerebrospinal fluid from the individual before and after the individual has received one or more doses of the anti-TREM2 antibody.

In some embodiments, the methods provided herein also comprise measuring the levels of brain amyloid burden in the brain of the individual before and after the individual has received one or more doses of the anti-TREM2 antibody. In some embodiments, the levels of brain amyloid burden in the brain of the individual are measured using amyloid-positron emission tomography. In some embodiments, the methods provided herein also comprise measuring tau burden in the brain of the individual, assessed by measuring the levels of tau in the brain of the individual before and after the individual has received one or more doses of the anti-TREM2 antibody. In some embodiments, the levels of tau in the brain of the individual are measured using tau-positron emission tomography. In some embodiments, the methods provided herein also comprise measuring one or more brain abnormalities in the brain of the individual before and after the individual has received one or more doses of the anti-TREM2 antibody. In some embodiments, the one or more brain abnormalities are measured using magnetic resonance imaging. In some embodiments, the one or more brain abnormalities is brain volume. In some embodiments, the methods provided herein also comprise measuring brain volume of the individual before and after the individual has received one or more doses of the anti-TREM2 antibody. In some embodiments, brain volume is measured using magnetic resonance imaging. In some embodiments, brain volume is measured using volumetric magnetic resonance imaging.

In some embodiments, the methods provided herein also comprise detecting the presence of an alteration in one or more genes in the individual selected from APOE, TREM2, CD33, TMEM106b, or CLUSTERIN.

In some embodiments, the methods provided herein also comprise measuring the levels of one or more biomarkers of neuroinflammation in a sample of blood, plasma, and/or cerebrospinal fluid from the individual before and after the individual has received one or more doses of the anti-TREM2 antibody.

In some embodiments, the methods provided herein also comprise measuring the levels of one or more biomarkers of neurodegeneration in a sample of blood, plasma, and/or cerebrospinal fluid from the individual before and after the individual has received one or more doses of the anti-TREM2 antibody.

In some embodiments, the one or more biomarkers of neurodegeneration is neurofilament light.

In some embodiments, the methods provided herein also comprise measuring the expression levels of TREM2, CSF1R, YKL40, IL-1RA, or osteopontin in a sample of blood, plasma, and/or cerebrospinal fluid from the individual before and after the individual has received one or more doses of the anti-TREM2 antibody. In some embodiments, the expression levels of TREM2, CSF1R, YKL40, IL-1RA, or osteopontin refer to mRNA expression levels. In some embodiments, the expression levels of TREM2, CSF1R, YKL40, IL-1RA, or osteopontin refer to protein expression levels. In some embodiments, the method comprises measuring protein expression levels of sTREM2 or sCSF1R in the sample of blood, plasma, and/or cerebrospinal fluid from the individual before and after the individual has received one or more doses of the anti-TREM2 antibody.

In some embodiments, the methods provided herein also comprise measuring the levels of one or more biomarkers of Alzheimer's disease in a sample of cerebrospinal fluid from the individual before and after the individual has received one or more doses of the anti-TREM2 antibody. In some embodiments, the methods provided herein also comprise measuring the levels of one or more biomarkers of Alzheimer's disease in a sample of blood from the individual before and after the individual has received one or more doses of the anti-TREM2 antibody. In some embodiments, the methods provided herein also comprise measuring the levels of one or more biomarkers of Alzheimer's disease in a sample of plasma from the individual before and after the individual has received one or more doses of the anti-TREM2 antibody. In some embodiments, the one or more biomarkers of Alzheimer's disease are selected from Aβ42, Aβ40, total tau, pTau, neurofilament light, or any combination thereof. In some embodiments, the one or more biomarkers of Alzheimer's disease are Aβ40, Aβ42, pTau, and/or total tau.

In some embodiments, the methods provided herein also comprise measuring the levels of one or more biomarkers of microglia function in a sample of cerebrospinal fluid from the individual before and after the individual has received one or more doses of the anti-TREM2 antibody. In some embodiments, the methods provided herein also comprise measuring the levels of one or more biomarkers of microglia function in a sample of blood from the individual before and after the individual has received one or more doses of the anti-TREM2 antibody. In some embodiments, the methods provided herein also comprise measuring the levels of one or more biomarkers of microglia function in a sample of plasma from the individual before and after the individual has received one or more doses of the anti-TREM2 antibody. In some embodiments, the one or more biomarkers of microglia function are CSF1R, IL1RN, YKL40 and/or osteopontin.

In some embodiments, the methods provided herein also comprise determining a score of one or more clinical assessments of the individual before and after the individual has received one or more doses of the anti-TREM2 antibody, wherein the one or more clinical assessments are selected from the Mini-Mental State Examination (MMSE) score, the Clinical Dementia Rating-Global Score (CDR-GS), the Clinical Dementia Rating Sum of Boxes (CDR-SB), or the Repeatable Battery for the Assessment of Neuropsychological Status (RBANS).

In some embodiments, the methods provided herein also comprise performing tau or amyloid positron emission tomography (PET) imaging assessments in the individual before and after the individual has received one or more doses of the anti-TREM2 antibody.

In some embodiments, the disease or injury is Alzheimer's disease, and wherein the Alzheimer's disease is early Alzheimer's disease. In some embodiments, the individual has brain amyloidosis prior to administration of the anti-TREM2 antibody, wherein brain amyloidosis is assessed in a sample of cerebrospinal fluid obtained from the individual, or by positron emission tomography (PET). In some embodiments, the individual has a Mini-Mental State Examination (MMSE) score of at least about 22 points prior to administration of the anti-TREM2 antibody. In some embodiments, the individual has a Clinical Dementia Rating-Global Score (CDR-GS) of between about 0.5 and about 1.0 prior to administration of the anti-TREM2 antibody. In some embodiments, the individual has a Repeatable Battery for the Assessment of Neuropsychological Status on the Delayed Memory Index (RBANS DMI) score of 85 or less prior to administration of the anti-TREM2 antibody. In some embodiments, the individual has a positive amyloid or tau blood test prior to administration of the anti-TREM2 antibody. In some embodiments, the methods provided herein also comprise determining a score of one or more clinical assessments of the individual before and after the individual has received one or more doses of the anti-TREM2 antibody, wherein the one or more clinical assessments are selected from the Clinical Dementia Rating Sum of Boxes (CDR-SB), the Mini-Mental State Examination (MMSE), the Repeatable Battery for the Assessment of Neuropsychological Status (RBANS), the Alzheimer's Disease Assessment Scale-Cognitive Subscale-13 (ADAS-Cog13), the Alzheimer's Disease Cooperative Study-Activities of Daily Living adapted to Mild Cognitive Impairment (ADCS-ADL-MCI), and the Alzheimer's Disease Composite Score (ADCOMS). In some embodiments, the methods provided herein also comprise measuring the levels of one or more biomarkers of Alzheimer's disease, including but not limited to any of the biomarkers described herein, before and after the individual has received one or more doses of the anti-TREM2 antibody, wherein the one or more biomarkers of Alzheimer's disease are measured by magnetic resonance imaging (MRI), or in a sample of blood, plasma or cerebrospinal fluid obtained from the individual. In some embodiments, the methods provided herein also comprise performing tau or amyloid positron emission tomography (PET) imaging assessments in the individual before and after the individual has received one or more doses of the anti-TREM2 antibody. In some embodiments, the methods provided herein also comprise performing one or more speech assessments in the individual before and after the individual has received one or more doses of the anti-TREM2 antibody.

In some embodiments of the methods provided herein, (a) the dose is about 15 mg/kg and the terminal half-life of the antibody in the plasma of the individual is about 8.63 days; (b) the dose is about 30 mg/kg and the terminal half-life of the antibody in the plasma of the individual is about 7.44 days; (c) the dose is about 45 mg/kg and the terminal half-life of the antibody in the plasma of the individual is about 8.40 days; or (d) the dose is about 60 mg/kg and the terminal half-life of the antibody in the plasma of the individual is about 9.93 days.

In some embodiments of the methods provided herein, the method further comprises performing an amyloid or tau blood test on a sample obtained from an individual before and after the individual has received one or more doses of the anti-TREM2 antibody.

In another aspect, provided herein is a method of monitoring the treatment of an individual being administered an anti-TREM2 antibody, comprising measuring the levels of soluble TREM2 in a sample of cerebrospinal fluid from the individual before and after the individual has received one or more doses of an anti-TREM2 antibody. In some embodiments, the method of monitoring treatment provided herein also comprises a step of assessing the activity of the anti-TREM2 antibody in the individual based on the levels of soluble TREM2 in the sample of cerebrospinal fluid. In some embodiments, the anti-TREM2 antibody is determined to be active in the individual if the levels of soluble TREM2 in the sample of cerebrospinal fluid are decreased after the individual has received one or more doses of the anti-TREM2 antibody, compared to the levels of soluble TREM2 in the sample of cerebrospinal fluid before the individual received a dose of the anti-TREM2 antibody.

In another aspect, provided herein is a method of monitoring the treatment of an individual being administered an anti-TREM2 antibody, comprising measuring the levels of soluble TREM2 in a sample of blood or plasma from the individual before and after the individual has received one or more doses of an anti-TREM2 antibody. In some embodiments, the method of monitoring treatment provided herein also comprises a step of assessing the activity of the anti-TREM2 antibody in the individual based on the levels of soluble TREM2 in the sample of blood or plasma. In some embodiments, the anti-TREM2 antibody is determined to be active in the individual if the levels of soluble TREM2 in the sample of blood or plasma are decreased after the individual has received one or more doses of the anti-TREM2 antibody, compared to the levels of soluble TREM2 in the sample of blood or plasma before the individual received a dose of the anti-TREM2 antibody.

In another aspect, provided herein is a method of monitoring the treatment of an individual being administered an anti-TREM2 antibody, comprising measuring the levels of soluble CSF1R in a sample of cerebrospinal fluid, blood, or plasma from the individual before and after the individual has received one or more doses of an anti-TREM2 antibody. In some embodiments, the method of monitoring treatment provided herein also comprises a step of assessing the activity of the anti-TREM2 antibody in the individual based on the levels of soluble CSF1R in the sample of cerebrospinal fluid, blood, or plasma. In some embodiments, the anti-TREM2 antibody is determined to be active in the individual if the levels of soluble CSF1R in the sample of cerebrospinal fluid, blood, or plasma are increased after the individual has received one or more doses of the anti-TREM2 antibody, compared to the levels of soluble CSF1R in the sample of cerebrospinal fluid, blood, or plasma before the individual received a dose of the anti-TREM2 antibody.

In another aspect, provided herein is a method of monitoring the treatment of an individual being administered an anti-TREM2 antibody, comprising measuring the levels of YKL40 in a sample of cerebrospinal fluid, blood, or plasma from the individual before and after the individual has received one or more doses of an anti-TREM2 antibody. In some embodiments, the method of monitoring treatment provided herein also comprises a step of assessing the activity of the anti-TREM2 antibody in the individual based on the levels of YKL40 in the sample of cerebrospinal fluid, blood, or plasma. In some embodiments, the anti-TREM2 antibody is determined to be active in the individual if the levels of YKL40 in the sample of cerebrospinal fluid, blood, or plasma are increased after the individual has received one or more doses of the anti-TREM2 antibody, compared to the levels of YKL40 in the sample of cerebrospinal fluid, blood, or plasma before the individual received a dose of the anti-TREM2 antibody.

In another aspect, provided herein is a method of monitoring the treatment of an individual being administered an anti-TREM2 antibody, comprising measuring the levels of IL-1RA in a sample of cerebrospinal fluid, blood, or plasma from the individual before and after the individual has received one or more doses of an anti-TREM2 antibody. In some embodiments, the method of monitoring treatment provided herein also comprises a step of assessing the activity of the anti-TREM2 antibody in the individual based on the levels of IL-1RA in the sample of cerebrospinal fluid, blood, or plasma. In some embodiments, the anti-TREM2 antibody is determined to be active in the individual if the levels of IL-1RA in the sample of cerebrospinal fluid, blood, or plasma are increased after the individual has received one or more doses of the anti-TREM2 antibody, compared to the levels of IL-1RA in the sample of cerebrospinal fluid, blood, or plasma before the individual received a dose of the anti-TREM2 antibody.

In another aspect, provided herein is a method of monitoring the treatment of an individual being administered an anti-TREM2 antibody, comprising measuring the levels of osteopontin in a sample of cerebrospinal fluid, blood, or plasma from the individual before and after the individual has received one or more doses of an anti-TREM2 antibody. In some embodiments, the method of monitoring treatment provided herein also comprises a step of assessing the activity of the anti-TREM2 antibody in the individual based on the levels of osteopontin in the sample of cerebrospinal fluid, blood, or plasma. In some embodiments, the anti-TREM2 antibody is determined to be active in the individual if the levels of osteopontin in the sample of cerebrospinal fluid, blood, or plasma are increased after the individual has received one or more doses of the anti-TREM2 antibody, compared to the levels of osteopontin in the sample of cerebrospinal fluid, blood, or plasma before the individual received a dose of the anti-TREM2 antibody.

In another aspect, provided herein is a method of monitoring the treatment of an individual being administered an anti-TREM2 antibody, comprising measuring the levels of one or more biomarkers of Alzheimer's disease in a sample of cerebrospinal fluid, plasma, or blood from the individual before and after the individual has received one or more doses of an anti-TREM2 antibody. In some embodiments, the method of monitoring treatment provided herein also comprises a step of assessing the activity of the anti-TREM2 antibody in the individual based on the levels of the one or more biomarkers of Alzheimer's disease in the sample of cerebrospinal fluid, plasma, or blood. In some embodiments, the one or more biomarkers of Alzheimer's disease are selected from Aβ42, Aβ40, total tau, pTau, neurofilament light, or any combination thereof.

In another aspect, provided herein is a method of monitoring the treatment of an individual being administered an anti-TREM2 antibody, comprising measuring the levels of one or more biomarkers of microglia function in a sample of cerebrospinal fluid, plasma, or blood from the individual before and after the individual has received one or more doses of an anti-TREM2 antibody. In some embodiments, the method of monitoring treatment provided herein also comprises a step of assessing the activity of the anti-TREM2 antibody in the individual based on the levels of the one or more biomarkers of microglia function in the sample of cerebrospinal fluid, plasma, or blood. In some embodiments, the one or more biomarkers of microglia function are CSF1R, IL1RN, YKL40, and/or osteopontin.

In another aspect, provided herein is a method of monitoring the treatment of an individual being administered an anti-TREM2 antibody, comprising measuring the levels of one or more biomarkers of neurodegeneration in a sample of cerebrospinal fluid, plasma, or blood from the individual before and after the individual has received one or more doses of an anti-TREM2 antibody. In some embodiments, the method of monitoring treatment provided herein also comprises a step of assessing the activity of the anti-TREM2 antibody in the individual based on the levels of the one or more biomarkers of neurodegeneration in the sample of cerebrospinal fluid, plasma, or blood. In some embodiments, the one or more biomarkers of neurodegeneration comprise NfL.

In another aspect, provided herein is a method of monitoring the treatment of an individual being administered an anti-TREM2 antibody, comprising measuring the expression levels of TREM2, CSF1R, YKL40, IL-1RA, or osteopontin in a sample of cerebrospinal fluid, plasma, or blood from the individual before and after the individual has received one or more doses of an anti-TREM2 antibody. In some embodiments, the method of monitoring treatment provided herein also comprises a step of assessing the activity of the anti-TREM2 antibody in the individual based on the expression levels of TREM2, CSF1R, YKL40, IL-1RA, or osteopontin in the sample of cerebrospinal fluid, plasma, or blood. In some embodiments, the expression levels of TREM2, CSF1R, YKL40, IL-1RA, or osteopontin refer to mRNA expression levels. In some embodiments, the expression levels of TREM2, CSF1R, YKL40, IL-1RA, or osteopontin refer to protein expression levels. In some embodiments, the method comprises measuring the protein expression levels of sTREM2 or sCSF1R in the sample of cerebrospinal fluid, plasma, or blood from the individual before and after the individual has received one or more doses of the anti-TREM2 antibody.

In another aspect, provided herein is a method of monitoring the treatment of an individual being administered an anti-TREM2 antibody, comprising measuring the levels of amyloid burden in the brain of the individual before and after the individual has received one or more doses of an anti-TREM2 antibody. In some embodiments, the method of monitoring treatment provided herein also comprises a step of assessing the activity of the anti-TREM2 antibody in the individual based on the levels of amyloid burden in the brain of the individual.

In another aspect, provided herein is a method of monitoring the treatment of an individual being administered an anti-TREM2 antibody, comprising measuring tau burden in the brain of the individual, assessed by measuring the levels of tau in the brain of the individual, before and after the individual has received one or more doses of an anti-TREM2 antibody. In some embodiments, the method of monitoring treatment provided herein also comprises a step of assessing the activity of the anti-TREM2 antibody in the individual based on the levels of tau in the brain of the individual.

In another aspect, provided herein is a method of monitoring the treatment of an individual being administered an anti-TREM2 antibody, comprising measuring brain volume of the individual before and after the individual has received one or more doses of an anti-TREM2 antibody. In some embodiments, the method of monitoring treatment provided herein also comprises a step of assessing the activity of the anti-TREM2 antibody in the individual based on the brain volume of the individual.

In any of the foregoing methods of monitoring treatment, the anti-TREM2 antibody is an agonist.

### BRIEF DESCRIPTION OF THE DRAWINGS

FIG. 1 shows a diagram of the Phase 1 study described in Example 1 assessing the safety, tolerability, pharmacokinetics (PK), and pharmacodynamics (PD) of AT.1FM when administered as single ascending doses in healthy participants and as multiple doses in participants with mild to moderate AD. SAD=single ascending dose; MD=multiple dose. The arrows indicate administration of AT.1FM at the indicated doses. The ratio of participants administered active drug (AT.1FM) to participants administered placebo is provided for each cohort (active drug:placebo). The asterisk (*) indicates that lumbar punctures are performed to obtain a cerebrospinal fluid (CSF) baseline sample (SAD Cohorts F, G, H, I, K and N; MD Cohorts J, L and M). The plus sign (+) indicates open-label cohorts.

FIG. 2 provides safety results for the SAD phase of the Phase 1 study described in Examples 1 and 2. TEAE=treatment emergent adverse event; SAE=serious adverse event; “disc.”=discontinuation. The asterisk (*) indicates a traumatic injury event unrelated to study treatment.

FIGS. 3A-3B show results of experiments that assessed the effect of AT.1FM on the levels of soluble TREM2 (sTREM2) and soluble CSF1R (sCSF1R) in the cerebrospinal fluid (CSF) of participants in the SAD phase of the study described in Examples 1 and 2. FIG. 3A shows the percent change in the levels of sTREM2 in CSF two days after administration of the indicated dose of AT.1FM or placebo compared to baseline sTREM2 levels in CSF. FIG. 3B shows the percent change in the levels of sCSF1R in CSF two days after administration of the indicated dose of AT.1FM or placebo compared to baseline sCSF1R levels in CSF.

FIG. 4 shows the levels of sTREM2 in the CSF of healthy human volunteers administered AT.1FM or placebo. The percent change from baseline (mean±standard deviation) of sTREM2 levels in CSF is provided at day 2 (D2) and day 12 (D12) after administration of the indicated dose of AT.1FM or placebo.

FIG. 5 shows the levels of soluble CSF1R (sCSF1R) in the CSF of healthy human volunteers administered AT.1FM or placebo. The percent change from baseline (mean±standard deviation) of sCSF1R levels in CSF is provided at day 2 (D2) and day 12 (D12) after administration of the indicated dose of AT.1FM or placebo.

FIG. 6 shows the levels of YKL40 in the CSF of healthy human volunteers administered AT.1FM or placebo. The percent change from baseline (mean±standard deviation) of YKL40 levels in CSF is provided at day 2 and day 12 after administration of the indicated dose of AT.1FM or placebo.

FIG. 7 shows the levels of IL-1RA in the CSF of healthy human volunteers administered AT.1FM or placebo. The percent change from baseline (mean±standard deviation) of IL-1RA levels in CSF is provided at day 2 and day 12 after administration of the indicated dose of AT.1FM or placebo.

FIG. 8 shows the levels of osteopontin (OPN) in the CSF of healthy human volunteers administered AT.1FM or placebo. The percent change from baseline (mean±standard deviation) of OPN levels in CSF is provided at day 2 and day 12 after administration of the indicated dose of AT.1FM or placebo.

FIG. 9 shows the concentration of AT.1FM in the CSF of healthy human volunteers. The concentration of AT.1FM in CSF (ng/mL; mean+standard deviation) is provided at day 2 and day 12 after administration of the indicated dose of AT.1FM.

FIGS. 10A-10B show the concentration of TREM2 protein in the frontal cortex and hippocampus of non-human primates administered AT.1FM or control. The non-human primates were administered control or AT.1FM at the indicated doses intravenously once weekly for a total of 5 doses. FIG. 10A shows the concentration of TREM2 protein in the frontal cortex at 48 hours after the fifth administration of AT.1FM or control (mean±standard error of the mean). FIG. 10B shows the concentration of TREM2 protein in the hippocampus at 48 hours after the fifth administration of AT.1FM or control (mean±standard error of the mean). In FIGS. 10A-10B, measurements were normalized to tissue protein concentration (N=6 per dose group, *, p<0.05; ***, p<0.001; ****, p<0.0001 by one-way ANOVA).

FIG. 11 shows the levels of sTREM2 in the CSF of non-human primates administered AT.1FM or control once per week for 3 weeks. The levels of sTREM2 in CSF (percent of baseline; mean±standard error of the mean) are provided at the indicated times (hours) after the first administration of control or AT.1FM. The arrows indicate the time of administration of control or AT.1FM.

FIG. 12 shows the levels of osteopontin in the CSF of non-human primates administered AT.1FM or control once per month for 2 months for a total of three doses (N=5 per group). The levels of osteopontin in CSF (percent of baseline; mean±standard error of the mean) are provided at the indicated times (hours) after the first administration of control or AT.1FM. The arrows indicate the time of administration of control or AT.1FM at 250 mg/kg. The gray dashed line denotes the baseline (pre-dose) level of osteopontin.

FIG. 13 shows the concentration of CSF1R protein in the frontal cortex and hippocampus of non-human primates administered AT.1F or control. The non-human primates were administered control or AT.1F intravenously at a dose of 80 mg/kg once weekly for a total of 5 doses (N=5 per group). The concentrations of CSF1R protein (ng CSF1R protein/mg total protein) in the frontal cortex (left panel) and in the hippocampus (right panel) at 48 hours after the fifth administration of AT.1F or control are provided.

### DETAILED DESCRIPTION

Provided herein are methods of treating or delaying the progressing of a disorder or injury by administering an agonist of TREM2. Such diseases or injuries include dementia, frontotemporal dementia, Alzheimer's disease, Nasu-Hakola disease, cognitive deficit, memory loss, spinal cord injury, traumatic brain injury, demyelination disorders, multiple sclerosis, Parkinson's disease, amyotrophic lateral sclerosis (ALS), Huntington's disease, adult-onset leukoencephalopathy with axonal spheroids and pigmented glia (ALSP), and tauopathy diseases. Agonists of TREM2 include anti-TREM2 antibodies that induce or increase one or more TREM2 activities, and/or enhance one or more activities induced by binding of one or more ligands to TREM2. For example, agonist anti-TREM2 antibodies may decrease soluble TREM2, induce spleen tyrosine kinase (Syk) phosphorylation, induce binding of TREM2 to DAP12, induce DAP12 phosphorylation, increase the proliferation, survival, and/or function of dendritic cells, macrophages, monocytes, osteoclasts, Langerhans cells of skin, Kupffer cells, and microglial cells (microglia), or increase the activity and/or expression of TREM2-dependent genes.

### Definitions

As used herein, the term “preventing” includes providing prophylaxis with respect to occurrence or recurrence of a particular disease, disorder, or condition, including delaying onset of a particular disease, disorder, or condition, in an individual that may be predisposed to, susceptible to, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W.B. Saunders Co., 2000).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

An “isolated” antibody, such as an isolated anti-TREM2 antibody of the present disclosure, is one that has been identified, separated and/or recovered from a component of its production environment (e.g., naturally or recombinantly). Preferably, the isolated polypeptide is free from association with substantially all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain.

The “variable region” or “variable domain” of an antibody, such as an anti-TREM2 antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-TREM2 antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular cytotoxicity.

The term “monoclonal antibody” as used herein refers to an antibody, such as a monoclonal anti-TREM2 antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, monoclonal antibodies are advantageous in that they may be synthesized by a hybridoma culture, substantially uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5):1073-1093 (2004); Fellouse, Proc. Nat'l Acad. Sci. USA 101(34):12467-472 (2004); and Lee et al., J. Immunol. Methods 284(1-2):119-132 (2004), yeast presentation technologies (see, e.g., WO2009/036379A2; WO2010105256; WO2012009568, and Xu et al., Protein Eng. Des. Sel., 26(10): 663-70 (2013), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The terms “full-length antibody,” “intact antibody” or “whole antibody” are used interchangeably to refer to an antibody, such as an anti-TREM2 antibody of the present disclosure, in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-TREM2 antibodies of the present disclosure, produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments that are capable of binding and cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments may be produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which is recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) may have the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains, which enables the sFv to form the desired structure for antigen binding. For a review of the sFv, see Plückthun in The Pharmacology of Monoclonal Antibodies, vol. 113, Rosenburg and Moore eds., Springer-VerLAG-3, New York, pp. 269-315 (1994).

“Functional fragments” of antibodies, such as anti-TREM2 antibodies of the present disclosure, comprise a portion of an intact antibody, generally including the antigen binding or variable region of the intact antibody or the Fc region of an antibody which retains or has modified FcR binding capability.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the variable domains is achieved, thereby resulting in a bivalent fragment, i.e., a fragment having two antigen-binding sites. Diabodies are described in greater detail in, for example, EP 404,097; WO 93/11161; Hollinger et al., Proc. Nat'l Acad. Sci. USA 90:6444-48 (1993).

As used herein, a “chimeric antibody” refers to an antibody, such as a chimeric anti-TREM2 antibody of the present disclosure, in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies include antibodies in which the variable region of the antibody is derived from a murine antibody, and the constant region is derived from a human antibody. As used herein, “humanized antibody” is a subset of “chimeric antibodies.”

“Humanized” forms of non-human (e.g., murine) antibodies, such as humanized forms of anti-TREM2 antibodies of the present disclosure, are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody, such as an anti-TREM2 antibody of the present disclosure, that has been made using any of the techniques for making human antibodies as disclosed herein or otherwise known in the art. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology. Alternatively, human antibodies can also be prepared by employing yeast libraries and methods as disclosed in, for example, WO2009/036379A2; WO2010105256; WO2012009568; and Xu et al., Protein Eng. Des. Sel., 26(10): 663-70 (2013).

The term “hypervariable region” or “HVR” when used herein refers to the regions of an antibody-variable domain, such as that of an anti-TREM2 antibody of the present disclosure, that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable domain residues are numbered according to Kabat et al., supra, for each of these extended HVR definitions.

“Framework” or “FR” residues are those variable domain residues other than the HVR residues as herein defined.

The phrase “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system,” “EU numbering” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. References to residue numbers in the variable domain of antibodies means residue numbering by the Kabat numbering system. References to residue numbers in the constant domain of antibodies means residue numbering by the EU numbering system (e.g., see United States Patent Publication No. 2010-280227).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferably those changes occur at only three, two, or one of positions 71H, 73H and 78H; for instance, the amino acid residues at those positions may by 71A, 73T and/or 78A. In one embodiment, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, Md. (1991). For the VL, the subgroup may be, e.g., subgroup kappa I, kappa II, kappa III or kappa IV as in Kabat et al., supra. Additionally, for the VH, the subgroup may be, e.g., subgroup I, subgroup II, or subgroup III as in Kabat et al., supra.

An “amino acid modification” at a specified position, e.g., of an anti-TREM2 antibody of the present disclosure, refers to the substitution or deletion of the specified residue, or the insertion of at least one amino acid residue adjacent to the specified residue. Insertion “adjacent” to a specified residue means insertion within one to two residues thereof. The insertion may be N-terminal or C-terminal to the specified residue. The preferred amino acid modification herein is a substitution.

An “affinity matured” antibody, such as an affinity matured anti-TREM2 antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity matured antibodies may be produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As used herein, the term “specifically binds” or “specifically recognizes” refers to measurable and reproducible binding interactions between a target and an antibody, such as between an anti-TREM2 antibody and TREM2, that is determinative of the presence of the target within a heterogeneous population of molecules, such as biological molecules. For example, an antibody, such as an anti-TREM2 antibody of the present disclosure, that specifically binds to a target or an epitope of the target is an antibody that preferentially binds this target or epitope, e.g., with greater affinity or avidity, than it binds to other unrelated targets or epitopes. It is also understood that an antibody that specifically binds to a first target may or may not specifically bind to a second target. As such, “specific binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻¹, in other instances about 10⁶ M⁻¹ or 10⁷ M⁻¹, about 10⁸ M⁻¹ to 10⁹ M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, or Vashist and Luong (2018) Handbook of Immunoassay Technologies, Approaches, Performances, and Applications, Academic Press, for a description of immunoassay formats and conditions that can be used to determine specific immunoreactivity.

As used herein, an antibody “inhibits interaction” between two proteins when the antibody disrupts, reduces, or completely eliminates an interaction between the two proteins by binding to one of the two proteins.

An “agonist” antibody is an antibody that induces (e.g., increases) one or more activities or functions of a target upon binding to the target.

An “antagonist” antibody or a “blocking” antibody is an antibody that reduces or eliminates (e.g., decreases) antigen binding to one or more binding partners after the antibody binds the antigen, and/or that reduces or eliminates (e.g., decreases) one or more activities or functions of the antigen after the antibody binds the antigen. In some embodiments, antagonist antibodies, or blocking antibodies substantially or completely inhibit antigen binding to one or more binding partners and/or one or more activities or functions of the antigen.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region, e.g. from about one to about ten amino acid substitutions, and preferably from about one to about five amino acid substitutions in a native sequence Fc region. The variant Fc region herein will preferably possess at least about 80% homology with a native sequence Fc region, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (see, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs are encompassed by the term “FcR” herein. FcRs can also increase the serum half-life of antibodies.

Binding to FcR in vivo and serum half-life of human FcR high-affinity binding polypeptides can be assayed, e.g., in transgenic mice or transfected human cell lines expressing human FcR, or in primates to which the polypeptides having a variant Fc region are administered. WO 2004/42072 (Presta) describes antibody variants with improved or diminished binding to FcRs. See also, e.g., Shields et al., J. Biol. Chem. 9(2):6591-6604 (2001).

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

An “isolated” nucleic acid molecule, e.g., encoding an antibody such as an anti-TREM2 antibody of the present disclosure, is a nucleic acid molecule that is identified and separated from at least one contaminant nucleic acid molecule with which it is ordinarily associated in the environment in which it was produced. Preferably, the isolated nucleic acid is free of association with substantially all components associated with the production environment. The isolated nucleic acid molecules encoding the polypeptides and antibodies herein are distinguished from nucleic acid existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps”; substitution of one or more of the naturally occurring nucleotides with an analog; and internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “host cell” includes an individual cell or cell culture that can contain or contains a vector(s) or other exogenous nucleic acid, e.g., that incorporates a polynucleotide insert(s). In some embodiments, the vector or other exogenous nucleic acid is incorporated into the genome of the host cell. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The terms “TREM2”, “TREM2 protein”, or “TREM2 polypeptide” are used interchangeably herein to refer to any native TREM2 from any mammalian source, including primates (e.g., humans and cynomolgus monkeys) and rodents (e.g., mice and rats), unless otherwise indicated. In some embodiments, the term encompasses both wild-type sequences and naturally occurring variant sequences, e.g., splice variants or allelic variants. In some embodiments, the term encompasses “full-length,” unprocessed TREM2, as well as any form of TREM2 that results from processing in the cell (e.g., soluble TREM2). In some embodiments, the TREM2 is human TREM2. In some embodiments, the amino acid sequence of an exemplary human TREM2 is SEQ ID NO: 1.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the present disclosure described herein include “comprising,” “consisting,” and “consisting essentially of” aspects and embodiments.

### Overview

The present disclosure relates to methods of treating or delaying the progressing of a disorder or injury by administering an agonist of TREM2. Agonists of TREM2 include anti-TREM2 antibodies that induce or increase one or more TREM2 activities and/or enhance one or more activities induced by binding of one or more ligands to TREM2. For example, agonist anti-TREM2 antibodies may decrease soluble TREM2, induce spleen tyrosine kinase (Syk) phosphorylation, induce binding of TREM2 to DAP12, induce DAP12 phosphorylation, increase the proliferation, survival, and/or function of dendritic cells, macrophages, monocytes, osteoclasts, Langerhans cells of skin, Kupffer cells, and microglial cells (microglia), or increase the activity and/or expression of TREM2-dependent genes. Without wishing to be bound by theory, it is believed that agonizing TREM2 (e.g., by administering an anti-TREM2 antibody of the disclosure) may promote or increase microglial activity in the individual, resulting in an improvement in the pathology and/or in one or more symptoms of dementia, frontotemporal dementia, Alzheimer's disease, Nasu-Hakola disease, cognitive deficit, memory loss, spinal cord injury, traumatic brain injury, a demyelination disorder, multiple sclerosis, Parkinson's disease, amyotrophic lateral sclerosis (ALS), Huntington's disease, adult-onset leukoencephalopathy with axonal spheroids and pigmented glia (ALSP), or a tauopathy disease. Accordingly, as described below, the methods of the present disclosure meet the need in the art for identifying methods of treating patients with agonizing anti-TREM2 antibodies.

An analysis of the terminal half-life of an anti-TREM2 antibody of the disclosure in the plasma of healthy humans showed that, at the doses tested, the anti-TREM2 antibody unexpectedly exhibited a short terminal half-life compared to other antibodies of a similar class (Ovacik, M and Lin, L, (2018) Clin Transl Sci 11, 540-552) (see, e.g., Example 4). The relatively short terminal half-life of the anti-TREM2 antibody suggested that the antibody may not have a sufficiently robust therapeutic efficacy.

Advantageously, intravenous administration of a single dose of the anti-TREM2 antibody (see, e.g., Example 1) resulted in a decrease in the levels of soluble TREM2 (e.g., at least about a 10% decrease) and an increase in the levels of soluble CSF1R (e.g., at least about a 5% increase) in the cerebrospinal fluid of healthy humans (see, e.g., Examples 2-3). These results indicated that the anti-TREM2 antibody engaged its target (i.e., TREM2) in the individuals. Additional analyses of the anti-TREM2 antibody in the cerebrospinal fluid of healthy humans showed that the antibody was present in cerebrospinal fluid at 12 days after administration of the antibody at the doses tested (see, e.g., Example 5). Furthermore, measurements of biomarkers of microglial activation revealed, unexpectedly, that administration of the anti-TREM2 antibody resulted in an increase in the levels of soluble CSF1R, YKL40a, IL-1RA, and osteopontin in the CSF of healthy humans administered the anti-TREM2 antibody (see, e.g., Example 3). These results suggested that the anti-TREM2 antibody promoted activation of microglia subsequent to target engagement.

Thus, while the anti-TREM2 antibody exhibited a relatively short half-life and thus may not be expected to have a sufficiently robust therapeutic efficacy, the anti-TREM2 antibody unexpectedly exhibited relatively long-lasting pharmacodynamic (PD) effects that, in some cases, were present at 12 days after administration of the antibody (e.g., a decrease in the levels of soluble TREM2, and increases in the levels of soluble CSF1R, YKL40a, IL-1RA, and osteopontin in cerebrospinal fluid) (see, e.g., Examples 2-3).

Advantageously, administration of multiple doses of the anti-TREM2 antibody to non-human primates also reduced the levels of soluble TREM2 in the hippocampus and frontal cortex (see, e.g., Example 6), and in the cerebrospinal fluid (see, e.g., Example 7). In addition, biomarkers of microglial activity (e.g., osteopontin and CSF1R) were also increased in the cerebrospinal fluid (see, e.g., Example 8), hippocampus, and frontal cortex (see, e.g., Example 8) of non-human primates administered multiple doses of the anti-TREM2 antibody.

Accordingly, the methods provided herein advantageously permit relatively infrequent administration of an anti-TREM2 antibody of the disclosure, which is particularly beneficial for patients with neurodegenerative diseases, such as Alzheimer's disease, that typically affect patients for long periods of time and thus require regular treatment over the course of many years. As intravenous administration of therapeutics cannot be done at home, patients must be transported to infusion centers, which is a burden on both the patient and caregiver. Finally, the memory loss, mood swings, aggression, and other behavioral symptoms of these diseases make patient compliance difficult.

All references cited herein, including patents, patent applications and publications, are hereby incorporated by reference in their entirety.

### Methods of Treatment

The present disclosure provides methods of treating and/or delaying the progression of a disease or injury in an individual, comprising administering to the individual in need thereof an antibody that binds to a TREM2 protein, where the antibody is an agonist.

As disclosed herein, anti-TREM2 antibodies of the present disclosure may be used for treating and/or delaying progression of dementia, frontotemporal dementia, Alzheimer's disease, Nasu-Hakola disease, cognitive deficit, memory loss, spinal cord injury, traumatic brain injury, a demyelination disorder, multiple sclerosis, Parkinson's disease, amyotrophic lateral sclerosis (ALS), Huntington's disease, adult-onset leukoencephalopathy with axonal spheroids and pigmented glia (ALSP), or a tauopathy disease. TREM2 activity has been implicated in such diseases, disorders, and conditions, as described, e.g., in Neumann, H et al., (2007) J Neuroimmunol 184: 92-99; Takahashi, K et al., (2005) J Exp Med 201: 647-657; Takahashi, K et al., (2007) PLoS Med 4: e124; Hsieh, C L et al., (2009) J Neurochem 109: 1144-1156; Malm, T M et al, Neurotherapeutics. 2014 Nov. 18; Paloneva, J et al., (2002) Am J Hum Genet 71: 656-662; Paloneva, J et al., (2003) J Exp Med 198: 669-675; Guerreiro, R J et al., (2013) JAMA Neurol 70: 78-84; Guerreiro, R J et al., (2012) Arch Neurol: 1-7; Guerreiro, R et al., (2013) N Engl J Med 368: 117-127; Jonsson, T et al., (2013) N Engl J Med 368: 107-116; Neumann, H et al., (2013) N Engl J Med 368: 182-184; Wang Y, et al., (2015) Cell 160(6):1061-71; Cady, J et al., (2014) JAMA Neurol. 71: 449-452; Cooper-Knock, J et al., (2017) Acta Neuropathol. Commun. 5:23; Cantoni, C et al., (2015) Acta Neuropathol. 129: 429-447; Ren, M, et al., (2018) Exp. Neurol. 302: 205-213; and Vuono, R et al., (2020) Mov Disord 35:401-408.

In some embodiments, the methods of treatment provided herein comprise administering to the individual an anti-TREM2 antibody at a dose of at least about 15 mg/kg, wherein the antibody comprises a heavy chain variable region comprising an HVR-H1, HVR-H2, and HVR-H3 and a light chain variable region comprising an HVR-L1, HVR-L2, and HVR-L3, and wherein: (i) the HVR-H1 comprises the amino acid sequence YAFSSQWMN (SEQ ID NO: 34), the HVR-H2 comprises the amino acid sequence RIYPGGGDTNYAGKFQG (SEQ ID NO: 35), the HVR-H3 comprises the amino acid sequence ARLLRNQPGESYAMDY (SEQ ID NO: 31), the HVR-L1 comprises the amino acid sequence RSSQSLVHSNRYTYLH (SEQ ID NO: 41), the HVR-L2 comprises the amino acid sequence KVSNRFS (SEQ ID NO: 33), and the HVR-L3 comprises the amino acid sequence SQSTRVPYT (SEQ ID NO: 32); or (ii) the HVR-H1 comprises the amino acid sequence YAFSSDWMN (SEQ ID NO: 36), the HVR-H2 comprises the amino acid sequence RIYPGEGDTNYARKFHG (SEQ ID NO: 37), the HVR-H3 comprises the amino acid sequence ARLLRNKPGESYAMDY (SEQ ID NO: 38), the HVR-L1 comprises the amino acid sequence RTSQSLVHSNAYTYLH (SEQ ID NO: 39), the HVR-L2 comprises the amino acid sequence KVSNRVS (SEQ ID NO: 40), and the HVR-L3 comprises the amino acid sequence SQSTRVPYT (SEQ ID NO: 32).

In some embodiments, the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSQWMN (SEQ ID NO: 34), the HVR-H2 comprises the amino acid sequence RIYPGGGDTNYAGKFQG (SEQ ID NO: 35), the HVR-H3 comprises the amino acid sequence ARLLRNQPGESYAMDY (SEQ ID NO: 31), the HVR-L1 comprises the amino acid sequence RSSQSLVHSNRYTYLH (SEQ ID NO: 41), the HVR-L2 comprises the amino acid sequence KVSNRFS (SEQ ID NO: 33), and the HVR-L3 comprises the amino acid sequence SQSTRVPYT (SEQ ID NO: 32). In some embodiments, the antibody comprises a heavy chain variable region comprising the amino acid sequence of SEQ ID NO: 27 and a light chain variable region comprising the amino acid sequence of SEQ ID NO: 30.

In some embodiments, the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSDWMN (SEQ ID NO: 36), the HVR-H2 comprises the amino acid sequence RIYPGEGDTNYARKFHG (SEQ ID NO: 37), the HVR-H3 comprises the amino acid sequence ARLLRNKPGESYAMDY (SEQ ID NO: 38), the HVR-L1 comprises the amino acid sequence RTSQSLVHSNAYTYLH (SEQ ID NO: 39), the HVR-L2 comprises the amino acid sequence KVSNRVS (SEQ ID NO: 40), and the HVR-L3 comprises the amino acid sequence SQSTRVPYT (SEQ ID NO: 32). In some embodiments, the antibody comprises a heavy chain variable region comprising the amino acid sequence of SEQ ID NO: 28 and a light chain variable region comprising the amino acid sequence of SEQ ID NO: 29.

In some embodiments, the antibody has a human IgG1 isotype. In some embodiments, the antibody has a human IgG1 isotype and comprises amino acid substitutions in the Fc region at the residue positions P331S and E430G, wherein the numbering of the residues is according to EU numbering.

In some embodiments, the antibody comprises (a) a heavy chain comprising the amino acid sequence of SEQ ID NO: 43, and a light chain comprising the amino acid sequence of SEQ ID NO: 47; or (b) a heavy chain comprising the amino acid sequence of SEQ ID NO: 44, and a light chain comprising the amino acid sequence of SEQ ID NO: 47.

In some embodiments, the antibody comprises (a) a heavy chain comprising the amino acid sequence of SEQ ID NO: 45, and a light chain comprising the amino acid sequence of SEQ ID NO: 48; or (b) a heavy chain comprising the amino acid sequence of SEQ ID NO: 46, and a light chain comprising the amino acid sequence of SEQ ID NO: 48.

Without wishing to be bound by theory, it is believed that agonizing TREM2 (e.g., by administering an anti-TREM2 antibody of the disclosure) may promote or increase microglial activity in the individual, resulting in an improvement in the pathology and/or in one or more symptoms of dementia, frontotemporal dementia, Alzheimer's disease, Nasu-Hakola disease, cognitive deficit, memory loss, spinal cord injury, traumatic brain injury, a demyelination disorder, multiple sclerosis, Parkinson's disease, amyotrophic lateral sclerosis (ALS), Huntington's disease, adult-onset leukoencephalopathy with axonal spheroids and pigmented glia (ALSP), or a tauopathy disease.

### Dementia

Dementia is a non-specific syndrome (i.e., a set of signs and symptoms) that presents as a serious loss of global cognitive ability in a previously unimpaired person, beyond what might be expected from normal ageing. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months to before an individual is diagnosed with dementia.

Exemplary forms of dementia include, without limitation, frontotemporal dementia, Alzheimer's disease, vascular dementia, semantic dementia, and dementia with Lewy bodies.

Without wishing to be bound by theory, it is believed that administering an anti-TREM2 antibody of the present disclosure can treat and/or delay the progression of dementia. In some embodiments, administering an anti-TREM2 antibody may induce or increase one or more TREM2 activities (e.g., DAP12 phosphorylation, PI3K activation, increased expression of one or more anti-inflammatory mediators, and reduced expression of one or more pro-inflammatory mediators) in an individual having dementia. In some embodiments, administering an anti-TREM2 antibody of the disclosure may promote or increase microglial activity in an individual having dementia, e.g., compared to baseline.

### Frontotemporal Dementia

Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. The clinical features of FTD include memory deficits, behavioral abnormalities, personality changes, and language impairments (Cruts, M. & Van Broeckhoven, C., Trends Genet. 24:186-194 (2008); Neary, D., et al., Neurology 51:1546-1554 (1998); Ratnavalli, E., Brayne, C., Dawson, K. & Hodges, J. R., Neurology 58:1615-1621 (2002)).

A substantial portion of FTD cases are inherited in an autosomal dominant fashion, but even in one family, symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain. Historically, the first descriptions of FTD recognized the presence of intraneuronal accumulations of hyperphosphorylated Tau protein in neurofibrillary tangles or Pick bodies. A causal role for the microtubule associated protein Tau was supported by the identification of mutations in the gene encoding the Tau protein in several families (Hutton, M., et al., Nature 393:702-705 (1998). However, the majority of FTD brains show no accumulation of hyperphosphorylated Tau but do exhibit immunoreactivity to ubiquitin (Ub) and TAR DNA binding protein (TDP43) (Neumann, M., et al., Arch. Neurol. 64:1388-1394 (2007)).

In some embodiments, administering an anti-TREM2 antibody of the present disclosure, can treat and/or delay the progression of FTD. In some embodiments, administering an anti-TREM2 antibody of the disclosure may promote microglial activity in an individual having FTD, e.g., compared to baseline. In some embodiments, administering an anti-TREM2 antibody may induce or increase one or more TREM2 activities (e.g., DAP12 phosphorylation, PI3K activation, increased expression of one or more anti-inflammatory mediators, and reduced expression of one or more pro-inflammatory mediators) in an individual having FTD.

In some embodiments, treatment and/or delay of FTD progression is determined by a change from baseline in neurocognitive and/or functional tests or assessments (i.e., clinical outcome assessments). Non-limiting examples of neurocognitive and functional tests that may be used to evaluate the treatment and/or delay of FTD progression include the Frontotemporal Dementia Clinical Rating Scale (FCRS), the Frontotemporal Dementia Rating Scale (FRS), the Clinical Global Impression-Improvement (CGI-I) assessment, the Neuropsychiatric Inventory (NPI) assessment, the Color Trails Test (CTT) Part 2, the Repeatable Battery for the Assessment of Neuropsychological Status (RBANS), the Delis-Kaplan Executive Function System Color-Word Interference Test, the Interpersonal Reactivity Index, the Winterlight Lab Speech Assessment (WLA), and the Summerlight Lab Speech Assessment (SLA). In some embodiments, treatment and/or delay of FTD progression is determined by a change from baseline in one neurocognitive and/or functional test or assessment. In some embodiments, treatment and/or delay of FTD progression is determined by a change from baseline in more than one neurocognitive and/or functional tests or assessments (e.g., 2, 3, 4, 5, 6, 7, 8, 9 or more neurocognitive and/or functional tests or assessments).

In some embodiments, treatment and/or delay of FTD progression is determined by a change from baseline in global and/or regional brain volumes, volume of white matter hyperintensities, brain perfusion, fractional anisotropy, mean diffusivity, axial diffusivity, and radial diffusivity, and/or functional brain activity. In certain embodiments, brain perfusion is measured by arterial spin labeling MRI. In certain embodiments, radial diffusivity is measured by diffusion tensor imaging. In certain embodiments, functional brain activity is measured by functional MRI.

In some embodiments, treatment and/or delay of FTD progression is determined by a change from baseline in markers of neurodegeneration in whole blood, plasma, and CSF. Makers of neurodegeneration may include, without limitation, neurofilament-light [Nfl], Tau, and/or pTau. In some embodiments, treatment and/or delay of FTD progression is determined by a change from baseline in markers of lysosomal function. Markers of lysosomal function may be, without limitation, Cathepsins. In some embodiments, treatment and/or delay of FTD progression is determined by a change from baseline in markers of microglial activity. Markers of microglial activity may be, without limitation, Interleukin-6, sCSF1R, YKL40 (CHI3L1), IL-1RA (IL1RN), and osteopontin (SPP1). In some embodiments, treatment and/or delay of FTD progression is determined by a change from baseline of messenger ribonucleic acid (mRNA) expression in peripheral cells. In some embodiments, treatment and/or delay of FTD progression is determined by a change from baseline in analytes relevant to FTD disease biology and/or response to anti-TREM2 antibody.

In some embodiments, treatment and/or delay of FTD progression is determined by a change from baseline in neuroinflammation and/or microglial activation. Neuroinflammation and/or microglial activation may be measured by any known method in the art. In certain embodiments, Neuroinflammation and/or microglial activation may be measured using Translocator Protein-Positron Emission (TSPO-PET) imaging. In certain embodiments, [¹⁸F]PBR06 and/or [¹¹C]PBR28 PET are used as radiotracers in TSPO-PET imaging. In certain embodiments, [¹⁸F]PBR06 is used as a radiotracer in TSPO-PET imaging. In certain embodiments, [¹¹C]PBR28 PET is used as a radiotracer in TSPO-PET imaging.

In some embodiments, the individual is heterozygous for a mutation in GRN (the Granulin gene). In some embodiments, the mutation in GRN is a loss-of-function mutation. In some embodiments, the individual is heterozygous for a C9orf72 hexanucleotide repeat expansion. In some embodiments, the individual shows symptoms of FTD. In some embodiments, the individual does not show symptoms of FTD.

In some embodiments, the individual shows symptoms of FTD if the individual meets diagnostic criteria for possible behavioral variant FTD (bvFTD) or probable bvFTD or primary progressive aphasia (PPA). In some embodiments, the individual has one or more of the behavioral/cognitive symptoms required for a diagnosis of possible bvFTD (Rascovsky et al., (2011) Brain 134(9):2456-2477). In some embodiments, the individual has mild symptomatology not significantly affecting activities of daily living (e.g., mild cognitive impairment, mild behavioral impairment). In certain embodiments, the individual has bvFTD or PPa with concomitant motor neuron disease. In some embodiments, the individual has FTD of mild severity as defined by a Clinical Dementia Rating Scale (CDR) global score of 1 or less and a box score of 1 or less on both the Language domain, and the Behavior, Comportment and Personality domain of the Frontotemporal Dementia Clinical Rating Scale (FCRS).

### Alzheimer's Disease

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier.

Common symptoms of Alzheimer's disease include behavioral symptoms, such as difficulty in remembering recent events, cognitive symptoms, confusion, irritability and aggression, mood swings, trouble with language, and long-term memory loss. As the disease progresses, bodily functions are lost, ultimately leading to death. Alzheimer's disease develops for an unknown and variable amount of time before becoming fully apparent, and it can progress undiagnosed for years.

Studies have shown that Triggering Receptor Expressed on Myeloid cells-2 (TREM2), an immunoglobulin-like receptor, may play a key role in AD. For example, heterozygous mutations in the TREM2 gene have been found to increase the risk of AD by up to 3-fold (Guerreiro et al (2013), N Engl J Med, 368:117-127; Jonsson et al (2013) N Engl J Med, 368:107-116), and increase the rate at which brain volume shrinks (Rajagopalan et al (2013) N Engl J Med, 369:1565-1567). Recent mouse genetic model studies also strongly support a key role for TREM2 in AD, with loss or deficiency of TREM2 being associated with increased pathology (Cheng-Hathaway et al (2018) Mol Neurodegener, 13(1):29; Wang et al (2015) Cell, 160:1061-1071; Wang et al (2016) J Exp Med, 213:667-675; Yuan et al (2016) Neuron, 90:724-739; Jay et al (2017) J Neurosci, 37:637-647). It has been shown that TREM2 expression enhances microglial cell survival, proliferation and differentiation, regulates microglial chemotaxis and phagocytosis, and is required to sustain microglial trophic function in the aging brain. In addition, animal studies have revealed an overlap between aged microglia phenotype and microglial molecular signatures found in models of AD, which include TREM2 pathways (Krasemann et al (2017) Immunity, 47(3):566-581).

Accordingly, without wishing to be bound by theory, it is believed that activation of TREM2 (e.g., using an agonist anti-TREM2 antibody provided herein) may ameliorate AD pathology and result in improvements in cognitive function by increasing microglial activity. In some embodiments, administering an anti-TREM2 antibody of the disclosure promotes or increases microglial activity in an individual having AD, e.g., compared to baseline. In some embodiments, administering an anti-TREM2 antibody of the present disclosure can treat and/or delay the progression of Alzheimer's disease. In some embodiments, administering an anti-TREM2 antibody may induce or increase one or more TREM2 activities (e.g., DAP12 phosphorylation, PI3K activation, increased expression of one or more anti-inflammatory mediators, and reduced expression of one or more pro-inflammatory mediators) in an individual having AD.

In some embodiments of the methods of treatment provided herein, the disease or injury is Alzheimer's disease. In some embodiments, the individual has a clinical diagnosis of probable Alzheimer's disease dementia based on the National Institute on Aging Alzheimer's Association criteria. In some embodiments, the individual has a Mini-Mental State Examination (MMSE) score of 16-28 points (e.g., any of 16 points, 17 points, 18 points, 19 points, 20 points, 21 points, 22 points, 23 points, 24 points, 25 points, 26 points, 27 points, or 28 points). In some embodiments, the individual has a Clinical Dementia Rating-global Score (CDR-GS) of 0.5, 1.0, or 2.0. In some embodiments, the individual has a positive amyloid-positon emission tomography (PET) scan. In some embodiments, the positive amyloid-PET scan is determined by qualitative read using ¹⁸F-Florbeta PET/computed tomography (CT) imaging. In some embodiments, the individual is taking or is being administered a cholinesterase inhibitor, e.g., for treatment of Alzheimer's disease. In some embodiments, the individual is taking or is being administered a memantine therapy, e.g., for treatment of Alzheimer's disease. In some embodiments, the individual comprises an amino acid substitution in a human TREM2 protein at residue position R47H. In some embodiments, the individual comprises an amino acid substitution in a human TREM2 protein at residue position R62H. In some embodiments, the individual comprises an amino acid substitution in a human TREM2 protein at residue position R47H and R62H. In some embodiments, the presence of one or more TREM2 mutations in the individual is determined using any method known in the art, such as sequencing (e.g., whole genome sequencing, targeted sequencing, next generation sequencing, or Sanger sequencing) or polymerase chain reaction (e.g., PCR or qPCR). In some embodiments, the individual has or is exhibiting one or more symptoms of Alzheimer's disease. In some embodiments, the individual does not have or is not exhibiting symptoms of Alzheimer's disease.

In some embodiments, treatment and/or delay of Alzheimer's disease is determined by a change from baseline in the levels of one or more biomarkers of microglial activity in the individual, e.g., in the cerebrospinal fluid or blood of the individual. Biomarkers of microglial activity include, without limitation, sCSF1R, sTREM2, YKL40 (CHI3L1), IL-1RA (IL1RN), and osteopontin (SPP1).

In certain embodiments, treatment and/or delay of Alzheimer's disease is determined by a change from baseline in one or more symptoms of Alzheimer's disease.

In certain embodiments, treatment and/or delay of Alzheimer's disease is determined using one or more clinical assessment tools such as the Mini-Mental State Examination (MMSE), the Repeatable Battery for the Assessment of Neuropsychological Status (RBANS), the Clinical Dementia Rating (CDR), the Clinical Dementia Rating-Global Score (CDR-GS), and the Clinical Dementia Rating Sum of Boxes (CDR-SB). In some embodiments, administration of an antibody of the disclosure results in an improvement in a score of one or more clinical assessments compared to prior to administration of the anti-TREM2 antibody.

In certain embodiments, treatment and/or delay of Alzheimer's disease is determined by a change from baseline in one or more biomarkers of Alzheimer's disease in the cerebrospinal fluid of the individual, such as sTREM2, sCSF1R, Abeta, Tau, p-Tau, neurofilament light chain, neurogranin, and YKL40.

In certain embodiments, treatment and/or delay of Alzheimer's disease is determined by a change from baseline in one or more biomarkers of Alzheimer's disease in the blood of the individual, such as sTREM2, sCSF1R, biomarkers of neuroinflammation (e.g., IL-6, SPP1, IFI2712A or TOP2A), and expression levels (e.g., mRNA levels) of TREM2 and CSF1R.

In certain embodiments, treatment and/or delay of Alzheimer's disease is determined by a change from baseline in one or more brain abnormalities, such as cerebral vasogenic edema, superficial siderosis of the central nervous system, and cerebral micro- or macro-hemorrhages. In certain embodiments, the one or more brain abnormalities are measured using any method known in the art, such as magnetic resonance imaging.

In certain embodiments, treatment and/or delay of Alzheimer's disease is determined by a change from baseline in the levels of brain amyloid burden. In certain embodiments, the levels of brain amyloid burden are determined using any method known in the art, such as amyloid-positron emission tomography.

In some embodiments of the methods of treatment provided herein, the disease or injury is Alzheimer's disease, wherein the Alzheimer's disease is early Alzheimer's disease. In some embodiments, early Alzheimer's disease refers to Alzheimer's disease based on the Alzheimer's disease continuum as defined by the 2018 National Institute on Aging and Alzheimer's Association (NIA-AA) Research Framework (Jack et al., Alzheimers Dement (2018) 14(4):535-562), including evidence of cerebral amyloidosis (A+) and clinical severity consistent with Stages 2, 3, or early Stage 4. In some embodiments, an individual with early Alzheimer's disease has a Clinical Dementia Rating-Global Score (CDR-GS) of 0.5 or 1, a Mini-Mental State Examination (MMSE) score of between about 22 and about 30 points (e.g., any of about 22, 23, 24, 25, 26, 27, 28, 29, or 30 points), and a Repeatable Battery for the Assessment of Neuropsychological Status (RBANS) score on the Delayed Memory Index (DMI) of about 85 points or lower (e.g., an RBANS DMI score of any of about 85, about 80, about 75, about 70, about 65, about 60, about 55, about 50, about 45, or about 40). In some embodiments, early Alzheimer's disease refers to disease with a clinical severity that is consistent with early Alzheimer's disease as defined by the European Medicines Agency (European Medicines Agency: CPMP/EWP/553/95 Rev.2. Guideline on the clinical investigation of medicines for the treatment of Alzheimer's disease 2018, available at the website www[dot]ema.europa[dot]eu/en/documents/scientific-guideline/guideline-clinical-investigation-medicines-treatment-alzheimers-disease-revision-2_en.pdf, August 2020), or by the U.S. Food and Drug Administration (Food and Drug Administration Center for Drug Evaluation and Research. Guidance for industry: Early Alzheimer's Disease: Developing Drugs for Treatment (FDA Maryland), 2018).

In some embodiments, early Alzheimer's disease is diagnosed using one or more clinical assessment tools, such as the Mini-Mental State Examination (MMSE), the Clinical Dementia Rating-Global Score (CDR-GS), or the Repeatable Battery for the Assessment of Neuropsychological Status (RBANS) on the Delayed Memory Index (DMI). In some embodiments, early Alzheimer's disease is diagnosed based on the presence of brain amyloidosis. Brain amyloidosis may be assessed using any method known in the art, such as by cerebrospinal fluid assessment or by positron emission tomography (PET).

In some embodiments, an individual treated according to the methods provided herein has a diagnosis of early Alzheimer's disease. In some embodiments, the diagnosis of early Alzheimer's disease includes evidence of brain amyloidosis, determined by CSF or PET assessments. In some embodiments, the individual has evidence of brain amyloidosis, as determined by a positive amyloid or tau blood test prior to administration of the anti-TREM2 antibody. In some embodiments, the individual has a positive amyloid or tau blood test prior to administration of the anti-TREM2 antibody. In some embodiments, the amyloid or tau blood test is the PrecivityAD™-Aβ blood test, or a test for phosphorylated tau 217 (p-tau217), a test for phosphorylated tau 181 (p-tau181), a test for neurofilament light, or a test for Aβ42/40 ratio. In some embodiments, the amyloid or tau blood test is an immunoassay-based test for Aβ42/40 ratio (see, e.g., Yamashita et al., Alzheimer's Association International Conference (2019) 15(7S), part 29, P4-548). In some embodiments, the amyloid or tau blood test is a mass spectrometry-based test for Aβ42/40 ratio (see, e.g., Schindler et al., Neurology (2019) 93(17)). In some embodiments, the amyloid or tau blood test is an immunoassay-based test for p-tau217 (see, e.g., Palmqvist et al., JAMA (2020) 324(8):772-781).

In some embodiments, the amyloid or tau blood test is the PrecivityAD™-Aβ blood test. The PrecivityAD™-Aβ blood test is based on the assessment by mass spectrometry of proteins in blood that indicate the probability of amyloid deposits in the brain, as measured by amyloid PET scans. The test incorporates the Aβ42/40 ratio, ApoE genotype, and the individual's age into a statistical algorithm to estimate an Amyloid Probability Score (APS). In some embodiments, the individual has evidence of brain amyloidosis, as determined by a positive PrecivityAD™-Aβ blood test, e.g., the individual has a high APS (www.c2ndiagnostics.com/products/home). See, e.g., Schindler et al., Neurology (2019) 93(17):e1647-e1659. In some embodiments, the individual has evidence of brain amyloidosis, as determined by an intermediate APS and confirmation of brain amyloidosis by Amyloid PET or CSF pTau/Aβ42 ratio. In some embodiments, the individual does not have a low APS. In some embodiments, the individual has evidence of brain amyloidosis, as determined by Amyloid PET scan. In some embodiments, the individual has evidence of brain amyloidosis, as determined by CSF studies. In some embodiments, the individual has evidence of brain amyloidosis, as determined by the presence of amyloid beta (Aβ) pathology. In some embodiments, the presence of amyloid beta (Aβ) pathology is assessed by a positive PrecivityAD™-Aβ blood test, e.g., the individual has a high Amyloid Probability Score (APS). In some embodiments, the presence of amyloid beta (Aβ) pathology is assessed by Amyloid PET scans. In some embodiments, the presence of amyloid beta (Aβ) pathology is assessed by CSF studies. In some embodiments, the individual has evidence of brain amyloidosis, as determined by the presence of amyloid pathology. In some embodiments, the presence of amyloid pathology is assessed by Amyloid PET scans. In some embodiments, the presence of amyloid pathology is assessed by CSF phosphorylated tau (pTau)/amyloid beta (1-42) (Aβ42) ratio measurements. In some embodiments, the individual has evidence of brain amyloidosis, as determined by a positive historical Amyloid PET scan, e.g., collected ≤24 months prior to the start of treatment according to the methods of the disclosure. In some embodiments, the individual has evidence of Alzheimer's disease amyloid pathology, as determined by a positive Amyloid PET scan and/or by a CSF pTau/Aβ42 ratio of greater than 0.024. Any suitable method known in the art may be used to measure the CSF pTau/Aβ42 ratio, such as immunoassays, e.g., the Roche Elecsys assay. In some embodiments, the individual has early Alzheimer's disease with clinical severity consistent with Stages 2, 3 or early Stage 4 as defined in the 2018 NIA-AA Research Framework (Jack et al., Alzheimers Dement (2018) 14(4):535-562), also described as mild cognitive impairment and mild dementia in the 2018 NIA-AA Research Framework. In some embodiments, the individual has a Mini-Mental State Examination (MMSE) score of at least about 22 points (e.g., any of about 22, 23, 24, 25, 26, 27, 28, 29, or 30 points). In some embodiments, the individual has early Alzheimer's disease with mild symptomatology, defined by a Mini-Mental State Examination (MMSE) score of at least about 22 points (e.g., any of about 22, 23, 24, 25, 26, 27, 28, 29, or 30 points). In some embodiments, the individual has a Clinical Dementia Rating-Global Score (CDR-GS) of between about 0.5 and about 1.0. In some embodiments, the individual has a Repeatable Battery for the Assessment of Neuropsychological Status (RBANS) score on the Delayed Memory Index (DMI) of about 85 or less (e.g., an RBANS DMI score of any of about 85, about 80, about 75, about 70, about 65, about 60, about 55, about 50, about 45, or about 40). In some embodiments, the individual has a Repeatable Battery for the Assessment of Neuropsychological Status (RBANS) score on the Delayed Memory Index (DMI) that is about one standard deviation below population-based normative data. In some embodiments, the individual demonstrates amnestic deficits, assessed by the Delayed Memory Index (DMI) of the Repeatable Battery for the Assessment of Neuropsychological Status (RBANS). In some embodiments, the individual has evidence of episodic memory impairment, as defined by an RBANS score on the DMI of about 85 or less (e.g., an RBANS DMI score of any of about 85, about 80, about 75, about 70, about 65, about 60, about 55, about 50, about 45, or about 40). In some embodiments, the individual does not have frontotemporal dementia (FTD), Parkinson's disease, dementia with Lewy bodies, Huntington disease, or vascular dementia. In some embodiments, the individual does not have a condition other than Alzheimer's disease that has the potential to affect cognition. Examples of conditions with the potential to affect cognition include, without limitation, frontotemporal dementia, dementia with Lewy bodies, vascular dementia, Parkinson's disease, corticobasal degeneration, Creutzfeldt-Jakob disease, progressive supranuclear palsy, frontotemporal degeneration, Huntington disease, normal pressure hydrocephalus, hypoxic injury, seizure disorder, static encephalopathy, closed brain injury, and developmental disability. In some embodiments, the individual does not have uncontrolled hypertension, diabetes mellitus or thyroid disease. In some embodiments, the individual does not have significant heart disease, cardiovascular disease or disorder, liver disease or disorder, or kidney disease or disorder. In some embodiments, the individual does not have evidence of clinically significant brain disease other than Alzheimer's disease. In some embodiments, the individual is not being administered an anticoagulant medication. In some embodiments, the individual does not have history or presence of vascular disease that has the potential to affect cognitive function, such as clinically significant carotid, vertebral stenosis, or plaque; aortic aneurysm; intracranial aneurysm; macro-hemorrhage; or arteriovenous malformation. In some embodiments, the individual does not have history or presence of clinical stroke within the past 2 years prior to treatment according to the methods of the disclosure. In some embodiments, the individual does not have history or presence of an acute event consistent with a transient ischemic attack within the last 180 days prior to treatment according to the methods of the disclosure. In some embodiments, the individual does not have presence on MRI of any cortical stroke. In some embodiments, the individual does not have history of severe, clinically significant (e.g., persistent neurologic deficit or structural brain damage) CNS trauma (e.g., cerebral contusion). In some embodiments, the individual does not have history or presence of intracranial tumor, e.g., glioma, with the exception of benign brain tumors which do not cause cognitive symptoms. In some embodiments, the individual does not have the presence of an infection that affects brain function. In some embodiments, the individual does not have history of infections that resulted in neurologic sequelae. Examples of such infections include, without limitation, human immunodeficiency virus, syphilis, neuroborreliosis, viral or bacterial meningitis/encephalitis. In some embodiments, the individual does not have or has not had an acute illness that requires or required intravenous antibiotics within 30 days prior to treatment according to the methods of the disclosure. In some embodiments, the individual does not have history or presence of systemic autoimmune disorders that have the potential to cause progressive neurologic disease with associated cognitive deficits. Examples of such autoimmune disorders include, without limitation, multiple sclerosis, lupus erythematosus, antiphospholipid antibody syndrome, and Behçet disease. In some embodiments, the individual does not have history or presence of uveitis requiring medical intervention, chronic inflammatory or degenerative condition of the eye, current eye infection, any ongoing eye disorder (e.g., degeneration, cataract, or diabetic retinopathy) requiring injectable medical therapy (e.g., ranibizumab or aflibercept for macular degeneration). In some embodiments, the individual does not have any history of schizophrenia, schizoaffective disorder, major depression, or bipolar disorder. In some embodiments, the individual is not at risk of suicide. In some embodiments, the individual does not have history of alcohol and/or moderate to severe substance use disorder (according to the Diagnostic and Statistical Manual of Mental Disorders, 5th Edition) within the past 2 years prior to treatment according to the methods of the disclosure. In some embodiments, the individual does not have MRI evidence of >2 lacunar infarcts, any territorial infarct >1 cm³, or white matter hyperintense lesions on the FLAIR sequence that correspond to an overall Fazekas score of 3. In some embodiments, the individual does not have presence on MRI of >5 microbleeds and/or areas of leptomeningeal hemosiderosis. In some embodiments, the individual does not have significant cerebral vascular pathology as assessed by MRI. In some embodiments, the individual is not positive for hepatitis B surface antigen, total hepatitis B core antibody, HIV-1 or -2 antibodies or antigen. In some embodiments, the individual does not have history of spirochetal infection of the central nervous system (e.g., syphilis, borreliosis, or Lyme disease). In some embodiments, the individual is positive for hepatitis C virus antibody and is negative for hepatitis C ribonucleic acid (RNA). In some embodiments, the individual does not have active or latent tuberculosis disease. In some embodiments, the individual does not have a chronic active immune disorder requiring systemic immunosuppressive therapy within 1 year prior to treatment according to the methods of the disclosure. In some embodiments, the individual does not have bone marrow dysfunction based upon hemoglobin<10 g/dL, absolute neutrophil count>1000/mm³, or platelet count<150000/mm³. In some embodiments, the individual does not have abnormal thyroid stimulating hormone (TSH) levels. In some embodiments, the individual does not have folic acid or vitamin B12 levels that are sufficiently low such that the deficiency could contribute to cognitive impairment. In some embodiments, the individual does not have hemoglobin A1c>8% or poorly controlled diabetes (including hypoglycemic episodes). In some embodiments, the individual is not on a continuous regimen of medications known to impair consciousness or cognition. In some embodiments, the individual has not been treated with a medication for Parkinsonian symptoms or any other neurodegenerative disorder, except for treatments for Alzheimer's disease, within 1 year prior to treatment according to the methods of the disclosure. In some embodiments, the individual may be taking a medication used for treating a neurodegenerative disorder if the medication is being taken by the individual for a non-neurodegenerative disorder, e.g., restless leg disorder, e.g., pramipexole. In some embodiments, the individual has not taken a typical antipsychotic or neuroleptic medication within 180 days prior to treatment according to the methods of the disclosure, except as brief treatment for a nonpsychiatric indication (e.g., emesis). In some embodiments, the individual has not taken an atypical antipsychotic medication, except with intermittent short-term use (e.g., <1 week). In some embodiments, the individual has not taken an anticoagulation medication within 90 days prior to treatment according to the methods of the disclosure. In some embodiments, the individual is not taking a systemic immunosuppressive therapy. In some embodiments, the individual does not have chronic use of opiates or opioids, including long-acting opioid medications, within 90 days prior to treatment according to the methods provided herein. In some embodiments, the individual has not taken stimulant medications (e.g., amphetamine, methylphenidate preparations, or modafinil) within 30 days prior to treatment according to the methods of the disclosure. In some embodiments, the individual does not have chronic use of benzodiazepines, barbiturates, or hypnotics starting from 90 days before treatment according to the methods of the disclosure.

In certain embodiments, treatment and/or delay of early Alzheimer's disease is determined using one or more clinical assessment tools such as the Clinical Dementia Rating (CDR), the Clinical Dementia Rating Sum of Boxes (CDR-SB), the Mini-Mental State Examination (MMSE), the Repeatable Battery for the Assessment of Neuropsychological Status (RBANS), the Alzheimer's Disease Assessment Scale-Cognitive Subscale-13 (ADAS-Cog13), the Alzheimer's Disease Cooperative Study-Activities of Daily Living adapted to Mild Cognitive Impairment (ADCS-ADL-MCI), the Alzheimer's Disease Composite Score (ADCOMS), or the Winterlight Labs Speech Assessment (WLSA). In some embodiments, administration of an antibody of the disclosure results in an improvement in a score of one or more clinical assessments compared to prior to administration of the anti-TREM2 antibody. In certain embodiments, treatment and/or delay of early Alzheimer's disease is assessed based on the rate of change of a score of one or more clinical assessment tools such as the CDR-SB, the MMSE, the RBANS, the ADAS-Cog13, the ADCS-ADL-MCI, the ADCOMS, or the WLSA, e.g., compared to prior to administration of the anti-TREM2 antibody. In certain embodiments, treatment and/or delay of early Alzheimer's disease is assessed based on the rate of change of a score of one or more clinical assessment tools such as the CDR-SB, the MMSE, the RBANS, the ADAS-Cog13, the ADCS-ADL-MCI, the ADCOMS, or the WLSA, i.e., based on a comparison of a score of the one or more clinical assessment tools obtained prior to administration of the anti-TREM2 antibody to a corresponding score of the one or more clinical assessment tools obtained after the individual has received one or more doses of the anti-TREM2 antibody; or based on a comparison of two or more scores of the one or more clinical assessment tools obtained during the course of treatment with the anti-TREM2 antibody according to the methods of the disclosure. In certain embodiments, treatment and/or delay of early Alzheimer's disease is determined by a change from baseline in one or more biomarkers of Alzheimer's disease in the cerebrospinal fluid of the individual, such as soluble TREM2 (sTREM2), and other CSF biomarkers relevant to Alzheimer's disease (e.g., Aβ42, Aβ40, total tau, pTau, or NfL) or microglial function (e.g., CSF1R, IL1RN, YKL40 and osteopontin). In certain embodiments, treatment and/or delay of early Alzheimer's disease is determined by a change from baseline in one or more biomarkers of Alzheimer's disease in the blood of the individual, such as soluble TREM2 (sTREM2) in plasma, plasma biomarkers relevant to Alzheimer's disease (e.g., Aβ42, Aβ40, total tau, pTau, NfL), or TREM2 RNA expression. In certain embodiments, treatment and/or delay of early Alzheimer's disease is determined by a change from baseline in one or more biomarkers of microglial function in the plasma or the cerebrospinal fluid of the individual, such as CSF1R, IL1RN, osteopontin, or YKL40. In certain embodiments, treatment and/or delay of early Alzheimer's disease is determined by a change from baseline in one or more biomarkers of neurodegeneration in the plasma or the cerebrospinal fluid of the individual, such as NfL. In certain embodiments, treatment and/or delay of early Alzheimer's disease is determined by a change from baseline in brain volume, e.g., assessed by volumetric MRI. In certain embodiments, treatment and/or delay of early Alzheimer's disease is determined by a change from baseline in brain pathological tau burden, e.g., assessed by Tau-PET, e.g., using the [¹⁸F]MK-6240 Tau-PET radiotracer. In certain embodiments, treatment and/or delay of early Alzheimer's disease is determined by a change from baseline in brain amyloid burden, e.g., assessed by longitudinal Amyloid-PET, e.g., using [₁₈F]florbetaben (Neuraceq), [¹⁸F]florbetapir (Amyvid), or [¹⁸F]flutametamol (Vizamyl) as radiotracers. In certain embodiments, treatment and/or delay of early Alzheimer's disease is determined by a change from baseline in one or more biomarkers of Alzheimer's disease, assessed by magnetic resonance imaging (MRI), such as Amyloid PET imaging (e.g., longitudinal Amyloid PET), e.g., using [¹⁸F]florbetaben (Neuraceq), [¹⁸F]florbetapir (Amyvid), or [¹⁸F]flutametamol (Vizamyl) as radiotracers, or Tau-PET imaging, e.g., using the [¹⁸F]MK-6240 Tau-PET radiotracer. In certain embodiments, treatment and/or delay of early Alzheimer's disease is determined by tau and/or amyloid positron emission tomography (PET) imaging. In certain embodiments, treatment and/or delay of early Alzheimer's disease is determined by a change from baseline in the speech of the individual, e.g., using the Winterlight Labs Speech Assessment (WLSA).

In certain embodiments, the methods of the disclosure comprise performing genomic assessments to determine whether an individual is an APOE e4 carrier or non-carrier, and/or has one or more of a TREM2 variant, a CD33 variant, a TMEM106b variant, or a CLUSTERIN variant. In certain embodiments, the methods of the disclosure comprise performing an amyloid or tau blood test on a sample obtained from an individual (e.g., an individual having early Alzheimer's disease) prior to treatment according to the methods of the disclosure. In certain embodiments, the methods of the disclosure comprise determining that an individual (e.g., an individual having early Alzheimer's disease) has a positive amyloid or tau blood test result prior to treatment according to the methods of the disclosure. In certain embodiments, the methods of the disclosure comprise performing an amyloid or tau blood test on a sample obtained from an individual before and after the individual has received one or more doses of the anti-TREM2 antibody. In some embodiments, the amyloid or tau blood test is the PrecivityAD™-Aβ blood test, or a test for phosphorylated tau 217 (p-tau217), a test for phosphorylated tau 181 (p-tau181), a test for neurofilament light, or a test for Aβ42/40 ratio. In some embodiments, the amyloid or tau blood test is an immunoassay-based test for Aβ42/40 ratio (see, e.g., Yamashita et al., Alzheimer's Association International Conference (2019) 15(7S), part 29, P4-548). In some embodiments, the amyloid or tau blood test is a mass spectrometry-based test for Aβ42/40 ratio (see, e.g., Schindler et al., Neurology (2019) 93(17)). In some embodiments, the amyloid or tau blood test is an immunoassay-based test for p-tau217 (see, e.g., Palmqvist et al., JAMA (2020) 324(8):772-781).

### Nasu-Hakola Disease

Nasu-Hakola disease (NHD), which may alternatively be referred to as polycystic lipomembranous osteodysplasia with sclerosing leukoencephalopathy (PLOSL), is a rare inherited leukodystrophy characterized by progressive presenile dementia associated with recurrent bone fractures due to polycystic osseous lesions of the lower and upper extremities. NHD disease course is generally divided into four stages: latent, osseous, early neurologic, and late neurologic. After a normal development during childhood (latent stage), NHD starts manifesting during adolescence or young adulthood (typical age of onset 20-30 years) with pain in the hands, wrists, ankles, and feet. Patients then start suffering from recurrent bone fractures due to polycystic osseous and osteroporotic lesions in the limb bones (osseous stage). During the third or fourth decade of life (early neurologic stage), patients present with pronounced personality changes (e.g., euphoria, lack of concentration, loss of judgment, and social inhibitions) characteristic of a frontal lobe syndrome. Patients also typically suffer from progressive memory disturbances. Epileptic seizures are also frequently observed. Finally (late neurologic stage), patients progress to a profound dementia, are unable to speak and move, and usually die by the age of 50.

In some embodiments, administering an anti-TREM2 antibody of the present disclosure can treat and/or delay Nasu-Hakola disease (NHD). In some embodiments, administering an anti-TREM2 antibody may promote or increase microglial activity in an individual having NHD, e.g., compared to baseline. In some embodiments, administering an anti-TREM2 antibody may induce or increase one or more TREM2 activities (e.g., DAP12 phosphorylation, PI3K activation, increased expression of one or more anti-inflammatory mediators, and reduced expression of one or more pro-inflammatory mediators) in an individual having NHD.

### Amyotrophic Lateral Sclerosis (ALS)

As used herein, amyotrophic lateral sclerosis (ALS) or, motor neuron disease or, Lou Gehrig's disease are used interchangeably and refer to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

It has been shown that progranulin plays a role in ALS (Schymick, J C et al., (2007) J Neurol Neurosurg Psychiatry; 78:754-6) and protects again the damage caused by ALS causing proteins such as TDP-43 (Laird, A S et al., (2010). PLoS ONE 5: e13368). It was also demonstrated that pro-NGF induces p75 mediated death of oligodendrocytes and corticospinal neurons following spinal cord injury (Beatty et al., Neuron (2002), 36, pp. 375-386; Giehl et al, Proc. Natl. Acad. Sci USA (2004), 101, pp 6226-30).

In some embodiments, administering an anti-TREM2 antibody of the present disclosure can treat and/or delay ALS. In some embodiments, administering an anti-TREM2 antibody may promote or increase microglial activity in an individual having ALS, e.g., compared to baseline. In some embodiments, administering an anti-TREM2 antibody may induce or increase one or more TREM2 activities (e.g., DAP12 phosphorylation, PI3K activation, increased expression of one or more anti-inflammatory mediators, and reduced expression of one or more pro-inflammatory mediators) in an individual having ALS.

In some embodiments, treatment and/or delay of ALS progression is determined by a change from baseline in brain atrophy, brain connectivity, brain free water and/or brain inflammation. Any method known in the art including, without limitation, MRI, may be used to measure brain atrophy, brain connectivity, brain free water and/or brain inflammation. In certain embodiments, brain atrophy is measured using structural MRI. In certain embodiments, brain free water and/or brain inflammation are measured using diffusion tensor imaging (DTI). In some embodiments, treatment and/or delay of ALS progression is determined by a change from baseline in one or more markers of neurodegeneration, one or more markers of glial activity, progranulin, and/or one or more markers of TDP-43 pathology.

### Parkinson's Disease

Parkinson's disease (PD), which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include, without limitation, tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems.

In some embodiments, administering an anti-TREM2 antibody of the present disclosure can treat and/or delay PD. In some embodiments, administering an anti-TREM2 antibody may promote or increase microglial activity in an individual having PD, e.g., compared to baseline. In some embodiments, administering an anti-TREM2 antibody may induce or increase one or more TREM2 activities (e.g., DAP12 phosphorylation, PI3K activation, increased expression of one or more anti-inflammatory mediators, and reduced expression of one or more pro-inflammatory mediators) in an individual having PD.

### Huntington's Disease

Huntington's disease (HD) is an inherited neurodegenerative disease caused by an autosomal dominant mutation in the Huntingtin gene (HTT). Expansion of a cytokine-adenine-guanine (CAG) triplet repeat within the Huntingtin gene results in production of a mutant form of the Huntingtin protein (Htt) encoded by the gene. This mutant Huntingtin protein (mHtt) is toxic and contributes to neuronal death. Symptoms of Huntington's disease most commonly appear between the ages of 35 and 44, although they can appear at any age.

Symptoms of Huntington's disease, include, without limitation, motor control problems, jerky, random movements (chorea), abnormal eye movements, impaired balance, seizures, difficulty chewing, difficulty swallowing, cognitive problems, altered speech, memory deficits, thinking difficulties, insomnia, fatigue, dementia, changes in personality, depression, anxiety, and compulsive behavior.

In some embodiments, administering an anti-TREM2 antibody of the present disclosure can treat and/or delay HD. In some embodiments, administering an anti-TREM2 antibody may promote or increase microglial activity in an individual having HD, e.g., compared to baseline. In some embodiments, administering an anti-TREM2 antibody may induce or increase one or more TREM2 activities (e.g., DAP12 phosphorylation, PI3K activation, increased expression of one or more anti-inflammatory mediators, and reduced expression of one or more pro-inflammatory mediators) in an individual having HD.

### Tauopathy Disease

Tauopathy diseases, or Tauopathies, are a class of neurodegenerative disease caused by aggregation of the microtubule-associated protein tau within the brain. Alzheimer's disease (AD) is the most well-known tauopathy disease, and involves an accumulation of tau protein within neurons in the form of insoluble neurofibrillary tangles (NFTs). Other tauopathy diseases and disorders include progressive supranuclear palsy, dementia pugilistica (chromic traumatic encephalopathy), Frontotemporal dementia and parkinsonism linked to chromosome 17, Lytico-Bodig disease (Parkinson-dementia complex of Guam), Tangle-predominant dementia, Ganglioglioma and gangliocytoma, Meningioangiomatosis, Subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), Huntington's disease, frontotemporal dementia, and frontotemporal lobar degeneration.

In some embodiments, administering an anti-TREM2 antibody of the present disclosure can treat and/or delay tauopathy disease. In some embodiments, administering an anti-TREM2 antibody may promote or increase microglial activity in an individual having tauopathy disease, e.g., compared to baseline. In some embodiments, administering an anti-TREM2 antibody may induce or increase one or more TREM2 activities (e.g., DAP12 phosphorylation, PI3K activation, increased expression of one or more anti-inflammatory mediators, and reduced expression of one or more pro-inflammatory mediators) in an individual having tauopathy disease.

### Multiple Sclerosis

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. MS affects the ability of nerve cells in the brain and spinal cord to communicate with each other effectively. Nerve cells communicate by sending electrical signals called action potentials down long fibers called axons, which are contained within an insulating substance called myelin. In MS, the body's own immune system attacks and damages the myelin. When myelin is lost, the axons can no longer effectively conduct signals. MS onset usually occurs in young adults, and is more common in women.

Symptoms of MS include, without limitation, changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoffs phenomenon, which is an exacerbation of extant symptoms due to an exposure to higher than usual ambient temperatures; and Lhermitte's sign, which is an electrical sensation that runs down the back when bending the neck.

In some embodiments, administering an anti-TREM2 antibody of the present disclosure can treat and/or delay MS. In some embodiments, administering an anti-TREM2 antibody may promote or increase microglial activity in an individual having MS, e.g., compared to baseline. In some embodiments, administering an anti-TREM2 antibody may induce or increase one or more TREM2 activities (e.g., DAP12 phosphorylation, PI3K activation, increased expression of one or more anti-inflammatory mediators, and reduced expression of one or more pro-inflammatory mediators) in an individual having MS.

### Traumatic Brain Injuries and Spinal Cord Injuries

Traumatic brain injuries (TBI), may also be known as intracranial injuries. Traumatic brain injuries occur when an external force traumatically injures the brain. Traumatic brain injuries can be classified based on severity, mechanism (closed or penetrating head injury), or other features (e.g., occurring in a specific location or over a widespread area).

In some embodiments, administering an anti-TREM2 antibody of the present disclosure can treat a TBI. In some embodiments, administering an anti-TREM2 antibody may promote or increase microglial activity in an individual having a TBI, e.g., compared to baseline. In some embodiments, administering an anti-TREM2 antibody may induce or increase one or more TREM2 activities (e.g., DAP12 phosphorylation, PI3K activation, increased expression of one or more anti-inflammatory mediators, and reduced expression of one or more pro-inflammatory mediators) in an individual having a TBI.

Spinal cord injuries (SCI) include any injury to the spinal cord that is caused by trauma instead of disease. Depending on where the spinal cord and nerve roots are damaged, the symptoms can vary widely, from pain to paralysis to incontinence. Spinal cord injuries are described at various levels of “incomplete”, which can vary from having no effect on the patient to a “complete” injury which means a total loss of function.

In some embodiments, administering an anti-TREM2 antibody of the present disclosure can treat an SCI. In some embodiments, administering an anti-TREM2 antibody may promote or increase microglial activity in an individual having an SCI, e.g., compared to baseline. In some embodiments, administering an anti-TREM2 antibody may induce or increase one or more TREM2 activities (e.g., DAP12 phosphorylation, PI3K activation, increased expression of one or more anti-inflammatory mediators, and reduced expression of one or more pro-inflammatory mediators) in an individual having an SCI.

Adult-Onset Leukoencephalopathy with Axonal Spheroids and Pigmented Glia (ALSP) and Pediatric-Onset Leukoencephalopathy

Adult-onset leukoencephalopathy with axonal spheroids and pigmented glia (ALSP), and pediatric-onset leukoencephalopathy are rare, fatal neurological diseases which alter the “white matter” of the central nervous system in afflicted individuals (Freeman et al. (2009) “Adult onset leukodystrophy with neuroaxonal spheroids: Clinical, neuroimaging and neuropathologic observations.” Brain Pathol. 19(1): 39-47. PMID: 18422757; Rademakers et al. (2011) “Mutations in the colony stimulating factor 1 receptor (CSF1R) gene cause hereditary diffuse leukoencephalopathy with spheroids.” Nat Genet. 44(2):200-205. PMID: 22197934; Oosterhof et al. (2019) “Homozygous Mutations in CSF1R Cause a Pediatric-Onset Leukoencephalopathy and Can Result in Congenital Absence of Microglia.” Am J Hum Genet. 104(5):936-947. PMID: 30982608). Previously, ALSP was thought to be two separate conditions, hereditary diffuse leukoencephalopathy (HDLS) and familial pigmentary orthochromatic leukoencephalopathy (POLD). However, given patients with HDLS and POLD both can have pigmented glial cells and spheroids, HDLS and POLD are considered part of the same spectrum of disease encompassed by ALSP (Nicholson et al. (2013) “CSF1R mutations link POLD and HDLS as a single disease entity.” Neurology 80(11): 1033-1040. PMID: 23408870). In some embodiments, administering an anti-TREM2 antibody of the present disclosure can treat ALSP or pediatric-onset leukoencephalopathy.

### Clinical Assessments

In some embodiments of the methods of treatment provided herein, the method comprises determining a score of one or more clinical assessments of the individual before and after the individual has received one or more doses of the anti-TREM2 antibody. In some embodiments, the clinical assessments are selected from the Mini-Mental State Examination (MMSE) score, the Clinical Dementia Rating-Global Score (CDR-GS), the Clinical Dementia Rating Sum of Boxes (CDR-SB), or the Repeatable Battery for the Assessment of Neuropsychological Status (RBANS). In some embodiments, administration of an antibody of the disclosure results in an improvement in a score of the one or more clinical assessments compared to prior to administration of the anti-TREM2 antibody, e.g., compared to a score of the one or more clinical assessments at between about 42 days to less than 1 day (e.g., any of 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less than 1 day) prior to administration of the anti-TREM2 antibody.

### Pharmaceutical Dosages

An antibody provided herein (and any additional therapeutic agent) can be administered by any suitable means, including parenteral, intrapulmonary, intranasal, intralesional administration, intracerobrospinal, intracranial, intraspinal, intrasynovial, intrathecal, oral, topical, or inhalation routes. Parenteral infusions include intramuscular, intravenous administration as a bolus or by continuous infusion over a period of time, intraarterial, intra-articular, intraperitoneal, or subcutaneous administration. In some embodiments, the administration is intravenous administration. In some embodiments, the administration is subcutaneous. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies provided herein are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

Dosages for a particular anti-TREM2 antibody may be determined empirically in individuals who have been given one or more administrations of the anti-TREM2 antibody. Individuals are given incremental doses of an anti-TREM2 antibody. To assess efficacy of an anti-TREM2 antibody, a clinical symptom of any of the diseases, disorders, or conditions of the present disclosure (e.g., dementia, frontotemporal dementia, Alzheimer's disease, Nasu-Hakola disease, cognitive deficit, memory loss, spinal cord injury, traumatic brain injury, a demyelination disorder, multiple sclerosis, Parkinson's disease, amyotrophic lateral sclerosis (ALS), Huntington's disease, adult-onset leukoencephalopathy with axonal spheroids and pigmented glia (ALSP), and a tauopathy disease) can be monitored.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

In some aspects, methods of the present disclosure comprise administering to an individual an anti-TREM2 antibody intravenously at a dose of at least about 15 mg/kg. In some embodiments, the dose is between about 15 mg/kg to about 60 mg/kg. In some embodiments, the dose is between about 15 mg/kg to about 50 mg/kg. In some embodiments, the dose is between about 20 mg/kg to about 50 mg/kg. In some embodiments, the dose is between about 20 mg/kg to about 60 mg/kg. In some embodiments, the dose is between about 15 mg/kg to about 20 mg/kg. In some embodiments, the dose is between about 45 mg/kg to about 50 mg/kg. In some embodiments, the dose is between about 50 mg/kg to about 60 mg/kg. In some embodiments, the dose is between about 20 mg/kg to about 30 mg/kg. In some embodiments, the dose is any of about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, or about 60 mg/kg.

In some embodiments, doses are administered intermittently, e.g., any of about once every week, once every two weeks, once every three weeks, once every four weeks, once every five weeks, once every six weeks, once every seven weeks, or once every eight weeks. In some embodiments, doses are administered q1w, q2w, q3w, q4w, q5w, q6w, q7w, or q8w.

In some embodiments, the dosing frequency is equal to or greater than q1w (i.e., doses are administered once every week or less frequently than once every week). In some embodiments, the dosing frequency is equal to or greater than q2w (i.e., doses are administered once every two weeks or less frequently than once every two weeks). In some embodiments, the dosing frequency is equal to or greater than q3w (i.e., doses are administered once every three weeks or less frequently than once every three weeks). In some embodiments, the dosing frequency is equal to or greater than q4w (i.e., doses are administered once every four weeks or less frequently than once every four weeks). In some embodiments, the dosing frequency is equal to or greater than q5w (i.e., doses are administered once every five weeks or less frequently than once every five weeks). In some embodiments, the dosing frequency is equal to or greater than q6w (i.e., doses are administered once every six weeks or less frequently than once every six weeks). In some embodiments, the dosing frequency is equal to or greater than q7w (i.e., doses are administered once every seven weeks or less frequently than once every seven weeks). In some embodiments, the dosing frequency is equal to or greater than q8w (i.e., doses are administered once every eight weeks or less frequently than once every eight weeks). In some embodiments, the dosing frequency is once every 2 weeks. In some embodiments, the dosing frequency is once every 3 weeks. In some embodiments, the dosing frequency is once every 4 weeks. In some embodiments, the dosing frequency is once every 5 weeks. In some embodiments, the dosing frequency is once every 6 weeks.

In some embodiments, the anti-TREM2 antibody is administered to the individual at a dose of at least about 15 mg/kg once every week. In some embodiments, the anti-TREM2 antibody is administered to the individual at a dose of about 15 mg/kg once every week. In some embodiments, the anti-TREM2 antibody is administered to the individual at a dose of about 20 mg/kg once every week. In some embodiments, the anti-TREM2 antibody is administered to the individual at a dose of about 25 mg/kg once every week. In some embodiments, the anti-TREM2 antibody is administered to the individual at a dose of about 30 mg/kg once every week. In some embodiments, the anti-TREM2 antibody is administered to the individual at a dose of about 35 mg/kg once every week. In some embodiments, the anti-TREM2 antibody is administered to the individual at a dose of about 40 mg/kg once every week. In some embodiments, the anti-TREM2 antibody is administered to the individual at a dose of about 45 mg/kg once every week. In some embodiments, the anti-TREM2 antibody is administered to the individual at a dose of about 50 mg/kg once every week. In some embodiments, the anti-TREM2 antibody is administered to the individual at a dose of about 55 mg/kg once every week. In some embodiments, the anti-TREM2 antibody is administered to the individual at a dose of about 60 mg/kg once every week.

In some embodiments, the anti-TREM2 antibody is administered to the individual at a dose of at least about 15 mg/kg once every two weeks. In some embodiments, the anti-TREM2 antibody is administered to the individual at a dose of about 15 mg/kg once every two weeks. In some embodiments, the anti-TREM2 antibody is administered to the individual at a dose of about 20 mg/kg once every two weeks. In some embodiments, the anti-TREM2 antibody is administered to the individual at a dose of about 25 mg/kg once every two weeks. In some embodiments, the anti-TREM2 antibody is administered to the individual at a dose of about 30 mg/kg once every two weeks. In some embodiments, the anti-TREM2 antibody is administered to the individual at a dose of about 35 mg/kg once every two weeks. In some embodiments, the anti-TREM2 antibody is administered to the individual at a dose of about 40 mg/kg once every two weeks. In some embodiments, the anti-TREM2 antibody is administered to the individual at a dose of about 45 mg/kg once every two weeks. In some embodiments, the anti-TREM2 antibody is administered to the individual at a dose of about 50 mg/kg once every two weeks. In some embodiments, the anti-TREM2 antibody is administered to the individual at a dose of about 55 mg/kg once every two weeks. In some embodiments, the anti-TREM2 antibody is administered to the individual at a dose of about 60 mg/kg once every two weeks.

In some embodiments, the anti-TREM2 antibody is administered to the individual at a dose of at least about 15 mg/kg once every three weeks. In some embodiments, the anti-TREM2 antibody is administered to the individual at a dose of about 15 mg/kg once every three weeks. In some embodiments, the anti-TREM2 antibody is administered to the individual at a dose of about 20 mg/kg once every three weeks. In some embodiments, the anti-TREM2 antibody is administered to the individual at a dose of about 25 mg/kg once every three weeks. In some embodiments, the anti-TREM2 antibody is administered to the individual at a dose of about 30 mg/kg once every three weeks. In some embodiments, the anti-TREM2 antibody is administered to the individual at a dose of about 35 mg/kg once every three weeks. In some embodiments, the anti-TREM2 antibody is administered to the individual at a dose of about 40 mg/kg once every three weeks. In some embodiments, the anti-TREM2 antibody is administered to the individual at a dose of about 45 mg/kg once every three weeks. In some embodiments, the anti-TREM2 antibody is administered to the individual at a dose of about 50 mg/kg once every three weeks. In some embodiments, the anti-TREM2 antibody is administered to the individual at a dose of about 55 mg/kg once every three weeks. In some embodiments, the anti-TREM2 antibody is administered to the individual at a dose of about 60 mg/kg once every three weeks.

In some embodiments, the anti-TREM2 antibody is administered to the individual at a dose of at least about 15 mg/kg once every four weeks. In some embodiments, the anti-TREM2 antibody is administered to the individual at a dose of about 15 mg/kg once every four weeks. In some embodiments, the anti-TREM2 antibody is administered to the individual at a dose of about 20 mg/kg once every four weeks. In some embodiments, the anti-TREM2 antibody is administered to the individual at a dose of about 25 mg/kg once every four weeks. In some embodiments, the anti-TREM2 antibody is administered to the individual at a dose of about 30 mg/kg once every four weeks. In some embodiments, the anti-TREM2 antibody is administered to the individual at a dose of about 35 mg/kg once every four weeks. In some embodiments, the anti-TREM2 antibody is administered to the individual at a dose of about 40 mg/kg once every four weeks. In some embodiments, the anti-TREM2 antibody is administered to the individual at a dose of about 45 mg/kg once every four weeks. In some embodiments, the anti-TREM2 antibody is administered to the individual at a dose of about 50 mg/kg once every four weeks. In some embodiments, the anti-TREM2 antibody is administered to the individual at a dose of about 55 mg/kg once every four weeks. In some embodiments, the anti-TREM2 antibody is administered to the individual at a dose of about 60 mg/kg once every four weeks.

In some embodiments, the anti-TREM2 antibody is administered to the individual at a dose of at least about 15 mg/kg once every five weeks. In some embodiments, the anti-TREM2 antibody is administered to the individual at a dose of about 15 mg/kg once every five weeks. In some embodiments, the anti-TREM2 antibody is administered to the individual at a dose of about 20 mg/kg once every five weeks. In some embodiments, the anti-TREM2 antibody is administered to the individual at a dose of about 25 mg/kg once every five weeks. In some embodiments, the anti-TREM2 antibody is administered to the individual at a dose of about 30 mg/kg once every five weeks. In some embodiments, the anti-TREM2 antibody is administered to the individual at a dose of about 35 mg/kg once every five weeks. In some embodiments, the anti-TREM2 antibody is administered to the individual at a dose of about 40 mg/kg once every five weeks. In some embodiments, the anti-TREM2 antibody is administered to the individual at a dose of about 45 mg/kg once every five weeks. In some embodiments, the anti-TREM2 antibody is administered to the individual at a dose of about 50 mg/kg once every five weeks. In some embodiments, the anti-TREM2 antibody is administered to the individual at a dose of about 55 mg/kg once every five weeks. In some embodiments, the anti-TREM2 antibody is administered to the individual at a dose of about 60 mg/kg once every five weeks.

In some embodiments, the anti-TREM2 antibody is administered to the individual at a dose of at least about 15 mg/kg once every six weeks. In some embodiments, the anti-TREM2 antibody is administered to the individual at a dose of about 15 mg/kg once every six weeks. In some embodiments, the anti-TREM2 antibody is administered to the individual at a dose of about 20 mg/kg once every six weeks. In some embodiments, the anti-TREM2 antibody is administered to the individual at a dose of about 25 mg/kg once every six weeks. In some embodiments, the anti-TREM2 antibody is administered to the individual at a dose of about 30 mg/kg once every six weeks. In some embodiments, the anti-TREM2 antibody is administered to the individual at a dose of about 35 mg/kg once every six weeks. In some embodiments, the anti-TREM2 antibody is administered to the individual at a dose of about 40 mg/kg once every six weeks. In some embodiments, the anti-TREM2 antibody is administered to the individual at a dose of about 45 mg/kg once every six weeks. In some embodiments, the anti-TREM2 antibody is administered to the individual at a dose of about 50 mg/kg once every six weeks. In some embodiments, the anti-TREM2 antibody is administered to the individual at a dose of about 55 mg/kg once every six weeks. In some embodiments, the anti-TREM2 antibody is administered to the individual at a dose of about 60 mg/kg once every six weeks.

In some embodiments, the anti-TREM2 antibody is administered to the individual at a dose of at least about 15 mg/kg once every seven weeks. In some embodiments, the anti-TREM2 antibody is administered to the individual at a dose of about 15 mg/kg once every seven weeks. In some embodiments, the anti-TREM2 antibody is administered to the individual at a dose of about 20 mg/kg once every seven weeks. In some embodiments, the anti-TREM2 antibody is administered to the individual at a dose of about 25 mg/kg once every seven weeks. In some embodiments, the anti-TREM2 antibody is administered to the individual at a dose of about 30 mg/kg once every seven weeks. In some embodiments, the anti-TREM2 antibody is administered to the individual at a dose of about 35 mg/kg once every seven weeks. In some embodiments, the anti-TREM2 antibody is administered to the individual at a dose of about 40 mg/kg once every seven weeks. In some embodiments, the anti-TREM2 antibody is administered to the individual at a dose of about 45 mg/kg once every seven weeks. In some embodiments, the anti-TREM2 antibody is administered to the individual at a dose of about 50 mg/kg once every seven weeks. In some embodiments, the anti-TREM2 antibody is administered to the individual at a dose of about 55 mg/kg once every seven weeks. In some embodiments, the anti-TREM2 antibody is administered to the individual at a dose of about 60 mg/kg once every seven weeks.

In some embodiments, the anti-TREM2 antibody is administered to the individual at a dose of at least about 15 mg/kg once every eight weeks. In some embodiments, the anti-TREM2 antibody is administered to the individual at a dose of about 15 mg/kg once every eight weeks. In some embodiments, the anti-TREM2 antibody is administered to the individual at a dose of about 20 mg/kg once every eight weeks. In some embodiments, the anti-TREM2 antibody is administered to the individual at a dose of about 25 mg/kg once every eight weeks. In some embodiments, the anti-TREM2 antibody is administered to the individual at a dose of about 30 mg/kg once every eight weeks. In some embodiments, the anti-TREM2 antibody is administered to the individual at a dose of about 35 mg/kg once every eight weeks. In some embodiments, the anti-TREM2 antibody is administered to the individual at a dose of about 40 mg/kg once every eight weeks. In some embodiments, the anti-TREM2 antibody is administered to the individual at a dose of about 45 mg/kg once every eight weeks. In some embodiments, the anti-TREM2 antibody is administered to the individual at a dose of about 50 mg/kg once every eight weeks. In some embodiments, the anti-TREM2 antibody is administered to the individual at a dose of about 55 mg/kg once every eight weeks. In some embodiments, the anti-TREM2 antibody is administered to the individual at a dose of about 60 mg/kg once every eight weeks.

In certain embodiments, each dose of an anti-TREM2 antibody is administered to the individual intravenously over about 60 minutes. In some embodiments, the anti-TREM2 antibody is administered to the individual at a dose of at least about 15 mg/kg over about 60 minutes. In some embodiments, the anti-TREM2 antibody is administered to the individual at a dose of about 15 mg/kg over about 60 minutes. In some embodiments, the anti-TREM2 antibody is administered to the individual at a dose of about 20 mg/kg over about 60 minutes. In some embodiments, the anti-TREM2 antibody is administered to the individual at a dose of about 25 mg/kg over about 60 minutes. In some embodiments, the anti-TREM2 antibody is administered to the individual at a dose of about 30 mg/kg over about 60 minutes. In some embodiments, the anti-TREM2 antibody is administered to the individual at a dose of about 35 mg/kg over about 60 minutes. In some embodiments, the anti-TREM2 antibody is administered to the individual at a dose of about 40 mg/kg over about 60 minutes. In some embodiments, the anti-TREM2 antibody is administered to the individual at a dose of about 45 mg/kg over about 60 minutes. In some embodiments, the anti-TREM2 antibody is administered to the individual at a dose of about 50 mg/kg over about 60 minutes. In some embodiments, the anti-TREM2 antibody is administered to the individual at a dose of about 55 mg/kg over about 60 minutes. In some embodiments, the anti-TREM2 antibody is administered to the individual at a dose of about 60 mg/kg over about 60 minutes.

In certain embodiments, each dose of the anti-TREM2 antibody is administered to the individual intravenously over at least about 60 minutes. In some embodiments, the anti-TREM2 antibody is administered to the individual at a dose of at least about 15 mg/kg over at least about 60 minutes. In some embodiments, the anti-TREM2 antibody is administered to the individual at a dose of about 15 mg/kg over at least about 60 minutes. In some embodiments, the anti-TREM2 antibody is administered to the individual at a dose of about 20 mg/kg over at least about 60 minutes. In some embodiments, the anti-TREM2 antibody is administered to the individual at a dose of about 25 mg/kg over at least about 60 minutes. In some embodiments, the anti-TREM2 antibody is administered to the individual at a dose of about 30 mg/kg over at least about 60 minutes. In some embodiments, the anti-TREM2 antibody is administered to the individual at a dose of about 35 mg/kg over at least about 60 minutes. In some embodiments, the anti-TREM2 antibody is administered to the individual at a dose of about 40 mg/kg over at least about 60 minutes. In some embodiments, the anti-TREM2 antibody is administered to the individual at a dose of about 45 mg/kg over at least about 60 minutes. In some embodiments, the anti-TREM2 antibody is administered to the individual at a dose of about 50 mg/kg over at least about 60 minutes. In some embodiments, the anti-TREM2 antibody is administered to the individual at a dose of about 55 mg/kg over at least about 60 minutes. In some embodiments, the anti-TREM2 antibody is administered to the individual at a dose of about 60 mg/kg over at least about 60 minutes.

In certain embodiments, at least 1 dose, at least 2 doses, at least 3 doses, at least 4 doses, at least 5 doses, at least 6 doses, at least 7 doses, at least 8 doses, at least 9 doses, at least 10 doses, at least 11 doses, at least 12 doses, at least 13 doses, at least 14 doses, at least 15 doses, at least 16 doses, at least 17 doses, at least 18 doses, at least 19 doses, or at least 20 doses of the anti-TREM2 antibody are administered to the individual intravenously.

In some embodiments, the individual is treated for a treatment period of at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 17 weeks, at least about 18 weeks, at least about 19 weeks, at least about 20 weeks, at least about 21 weeks, at least about 22 weeks, at least about 23 weeks, at least about 24 weeks, at least about 25 weeks, at least about 26 weeks, at least about 27 weeks, at least about 28 weeks, at least about 29 weeks, at least about 30 weeks, at least about 31 weeks, at least about 32 weeks, at least about 33 weeks, at least about 34 weeks, at least about 35 weeks, at least about 36 weeks, at least about 37 weeks, at least about 38 weeks, at least about 39 weeks, at least about 40 weeks, at least about 41 weeks, at least about 42 weeks, at least about 43 weeks, at least about 44 weeks, at least about 45 weeks, at least about 46 weeks, at least about 47 weeks, at least about 48 weeks, at least about 49 weeks, at least about 50 weeks, at least about 51 weeks, or at least about 52 weeks in length.

In some embodiments, the individual is treated for a treatment period of up to 4 weeks, up to 5 weeks, up to 6 weeks, up to 7 weeks, up to 8 weeks, up to 9 weeks, up to 10 weeks, up to 11 weeks, up to 12 weeks, up to 13 weeks, up to 14 weeks, up to 15 weeks, up to 16 weeks, up to 17 weeks, up to 18 weeks, up to 19 weeks, up to 20 weeks, up to 21 weeks, up to 22 weeks, up to 23 weeks, up to 24 weeks, up to 25 weeks, up to 26 weeks, up to 27 weeks, up to 28 weeks, up to 29 weeks, up to 30 weeks, up to 31 weeks, up to 32 weeks, up to 33 weeks, up to 34 weeks, up to 35 weeks, up to 36 weeks, up to 37 weeks, up to 38 weeks, up to 39 weeks, up to 40 weeks, up to 41 weeks, up to 42 weeks, up to 43 weeks, up to 44 weeks, up to 45 weeks, up to 46 weeks, up to 47 weeks, up to 48 weeks, up to 49 weeks, up to 50 weeks, up to 51 weeks, or up to 52 weeks in length.

In some embodiments, administration of the anti-TREM2 antibody occurs on the first day of the treatment period and every week thereafter. In some embodiments, administration of the anti-TREM2 antibody occurs on the first day of the treatment period and every two weeks thereafter. In some embodiments, administration of the anti-TREM2 antibody occurs on the first day of the treatment period and every three weeks thereafter. In some embodiments, administration of the anti-TREM2 antibody occurs on the first day of the treatment period and every four weeks thereafter. In some embodiments, administration of the anti-TREM2 antibody occurs on the first day of the treatment period and every five weeks thereafter. In some embodiments, administration of the anti-TREM2 antibody occurs on the first day of the treatment period and every six weeks thereafter. In some embodiments, administration of the anti-TREM2 antibody occurs on the first day of the treatment period and every seven weeks thereafter. In some embodiments, administration of the anti-TREM2 antibody occurs on the first day of the treatment period and every eight weeks thereafter.

An initial higher loading dose, followed by one or more lower doses may be administered. However, other dosage regimens may be useful. The progress of this therapy is easily monitored by conventional techniques and assays.

### Soluble TREM2 Levels

As used herein, “soluble TREM2” or “sTREM2” refer to any form of TREM2 that results from processing, e.g., cleavage, of a TREM2 protein, resulting in a soluble, processed form of TREM2, e.g., as described herein in the “TREM2 Proteins” section.

In some aspects, methods of the present disclosure comprise administering an anti-TREM2 antibody to an individual intravenously, wherein administration of the anti-TREM2 antibody to the individual results in a decrease in the levels of soluble TREM2 in the cerebrospinal fluid of the individual, compared to the levels of soluble TREM2 in the cerebrospinal fluid of the individual prior to administration of the anti-TREM2 antibody. In some embodiments, administration of the anti-TREM2 antibody to the individual results in a decrease of any of at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% in the levels of soluble TREM2 in the cerebrospinal fluid of the individual, compared to the levels of soluble TREM2 in the cerebrospinal fluid of the individual prior to administration of the anti-TREM2 antibody. In some embodiments, administration of the anti-TREM2 antibody to the individual results in at least about a 30% decrease in the levels of soluble TREM2 in the cerebrospinal fluid of the individual, compared to the levels of soluble TREM2 in the cerebrospinal fluid of the individual prior to administration of the anti-TREM2 antibody. In some embodiments, administration of the anti-TREM2 antibody to the individual results in at least about a 40% decrease in the levels of soluble TREM2 in the cerebrospinal fluid of the individual, compared to the levels of soluble TREM2 in the cerebrospinal fluid of the individual prior to administration of the anti-TREM2 antibody. In some embodiments, administration of the anti-TREM2 antibody to the individual results in at least about a 50% decrease in the levels of soluble TREM2 in the cerebrospinal fluid of the individual, compared to the levels of soluble TREM2 in the cerebrospinal fluid of the individual prior to administration of the anti-TREM2 antibody. In some embodiments, administration of the anti-TREM2 antibody to the individual results in at least about a 60% decrease in the levels of soluble TREM2 in the cerebrospinal fluid of the individual, compared to the levels of soluble TREM2 in the cerebrospinal fluid of the individual prior to administration of the anti-TREM2 antibody.

In some embodiments, the decrease in the levels of soluble TREM2 in the cerebrospinal fluid of the individual compared to the levels of soluble TREM2 in the cerebrospinal fluid of the individual prior to administration of the anti-TREM2 antibody is present at about 2 days to about 12 days (e.g., any of 2 days, 3 days, 4 days, 5 days, 6 days, 7 days, 8 days, 9 days, 10 days, 11 days, or 12 days) after administration of the antibody. In some embodiments, the decrease in the levels of soluble TREM2 in the cerebrospinal fluid of the individual compared to the levels of soluble TREM2 in the cerebrospinal fluid of the individual prior to administration of the anti-TREM2 antibody is present for at least about 2 days after administration of the antibody. In some embodiments, the decrease in the levels of soluble TREM2 in the cerebrospinal fluid of the individual compared to the levels of soluble TREM2 in the cerebrospinal fluid of the individual prior to administration of the anti-TREM2 antibody is present for at least about 12 days after administration of the antibody. In some embodiments, the decrease in the levels of soluble TREM2 in the cerebrospinal fluid of the individual compared to the levels of soluble TREM2 in the cerebrospinal fluid of the individual prior to administration of the anti-TREM2 antibody is present at about 2 days after administration of the antibody. In some embodiments the decrease in the levels of soluble TREM2 in the cerebrospinal fluid of the individual, compared to the levels of soluble TREM2 in the cerebrospinal fluid of the individual prior to administration of the anti-TREM2 antibody is present at about 12 days after administration of the antibody.

In some embodiments, administration of an anti-TREM2 antibody of the disclosure at a dose of about 15 mg/kg results in a decrease in the levels of soluble TREM2 in the cerebrospinal fluid of the individual of at least about 30% at 2 days after administration of the antibody, compared to the levels of soluble TREM2 in the cerebrospinal fluid of the individual prior to administration of the anti-TREM2 antibody. In some embodiments, administration of an anti-TREM2 antibody of the disclosure at a dose of about 15 mg/kg results in a decrease in the levels of soluble TREM2 in the cerebrospinal fluid of the individual of at least about 40% at 2 days after administration of the antibody, compared to the levels of soluble TREM2 in the cerebrospinal fluid of the individual prior to administration of the anti-TREM2 antibody. In some embodiments, administration of an anti-TREM2 antibody of the disclosure at a dose of about 30 mg/kg results in a decrease in the levels of soluble TREM2 in the cerebrospinal fluid of the individual of at least about 30% at 2 days after administration of the antibody, compared to the levels of soluble TREM2 in the cerebrospinal fluid of the individual prior to administration of the anti-TREM2 antibody. In some embodiments, administration of an anti-TREM2 antibody of the disclosure at a dose of about 30 mg/kg results in a decrease in the levels of soluble TREM2 in the cerebrospinal fluid of the individual of at least about 40% at 2 days after administration of the antibody, compared to the levels of soluble TREM2 in the cerebrospinal fluid of the individual prior to administration of the anti-TREM2 antibody. In some embodiments, administration of an anti-TREM2 antibody of the disclosure at a dose of about 45 mg/kg results in a decrease in the levels of soluble TREM2 in the cerebrospinal fluid of the individual of at least about 50% at 2 days after administration of the antibody, compared to the levels of soluble TREM2 in the cerebrospinal fluid of the individual prior to administration of the anti-TREM2 antibody. In some embodiments, administration of an anti-TREM2 antibody of the disclosure at a dose of about 60 mg/kg results in a decrease in the levels of soluble TREM2 in the cerebrospinal fluid of the individual of at least about 55% at 2 days after administration of the antibody, compared to the levels of soluble TREM2 in the cerebrospinal fluid of the individual prior to administration of the anti-TREM2 antibody.

In some embodiments, administration of an anti-TREM2 antibody of the disclosure at a dose of about 15 mg/kg results in a decrease in the levels of soluble TREM2 in the cerebrospinal fluid of the individual of at least about 30% at 12 days after administration of the antibody, compared to the levels of soluble TREM2 in the cerebrospinal fluid of the individual prior to administration of the anti-TREM2 antibody. In some embodiments, administration of an anti-TREM2 antibody of the disclosure at a dose of about 15 mg/kg results in a decrease in the levels of soluble TREM2 in the cerebrospinal fluid of the individual of at least about 40% at 12 days after administration of the antibody, compared to the levels of soluble TREM2 in the cerebrospinal fluid of the individual prior to administration of the anti-TREM2 antibody. In some embodiments, administration of an anti-TREM2 antibody of the disclosure at a dose of about 30 mg/kg results in a decrease in the levels of soluble TREM2 in the cerebrospinal fluid of the individual of at least about 30% at 12 days after administration of the antibody, compared to the levels of soluble TREM2 in the cerebrospinal fluid of the individual prior to administration of the anti-TREM2 antibody. In some embodiments, administration of an anti-TREM2 antibody of the disclosure at a dose of about 30 mg/kg results in a decrease in the levels of soluble TREM2 in the cerebrospinal fluid of the individual of at least about 40% at 12 days after administration of the antibody, compared to the levels of soluble TREM2 in the cerebrospinal fluid of the individual prior to administration of the anti-TREM2 antibody. In some embodiments, administration of an anti-TREM2 antibody of the disclosure at a dose of about 45 mg/kg results in a decrease in the levels of soluble TREM2 in the cerebrospinal fluid of the individual of at least about 30% at 12 days after administration of the antibody, compared to the levels of soluble TREM2 in the cerebrospinal fluid of the individual prior to administration of the anti-TREM2 antibody. In some embodiments, administration of an anti-TREM2 antibody of the disclosure at a dose of about 45 mg/kg results in a decrease in the levels of soluble TREM2 in the cerebrospinal fluid of the individual of at least about 40% at 12 days after administration of the antibody, compared to the levels of soluble TREM2 in the cerebrospinal fluid of the individual prior to administration of the anti-TREM2 antibody. In some embodiments, administration of an anti-TREM2 antibody of the disclosure at a dose of about 60 mg/kg results in a decrease in the levels of soluble TREM2 in the cerebrospinal fluid of the individual of at least about 40% at 12 days after administration of the antibody, compared to the levels of soluble TREM2 in the cerebrospinal fluid of the individual prior to administration of the anti-TREM2 antibody. In some embodiments, administration of an anti-TREM2 antibody of the disclosure at a dose of about 60 mg/kg results in a decrease in the levels of soluble TREM2 in the cerebrospinal fluid of the individual of at least about 50% at 12 days after administration of the antibody, compared to the levels of soluble TREM2 in the cerebrospinal fluid of the individual prior to administration of the anti-TREM2 antibody.

In some embodiments, the levels of soluble TREM2 in the cerebrospinal fluid of the individual are compared to the levels of soluble TREM2 in the cerebrospinal fluid of the individual at between about 42 days to less than 1 day (e.g., any of 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less than 1 day) prior to administration of the anti-TREM2 antibody. In some embodiments, the levels of soluble TREM2 in the cerebrospinal fluid of the individual are compared to the levels of soluble TREM2 in the cerebrospinal fluid of the individual at least about 4 days prior to administration of the anti-TREM2 antibody.

The levels of sTREM2 in the cerebrospinal fluid of the individual may be measured using any method known in the art, such as ELISA, immunoassays, immunoblotting, and mass spectrometry.

In certain embodiments, the levels of sTREM2 in the cerebrospinal fluid of the individual are measured with an immunoassay using an electrochemiluminescent methodology. For example, in some embodiments, an anti-human TREM2 antibody is diluted in coating buffer and immobilized onto a 96-well microtiter sample plate. After blocking and washing the plate, endogenous quality control and study samples are diluted with assay buffer, dispensed onto the sample plate, and incubated. A second anti-human TREM2 antibody that binds to a different epitope than the first antibody is then added as the capture antibody. The plate is subsequently washed, and Sulfo-Tag streptavidin is added and incubated, followed by addition of MSD Read Buffer T. Concentrations of sTREM2 (i.e., the levels of sTREM2) are determined on a standard curve obtained by relative light units versus concentration. The calibration curve is generated using a four-parameter curve fit with 1/y² weighting. The qualified range for this method in human CSF is from 0.400 ng/mL to 50.0 ng/mL.

### Soluble CSF1R Levels

As used herein, “soluble CSF1R” or “sCSF1R” refer to any form of CSF1R that results from processing, e.g., cleavage, of a CSF1R protein, resulting in a soluble, processed form of CSF1R, e.g., as described herein in Example 2.

In some aspects, methods of the present disclosure comprise administering an anti-TREM2 antibody to an individual intravenously, wherein administration of the anti-TREM2 antibody to the individual results in an increase in the levels of soluble CSF1R in the cerebrospinal fluid of the individual, compared to the levels of soluble CSF1R in the cerebrospinal fluid of the individual prior to administration of the anti-TREM2 antibody. In some embodiments, administration of the anti-TREM2 antibody results in an increase of any of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% in the levels of soluble CSF1R in the cerebrospinal fluid of the individual, compared to the levels of soluble CSF1R in the cerebrospinal fluid of the individual prior to administration of the anti-TREM2 antibody. In some embodiments, administration of the anti-TREM2 antibody results in at least about a 5% increase in the levels of soluble CSF1R in the cerebrospinal fluid of the individual, compared to the levels of soluble CSF1R in the cerebrospinal fluid of the individual prior to administration of the anti-TREM2 antibody. In some embodiments, administration of the anti-TREM2 antibody results in at least about a 10% increase in the levels of soluble CSF1R in the cerebrospinal fluid of the individual, compared to the levels of soluble CSF1R in the cerebrospinal fluid of the individual prior to administration of the anti-TREM2 antibody. In some embodiments, administration of the anti-TREM2 antibody results in at least about a 15% increase in the levels of soluble CSF1R in the cerebrospinal fluid of the individual, compared to the levels of soluble CSF1R in the cerebrospinal fluid of the individual prior to administration of the anti-TREM2 antibody. In some embodiments, administration of the anti-TREM2 antibody results in at least about a 20% increase in the levels of soluble CSF1R in the cerebrospinal fluid of the individual, compared to the levels of soluble CSF1R in the cerebrospinal fluid of the individual prior to administration of the anti-TREM2 antibody. In some embodiments, administration of the anti-TREM2 antibody results in at least about a 25% increase in the levels of soluble CSF1R in the cerebrospinal fluid of the individual, compared to the levels of soluble CSF1R in the cerebrospinal fluid of the individual prior to administration of the anti-TREM2 antibody.

In some embodiments, the increase in the levels of soluble CSF1R in the cerebrospinal fluid of the individual, compared to the levels of soluble CSF1R in the cerebrospinal fluid of the individual prior to administration of the anti-TREM2 antibody is present at about 2 days to about 12 days (e.g., any of 2 days, 3 days, 4 days, 5 days, 6 days, 7 days, 8 days, 9 days, 10 days, 11 days, or 12 days) administration of the antibody. In some embodiments, the increase in the levels of soluble CSF1R in the cerebrospinal fluid of the individual, compared to the levels of soluble CSF1R in the cerebrospinal fluid of the individual prior to administration of the anti-TREM2 antibody is present for at least about 2 days after administration of the antibody. In some embodiments, the increase in the levels of soluble CSF1R in the cerebrospinal fluid of the individual, compared to the levels of soluble CSF1R in the cerebrospinal fluid of the individual prior to administration of the anti-TREM2 antibody is present for at least about 12 days after administration of the antibody. In some embodiments, the increase in the levels of soluble CSF1R in the cerebrospinal fluid of the individual, compared to the levels of soluble CSF1R in the cerebrospinal fluid of the individual prior to administration of the anti-TREM2 antibody is present at about day 2 after administration of the antibody. In some embodiments, the increase in the levels of soluble CSF1R in the cerebrospinal fluid of the individual, compared to the levels of soluble CSF1R in the cerebrospinal fluid of the individual prior to administration of the anti-TREM2 antibody is present at about day 12 after administration of the antibody.

In some embodiments, administration of an anti-TREM2 antibody of the disclosure at a dose of about 15 mg/kg results in an increase in the levels of soluble CSF1R in the cerebrospinal fluid of the individual of at least about 5% at 2 days after administration of the antibody, compared to the levels of soluble CSF1R in the cerebrospinal fluid of the individual prior to administration of the anti-TREM2 antibody. In some embodiments, administration of an anti-TREM2 antibody of the disclosure at a dose of about 30 mg/kg results in an increase in the levels of soluble CSF1R in the cerebrospinal fluid of the individual of at least about 10% at 2 days after administration of the antibody, compared to the levels of soluble CSF1R in the cerebrospinal fluid of the individual prior to administration of the anti-TREM2 antibody. In some embodiments, administration of an anti-TREM2 antibody of the disclosure at a dose of about 45 mg/kg results in an increase in the levels of soluble CSF1R in the cerebrospinal fluid of the individual of at least about 15% at 2 days after administration of the antibody, compared to the levels of soluble CSF1R in the cerebrospinal fluid of the individual prior to administration of the anti-TREM2 antibody. In some embodiments, administration of an anti-TREM2 antibody of the disclosure at a dose of about 60 mg/kg results in an increase in the levels of soluble CSF1R in the cerebrospinal fluid of the individual of at least about 25% at 2 days after administration of the antibody, compared to the levels of soluble CSF1R in the cerebrospinal fluid of the individual prior to administration of the anti-TREM2 antibody.

In some embodiments, administration of an anti-TREM2 antibody of the disclosure at a dose of about 15 mg/kg results in an increase in the levels of soluble CSF1R in the cerebrospinal fluid of the individual of at least about 5% at 12 days after administration of the antibody, compared to the levels of soluble CSF1R in the cerebrospinal fluid of the individual prior to administration of the anti-TREM2 antibody. In some embodiments, administration of an anti-TREM2 antibody of the disclosure at a dose of about 30 mg/kg results in an increase in the levels of soluble CSF1R in the cerebrospinal fluid of the individual of at least about 10% at 12 days after administration of the antibody, compared to the levels of soluble CSF1R in the cerebrospinal fluid of the individual prior to administration of the anti-TREM2 antibody. In some embodiments, administration of an anti-TREM2 antibody of the disclosure at a dose of about 45 mg/kg results in an increase in the levels of soluble CSF1R in the cerebrospinal fluid of the individual of at least about 1% at 12 days after administration of the antibody, compared to the levels of soluble CSF1R in the cerebrospinal fluid of the individual prior to administration of the anti-TREM2 antibody. In some embodiments, administration of an anti-TREM2 antibody of the disclosure at a dose of about 60 mg/kg results in an increase in the levels of soluble CSF1R in the cerebrospinal fluid of the individual of at least about 10% at 12 days after administration of the antibody, compared to the levels of soluble CSF1R in the cerebrospinal fluid of the individual prior to administration of the anti-TREM2 antibody.

In some embodiments, the levels of soluble CSF1R in the cerebrospinal fluid of the individual are compared to the levels of soluble CSF1R in the cerebrospinal fluid of the individual at between about 42 days to less than 1 day (e.g., any of 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less than 1 day) prior to administration of the anti-TREM2 antibody. In some embodiments, the levels of soluble CSF1R in the cerebrospinal fluid of the individual are compared to the levels of soluble CSF1R in the cerebrospinal fluid of the individual at least about 4 days prior to administration of the anti-TREM2 antibody.

The levels of sCSF1R in the cerebrospinal fluid of the individual may be measured using any method known in the art, such as ELISA (e.g., an ELISA assay from R&D Systems), immunoassays, immunoblotting, and mass spectrometry. In certain embodiments, the levels of sCSF1R in the cerebrospinal fluid of the individual are measured with an ELISA assay from R&D Systems which has a qualified range in 100% human CSF of 125 pg/mL to 4000 pg/mL.

### YKL40 Levels

In some aspects, methods of the present disclosure comprise administering an anti-TREM2 antibody to an individual intravenously, wherein administration of the anti-TREM2 antibody to the individual results in an increase in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in an increase of any of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, or at least about 200% in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in at least about a 5% increase in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in at least about a 10% increase in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in at least about a 15% increase in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in at least about a 20% increase in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in at least about a 25% increase in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in at least about a 30% increase in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in at least about a 40% increase in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in at least about a 50% increase in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in at least about a 60% increase in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in at least about a 70% increase in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in at least about an 80% increase in the levels of YKL40 in the cerebrospinal fluid of the individual, compared to the levels of YKL40 in the cerebrospinal fluid of the individual prior to administration of the anti-TREM2 antibody. In some embodiments, administration of the anti-TREM2 antibody results in at least about a 90% increase in the levels of YKL40 in the cerebrospinal fluid of the individual, compared to the levels of YKL40 in the cerebrospinal fluid of the individual prior to administration of the anti-TREM2 antibody.

In some embodiments, the increase in the levels of YKL40 in the cerebrospinal fluid of the individual, compared to the levels of YKL40 in the cerebrospinal fluid of the individual prior to administration of the anti-TREM2 antibody is present at about 2 days to about 12 days (e.g., any of 2 days, 3 days, 4 days, 5 days, 6 days, 7 days, 8 days, 9 days, 10 days, 11 days, or 12 days) after administration of the antibody. In some embodiments, the increase in the levels of YKL40 in the cerebrospinal fluid of the individual, compared to the levels of YKL40 in the cerebrospinal fluid of the individual prior to administration of the anti-TREM2 antibody is present for at least about 2 days after administration of the antibody. In some embodiments, the increase in the levels of YKL40 in the cerebrospinal fluid of the individual, compared to the levels of YKL40 in the cerebrospinal fluid of the individual prior to administration of the anti-TREM2 antibody is present for at least about 12 days after administration of the antibody. In some embodiments, the increase in the levels of YKL40 in the cerebrospinal fluid of the individual, compared to the levels of YKL40 in the cerebrospinal fluid of the individual prior to administration of the anti-TREM2 antibody is present at about day 2 after administration of the antibody. In some embodiments, the increase in the levels of YKL40 in the cerebrospinal fluid of the individual, compared to the levels of YKL40 in the cerebrospinal fluid of the individual prior to administration of the anti-TREM2 antibody is present at about day 12 after administration of the antibody.

In some embodiments, administration of an anti-TREM2 antibody of the disclosure at a dose of about 15 mg/kg results in an increase in the levels of YKL40 in the cerebrospinal fluid of the individual of at least about 1% at 2 days after administration of the antibody, compared to the levels of YKL40 in the cerebrospinal fluid of the individual prior to administration of the anti-TREM2 antibody. In some embodiments, administration of an anti-TREM2 antibody of the disclosure at a dose of about 30 mg/kg results in an increase in the levels of YKL40 in the cerebrospinal fluid of the individual of at least about 10% at 2 days after administration of the antibody, compared to the levels of YKL40 in the cerebrospinal fluid of the individual prior to administration of the anti-TREM2 antibody. In some embodiments, administration of an anti-TREM2 antibody of the disclosure at a dose of about 45 mg/kg results in an increase in the levels of YKL40 in the cerebrospinal fluid of the individual of at least about 25% at 2 days after administration of the antibody, compared to the levels of YKL40 in the cerebrospinal fluid of the individual prior to administration of the anti-TREM2 antibody. In some embodiments, administration of an anti-TREM2 antibody of the disclosure at a dose of about 60 mg/kg results in an increase in the levels of YKL40 in the cerebrospinal fluid of the individual of at least about 75% at 2 days after administration of the antibody, compared to the levels of YKL40 in the cerebrospinal fluid of the individual prior to administration of the anti-TREM2 antibody.

In some embodiments, administration of an anti-TREM2 antibody of the disclosure at a dose of about 15 mg/kg results in an increase in the levels of YKL40 in the cerebrospinal fluid of the individual of at least about 1% at 12 days after administration of the antibody, compared to the levels of YKL40 in the cerebrospinal fluid of the individual prior to administration of the anti-TREM2 antibody. In some embodiments, administration of an anti-TREM2 antibody of the disclosure at a dose of about 30 mg/kg results in an increase in the levels of YKL40 in the cerebrospinal fluid of the individual of at least about 1% at 12 days after administration of the antibody, compared to the levels of YKL40 in the cerebrospinal fluid of the individual prior to administration of the anti-TREM2 antibody. In some embodiments, administration of an anti-TREM2 antibody of the disclosure at a dose of about 45 mg/kg results in an increase in the levels of YKL40 in the cerebrospinal fluid of the individual of at least about 1% at 12 days after administration of the antibody, compared to the levels of YKL40 in the cerebrospinal fluid of the individual prior to administration of the anti-TREM2 antibody. In some embodiments, administration of an anti-TREM2 antibody of the disclosure at a dose of about 60 mg/kg results in an increase in the levels of YKL40 in the cerebrospinal fluid of the individual of at least about 5% at 12 days after administration of the antibody, compared to the levels of YKL40 in the cerebrospinal fluid of the individual prior to administration of the anti-TREM2 antibody.

In some embodiments, the levels of YKL40 in the cerebrospinal fluid of the individual are compared to the levels of YKL40 in the cerebrospinal fluid of the individual at between about 42 days to less than 1 day (e.g., any of 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less than 1 day) prior to administration of the anti-TREM2 antibody. In some embodiments, the levels of YKL40 in the cerebrospinal fluid of the individual are compared to the levels of YKL40 in the cerebrospinal fluid of the individual at least about 4 days prior to administration of the anti-TREM2 antibody.

The levels of YKL40 in the cerebrospinal fluid of the individual may be measured using any method known in the art, such as ELISA, immunoassays, immunoblotting, and mass spectrometry. In certain embodiments, the levels of YKL40 in the cerebrospinal fluid of the individual are measured using an immunoassay from Roche.

### IL-1RA Levels

In some aspects, methods of the present disclosure comprise administering an anti-TREM2 antibody to an individual intravenously, wherein administration of the anti-TREM2 antibody to the individual results in an increase in the levels of IL-1RA in the cerebrospinal fluid of the individual, compared to the levels of IL-1RA in the cerebrospinal fluid of the individual prior to administration of the anti-TREM2 antibody. In some embodiments, administration of the anti-TREM2 antibody results in an increase of any of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 225%, at least about 250%, at least about 275%, at least about 300%, at least about 325%, at least about 350%, at least about 375%, at least about 400%, at least about 450%, at least about 500%, at least about 550%, at least about 600%, at least about 650%, at least about 700%, at least about 750%, at least about 800%, at least about 850%, or at least about 900% in the levels of IL-1RA in the cerebrospinal fluid of the individual, compared to the levels of IL-1RA in the cerebrospinal fluid of the individual prior to administration of the anti-TREM2 antibody. In some embodiments, administration of the anti-TREM2 antibody results in at least about a 10% increase in the levels of IL-1RA in the cerebrospinal fluid of the individual, compared to the levels of IL-1RA in the cerebrospinal fluid of the individual prior to administration of the anti-TREM2 antibody. In some embodiments, administration of the anti-TREM2 antibody results in at least about a 25% increase in the levels of IL-1RA in the cerebrospinal fluid of the individual, compared to the levels of IL-1RA in the cerebrospinal fluid of the individual prior to administration of the anti-TREM2 antibody. In some embodiments, administration of the anti-TREM2 antibody results in at least about a 50% increase in the levels of IL-1RA in the cerebrospinal fluid of the individual, compared to the levels of IL-1RA in the cerebrospinal fluid of the individual prior to administration of the anti-TREM2 antibody. In some embodiments, administration of the anti-TREM2 antibody results in at least about a 75% increase in the levels of IL-1RA in the cerebrospinal fluid of the individual, compared to the levels of IL-1RA in the cerebrospinal fluid of the individual prior to administration of the anti-TREM2 antibody. In some embodiments, administration of the anti-TREM2 antibody results in at least about a 100% increase in the levels of IL-1RA in the cerebrospinal fluid of the individual, compared to the levels of IL-1RA in the cerebrospinal fluid of the individual prior to administration of the anti-TREM2 antibody. In some embodiments, administration of the anti-TREM2 antibody results in at least about a 125% increase in the levels of IL-1RA in the cerebrospinal fluid of the individual, compared to the levels of IL-1RA in the cerebrospinal fluid of the individual prior to administration of the anti-TREM2 antibody. In some embodiments, administration of the anti-TREM2 antibody results in at least about a 150% increase in the levels of IL-1RA in the cerebrospinal fluid of the individual, compared to the levels of IL-1RA in the cerebrospinal fluid of the individual prior to administration of the anti-TREM2 antibody. In some embodiments, administration of the anti-TREM2 antibody results in at least about a 175% increase in the levels of IL-1RA in the cerebrospinal fluid of the individual, compared to the levels of IL-1RA in the cerebrospinal fluid of the individual prior to administration of the anti-TREM2 antibody. In some embodiments, administration of the anti-TREM2 antibody results in at least about a 200% increase in the levels of IL-1RA in the cerebrospinal fluid of the individual, compared to the levels of IL-1RA in the cerebrospinal fluid of the individual prior to administration of the anti-TREM2 antibody. In some embodiments, administration of the anti-TREM2 antibody results in at least about a 250% increase in the levels of IL-1RA in the cerebrospinal fluid of the individual, compared to the levels of IL-1RA in the cerebrospinal fluid of the individual prior to administration of the anti-TREM2 antibody. In some embodiments, administration of the anti-TREM2 antibody results in at least about a 300% increase in the levels of IL-1RA in the cerebrospinal fluid of the individual, compared to the levels of IL-1RA in the cerebrospinal fluid of the individual prior to administration of the anti-TREM2 antibody.

In some embodiments, the increase in the levels of IL-1RA in the cerebrospinal fluid of the individual, compared to the levels of IL-1RA in the cerebrospinal fluid of the individual prior to administration of the anti-TREM2 antibody is present at about 2 days to about 12 days (e.g., any of 2 days, 3 days, 4 days, 5 days, 6 days, 7 days, 8 days, 9 days, 10 days, 11 days, or 12 days) after administration of the antibody. In some embodiments, the increase in the levels of IL-1RA in the cerebrospinal fluid of the individual, compared to the levels of IL-1RA in the cerebrospinal fluid of the individual prior to administration of the anti-TREM2 antibody is present for at least about 2 days after administration of the antibody. In some embodiments, the increase in the levels of IL-1RA in the cerebrospinal fluid of the individual, compared to the levels of IL-1RA in the cerebrospinal fluid of the individual prior to administration of the anti-TREM2 antibody is present for at least about 12 days after administration of the antibody. In some embodiments, the increase in the levels of IL-1RA in the cerebrospinal fluid of the individual, compared to the levels of IL-1RA in the cerebrospinal fluid of the individual prior to administration of the anti-TREM2 antibody is present at about day 2 after administration of the antibody. In some embodiments, the increase in the levels of IL-1RA in the cerebrospinal fluid of the individual, compared to the levels of IL-1RA in the cerebrospinal fluid of the individual prior to administration of the anti-TREM2 antibody is present at about day 12 after administration of the antibody.

In some embodiments, administration of an anti-TREM2 antibody of the disclosure at a dose of about 15 mg/kg results in an increase in the levels of IL-1RA in the cerebrospinal fluid of the individual of at least about 50% at 2 days after administration of the antibody, compared to the levels of IL-1RA in the cerebrospinal fluid of the individual prior to administration of the anti-TREM2 antibody. In some embodiments, administration of an anti-TREM2 antibody of the disclosure at a dose of about 30 mg/kg results in an increase in the levels of IL-1RA in the cerebrospinal fluid of the individual of at least about 300% at 2 days after administration of the antibody, compared to the levels of IL-1RA in the cerebrospinal fluid of the individual prior to administration of the anti-TREM2 antibody. In some embodiments, administration of an anti-TREM2 antibody of the disclosure at a dose of about 45 mg/kg results in an increase in the levels of IL-1RA in the cerebrospinal fluid of the individual of at least about 125% at 2 days after administration of the antibody, compared to the levels of IL-1RA in the cerebrospinal fluid of the individual prior to administration of the anti-TREM2 antibody. In some embodiments, administration of an anti-TREM2 antibody of the disclosure at a dose of about 60 mg/kg results in an increase in the levels of IL-1RA in the cerebrospinal fluid of the individual of at least about 125% at 2 days after administration of the antibody, compared to the levels of IL-1RA in the cerebrospinal fluid of the individual prior to administration of the anti-TREM2 antibody.

In some embodiments, administration of an anti-TREM2 antibody of the disclosure at a dose of about 15 mg/kg results in an increase in the levels of IL-1RA in the cerebrospinal fluid of the individual of at least about 10% at 12 days after administration of the antibody, compared to the levels of IL-1RA in the cerebrospinal fluid of the individual prior to administration of the anti-TREM2 antibody. In some embodiments, administration of an anti-TREM2 antibody of the disclosure at a dose of about 30 mg/kg results in an increase in the levels of IL-1RA in the cerebrospinal fluid of the individual of at least about 175% at 12 days after administration of the antibody, compared to the levels of IL-1RA in the cerebrospinal fluid of the individual prior to administration of the anti-TREM2 antibody. In some embodiments, administration of an anti-TREM2 antibody of the disclosure at a dose of about 45 mg/kg results in an increase in the levels of IL-1RA in the cerebrospinal fluid of the individual of at least about 25% at 12 days after administration of the antibody, compared to the levels of IL-1RA in the cerebrospinal fluid of the individual prior to administration of the anti-TREM2 antibody. In some embodiments, administration of an anti-TREM2 antibody of the disclosure at a dose of about 60 mg/kg results in an increase in the levels of IL-1RA in the cerebrospinal fluid of the individual of at least about 25% at 12 days after administration of the antibody, compared to the levels of IL-1RA in the cerebrospinal fluid of the individual prior to administration of the anti-TREM2 antibody.

In some embodiments, the levels of IL-1RA in the cerebrospinal fluid of the individual are compared to the levels of IL-1RA in the cerebrospinal fluid of the individual at between about 42 days to less than 1 day (e.g., any of 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less than 1 day) prior to administration of the anti-TREM2 antibody. In some embodiments, the levels of IL-1RA in the cerebrospinal fluid of the individual are compared to the levels of IL-1RA in the cerebrospinal fluid of the individual at least about 4 days prior to administration of the anti-TREM2 antibody.

The levels of IL-1RA in the cerebrospinal fluid of the individual may be measured using any method known in the art, such as ELISA, immunoassays, immunoblotting, and mass spectrometry. In certain embodiments, the levels of IL-1RA in the cerebrospinal fluid of the individual are measured using an ECL immunoassay using the Meso Scale Discovery system.

### Osteopontin Levels

In some aspects, methods of the present disclosure comprise administering an anti-TREM2 antibody to an individual intravenously, wherein administration of the anti-TREM2 antibody to the individual results in an increase in the levels of osteopontin in the cerebrospinal fluid of the individual, compared to the levels of osteopontin in the cerebrospinal fluid of the individual prior to administration of the anti-TREM2 antibody. In some embodiments, administration of the anti-TREM2 antibody results in an increase of any of at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 150%, at least about 175%, or at least about 200% in the levels of osteopontin in the cerebrospinal fluid of the individual, compared to the levels of osteopontin in the cerebrospinal fluid of the individual prior to administration of the anti-TREM2 antibody. In some embodiments, administration of the anti-TREM2 antibody results in at least about a 25% increase in the levels of osteopontin in the cerebrospinal fluid of the individual, compared to the levels of osteopontin in the cerebrospinal fluid of the individual prior to administration of the anti-TREM2 antibody. In some embodiments, administration of the anti-TREM2 antibody results in at least about a 30% increase in the levels of osteopontin in the cerebrospinal fluid of the individual, compared to the levels of osteopontin in the cerebrospinal fluid of the individual prior to administration of the anti-TREM2 antibody. In some embodiments, administration of the anti-TREM2 antibody results in at least about a 40% increase in the levels of osteopontin in the cerebrospinal fluid of the individual, compared to the levels of osteopontin in the cerebrospinal fluid of the individual prior to administration of the anti-TREM2 antibody. In some embodiments, administration of the anti-TREM2 antibody results in at least about a 50% increase in the levels of osteopontin in the cerebrospinal fluid of the individual, compared to the levels of osteopontin in the cerebrospinal fluid of the individual prior to administration of the anti-TREM2 antibody. In some embodiments, administration of the anti-TREM2 antibody results in at least about a 60% increase in the levels of osteopontin in the cerebrospinal fluid of the individual, compared to the levels of osteopontin in the cerebrospinal fluid of the individual prior to administration of the anti-TREM2 antibody. In some embodiments, administration of the anti-TREM2 antibody results in at least about a 70% increase in the levels of osteopontin in the cerebrospinal fluid of the individual, compared to the levels of osteopontin in the cerebrospinal fluid of the individual prior to administration of the anti-TREM2 antibody. In some embodiments, administration of the anti-TREM2 antibody results in at least about an 80% increase in the levels of osteopontin in the cerebrospinal fluid of the individual, compared to the levels of osteopontin in the cerebrospinal fluid of the individual prior to administration of the anti-TREM2 antibody. In some embodiments, administration of the anti-TREM2 antibody results in at least about a 90% increase in the levels of osteopontin in the cerebrospinal fluid of the individual, compared to the levels of osteopontin in the cerebrospinal fluid of the individual prior to administration of the anti-TREM2 antibody. In some embodiments, administration of the anti-TREM2 antibody results in at least about a 100% increase in the levels of osteopontin in the cerebrospinal fluid of the individual, compared to the levels of osteopontin in the cerebrospinal fluid of the individual prior to administration of the anti-TREM2 antibody. In some embodiments, administration of the anti-TREM2 antibody results in at least about a 110% increase in the levels of osteopontin in the cerebrospinal fluid of the individual, compared to the levels of osteopontin in the cerebrospinal fluid of the individual prior to administration of the anti-TREM2 antibody. In some embodiments, administration of the anti-TREM2 antibody results in at least about a 120% increase in the levels of osteopontin in the cerebrospinal fluid of the individual, compared to the levels of osteopontin in the cerebrospinal fluid of the individual prior to administration of the anti-TREM2 antibody.

In some embodiments, the increase in the levels of osteopontin in the cerebrospinal fluid of the individual, compared to the levels of osteopontin in the cerebrospinal fluid of the individual prior to administration of the anti-TREM2 antibody is present at about 2 days to about 12 days (e.g., any of 2 days, 3 days, 4 days, 5 days, 6 days, 7 days, 8 days, 9 days, 10 days, 11 days, or 12 days) after administration of the antibody. In some embodiments, the increase in the levels of osteopontin in the cerebrospinal fluid of the individual, compared to the levels of osteopontin in the cerebrospinal fluid of the individual prior to administration of the anti-TREM2 antibody is present for at least about 2 days after administration of the antibody. In some embodiments, the increase in the levels of osteopontin in the cerebrospinal fluid of the individual, compared to the levels of osteopontin in the cerebrospinal fluid of the individual prior to administration of the anti-TREM2 antibody is present for at least about 12 days after administration of the antibody. In some embodiments, the increase in the levels of osteopontin in the cerebrospinal fluid of the individual, compared to the levels of osteopontin in the cerebrospinal fluid of the individual prior to administration of the anti-TREM2 antibody is present at about day 2 after administration of the antibody. In some embodiments, the increase in the levels of osteopontin in the cerebrospinal fluid of the individual, compared to the levels of osteopontin in the cerebrospinal fluid of the individual prior to administration of the anti-TREM2 antibody is present at about day 12 after administration of the antibody.

In some embodiments, administration of an anti-TREM2 antibody of the disclosure at a dose of about 15 mg/kg results in an increase in the levels of osteopontin in the cerebrospinal fluid of the individual of at least about 35% at 2 days after administration of the antibody, compared to the levels of osteopontin in the cerebrospinal fluid of the individual prior to administration of the anti-TREM2 antibody. In some embodiments, administration of an anti-TREM2 antibody of the disclosure at a dose of about 30 mg/kg results in an increase in the levels of osteopontin in the cerebrospinal fluid of the individual of at least about 60% at 2 days after administration of the antibody, compared to the levels of osteopontin in the cerebrospinal fluid of the individual prior to administration of the anti-TREM2 antibody. In some embodiments, administration of an anti-TREM2 antibody of the disclosure at a dose of about 45 mg/kg results in an increase in the levels of osteopontin in the cerebrospinal fluid of the individual of at least about 30% at 2 days after administration of the antibody, compared to the levels of osteopontin in the cerebrospinal fluid of the individual prior to administration of the anti-TREM2 antibody. In some embodiments, administration of an anti-TREM2 antibody of the disclosure at a dose of about 60 mg/kg results in an increase in the levels of osteopontin in the cerebrospinal fluid of the individual of at least about 100% at 2 days after administration of the antibody, compared to the levels of osteopontin in the cerebrospinal fluid of the individual prior to administration of the anti-TREM2 antibody.

In some embodiments, administration of an anti-TREM2 antibody of the disclosure at a dose of about 15 mg/kg results in an increase in the levels of osteopontin in the cerebrospinal fluid of the individual of at least about 20% at 12 days after administration of the antibody, compared to the levels of osteopontin in the cerebrospinal fluid of the individual prior to administration of the anti-TREM2 antibody. In some embodiments, administration of an anti-TREM2 antibody of the disclosure at a dose of about 30 mg/kg results in an increase in the levels of osteopontin in the cerebrospinal fluid of the individual of at least about 35% at 12 days after administration of the antibody, compared to the levels of osteopontin in the cerebrospinal fluid of the individual prior to administration of the anti-TREM2 antibody. In some embodiments, administration of an anti-TREM2 antibody of the disclosure at a dose of about 45 mg/kg results in an increase in the levels of osteopontin in the cerebrospinal fluid of the individual of at least about 1% at 12 days after administration of the antibody, compared to the levels of osteopontin in the cerebrospinal fluid of the individual prior to administration of the anti-TREM2 antibody. In some embodiments, administration of an anti-TREM2 antibody of the disclosure at a dose of about 60 mg/kg results in an increase in the levels of osteopontin in the cerebrospinal fluid of the individual of at least about 50% at 12 days after administration of the antibody, compared to the levels of osteopontin in the cerebrospinal fluid of the individual prior to administration of the anti-TREM2 antibody.

In some embodiments, the levels of osteopontin in the cerebrospinal fluid of the individual are compared to the levels of osteopontin in the cerebrospinal fluid of the individual at between about 42 days to less than 1 day (e.g., any of 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less than 1 day) prior to administration of the anti-TREM2 antibody. In some embodiments, the levels of osteopontin in the cerebrospinal fluid of the individual are compared to the levels of osteopontin in the cerebrospinal fluid of the individual at least about 4 days prior to administration of the anti-TREM2 antibody.

The levels of osteopontin in the cerebrospinal fluid of the individual may be measured using any method known in the art, such as ELISA, immunoassays, immunoblotting, and mass spectrometry. In certain embodiments, the levels of osteopontin in the cerebrospinal fluid of the individual are measured using an ECL immunoassay using the Meso Scale Discovery system.

### Pharmacokinetics of Anti-TREM2 Antibodies

In some embodiments, the terminal half-life of an anti-TREM2 antibody of the disclosure in blood (e.g., plasma) is around 5 days, around 6 days, around 7 days, around 8 days, around 9 days, or around 10 days. In some embodiments, the half-life of the anti-TREM2 antibody in plasma is around 7 days. In some embodiments, the terminal half-life of the anti-TREM2 antibody in blood (e.g., plasma) is around 8 days. In some embodiments, the terminal half-life of the anti-TREM2 antibody in blood (e.g., plasma) is around 9 days. In some embodiments, the terminal half-life of the anti-TREM2 antibody in blood (e.g., plasma) is around 10 days. In some embodiments, the dose of the anti-TREM2 antibody is about 15 mg/kg and the terminal half-life of the antibody in the plasma of the individual is about 8.63 days. In some embodiments, the dose of the anti-TREM2 antibody is about 30 mg/kg and the terminal half-life of the antibody in the plasma of the individual is about 7.44 days. In some embodiments, the dose of the anti-TREM2 antibody is about 45 mg/kg and the terminal half-life of the antibody in the plasma of the individual is about 8.40 days. In some embodiments, the dose of the anti-TREM2 antibody is about 60 mg/kg and the terminal half-life of the antibody in the plasma of the individual is about 9.93 days

The terminal half-life of an anti-TREM2 antibody of the disclosure in the blood (e.g., plasma) of an individual is determined using any method known in the art, such as immunoassays, immunoblots, and mass spectrometry. In certain embodiments, the half-life of an anti-TREM2 antibody of the disclosure in the blood (e.g., plasma) of an individual is determined using an ELISA assay.

In some embodiments, administration of an anti-TREM2 antibody of the disclosure to an individual results in the presence of the anti-TREM2 antibody in the cerebrospinal fluid of the individual. In some embodiments, administration of an anti-TREM2 antibody of the disclosure to an individual results in a concentration of any of at least about 10 ng/ml, at least about 25 ng/ml, at least about 50 ng/ml, at least about 75 ng/ml, at least about 100 ng/ml, at least about 125 ng/ml, at least about 150 ng/ml, at least about 175 ng/ml, at least about 200 ng/ml, at least about 225 ng/ml, at least about 250 ng/ml, at least about 275 ng/ml, at least about 300 ng/ml, at least about 325 ng/ml, at least about 350 ng/ml, at least about 375 ng/ml, at least about 400 ng/ml, at least about 425 ng/ml, at least about 450 ng/ml, at least about 475 ng/ml, at least about 500 ng/ml, at least bout 525 ng/ml, at least about 550 ng/ml, at least about 575 ng/ml, at least about 600 ng/ml, at least about 625 ng/ml, at least about 650 ng/ml, at least about 675 ng/ml, at least about 700 ng/ml, at least about 725 ng/ml, at least about 750 ng/ml, at least about 775 ng/ml, at least about 800 ng/ml, at least about 850 ng/ml, at least about 900 ng/ml, at least about 950 ng/ml, at least about 1000 ng/ml, at least about 1050 ng/ml, at least about 1100 ng/ml, at least about 1150 ng/ml, at least about 1200 ng/ml, at least about 1250 ng/ml, or at least about 1300 ng/ml of the anti-TREM2 antibody in the cerebrospinal fluid of the individual. In some embodiments, administration of an anti-TREM2 antibody of the disclosure to an individual results in a concentration of between bout 10 ng/ml to about 750 ng/ml of the anti-TREM2 antibody in the cerebrospinal fluid of the individual. In some embodiments, the anti-TREM2 antibody of the disclosure is present in the cerebrospinal fluid of the individual for any of at least about 2 days, at least about 3 days, at least about 4 days, at least about 5 days, at least about 6 days, at least about 7 days, at least about 8 days, at least about 9 days, at least about 10 days, at least about 11 days, or at least about 12 days after administration of the antibody. In some embodiments, the anti-TREM2 antibody of the disclosure is present in the cerebrospinal fluid of the individual for at least about 2 days after administration of the antibody. In some embodiments, the anti-TREM2 antibody of the disclosure is present in the cerebrospinal fluid of the individual for at least about 12 days after administration of the antibody.

In some embodiments, administration of an anti-TREM2 antibody of the disclosure to an individual at a dose of about 15 mg/kg results in a concentration of at least about 100 ng/ml of the anti-TREM2 antibody in the cerebrospinal fluid of the individual at 2 days after administration of the anti-TREM2 antibody. In some embodiments, administration of an anti-TREM2 antibody of the disclosure to an individual at a dose of about 30 mg/kg results in a concentration of at least about 250 ng/ml of the anti-TREM2 antibody in the cerebrospinal fluid of the individual at 2 days after administration of the anti-TREM2 antibody. In some embodiments, administration of an anti-TREM2 antibody of the disclosure to an individual at a dose of about 45 mg/kg results in a concentration of at least about 400 ng/ml of the anti-TREM2 antibody in the cerebrospinal fluid of the individual at 2 days after administration of the anti-TREM2 antibody. In some embodiments, administration of an anti-TREM2 antibody of the disclosure to an individual at a dose of about 60 mg/kg results in a concentration of at least about 600 ng/ml of the anti-TREM2 antibody in the cerebrospinal fluid of the individual at 2 days after administration of the anti-TREM2 antibody.

In some embodiments, administration of an anti-TREM2 antibody of the disclosure to an individual at a dose of about 15 mg/kg results in a concentration of at least about 50 ng/ml of the anti-TREM2 antibody in the cerebrospinal fluid of the individual at 12 days after administration of the anti-TREM2 antibody.

In some embodiments, administration of an anti-TREM2 antibody of the disclosure to an individual at a dose of about 30 mg/kg results in a concentration of at least about 125 ng/ml of the anti-TREM2 antibody in the cerebrospinal fluid of the individual at 12 days after administration of the anti-TREM2 antibody. In some embodiments, administration of an anti-TREM2 antibody of the disclosure to an individual at a dose of about 45 mg/kg results in a concentration of at least about 200 ng/ml of the anti-TREM2 antibody in the cerebrospinal fluid of the individual at 12 days after administration of the anti-TREM2 antibody. In some embodiments, administration of an anti-TREM2 antibody of the disclosure to an individual at a dose of about 60 mg/kg results in a concentration of at least about 300 ng/ml of the anti-TREM2 antibody in the cerebrospinal fluid of the individual at 12 days after administration of the anti-TREM2 antibody.

An anti-TREM2 antibody of the disclosure may be measured in the CSF of an individual using any method known in the art, such as immunoassays, immunoblots, and mass spectrometry. In certain embodiments, an anti-TREM2 antibody of the disclosure is measured in the CSF of an individual using an ELISA assay.

### Diagnostic Uses

The isolated antibodies of the present disclosure (e.g., an anti-TREM2 antibody described herein) also have diagnostic utility. This disclosure therefore provides for methods of using the antibodies of this disclosure, or functional fragments thereof, for diagnostic purposes, such as the detection of a TREM2 protein in an individual or in tissue samples derived from an individual.

In some embodiments, the individual is a human. In some embodiments, the individual is a human patient suffering from, or at risk for developing a disease, disorder, or injury of the present disclosure. In some embodiments, the diagnostic methods involve detecting a TREM2 protein in a biological sample, such as a biopsy specimen, a tissue, or a cell. An anti-TREM2 antibody described herein is contacted with the biological sample and antigen-bound antibody is detected. For example, a biopsy specimen may be stained with an anti-TREM2 antibody described herein in order to detect and/or quantify disease-associated cells. The detection method may involve quantification of the antigen-bound antibody. Antibody detection in biological samples may occur with any method known in the art, including immunofluorescence microscopy, immunocytochemistry, immunohistochemistry, ELISA, FACS analysis, immunoprecipitation, or micro-positron emission tomography. In certain embodiments, the antibody is radiolabeled, for example with ¹⁸F and subsequently detected utilizing micro-positron emission tomography analysis. Antibody-binding may also be quantified in an individual by non-invasive techniques such as positron emission tomography (PET), X-ray computed tomography, single-photon emission computed tomography (SPECT), computed tomography (CT), and computed axial tomography (CAT).

In other embodiments, an isolated antibody of the present disclosure (e.g., an anti-TREM2 antibody described herein) may be used to detect and/or quantify, for example, microglia in a brain specimen taken from a preclinical disease model (e.g., a non-human disease model). As such, an isolated antibody of the present disclosure (e.g., an anti-TREM2 antibody described herein) may be useful in evaluating therapeutic response after treatment in a model for a nervous system disease or injury such as dementia, frontotemporal dementia, Alzheimer's disease, Nasu-Hakola disease, cognitive deficit, memory loss, spinal cord injury, traumatic brain injury, a demyelination disorder, multiple sclerosis, Parkinson's disease, amyotrophic lateral sclerosis (ALS), Huntington's disease, adult-onset leukoencephalopathy with axonal spheroids and pigmented glia (ALSP), and a tauopathy disease, as compared to a control.

### TREM2 Proteins

The present disclosure provides methods of treating, preventing, or reducing risk in an individual having a disease or injury comprising administering to the individual an antibody that binds to a TREM2 protein, wherein the antibody is an agonist.

Triggering receptor expressed on myeloid cells-2 (TREM2) is variously referred to as TREM-2, TREM2a, TREM2b, TREM2c, triggering receptor expressed on myeloid cells-2a, and triggering receptor expressed on monocytes-2. TREM2 is a 230 amino acid membrane protein. TREM2 is an immunoglobulin-like receptor primarily expressed on myeloid lineage cells, including without limitation, macrophages, dendritic cells, monocytes, Langerhans cells of skin, Kupffer cells, osteoclasts, and microglia. In some embodiments, TREM2 forms a receptor signaling complex with DAP12. In some embodiments, TREM2 phosphorylates and signals through DAP12 (an ITAM domain adaptor protein). In some embodiments, TREM2 signaling results in the downstream activation of PI3K or other intracellular signals. On myeloid cells, Toll-like receptor (TLR) signals are important for the activation of TREM2 activities, e.g., in the context of an infection response. TLRs also play a key role in the pathological inflammatory response, e.g., TLRs expressed in macrophages and dendritic cells.

TREM2 proteins of the present disclosure include, without limitation, a human TREM2 protein (Uniprot Accession No. Q9NZC2; SEQ ID NO: 1), and a non-human mammalian TREM2 protein, such as mouse TREM2 protein (Uniprot Accession No. Q99NH8; SEQ ID NO: 2), rat TREM2 protein (Uniprot Accession No. D3ZZ89; SEQ ID NO: 3), Rhesus monkey TREM2 protein (Uniprot Accession No. F6QVF2; SEQ ID NO: 4), cynomolgus monkey TREM2 protein (NCBI Accession No. XP_015304909.1; SEQ ID NO: 5), equine TREM2 protein (Uniprot Accession No. F7D6LO; SEQ ID NO: 6), pig TREM2 protein (Uniprot Accession No. H2EZZ3; SEQ ID NO: 7), and dog TREM2 protein (Uniprot Accession No. E2RP46; SEQ ID NO: 8). As used herein, “TREM2 protein” refers to both wild-type sequences and naturally occurring variant sequences. In some embodiments, an agonist anti-TREM2 antibody of the disclosure binds to a wild-type TREM2 protein, a naturally occurring variant of a TREM2 protein, or a disease variant of a TREM2 protein.

In some embodiments, an example of a human TREM2 amino acid sequence is set forth below as SEQ ID NO: 1:

```
                10 20 30 40
MEPLRLLILL FVTELSGAHN TTVFQGVAGQ SLQVSCPYDS
                50 60 70 80
MKHWGRRKAW CRQLGEKGPC QRVVSTHNLW LLSFLRRWNG
                90 100 110 120
STAITDDTLG GTLTITLRNL QPHDAGLYQC QSLHGSEADT
              130 140 150 160
LRKVLVEVLA DPLDHRDAGD LWFPGESESF EDAHVEHSIS
              170 180 190 200
RSLLEGEIPF PPTSILLLLA CIFLIKILAA SALWAAAWHG
              210 220 230
QKPGTHPPSE LDCGHDPGYO LQTLPGLRDT
```

In some embodiments, the human TREM2 is a preprotein that includes a signal peptide. In some embodiments, the human TREM2 is a mature protein. In some embodiments, the mature TREM2 protein does not include a signal peptide. In some embodiments, the mature TREM2 protein is expressed on a cell. In some embodiments, TREM2 protein contains a signal peptide located at amino acid residues 1-18 of human TREM2 (SEQ ID NO: 1); an extracellular immunoglobulin-like variable-type (IgV) domain located at amino acid residues 29-112 of human TREM2 (SEQ ID NO: 1); additional extracellular sequences located at amino acid residues 113-174 of human TREM2 (SEQ ID NO: 1); a transmembrane domain located at amino acid residues 175-195 of human TREM2 (SEQ ID NO: 1); and an intracellular domain located at amino acid residues 196-230 of human TREM2 (SEQ ID NO: 1). The TREM2 cleavage site has been identified as occurring on the C-terminal side of Histidine 157 (see WO2018/015573), and cleavage at that site leads to shedding of the relevant portion of the TREM2 extracellular domain, detectable as an increase in soluble TREM2 (sTREM2) corresponding to that portion of TREM2.

The transmembrane domain of human TREM2 contains a lysine at amino acid residue 186 that can interact with an aspartic acid in DAP12, which is a key adaptor protein that transduces signaling from TREM2, TREM1, and other related IgV family members.

Certain aspects of the present disclosure relate to methods of treating a disease or injury in an individual, comprising administering to the individual an anti-TREM2 antibody of the disclosure according to the methods provided herein. In some embodiments, the individual is heterozygous or homozygous for a mutation in TREM2 (e.g., in a human TREM2 gene). The mutation may be of any type, including, for example, a missense mutation, an indel, or a mutation generating a truncated protein product. In some embodiments, the individual has the R47H TREM2 mutation. In some embodiments, the individual has the R62H TREM2 mutation. In some embodiments, the individual has the R47H and the R62H TREM2 mutations. In certain embodiments, the individual comprises one or more amino acid substitutions in a human TREM2 protein. In certain embodiments, the individual comprises an amino acid substitution in a human TREM2 protein at residue position R47H, R62H, or both. In certain embodiments, the individual comprises an amino acid substitution in a human TREM2 protein at residue position R47H. In certain embodiments, the individual comprises an amino acid substitution in a human TREM2 protein at residue position R62H. In certain embodiments, the individual comprises an amino acid substitution in a human TREM2 protein at residue position R47H and R62H. In certain embodiments, a mutation in a TREM2 gene or an amino acid substitution in a TREM2 protein results in reduced function of TREM2 in the affected individual, e.g., compared to a TREM2 protein considered to have “wild type” function or that has function considered to be within the normal range. In certain embodiments, the individual comprises at least one copy of a functional TREM2 gene. Any method known in the art may be used to determine whether an individual has a mutation in a TREM2 gene or in a TREM2 protein, such as targeted sequencing, whole genome sequencing, and polymerase chain reaction (e.g., qPCR).

### Anti-TREM2 Antibodies

Certain aspects of the present disclosure relate to antibodies (e.g., monoclonal antibodies) that bind to a TREM2 protein, where the anti-TREM2 antibody is an agonist. In some embodiments, antibodies of the present disclosure bind a mature TREM2 protein. In some embodiments, antibodies of the present disclosure bind a mature TREM2 protein, wherein the mature TREM2 protein is expressed on a cell. In some embodiments, antibodies of the present disclosure bind a TREM2 protein expressed on one or more human cells selected from human dendritic cells, human macrophages, human monocytes, human osteoclasts, human Langerhans cells of skin, human Kupffer cells, human microglia, and any combinations thereof. In some embodiments, antibodies of the present disclosure bind a TREM2 protein expressed on one or more human microglia. In some embodiments, antibodies of the present disclosure bind a TREM2 protein expressed on one or more human microglia.

Anti-TREM2 Antibodies that Induce Activity and/or Enhance Ligand-Induced Activity

In some embodiments, an anti-TREM2 antibody of the present disclosure is an agonist antibody that induces or increases one or more TREM2 activities. In some embodiments, the antibody induces or increases one or more activities of TREM2 after binding to a TREM2 protein that is expressed on a cell.

In some embodiments, anti-TREM2 antibodies of the present disclosure bind to a TREM2 protein without competing with, inhibiting, or otherwise blocking one or more TREM2 ligands from binding to the TREM2 protein. Examples of TREM2 ligands include, without limitation, TREM2 ligands expressed by E. coli cells, apoptotic cells, nucleic acids, anionic lipids, APOE, APOE2, APOE3, APOE4, anionic APOE, anionic APOE2, anionic APOE3, anionic APOE4, lipidated APOE, lipidated APOE2, lipidated APOE3, lipidated APOE4, zwitterionic lipids, negatively charged phospholipids, phosphatidylserine, sulfatides, phosphatidylcholin, sphingomyelin, membrane phospholipids, lipidated proteins, proteolipids, lipidated peptides, and lipidated amyloid beta peptide. Accordingly, in certain embodiments, the one or more TREM2 ligands comprise E. coli cells, apoptotic cells, nucleic acids, anionic lipids, zwitterionic lipids, negatively charged phospholipids, phosphatidylserine (PS), sulfatides, phosphatidylcholin, sphingomyelin (SM), phospholipids, lipidated proteins, proteolipids, lipidated peptides, and lipidated amyloid beta peptide.

Anti-TREM2 antibodies used in the methods of the present disclosure are agonist antibodies. In some embodiments, antibodies of the present disclosure that bind a TREM2 protein may include agonist antibodies that, due to their epitope specificity, bind TREM2 and activate one or more TREM2 activities. In some embodiments, such antibodies may bind to the ligand-binding site on TREM2 and mimic the action of one or more TREM2 ligands, or stimulate TREM2 to transduce signal by binding to one or more domains that are not the ligand-binding sites. In some embodiments, the antibodies do not compete with or otherwise block ligand binding to TREM2. In some embodiments, the antibodies, act additively or synergistically with one or more TREM2 ligands to activate and/or enhance one more TREM2 activities, as set forth below.

Agonist anti-TREM2 antibodies of the present disclosure may display the ability to bind TREM2 without blocking simultaneous binding of one or more TREM2 ligands. The anti-TREM2 antibodies of the present disclosure may further display additive and/or synergistic functional interactions with one or more TREM2 ligands. Thus, in some embodiments, the maximal activity of TREM2 when bound to anti-TREM2 antibodies of the present disclosure in combination with one or more TREM2 ligands of the present disclosure may be greater (e.g., enhanced) than the maximal activity of TREM2 when exposed to saturating concentrations of ligand alone or to saturating concentrations of the antibody alone. In addition, the activity of TREM2 at a given concentration of TREM2 ligand may be greater (e.g., enhanced) in the presence of the antibody.

Accordingly, in some embodiments, anti-TREM2 antibodies of the present disclosure have an additive effect with the one or more TREM2 ligands to enhance the one or more TREM2 activities when bound to the TREM2 protein. In some embodiments, anti-TREM2 antibodies of the present disclosure synergize with the one or more TREM2 ligands to enhance the one or more TREM2 activities. In some embodiments, anti-TREM2 antibodies of the present disclosure increase the potency of the one or more TREM2 ligands to induce the one or more TREM2 activities, as compared to the potency of the one or more TREM2 ligands to induce the one or more TREM2 activities in the absence of the antibody. In some embodiments, anti-TREM2 antibodies of the present disclosure enhance the one or more TREM2 activities in the absence of cell surface clustering of TREM2. In some embodiments, anti-TREM2 antibodies of the present disclosure enhance the one or more TREM2 activities by inducing or retaining cell surface clustering of TREM2. In some embodiments, anti-TREM2 antibodies of the present disclosure are clustered by one or more Fc-gamma receptors expressed on one or more immune cells, including without limitation, B cells and microglial cells. In some embodiments, enhancement of the one or more TREM2 activities induced by binding of one or more TREM2 ligands to the TREM2 protein is measured on primary cells, including without limitation, dendritic cells, bone marrow-derived dendritic cells, monocytes, microglia, macrophages, neutrophils, NK cells, osteoclasts, Langerhans cells of skin, and Kupffer cells, or on cell lines.

In certain embodiments, an anti-TREM2 antibody of the present disclosure that enhances one or more TREM2 activities induced by binding of one or more TREM2 ligands to the TREM2 protein induces at least a 2-fold, at least a 3-fold, at least a 4-fold, at least a 5-fold, at least a 6-fold, at least a 7-fold, at least a 8-fold, at least a 9-fold, at least a 10-fold, at least an 11-fold, at least a 12-fold, at least a 13-fold, at least a 14-fold, at least a 15-fold, at least a 16-fold, at least a 17-fold, at least an 18-fold, at least a 19-fold, at least a 20-fold or greater increase in the one or more TREM2 activities as compared to levels of the one or more TREM2 activities induced by binding of the one or more TREM2 ligands to the TREM2 protein in the absence of the anti-TREM2 antibody.

In some embodiments, TREM2 activities that may be induced and/or enhanced by anti-TREM2 antibodies of the present disclosure and/or one or more TREM2 ligands of the present disclosure include, without limitation, TREM2 binding to DAP12; DAP12 phosphorylation; activation of Syk kinase; modulation of one or more pro-inflammatory mediators selected from IFN-β, IL-1α, IL-1β, TNF-α, YM-1, IL-6, IL-8, CRP, CD86, MCP-1/CCL2, CCL3, CCL4, CCL5, CCR2, CXCL-10, Gata3, Rorc, IL-20 family members, IL-33, LIF, IFN-gamma, OSM, CNTF, GM-CSF, CSF-1, MHC-II, OPN, CD11c, GM-CSF, IL-11, IL-12, IL-17, IL-18, and IL-23, optionally where the modulation occurs in one or more cells selected from macrophages, M1 macrophages, activated M1 macrophages, M2 macrophages, dendritic cells, monocytes, osteoclasts, Langerhans cells of skin, Kupffer cells, and microglial cells; recruitment of Syk, ZAP70, or both to a DAP12/TREM2 complex; increasing activity of one or more TREM2-dependent genes, optionally where the one or more TREM2-dependent genes comprise nuclear factor of activated T-cells (NFAT) transcription factors; increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; modulated expression of one or more stimulatory molecules selected from CD83, CD86 MHC class II, CD40, and any combination thereof, optionally where the CD40 is expressed on dendritic cells, monocytes, macrophages, or any combination thereof, and optionally where the dendritic cells comprise bone marrow-derived dendritic cells; increasing memory; and reducing cognitive deficit. In some embodiments, anti-TREM2 antibodies of the present disclosure increase memory and/or reduce cognitive deficit when administered to an individual.

In some embodiments, an agonist anti-TREM2 antibody of the present disclosure induces or increases one or more TREM2 activities selected from TREM2 binding to DAP12, DAP12 phosphorylation, activation of Syk kinase, recruitment of Syk to a DAP12/TREM2 complex, increasing activity of one or more TREM2-dependent genes, or any combination thereof. In some embodiments, the one or more TREM2-dependent genes include nuclear factor of activated T-cells (NFAT) transcription factors.

Syk Phosphorylation

In some embodiments, the anti-TREM2 antibodies of the present disclosure may induce spleen tyrosine kinase (Syk) phosphorylation after binding to a TREM2 protein expressed in a cell.

Spleen tyrosine kinase (Syk) is an intracellular signaling molecule that functions downstream of TREM2 by phosphorylating several substrates, thereby facilitating the formation of a signaling complex leading to cellular activation and inflammatory processes.

In some embodiments, the ability of agonist TREM2 antibodies to induce Syk activation is determined by culturing mouse macrophages and measuring the phosphorylation state of Syk protein in cell extracts. In some embodiments, bone marrow-derived macrophages (BMDM) from wild-type (WT) mice, from TREM2 knockout (KO) mice, and from mice that lack expression of functional Fc receptor common gamma chain gene (FcgR KO; REF: Takai T 1994. Cell 76(3):519-29) are starved for 4 hours in 1% serum RPMI and then removed from tissue culture dishes with PBS-EDTA, washed with PBS, and counted. In some embodiments, the cells are coated with full-length TREM2 antibodies, or with control antibodies for 15 minutes on ice. In some embodiments, after washing with cold PBS, cells are incubated at 37° C. for the indicated period of time in the presence of goat anti-human IgG. In some embodiments, after stimulation, cells are lysed with lysis buffer (1% v/v NP-40%, 50 Mm Tris-HCl (pH 8.0), 150 mM NaCl, 1 mM EDTA, 1.5 mM MgCl₂, 10% glycerol, plus protease and phosphatase inhibitors) followed by centrifugation at 16,000 g for 10 min at 4° C. to remove insoluble materials. In some embodiments, lysates are then immunoprecipitated with anti-Syk antibody (N-19 for BMDM or 4D10 for human DCs, Santa Cruz Biotechnology). In some embodiments, precipitated proteins are fractionated by SDS-PAGE, transferred to PVDF membranes and probed with anti-phosphotyrosine antibody (4G10, Millipore). In some embodiments, to confirm that all substrates are adequately immunoprecipitated, immunoblots are reprobed with anti-Syk antibody (Abcam, for BMDM) or anti-Syk (Novus Biological, for human DCs). In some embodiments, visualization is performed with the enhanced chemiluminescence (ECL) system (GE healthcare), as described (e.g., Peng et al., (2010) Sci Signal., 3(122): ra38).

DAP12 Binding and Phosphorylation

In some embodiments, the anti-TREM2 antibodies of the present disclosure may induce binding of TREM2 to DAP12. In other embodiments, the anti-TREM2 antibodies of the present disclosure may induce DAP12 phosphorylation after binding to a TREM2 protein expressed in a cell. In other embodiments, TREM2-mediated DAP12 phosphorylation is induced by one or more SRC family tyrosine kinases. Examples of Src family tyrosine kinases include, without limitation, Src, Syk, Yes, Fyn, Fgr, Lck, Hck, Blk, Lyn, and Frk.

DAP12 is variously referred to as TYRO protein tyrosine kinase-binding protein, TYROBP, KARAP, and PLOSL. DAP12 is a transmembrane signaling protein that contains an immunoreceptor tyrosine-based activation motif (ITAM) in its cytoplasmic domain. In certain embodiments, the anti-TREM2 antibody may induce DAP12 phosphorylation in its ITAM motif. Any method known in the art for determining protein phosphorylation, such as DAP12 phosphorylation, may be used.

In some embodiments, DAP12 is phosphorylated by SRC family kinases, resulting in the recruitment and activation of the Syk kinase, ZAP70 kinase, or both, to a DAP12/TREM2 complex.

In some embodiments, the ability of TREM2 antibodies to induce DAP12 activation is determined by culturing mouse macrophages and measuring the phosphorylation state of DAP12 protein in cell extracts. In some embodiments, before stimulation with antibodies, mouse wild-type (WT) bone marrow-derived macrophages (BMDM) and TREM2 knockout (KO) BMDM are starved for 4 h in 1% serum RPMI. In some embodiments, 15×10⁶ cells are incubated in ice for 15 min with full-length TREM2 antibodies or control antibodies. In some embodiments, cells are washed and incubated at 37° C. for the indicated period of time in the presence of goat anti-human IgG. In some embodiments, after stimulation, cells are lysed with lysis buffer (1% v/v n-Dodecyl-□-D-maltoside, 50 Mm Tris-HCl (pH 8.0), 150 mM NaCl, 1 mM EDTA, 1.5 mM MgCl₂, 10% glycerol, plus protease and phosphatase inhibitors), followed by centrifugation at 16,000 g for 10 min at 4° C. to remove insoluble materials. In some embodiments, cell lysate is immunoprecipitated with a second TREM2 antibody (R&D Systems). In some embodiments, precipitated proteins are fractionated by SDS-PAGE, transferred to PVDF membranes, and probed with anti-phosphotyrosine Ab (4G10, Millipore). In some embodiments, the membrane is stripped and reprobed with anti-DAP12 antibody (Cells Signaling, D7G1X). In some embodiments, each cell lysate used for TREM2 immunoprecipitations contains an equal amount of proteins, as indicated by a control antibody (anti-Actin, Santa Cruz).

Proliferation, Survival and Functionality of TREM2-Expressing Cells

In some embodiments, the anti-TREM2 antibodies of the present disclosure may increase the proliferation, survival, and/or function of dendritic cells, macrophages, monocytes, osteoclasts, Langerhans cells of skin, Kupffer cells, and microglial cells (microglia) after binding to TREM2 protein expressed in a cell. In some embodiments, the anti-TREM2 antibodies of the present disclosure do not inhibit the growth (e.g., proliferation and/or survival) of one or more innate immune cells.

In some embodiments, the anti-TREM2 antibodies of the present disclosure may increase the proliferation, survival, and/or function of microglial cells (microglia) after binding to TREM2 protein expressed in a cell. Microglial cells are a type of glial cell that are the resident macrophages of the brain and spinal cord, and thus act as the first and main form of active immune defense in the central nervous system (CNS). Microglial cells constitute 20% of the total glial cell population within the brain. Microglial cells are constantly scavenging the CNS for plaques, damaged neurons and infectious agents. The brain and spinal cord are considered “immune privileged” organs in that they are separated from the rest of the body by a series of endothelial cells known as the blood-brain barrier, which prevents most infections from reaching the vulnerable nervous tissue. In the case where infectious agents are directly introduced to the brain or cross the blood-brain barrier, microglial cells must react quickly to decrease inflammation and destroy the infectious agents before they damage the sensitive neural tissue. Due to the unavailability of antibodies from the rest of the body (few antibodies are small enough to cross the blood brain barrier), microglia must be able to recognize foreign bodies, swallow them, and act as antigen-presenting cells activating T-cells. Since this process must be done quickly to prevent potentially fatal damage, microglial cells are extremely sensitive to even small pathological changes in the CNS. They achieve this sensitivity in part by having unique potassium channels that respond to even small changes in extracellular potassium.

As used herein, macrophages of the present disclosure include, without limitation, M1 macrophages, activated M1 macrophages, and M2 macrophages. As used herein, microglial cells of the present disclosure include, without limitation, M1 microglial cells, activated M1 microglial cells, and M2 microglial cells.

In some embodiments, anti-TREM2 antibodies of the present disclosure may increase the expression of CD83 and/or CD86 on dendritic cells, monocytes, and/or macrophages.

As used herein, the rate of proliferation, survival, and/or function of macrophages, dendritic cells, monocytes, and/or microglia may include increased expression if the rate of proliferation, survival, and/or function of dendritic cells, macrophages, monocytes, osteoclasts, Langerhans cells of skin, Kupffer cells, and/or microglia in a subject treated with an anti-TREM2 antibody of the present disclosure is greater than the rate of proliferation, survival, and/or function of dendritic cells, macrophages, monocytes, osteoclasts, Langerhans cells of skin, Kupffer cells, and/or microglia in a corresponding subject that is not treated with the anti-TREM2 antibody. In some embodiments, an anti-TREM2 antibody of the present disclosure may increase the rate of proliferation, survival, and/or function of dendritic cells, macrophages, monocytes, osteoclasts, Langerhans cells of skin, Kupffer cells, and/or microglia in a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the rate of proliferation, survival, and/or function of dendritic cells, macrophages, monocytes, osteoclasts, Langerhans cells of skin, Kupffer cells, and/or microglia in a corresponding subject that is not treated with the anti-TREM2 antibody. In other embodiments, an anti-TREM2 antibody of the present disclosure may increase the rate of proliferation, survival, and/or function of dendritic cells, macrophages, monocytes, osteoclasts, Langerhans cells of skin, Kupffer cells, and/or microglia in a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the rate of proliferation, survival, and/or function of dendritic cells, macrophages, monocytes, osteoclasts, Langerhans cells of skin, Kupffer cells, and/or microglia in a corresponding subject that is not treated with the anti-TREM2 antibody.

In some embodiments, to evaluate the ability of anti-TREM2 antibodies to induce or enhance cell survival in vitro, macrophages deficient in the gamma chain subunit of FcgRI, FcgRIII, and FceRI receptors (Fcgr1KO mice, REF: Takai T, Li M, Sylvestre D, Clynes R, Ravetch J. (1994). Cell, 76:519-529) are cultured in the presence of plate-bound anti-TREM2 antibodies and cell viability is determined when cells are cultured in suboptimal growth conditions. In some embodiments, murine bone marrow precursor cells from FcgR1 KO mice (Taconic, Model 584) are obtained by flushing tibial and femoral marrow cells with cold PBS. In some embodiments, after one wash with PBS, erythrocytes are lysed using ACK Lysing Buffer (Lonza), washed twice with PBS and suspended at 0.5×10⁶ cells/ml in complete RPMI media (10% FCS, Pen/Strep, Gln, neAA) with the indicated amount of M-CSF (Peprotech) to produce macrophages. In some embodiments, to analyze cell viability of bone marrow-derived macrophages, cells are prepared as above and plated at 2.5×10⁴/200 □l in a 96-well plate with suboptimal amounts of M-CSF (long/ml) in non-tissue culture treated plates for two days. In some embodiments, cells are then quantified using the ToxGlo™ kit (Promega) and luminescence is determined as a measure of cell viability. In some embodiments, all experiments are conducted in the presence or absence of anti-TREM2 antibodies or isotype control antibodies.

TREM2-Dependent Gene Expression

In some embodiments, anti-TREM2 antibodies of the present disclosure may increase the activity and/or expression of TREM2-dependent genes, such as one or more transcription factors of the nuclear factor of activated T-cells (NFAT) family of transcription factors.

In some embodiments, the ability of soluble full-length anti-TREM2 antibodies to activate mouse or human TREM2-dependent genes is evaluated using a luciferase reporter gene under the control of an NFAT (nuclear factor of activated T-cells) promoter. In some embodiments, the cell line BW5147.G.1.4 (ATCC® TIB48™), derived from mouse thymus lymphoma T lymphocytes, is infected with mouse TREM2 and DAP12, and with Cignal Lenti NFAT-Luciferase virus (Qiagen). In some embodiments, alternatively the BW5147.G.1.4 cell line is infected with a human TREM2/DAP12 fusion protein, and with Cignal Lenti NFAT-Luciferase virus (Qiagen). In some embodiments, as a positive control for signaling, PMA (0.05 ug/ml) and ionomycin (0.25 uM) are added together. In some embodiments, cells are incubated together with soluble anti-TREM2 and isotype control antibodies for 6 hours and luciferase activity is measured by adding OneGlo Reagent (Promega) to each well and incubating 3 min at room temperature on a plate shaker. In some embodiments, luciferase signal is measured using a BioTek plate reader. In some embodiments, the cells display tonic TREM2-dependent signaling due to either the presence of an endogenous ligand or to spontaneous receptor aggregation, which leads to TREM2 signaling.

In some embodiments, the enhancement of the one or more TREM2 activities induced by binding of one or more TREM2 ligands to the TREM2 protein is measured, for example, utilizing an in vitro cell assay. In some embodiments, the increase in one or more TREM2 activities may be measured by any suitable in vitro cell-based assays or suitable in vivo model described herein or known in the art, for example, by utilizing a luciferase-based reporter assay to measure TREM2-dependent gene expression, using Western blot analysis to measure increase in TREM2-induced phosphorylation of downstream signaling partners, such as Syk, or by utilizing flow cytometry, such as fluorescence-activated cell sorting (FACS) to measure changes in cell surface levels of markers of TREM2 activation. Any in vitro cell-based assays or suitable in vivo model described herein or known in the art may be used to measure interaction (e.g., binding) between TREM2 and one or more TREM2 ligands.

In some embodiments, the increase in one or more TREM2 activities is measured by an in vitro cell-based assay. In some embodiments, to evaluate the ability of anti-TREM2 antibodies to enhance cell survival in vitro, macrophages deficient in the gamma chain subunit of FcgRI, FcgRIII, and FceRI receptors (Fcgr1KO mice, REF: Takai T, Li M, Sylvestre D, Clynes R, Ravetch J. (1994). Cell, 76:519-529) are cultured in the presence of plate-bound anti-TREM2 antibodies and cell viability is determined when cells are cultured in suboptimal growth conditions. In some embodiments, murine bone marrow precursor cells from FcgR1 KO mice (Taconic, Model 584) are obtained by flushing tibial and femoral marrow cells with cold PBS. In some embodiments, after one wash with PBS, erythrocytes are lysed using ACK Lysing Buffer (Lonza), washed twice with PBS and suspended at 0.5×10⁶ cells/ml in complete RPMI media (10% FCS, Pen/Strep, Gln, neAA) with the indicated amount of M-CSF (Peprotech) to produce macrophages. In some embodiments, to analyze cell viability of bone marrow-derived macrophages, cells are prepared as above and plated at 2.5×10⁴/200 □l in a 96-well plate with suboptimal amounts of M-CSF (long/ml) in non-tissue culture treated plates for two days. In some embodiments, cells are then quantified using the ToxGlo™ kit (Promega) and luminescence is determined as a measure of cell viability. In some embodiments, all experiments are conducted in the presence or absence of anti-TREM2 antibodies or isotype control antibodies.

In some embodiments, the increase in one or more TREM2 activities is measured by an in vivo cell-based assay. In some embodiments, to evaluate the ability of anti-TREM2 antibodies to increase the number of immune cells in vivo, C57Bl6 mice are injected intraperitoneally (IP) with an anti-TREM2 antibody or a mouse IgG1 isotype control antibody, and the number of immune cells in the brain is quantified by FACS. In some embodiments, three to four mice per group receive an IP injection of 40 mg/kg anti-TREM2 antibody or isotype control antibody mIgG1 (clone MOPC-21, Bioxcell). In some embodiments, 48 hours later, the entire brains are harvested, rinsed with PBS, incubated at 37° C. in PBS containing 1 mg/ml collagenase and processed through a cell strainer to obtain a single cell suspension. In some embodiments, cells are then incubated with anti-CD45-PerCp-Cy7, anti-CD11b-PerCP-Cy5.5, anti-Gr1-FITC antibodies and a cell viability dye (Life Technologies, Cat #L34957) for 30 min on ice, then washed twice with cold FACS buffer. In some embodiments, 4% PFA-fixed samples are then analyzed by FACS. In some embodiments, data are acquired on a BD FACSCanto™ II cytometer (Becton Dickinson) and analyzed with FlowJo software.

In some embodiments, an anti-TREM2 antibody of the present disclosure enhances one or more TREM2 activities induced by binding of a TREM2 ligand to the TREM2 protein if it induces an increase that ranges from about 1.5-fold to about 6-fold, or more than 6-fold in ligand-induced TREM2-dependent gene transcription when used at a concentration that ranges from about 0.5 nM to about 50 nM, or greater than 50 nM, and as compared to the level of TREM2-dependent gene transcription induced by binding of the TREM2 ligand to the TREM2 protein in the absence of the anti-TREM2 antibody when the TREM2 ligand is used at its EC₅₀ concentration. In some embodiments, the increase in ligand-induced TREM2-dependent gene transcription is at least 1.5-fold, at least 2-fold, at least a 3-fold, at least a 4-fold, at least a 5-fold, at least a 6-fold, at least a 7-fold, at least a 8-fold, at least a 9-fold, at least a 10-fold, at least an 11-fold, at least a 12-fold, at least a 13-fold, at least a 14-fold, at least a 15-fold, at least a 16-fold, at least a 17-fold, at least an 18-fold, at least a 19-fold, at least a 20-fold or greater when used at a concentration that ranges from about 0.5 nM to about 50 nM, or greater than 50 nM, and as compared to the level of TREM2-dependent gene transcription induced by binding of the TREM2 ligand to the TREM2 protein in the absence of the anti-TREM2 antibody when the TREM2 ligand is used at its EC₅₀ concentration.

In some embodiments, the anti-TREM2 antibody is used at a concentration of at least 0.5 nM, at least 0.6 nM, at least 0.7 nM, at least 0.8 nM, at least 0.9 nM, at least 1 nM, at least 2 nM, at least 3 nM, at least 4 nM, at least 5 nM, at least 6 nM, at least 7 nM, at least 8 nM, at least 9 nM, at least 10 nM, at least 15 nM, at least 20 nM, at least 25 nM, at least 30 nM, at least 35 nM, at least 40 nM, at least 45 nM, at least 46 nM, at least 47 nM, at least 48 nM, at least 49 nM, or at least 50 nM. In some embodiments, the TREM2 ligand is phosphatidylserine (PS). In some embodiments, the TREM2 ligand is sphingomyelin (SM). In some embodiments, the increase in one more TREM2 activities may be measured by any suitable in vitro cell-based assays or suitable in vivo model described herein or known in the art. In some embodiments, a luciferase-based reporter assay is used to measure the fold increase of ligand-induced TREM2-dependent gene expression in the presence and absence of antibody, as described in, for example, WO2017/062672 and WO2019/028292.

As used herein, an anti-TREM2 antibody of the present disclosure does not compete with, inhibit, or otherwise block the interaction (e.g., binding) between one or more TREM2 ligands and TREM2 if it decreases ligand binding to TREM2 by less than 20% at saturating antibody concentrations utilizing any in vitro assay or cell-based culture assay described herein or known in the art. In some embodiments, anti-TREM2 antibodies of the present disclosure inhibit interaction (e.g., binding) between one or more TREM2 ligands and TREM2 by less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% at saturating antibody concentrations utilizing any in vitro assay or cell-based culture assay described herein or known in the art.

Anti-TREM2 Antibodies that Decrease Soluble TREM2

In some embodiments, an agonist anti-TREM2 antibody decreases soluble TREM2 (sTREM2). In some embodiments, an agonist anti-TREM2 antibody decreases the level sTREM2 that is “shed” from the cell surface of a cell into an extracellular sample (e.g. shedding). In some embodiments, such an antibody binds to a region of TREM2 such that it blocks cleavage of TREM2. In such embodiments, the antibody binds to a region comprising His157, the cleavage site of TREM2.

The degree of inhibition of cleavage of TREM2 by an anti-TREM2 antibody negatively correlates with the amount of soluble TREM2 (sTREM2) in the presence of the anti-TREM2 antibody as compared to the amount of sTREM2 in the absence of the anti-TREM2 antibody. For example, an anti-TREM2 antibody may be considered as an anti-TREM2 antibody that inhibits cleavage of TREM2, if in the presence of said anti-TREM2 antibody the amount of sTREM2 is 0-90%, preferably 0-80%, more preferably 0-70%, even more preferably 0-60%, even more preferably 0-50% and even more preferable 0-20% of the amount of sTREM2 in the absence of the anti-TREM2 antibody, as assayed, e.g., by ELISA-based quantification of sTREM2.

In some embodiments, an anti-TREM2 antibody decreases levels of sTREM2 if the amount of sTREM2 in a treated sample is decreased by at least 10%, at least 20%, at least 30%, at least 40%, at least 50%, at least 60%, at least 70%, at least 80%, at least 90% or more as compared to a control value. In some embodiments, the control value is the amount of sTREM2 in an untreated sample (e.g., a supernatant from a TREM2-expressing cell that has not been treated with an anti-TREM2 antibody, or a sample from a subject that has not been treated with an anti-TREM2 antibody) or a sample treated with an appropriate non-TREM2-binding antibody.

In some embodiments, sTREM2 shedding is measured using a sample that comprises a fluid, e.g., blood, plasma, serum, urine, or cerebrospinal fluid. In some embodiments, the sample comprises cerebrospinal fluid. In some embodiments, the sample comprises supernatant from cell cultures (e.g., supernatant from a primary cell or cell line that endogenously expresses TREM2, such as human macrophages, or a primary cell or cell line that has been engineered to express TREM2).

In some embodiments, the level of sTREM2 in a sample is measured using an immunoassay. Immunoassays are known in the art and include, but are not limited to, enzyme immunoassays (EIA) such as enzyme multiplied immunoassay (EMIA), enzyme linked immunosorbent assay (ELISA), microparticle enzyme immunoassay (MEIA), immunohistochemistry (IHC), immunocytochemistry, capillary electrophoresis immunoassays (CEIA), radioimmunoassays (RIA), immunofluorescence, chemiluminescence immunoassays (CL), and electrochemiluminescence immunoassays (ECL). In some embodiments, sTREM2 levels are measured using an ELISA assay.

In some embodiments, an ELISA assay can be used for quantitation of levels of sTREM2 in cell culture supernatants. In some embodiments, an ELISA for human sTREM2 is conducted using the Meso Scale Discovery SECTOR Imager 2400. In some embodiments, Streptavidin-coated 96-well plates are blocked overnight at 4° C. in 0.5% bovine serum albumin (BSA) and 0.05% Tween 20 in PBS (pH 7.4) (blocking buffer). In some embodiments, plates are shaken for 1 hour at room temperature with biotinylated polyclonal goat anti-human TREM2 capture antibody (0.25 mg/ml; R&D Systems) diluted in blocking buffer. In some embodiments, plates are washed subsequently four times with 0.05% Tween 20 in PBS (washing buffer) and incubated for 2 hours at room temperature with samples diluted 1:4 in 0.25% BSA and 0.05% Tween 20 in PBS (pH 7.4) (assay buffer) supplemented with protease inhibitors (Sigma). In some embodiments, recombinant human TREM2 protein (Holzel Diagnostika) is diluted in assay buffer in a two-fold serial dilution and used for the standard curve (concentration range, 4000 to 62.5 pg/ml). In some embodiments, plates are washed three times for 5 min with washing buffer before incubation for 1 hour at room temperature with mouse monoclonal anti-TREM2 antibody (1 mg/ml; Santa Cruz Biotechnology; B-3) diluted in blocking buffer. In some embodiments, after three additional washing steps, plates are incubated with a SULFO-TAG-labeled anti-mouse secondary antibody (1:1000; Meso Scale Discovery) and incubated for 1 hour in the dark. In some embodiments, plates are washed three times with washing buffer followed by two washing steps in PBS and developed by adding Meso Scale Discovery Read buffer. In some embodiments, the light emission at 620 nm after electrochemical stimulation is measured using the Meso Scale Discovery SECTOR Imager 2400 reader. In some embodiments, to quantify the levels of sTREM2 secreted, conditioned media from biological replicates are analyzed in duplicates. In some embodiments, sTREM2 standard curves are generated using the MasterPlex ReaderFit software (MiraiBio Group, Hitachi Solutions America) through a five-parameter logistic fit. In some embodiments, levels of sTREM2 are subsequently normalized to levels of immature TREM2 as quantified from Western Blots.

In some embodiments, sTREM2 may be inactive variants of cellular TREM2 receptors. In some embodiments, sTREM2 may be present in the periphery, such as in the plasma, or brain of the subject, and may sequester anti-TREM2 antibodies. Such sequestered antibodies would be unable to bind to and activate, for example, the cellular TREM2 receptor present on cells. Accordingly, in certain embodiments, anti-TREM2 antibodies of the present disclosure, such as agonist anti-TREM2 antibodies of the present disclosure, do not bind to soluble TREM2. In some embodiments, anti-TREM2 antibodies of the present disclosure, such as agonist anti-TREM2 antibodies of the present disclosure, do not bind to soluble TREM2 in vivo. In some embodiments, agonist anti-TREM2 antibodies of the present disclosure that do not bind soluble TREM2 may bind to an epitope on TREM2 that, for example, may include a portion of the extracellular domain of cellular TREM2 that is not contained in sTREM2, for example one or more amino acid residues within amino acid residues 161-175; may be at or near a transmembrane portion of TREM2; or may include a transmembrane portion of TREM2.

Antibodies that Affect TREM2 Clustering

In vivo, anti-TREM2 antibodies of the present disclosure may activate receptors by multiple potential mechanisms. In some embodiments, agonistic anti-TREM2 antibodies of the present disclosure, have, due to appropriate epitope specificity, the ability to activate TREM2 in solution without having to be clustered with a secondary antibody, bound on plates, or clustered through Fcg receptors. In some embodiments, anti-TREM2 antibodies of the present disclosure have isotypes of human antibodies, such as IgG2, that have, due to their unique structure, an intrinsic ability to cluster receptors or retain receptors in a clustered configuration, thereby activating receptors such as TREM2 without binding to an Fc receptor (e.g., White et al., (2015) Cancer Cell 27, 138-148).

In certain embodiments, agonist anti-TREM2 antibodies may induce or maintain clustering on the cell surface in order to activate TREM2 and transduce a signal. In certain embodiments, agonist anti-TREM2 antibodies with appropriate epitope specificity may induce or maintain clustering of TREM2 on the cell surface and/or activate TREM2. In some embodiments, agonist anti-TREM2 antibodies bind to one or more amino acids within amino acid residues 124-153 of SEQ ID NO: 1, or amino acid residues on a TREM2 protein corresponding to amino acid residues 124-153 of SEQ ID NO: 1; within amino acid residues 129-153 of SEQ ID NO: 1, or amino acid residues on a TREM2 protein corresponding to amino acid residues 129-153 of SEQ ID NO: 1; within amino acid residues 140-149 of SEQ ID NO: 1, or amino acid residues on a TREM2 protein corresponding to amino acid residues 140-149 of SEQ ID NO: 1; within amino acid residues 149-157 of SEQ ID NO: 1, or amino acid residues on a TREM2 protein corresponding to amino acid residues 149-157 of SEQ ID NO: 1; or within amino acid residues 153-162 of SEQ ID NO: 1, or amino acid residues on a TREM2 protein corresponding to amino acid residues 153-162 of SEQ ID NO: 1. In some embodiments, agonist anti-TREM2 antibodies bind to one or more amino acid residues selected from the group consisting of D140, L141, W142, F143, P144, E151, D152, H154, E156, and H157 of SEQ ID NO: 1, or one or more amino acid residues on a mammalian TREM2 protein corresponding to an amino acid residue selected from the group consisting of D140, L141, W142, F143, P144, E151, D152, H154, E156, and H157 of SEQ ID NO: 1. In some embodiments, anti-TREM2 antibodies of the present disclosure cluster receptors (e.g., TREM2) by binding to Fcg receptors on adjacent cells. Binding of the constant IgG Fc part of the antibody to Fcg receptors leads to aggregation of the antibodies, and the antibodies in turn aggregate the receptors to which they bind through their variable region (Chu et al (2008) Mol Immunol, 45:3926-3933; and Wilson et al., (2011) Cancer Cell 19, 101-113). Any suitable assay known to one of skill in the art (such as those described in WO2017/062672 and WO2019/028292) may be used to determine antibody clustering.

Other mechanisms may also be used to cluster receptors (e.g., TREM2). For example, in some embodiments, antibody fragments (e.g., Fab fragments) that are cross-linked together may be used to cluster receptors (e.g., TREM2) in a manner similar to antibodies with Fc regions that bind Fcg receptors, as described above. In some embodiments, cross-linked antibody fragments (e.g., Fab fragments) may function as agonist antibodies if they induce receptor clustering on the cell surface and bind an appropriate epitope on the target (e.g., TREM2).

An antibody dependent on binding to FcgR receptor to activate targeted receptors may lose its agonist activity if engineered to eliminate FcgR binding (see, e.g., Wilson et al., (2011) Cancer Cell 19, 101-113; Armour at al., (2003) Immunology 40 (2003) 585-593); and White et al., (2015) Cancer Cell 27, 138-148). In certain embodiments, it is thought that an anti-TREM2 antibody of the present disclosure with appropriate epitope specificity can activate TREM2 when the antibody has an Fc domain.

Exemplary antibody Fc isotypes and modifications are provided in Table A below. In some embodiments, the antibody has an Fc isotype listed in Table A below.

TABLE A
Exemplary antibody Fc isotypes that are capable
of binding Fc gamma receptor

| Fc Isotype | Mutation (EU numbering scheme) |
|---|---|
| IgG1 | N297A |
| IgG1 | D265A and N297A |
| IgG1 | D270A |
| IgG1 | L234A and L235A |
| | L234A and G237A |
| | L234A and L235A and G237A |
| IgG1 | P238D and/or L328E and/or S267E/L328F |
| | and/or E233 and or/ G237D and/or |
| | H268D and/or P271G and/or A330R |
| IgG1 | P238D and L328E and E233D and G237D |
| | and H268D and P271G and A330R |
| IgG1 | P238D and L328E and G237D and H268D |
| | and P271G and A330R |
| IgG1 | P238D and S267E and L328F and E233D |
| | and G237D and H268D and P271G and |
| | A330R |
| IgG1 | P238D and S267E and L328F and G237D |
| | and H268D and P271G and A330R |
| IgG2 | V234A and G237A |
| IgG4 | L235A and G237A and E318A |
| IgG4 | S228P and L236E |
| IgG2/ | IgG2 aa 118 to 260 and IgG4 aa 261 to |
| 4 hybrid | 447 |
| | H268Q and V309L; and A330S and P331S |
| IgG1 | C226S and C229S and E233P and L234V |
| | and L235A |
| IgG1 | L234F and L235E and P331S |
| IgG2 | C232S or C233S |
| IgG2 | A330S and P331S |
| IgG1 | S267E, and L328F |
| | S267E alone |
| IgG2 | S267E and L328F |
| IgG4 | S267E and L328F |
| IgG2 | WT HC with Kappa (light chain) LC |
| | HC C127S with Kappa LC |
| | Kappa LC C214S |
| | Kappa LC C214S and HC C233S |
| | Kappa LC C214S and HC C232S |
| | Any of the above listed mutations |
| | together with A330S and P331S |
| | mutations |
| | F(ab′)2 fragment of WT IgG1 and any |
| | of the above listed mutations |
| IgG1 | Substitute the Constant Heavy 1 (CH1) |
| | and hinge region of IgG1 With CH1 |
| | and hinge region of IGg2 |
| | ASTKGPSVFP LAPCSRSTSE STAALGCLVK |
| | DYFPEPVTVS WNSGALTSGV HTFPAVLQSS |
| | GLYSLSSVVT VPSSNFGTQT YTCNVDHKPS |
| | NTKVDKTVER KCCVECPPCP (SEQ ID NO: 42) |
| | With a Kappa LC |
| IgG1 | Any of the above listed mutations |
| | together with A330L/A330S and/or |
| | L234F and/or L235E and/or P331S |
| IgG1, IgG2, | Any of the above listed mutations |
| or IgG4 | together with M252Y and/or S254T |
| | and/or T256E |
| Mouse IgG1 | For mouse disease models |
| IgG4 | WT |
| IgG1 | Any of the above listed mutation |
| | together with E430G, E430S, E430F, |
| | E430T, E345K, E345Q, E345R, E345Y, |
| | S440Y, S440W and/or any combination |
| | thereof. |
| IgG2 | Any of the above listed mutation |
| | together with E430G, E430S, E430F, |
| | E430T, E345K, E345Q, E345R, E345Y, |
| | S440Y, S440W and/or any combination |
| | thereof. |

In some embodiments, the antibody is of the IgG class, the IgM class, or the IgA class. In some embodiments, the antibody has an IgG1, IgG2, IgG3, or IgG4 isotype.

Antibodies with human IgG1 or IgG3 isotypes and mutants thereof (e.g. Strohl (2009) Current Opinion in Biotechnology 2009, 20:685-691) that bind the activating Fcg Receptors I, IIA, IIC, IIIA, IIIB in human and/or Fcg Receptors I, III and IV in mouse, may also act as agonist antibodies in vivo but may be associated with effects related to ADCC. However, such Fcg receptors appear to be less available for antibody binding in vivo, as compared to the inhibitory Fcg receptor FcgRIIB (see, e.g., White, et al., (2013) Cancer Immunol. Immunother. 62, 941-948; and Li et al., (2011) Science 333(6045):1030-1034).

In certain embodiments, the antibody has an IgG2 isotype. In some embodiments, the antibody contains a human IgG2 constant region. In some embodiments, the human IgG2 constant region includes an Fc region. In some embodiments, the antibody induces the one or more TREM2 activities, the DAP12 activities, or both independently of binding to an Fc receptor. In some embodiments, the antibody binds an inhibitory Fc receptor. In certain embodiments, the inhibitory Fc receptor is inhibitory Fc-gamma receptor IIB (FcγIIB), which minimizes or eliminates ADCC. In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from V234A (Alegre et al., (1994) Transplantation 57:1537-1543. 31; Xu et al., (2000) Cell Immunol, 200:16-26), G237A (Cole et al. (1999) Transplantation, 68:563-571), H268Q, V309L, A330S, P331S (US 2007/0148167; Armour et al. (1999) Eur J Immunol 29: 2613-2624; Armour et al. (2000) The Haematology Journal 1(Suppl. 1):27; Armour et al. (2000) The Haematology Journal 1(Suppl. 1):27), C232S, and/or C233S (White et al., (2015) Cancer Cell 27, 138-148), S267E, L328F (Chu et al., (2008) Mol Immunol, 45:3926-3933), M252Y, S254T, and/or T256E, where the amino acid position is according to the EU numbering convention.

In some embodiments, the antibody has an IgG2 isotype with a heavy chain constant domain that contains a C127S amino acid substitution, where the amino acid position is according to the EU numbering convention (White et al., (2015) Cancer Cell 27, 138-148; Lightle et al., (2010) PROTEIN SCIENCE 19:753-762; and WO2008079246).

In some embodiments, the antibody has an IgG2 isotype with a Kappa light chain constant domain that contains a C214S amino acid substitution, where the amino acid position is according to the EU numbering convention (White et al., (2015) Cancer Cell 27, 138-148; Lightle et al., (2010) PROTEIN SCIENCE 19:753-762; and WO2008079246).

In certain embodiments, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the antibody binds an inhibitory Fc receptor. In certain embodiments, the inhibitory Fc receptor is inhibitory Fc-gamma receptor IIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention.

In some embodiments, the antibody includes an IgG2 isotype heavy chain constant domain 1(CH1) and hinge region (White et al., (2015) Cancer Cell 27, 138-148). In certain embodiments, the IgG2 isotype CH1 and hinge region contain the amino acid sequence of ASTKGPSVFPLAPCSRSTSESTAALGCLVKDYFPEPVTVSWNSGALTSGVHTFPAVLQSSGLYSLSSV VTVPSSNFGTQTYTCNVDHKPSNTKVDKTVERKCCVECPPCP (SEQ ID NO: 42). In some embodiments, the antibody Fc region contains a S267E amino acid substitution, a L328F amino acid substitution, or both, and/or a N297A or N297Q amino acid substitution, where the amino acid position is according to the EU numbering convention.

In certain embodiments, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor IIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from L235A, G237A, S228P, L236E (Reddy et al., (2000) J Immunol, 164: 1925-1933), S267E, E318A, L328F, M252Y, S254T, and/or T256E, where the amino acid position is according to the EU numbering convention.

In certain embodiments, the antibody has a hybrid IgG2/4 isotype. In some embodiments, the antibody includes an amino acid sequence containing amino acids 118 to 260 according to EU numbering of human IgG2 and amino acids 261-447 according to EU numbering of human IgG4 (WO 1997/11971; WO 2007/106585).

In certain embodiments, the antibody contains a mouse IgG4 constant region (Bartholomaeus, et al. (2014). J. Immunol. 192, 2091-2098).

In some embodiments, the Fc region further contains one or more additional amino acid substitutions selected from A330L, L234F; L235E, or P331S according to EU numbering; and any combination thereof.

In certain embodiments, the antibody contains one or more amino acid substitutions in the Fc region at a residue position selected from C127S, L234A, L234F, L235A, L235E, S267E, K322A, L328F, A330S, P331S, E345R, E430G, S440Y, and any combination thereof, where the numbering of the residues is according to EU numbering. In some embodiments, the Fc region contains an amino acid substitution at positions E430G, L243A, L235A, and P331S, where the numbering of the residue position is according to EU numbering. In some embodiments, the Fc region contains an amino acid substitution at positions E430G and P331S, where the numbering of the residue position is according to EU numbering. In some embodiments, the Fc region contains an amino acid substitution at positions E430G and K322A, where the numbering of the residue position is according to EU numbering. In some embodiments, the Fc region contains an amino acid substitution at positions E430G, A330S, and P331S, where the numbering of the residue position is according to EU numbering. In some embodiments, the Fc region contains an amino acid substitution at positions E430G, K322A, A330S, and P331S, where the numbering of the residue position is according to EU numbering. In some embodiments, the Fc region contains an amino acid substitution at positions E430G, K322A, and A330S, where the numbering of the residue position is according to EU numbering. In some embodiments, the Fc region contains an amino acid substitution at positions E430G, K322A, and P331S, where the numbering of the residue position is according to EU numbering. In some embodiments, the Fc region contains an amino acid substitution at positions S267E and L328F, where the numbering of the residue position is according to EU numbering. In some embodiments, the Fc region contains an amino acid substitution at position C127S, where the numbering of the residue position is according to EU numbering. In some embodiments, the Fc region contains an amino acid substitution at positions E345R, E430G and S440Y, where the numbering of the residue position is according to EU numbering.

In some embodiments, the antibody has a human IgG1 isotype and comprises amino acid substitutions in the Fc region at the residue positions P331S and E430G, wherein the numbering of the residues is according to EU numbering. An Fc region comprising amino acid substitutions at the residue positions P331S and E430G may be referred to as “PSEG.”

Further IgG Mutations

In some embodiments, one or more of the IgG1 variants described herein may be combined with an A330L mutation (Lazar et al., (2006) Proc Natl Acad Sci USA, 103:4005-4010), or one or more of L234F, L235E, and/or P331S mutations (Sazinsky et al., (2008) Proc Natl Acad Sci USA, 105:20167-20172), where the amino acid position is according to the EU numbering convention, to eliminate complement activation. In some embodiments, the IgG variants described herein may be combined with one or more mutations to enhance the antibody half-life in human serum (e.g. M252Y, S254T, T256E mutations according to the EU numbering convention) (Dall'Acqua et al., (2006) J Biol Chem, 281:23514-23524; and Strohl et al., (2009) Current Opinion in Biotechnology, 20:685-691).

In some embodiments, an IgG4 variant of the present disclosure may be combined with an S228P mutation according to the EU numbering convention (Angal et al., (1993) Mol Immunol, 30:105-108) and/or with one or more mutations described in Peters et al., (2012) J Biol Chem. 13; 287(29):24525-33) to enhance antibody stabilization.

### Exemplary Anti-TREM2 Antibodies

In some embodiments, an anti-TREM2 antibody of the present disclosure binds to TREM2 with high affinity, is an agonist, and induces or increases one or more TREM2 activities. In some embodiments, the anti-TREM2 antibody enhances one or more TREM2 activities induced by binding of one or more TREM2 ligands to the TREM2 protein, as compared to the one or more TREM2 activities induced by binding of the one or more TREM2 ligands to the TREM2 protein in the absence of the isolated antibody. In some embodiments, the anti-TREM2 antibody enhances the one or more TREM2 activities without competing with or otherwise blocking binding of the one or more TREM2 ligands to the TREM2 protein. In some embodiments, the antibody is a humanized antibody, a bispecific antibody, a multivalent antibody, or a chimeric antibody. Exemplary descriptions of such antibodies are found throughout the present disclosure. In some embodiments, the antibody is a bispecific antibody recognizing a first antigen and a second antigen.

In some embodiments, anti-TREM2 antibodies of the present disclosure bind to a human TREM2, or a homolog thereof, including without limitation a mammalian (e.g., non-human mammalian) TREM2 protein, mouse TREM2 protein (Uniprot Accession No. Q99NH8), rat TREM2 protein (Uniprot Accession No. D3ZZ89), Rhesus monkey TREM2 protein (Uniprot Accession No. F6QVF2), cynomolgus monkey TREM2 protein (NCBI Accession No. XP_015304909.1), equine TREM2 protein (Uniprot Accession No. F7D6L0), pig TREM2 protein (Uniprot Accession No. H2EZZ3), and dog TREM2 protein (Uniprot Accession No. E2RP46). In some embodiments, anti-TREM2 antibodies of the present disclosure specifically bind to human TREM2. In some embodiments, anti-TREM2 antibodies of the present disclosure specifically bind to cynomolgus monkey TREM2. In some embodiments, anti-TREM2 antibodies of the present disclosure specifically bind to both human TREM2 and cynomolgus monkey TREM2. In some embodiments, anti-TREM2 antibodies of the present disclosure induce at least one TREM2 activity of the present disclosure.

Anti-TREM2 Antibody-Binding Regions

In some embodiments, anti-TREM2 antibodies of the present disclosure bind to one or more amino acids within amino acid residues 124-153 of SEQ ID NO: 1, or amino acid residues on a TREM2 protein corresponding to amino acid residues 124-153 of SEQ ID NO: 1; one or more amino acids within amino acid residues 129-153 of SEQ ID NO: 1, or amino acid residues on a TREM2 protein corresponding to amino acid residues 129-153 of SEQ ID NO: 1; one or more amino acids within amino acid residues 140-149 of SEQ ID NO: 1, or amino acid residues on a TREM2 protein corresponding to amino acid residues 140-149 of SEQ ID NO: 1; one or more amino acids within amino acid residues 149-157 of SEQ ID NO: 1, or amino acid residues on a TREM2 protein corresponding to amino acid residues 149-157 of SEQ ID NO: 1; or one or more amino acids within amino acid residues 153-162 of SEQ ID NO: 1, or amino acid residues on a TREM2 protein corresponding to amino acid residues 153-162 of SEQ ID NO: 1. In some embodiments, anti-TREM2 antibodies of the present disclosure bind one or more of amino acid residues D140, L141, W142, F143, P144, E151, D152, H154, E156, and H157 of SEQ ID NO: 1, or one or more amino acid residues on a mammalian TREM2 protein corresponding to an amino acid residue selected from the group consisting of D140, L141, W142, F143, P144, E151, D152, H154, E156, and H157 of SEQ ID NO: 1.

Anti-TREM2 Antibody Light Chain and Heavy Chain Variable Regions

In some embodiments, anti-TREM2 antibodies to be used in the methods of the present disclosure are described in WO2019/028292, WO2018/015573, WO2018/195506, or WO2019/055841, each of which is hereby incorporated by reference herein. In some embodiments, the anti-TREM2 antibodies to be used in the methods of the present disclosure induce or enhance one or more of the following TREM2 activities: TREM2 binding to DAP12; DAP12 phosphorylation; activation of Syk kinase; modulation of one or more pro-inflammatory mediators selected from IFN-β, IL-1α, IL-1β, TNF-α, YM-1, IL-6, IL-8, CRP, CD86, MCP-1/CCL2, CCL3, CCL4, CCL5, CCR2, CXCL-10, Gata3, Rorc, IL-20 family members, IL-33, LIF, IFN-gamma, OSM, CNTF, GM-CSF, CSF-1, MHC-II, OPN, CD11c, GM-CSF, IL-11, IL-12, IL-17, IL-18, and IL-23, optionally where the modulation occurs in one or more cells selected from macrophages, M1 macrophages, activated M1 macrophages, M2 macrophages, dendritic cells, monocytes, osteoclasts, Langerhans cells of skin, Kupffer cells, and microglial cells; recruitment of Syk, ZAP70, or both to a DAP12/TREM2 complex; increasing activity of one or more TREM2-dependent genes, optionally where the one or more TREM2-dependent genes comprise nuclear factor of activated T-cells (NFAT) transcription factors; increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; modulated expression of one or more stimulatory molecules selected from CD83, CD86 MHC class II, CD40, and any combination thereof, optionally where the CD40 is expressed on dendritic cells, monocytes, macrophages, or any combination thereof, and optionally where the dendritic cells comprise bone marrow-derived dendritic cells; increasing memory; and reducing cognitive deficit. In some embodiments, anti-TREM2 antibodies of the present disclosure increase memory and/or reduce cognitive deficit when administered to an individual.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 34; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 35; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 31; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 41; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 33; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 32.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 36; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 37; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 38; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 39; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 40; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 32.

In some embodiments, anti-TREM2 antibodies of the present disclosure comprise a light chain variable domain and a heavy chain variable domain, wherein the heavy chain variable region comprises an HVR-H1 comprising the amino acid sequence YAFSSDWMN (SEQ ID NO: 36), an HVR-H2 comprising the amino acid sequence RIYPGEGDTNYARKFHG (SEQ ID NO: 37), an HVR-H3 comprising the amino acid sequence ARLLRNKPGESYAMDY (SEQ ID NO: 38), and the light chain variable region comprises an HVR-L1 comprising the amino acid sequence RTSQSLVHSNAYTYLH (SEQ ID NO: 39), an HVR-L2 comprising the amino acid sequence KVSNRVS (SEQ ID NO: 40), and an HVR-L3 comprising the amino acid sequence SQSTRVPYT (SEQ ID NO: 32). In some embodiments, anti-TREM2 antibodies of the present disclosure comprise a light chain variable domain and a heavy chain variable domain, wherein the heavy chain variable region comprises an HVR-H1 comprising the amino acid sequence YAFSSQWMN (SEQ ID NO: 34), an HVR-H2 comprising the amino acid sequence RIYPGGGDTNYAGKFQG (SEQ ID NO: 35), an HVR-H3 comprising the amino acid sequence ARLLRNQPGESYAMDY (SEQ ID NO: 31), and the light chain variable region comprises an HVR-L1 comprising the amino acid sequence RSSQSLVHSNRYTYLH (SEQ ID NO: 41), an HVR-L2 comprising the amino acid sequence KVSNRFS (SEQ ID NO: 33), and an HVR-L3 comprising the amino acid sequence SQSTRVPYT (SEQ ID NO: 32). In some embodiments, anti-TREM2 antibodies of the present disclosure comprise a heavy chain variable region and a light chain variable region, wherein the heavy chain variable region comprises one, two, three or four frame work regions selected from VH FR1, VH FR2, VH FR3, and VH FR4, wherein: the VH FR1 comprises a sequence selected from the group consisting of SEQ ID NOs: 9-11, the VH FR2 comprises a sequence selected from the group consisting of SEQ ID NOs: 12 and 13, the VH FR3 comprises a sequence selected from the group consisting of SEQ ID NOs: 14 and 15, and the VH FR4 comprises the sequence of SEQ ID NO: 16; and/or the light chain variable region comprises one, two, three or four frame work regions selected from VL FR1, VL FR2, VL FR3, and VL FR4, wherein: the L FR1 comprises a sequence selected from the group consisting of SEQ ID NOs: 17-20, the VL FR2 comprises a sequence selected from the group consisting of SEQ ID NOs: 21 and 22, the VL FR3 comprises a sequence selected from the group consisting of SEQ ID NOs: 23 and 24, and the VL FR4 comprises a sequence selected from the group consisting of SEQ ID NOs: 25 and 26.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-47 (referred to herein as “AT.2V”) or to the amino acid sequence of SEQ ID NO: 28; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AT.2V or to the amino acid sequence of SEQ ID NO: 29. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AT.2V or to the amino acid sequence of SEQ ID NO: 28, wherein the heavy chain variable domain comprises the HVR-H1, HVR-H2, and HVR-H3 amino acid sequences of antibody AT.2V. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AT.2V or to the amino acid sequence of SEQ ID NO: 29, wherein the light chain variable domain comprises the HVR-L1, HVR-L2, and HVR-L3 amino acid sequences of antibody AT.2V. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AT.2V or to the amino acid sequence of SEQ ID NO: 28 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody AT.2V or the amino acid sequence of SEQ ID NO: 28. In certain embodiments, a total of 1 to 5 amino acids have been substituted, inserted and/or deleted in the heavy chain variable domain amino acid sequence of antibody AT.2V or the amino acid sequence of SEQ ID NO: 28. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody AT.2V or of SEQ ID NO: 28, including post-translational modifications of that sequence. In a particular embodiment, the VH comprises one, two or three HVRs selected from: (a) the HVR-H1 amino acid sequence of antibody AT.2V, (b) the HVR-H2 amino acid sequence of antibody AT.2V, and (c) the HVR-H3 amino acid sequence of antibody AT.2V. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AT.2V or to the amino acid sequence of SEQ ID NO: 29 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody AT.2V or the amino acid sequence of SEQ ID NO: 29. In certain embodiments, a total of 1 to 5 amino acids have been substituted, inserted and/or deleted in the light chain variable domain amino acid sequence of antibody AT.2V or the amino acid sequence of SEQ ID NO: 29. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody AT.2V or of SEQ ID NO: 29, including post-translational modifications of that sequence. In a particular embodiment, the VL comprises one, two or three HVRs selected from: (a) the HVR-L1 amino acid sequence of antibody AT.2V, (b) the HVR-L2 amino acid sequence of antibody AT.2V, and (c) the HVR-L3 amino acid sequence of antibody AT.2V. In some embodiments, the antibody comprises a heavy chain variable region comprising the amino acid sequence of SEQ ID NO: 28 and a light chain variable region comprising the amino acid sequence of SEQ ID NO: 29.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-58 (referred to herein as “AT.1V”) or to the amino acid sequence of SEQ ID NO: 27; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AT.1V or to the amino acid sequence of SEQ ID NO: 30. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AT.1V or to the amino acid sequence of SEQ ID NO: 27, wherein the heavy chain variable domain comprises the HVR-H1, HVR-H2, and HVR-H3 amino acid sequences of antibody AT.1V. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AT.1V or to the amino acid sequence of SEQ ID NO: 30, wherein the light chain variable domain comprises the HVR-L1, HVR-L2, and HVR-L3 amino acid sequences of antibody AT.1V. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AT.1V or to the amino acid sequence of SEQ ID NO: 27 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody AT.1V or the amino acid sequence of SEQ ID NO: 27. In certain embodiments, a total of 1 to 5 amino acids have been substituted, inserted and/or deleted in the heavy chain variable domain amino acid sequence of antibody AT.1V or the amino acid sequence of SEQ ID NO: 27. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody AT.1V or of SEQ ID NO: 27, including post-translational modifications of that sequence. In a particular embodiment, the VH comprises one, two or three HVRs selected from: (a) the HVR-H1 amino acid sequence of antibody AT.1V, (b) the HVR-H2 amino acid sequence of antibody AT.1V, and (c) the HVR-H3 amino acid sequence of antibody AT.1V. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AT.1V or to the amino acid sequence of SEQ ID NO: 30 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody AT.1V or the amino acid sequence of SEQ ID NO: 30. In certain embodiments, a total of 1 to 5 amino acids have been substituted, inserted and/or deleted in the light chain variable domain amino acid sequence of antibody AT.1V or the amino acid sequence of SEQ ID NO: 30. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody AT.1V or of SEQ ID NO: 30, including post-translational modifications of that sequence. In a particular embodiment, the VL comprises one, two or three HVRs selected from: (a) the HVR-L1 amino acid sequence of antibody AT.1V, (b) the HVR-L2 amino acid sequence of antibody AT.1V, and (c) the HVR-L3 amino acid sequence of antibody AT.1V. In some embodiments, the antibody comprises a heavy chain variable region comprising the amino acid sequence of SEQ ID NO: 27 and a light chain variable region comprising the amino acid sequence of SEQ ID NO: 30.

In some embodiments, the antibody comprises a heavy chain comprising the amino acid of SEQ ID NO: 43, and a light chain comprising the amino acid sequence of SEQ ID NO: 47; or a heavy chain comprising the amino acid of SEQ ID NO: 44, and a light chain comprising the amino acid sequence of SEQ ID NO: 47.

In some embodiments, the antibody comprises a heavy chain comprising the amino acid of SEQ ID NO: 45, and a light chain comprising the amino acid sequence of SEQ ID NO: 48; or a heavy chain comprising the amino acid of SEQ ID NO: 46, and a light chain comprising the amino acid sequence of SEQ ID NO: 48.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 50; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 51; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 52; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 53; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 54; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 55.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 58; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 59; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 60; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 61; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 62; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 63.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 66; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 67; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 68; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 69; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 70; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 71.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 42E8.H1 or to the amino acid sequence of SEQ ID NO: 56; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 42E8.H1 or to the amino acid sequence of SEQ ID NO: 57. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 42E8.H1 or to the amino acid sequence of SEQ ID NO: 56, wherein the heavy chain variable domain comprises the HVR-H1, HVR-H2, and HVR-H3 amino acid sequences of antibody 42E8.H1. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 42E8.H1 or to the amino acid sequence of SEQ ID NO: 57, wherein the light chain variable domain comprises the HVR-L1, HVR-L2, and HVR-L3 amino acid sequences of antibody 42E8.H1. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 42E8.H1 or to the amino acid sequence of SEQ ID NO: 56 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 42E8.H1 or the amino acid sequence of SEQ ID NO: 56. In certain embodiments, a total of 1 to 5 amino acids have been substituted, inserted and/or deleted in the heavy chain variable domain amino acid sequence of antibody 42E8.H1 or the amino acid sequence of SEQ ID NO: 56. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody 42E8.H1 or of SEQ ID NO: 56, including post-translational modifications of that sequence. In a particular embodiment, the VH comprises one, two or three HVRs selected from: (a) the HVR-H1 amino acid sequence of antibody 42E8.H1, (b) the HVR-H2 amino acid sequence of antibody 42E8.H1, and (c) the HVR-H3 amino acid sequence of antibody 42E8.H1. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 42E8.H1 or to the amino acid sequence of SEQ ID NO: 57 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 42E8.H1 or the amino acid sequence of SEQ ID NO: 57. In certain embodiments, a total of 1 to 5 amino acids have been substituted, inserted and/or deleted in the light chain variable domain amino acid sequence of antibody 42E8.H1 or the amino acid sequence of SEQ ID NO: 57. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody 42E8.H1 or of SEQ ID NO: 57, including post-translational modifications of that sequence. In a particular embodiment, the VL comprises one, two or three HVRs selected from: (a) the HVR-L1 amino acid sequence of antibody 42E8.H1, (b) the HVR-L2 amino acid sequence of antibody 42E8.H1, and (c) the HVR-L3 amino acid sequence of antibody 42E8.H1. In some embodiments, the antibody comprises a heavy chain variable region comprising the amino acid sequence of SEQ ID NO: 56 and a light chain variable region comprising the amino acid sequence of SEQ ID NO: 57.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody RS9.F6 or to the amino acid sequence of SEQ ID NO: 64; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody RS9.F6 or to the amino acid sequence of SEQ ID NO: 65. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody RS9.F6 or to the amino acid sequence of SEQ ID NO: 64, wherein the heavy chain variable domain comprises the HVR-H1, HVR-H2, and HVR-H3 amino acid sequences of antibody RS9.F6. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody RS9.F6 or to the amino acid sequence of SEQ ID NO: 65, wherein the light chain variable domain comprises the HVR-L1, HVR-L2, and HVR-L3 amino acid sequences of antibody RS9.F6. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody RS9.F6 or to the amino acid sequence of SEQ ID NO: 64 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody RS9.F6 or the amino acid sequence of SEQ ID NO: 64. In certain embodiments, a total of 1 to 5 amino acids have been substituted, inserted and/or deleted in the heavy chain variable domain amino acid sequence of antibody RS9.F6 or the amino acid sequence of SEQ ID NO: 64. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody RS9.F6 or of SEQ ID NO: 64, including post-translational modifications of that sequence. In a particular embodiment, the VH comprises one, two or three HVRs selected from: (a) the HVR-H1 amino acid sequence of antibody RS9.F6, (b) the HVR-H2 amino acid sequence of antibody RS9.F6, and (c) the HVR-H3 amino acid sequence of antibody RS9.F6. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody RS9.F6 or to the amino acid sequence of SEQ ID NO: 65 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody RS9.F6 or the amino acid sequence of SEQ ID NO: 65. In certain embodiments, a total of 1 to 5 amino acids have been substituted, inserted and/or deleted in the light chain variable domain amino acid sequence of antibody RS9.F6 or the amino acid sequence of SEQ ID NO: 65. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody RS9.F6 or of SEQ ID NO: 65, including post-translational modifications of that sequence. In a particular embodiment, the VL comprises one, two or three HVRs selected from: (a) the HVR-L1 amino acid sequence of antibody RS9.F6, (b) the HVR-L2 amino acid sequence of antibody RS9.F6, and (c) the HVR-L3 amino acid sequence of antibody RS9.F6. In some embodiments, the antibody comprises a heavy chain variable region comprising the amino acid sequence of SEQ ID NO: 64 and a light chain variable region comprising the amino acid sequence of SEQ ID NO: 65.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 72; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 73. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 72 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the amino acid sequence of SEQ ID NO: 72. In certain embodiments, a total of 1 to 5 amino acids have been substituted, inserted and/or deleted in the amino acid sequence of SEQ ID NO: 72. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of SEQ ID NO: 72, including post-translational modifications of that sequence. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 73 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the amino acid sequence of SEQ ID NO: 73. In certain embodiments, a total of 1 to 5 amino acids have been substituted, inserted and/or deleted in the amino acid sequence of SEQ ID NO: 73. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of SEQ ID NO: 73, including post-translational modifications of that sequence. In some embodiments, the antibody comprises a heavy chain variable region comprising the amino acid sequence of SEQ ID NO: 72 and a light chain variable region comprising the amino acid sequence of SEQ ID NO: 73.

In some embodiments, an agonist anti-TREM2 antibody of the present disclosure is AL2p-58 huIgG1 PSEG (referred to herein as “AT.1FM”). In some embodiments, an agonist anti-TREM2 antibody of the present disclosure is AL2p-47 huIgG1 (referred to herein as “AT.2F”).

TABLE B
Sequences

| SEQ ID | | |
|---|---|---|
| NO | Sequence | Description |
| 1 | MEPLRLLILLFVTELSGAHNTTVFQGVAGQSLQVSCPYDS | Human TREM2 |
| | MKHWGRRKAWCRQLGEKGPCQRVVSTHNLWLLSFLRRWNG | protein |
| | STAITDDTLGGTLTITLRNLQPHDAGLYQCQSLHGSEADT | |
| | LRKVLVEVLADPLDHRDAGDLWFPGESESFEDAHVEHSIS | |
| | RSLLEGEIPFPPTSILLLLACIFLIKILAASALWAAAWHG | |
| | QKPGTHPPSELDCGHDPGYQLQTLPGLRDT | |
| 2 | MGPLHQFLLLLITALSQALNTTVLQGMAGQSLRVSCTYDA | Mouse TREM2 |
| | LKHWGRRKAWCRQLGEEGPCQRVVSTHGVWLLAFLKKRNG | protein |
| | STVIADDTLAGTVTITLKNLQAGDAGLYQCQSLRGREAEV | |
| | LQKVLVEVLEDPLDDQDAGDLWVPEESSSFEGAQVEHSTS | |
| | RNQETSFPPTSILLLLACVLLSKFLAASILWAVARGRQKP | |
| | GTPVVRGLDCGQDAGHQLQILTGPGGT | |
| 3 | MEPLHVFVLLLVTELSQALNTTVLQGVAGQSLRVSCTYDA | Rat TREM2 |
| | LRHWGRRKAWCRQLAEEGPCQRVVSTHGVWLLAFLRKQNG | protein |
| | STVITDDTLAGTVTITLRNLQAGDAGLYQCQSLRGREAEV | |
| | LQKVVVEVLEDPLDDQDAGDLWVPEESESFEGAQVEHSTS | |
| | SQVSSCGSPLTYHLPPKEPIRKDLLPTHFHSSPPGLCPPE | |
| | QASYSQHPLGCGQGQAEAGDTCGQWARL | |
| 4 | MPDPLFSAVQGKDKILHKALCICPWPGKGGMEPLRLLILL | Rhesus monkey |
| | FATELSGAHNTTVFQGVEGQSLQVSCPYDSMKHWGRRKAW | TREM2 protein |
| | CRQLGEKGPCQRVVSTHNLWLLSFLRRRNGSTAITDDTLG | |
| | GTLTITLRNLQPHDAGFYQCQSLHGSEADTLRKVLVEVLA | |
| | DPLDHRDAGDLWVPGESESFEDAHVEHSISRSLLEGEIPF | |
| | PPTSVLLLLACIFLIKILAASALWAAAWHGQKPGTHPPSE | |
| | PDCGHDPGHQLQTLPGLRDT | |
| 5 | MEPLRLLILLFATELSGAHNTTVFQGVEGQSLQVSCPYDS | Cynomolgus |
| | MKHWGRRKAWCRQLGEKGPCQRVVSTHNLWLLSFLRRRNG | monkey TREM2 |
| | STAITDDTLGGTLTITLRNLQPHDAGFYQCQSLHGSEADT | protein |
| | LRKVLVEVLADPLDHRDAGDLWVPGESESFEDAHVEHSIS | |
| | RSLLEGEIPFPPTSVLLLLACIFLIKILAASALWAAAWHG | |
| | QKPGTHPPSEPDCGHDPGHQLQTLPGLRDT | |
| 6 | MEPLPLLILLSVAELSRGHNTTVFQGTAGRSLKVSCPYNS | Equine TREM2 |
| | LMHWGRRKAWCRQLGEDGPCQQVVSTHSLWLLSFLKRRNG | protein |
| | STVITDDALGGILTITLRNLQAHDAGFYQCQSLHGGEADT | |
| | LRKVLVEVLADPLDHQEPGDLWIPKESESFEDAQVEHSIS | |
| | RSLVEEEIPSLPTSILLLLACIFLSKLLAASAIWAAAWHG | |
| | QKQETPPASEPDRGHDPGYQLHTLTGERDT | |
| 7 | METLGLLLLLWVAELSRAHNTSVFQGTAGQSLRVSCSYNS | Pig TREM2 |
| | LKHWGRRKAWCRQLSEEGLCQHVVSTHPTWLLSFLKRRNG | protein |
| | STAITDDALGGTLTITLRNLQAHDAGLYQCQSLHGSEADT | |
| | LKKVLVEVLADPLESQSKSFQDVQMEHSISRNLSEESLFP | |
| | PTSTLFLLACVFLSKLLVASALWAAAWHGHKQRTSPAGGL | |
| | DCGRDPGDQDQTLTDELGESSDQDQTLTELRDT | |
| 8 | MEPLWLLILLAVTELSGAHNTTVFQGMAGRSLQVSCPYNS | Dog TREM2 |
| | LKHWGRRKAWCRQVDKEGPCQRVVSTHRSWLLSFLKRWNG | protein |
| | STAIVDDALGGTLTITLRNLQAHDAGLYQCQSLYGDEADT | |
| | LRKVLVEVLADPLDHLDPGDLWIPEESKGFEDAHVEPSVS | |
| | RSLSEEEIPFPPTSILFLLACIFLSKFLAASALWAAAWRG | |
| | QKLGTPQASELDCSCDPGYQLQTLTEPRDM | |
| 9 | QVQLVQSGAEVKKPGSSVKVSCKASG | VH FR1 |
| 10 | EVQLVQSGAEVKKPGSSVKVSCKASG | VH FR1 |
| 11 | QVQLVQSGAEVKKPGASVKVSCKASG | VH FR1 |
| 12 | WVRQAPGQGLEWMG | VH FR2 |
| 13 | WVRQAPGQRLEWIG | VH FR2 |
| 14 | RVTITADESTSTAYMELSSLRSEDTAVYYC | VH FR3 |
| 15 | RVTITADTSASTAYMELSSLRSEDTAVYYC | VH FR3 |
| 16 | WGQGTLVTVSS | VH FR4 |
| 17 | DVVMTQTPLSLSVTPGQPASISC | VL FR1 |
| 18 | GVVMTQTPLSLSVTPGQPASISC | VL FR1 |
| 19 | GVVMAQTPLSLSVTPGQPASISC | VL FR1 |
| 20 | DVVMTQSPDSLAVSLGERATINC | VL FR1 |
| 21 | WYLQKPGQSPQLLIY | VL FR2 |
| 22 | WYQQKPGQSPKLLIY | VL FR2 |
| 23 | GVPDRFSGSGSGTDFTLKISRVEAEDVGVYYC | VL FR3 |
| 24 | GVPDRFSGSGSGTDFTLTISSLQAEDVAVYYC | VL FR3 |
| 25 | FGQGTKLEIK | VL FR4 |
| 26 | FGGGTKVEIK | VL FR4 |
| 27 | QVQLVQSGAEVKKPGASVKVSCKASGYAFSSQWMNWVRQA | AL2p-58 (AT.1V) - |
| | PGQRLEWIGRIYPGGGDTNYAGKFQGRVTITADTSASTAY | Heavy chain |
| | MELSSLRSEDTAVYYCARLLRNQPGESYAMDYWGQGTLVT | variable domain |
| | VSS | |
| 28 | QVQLVQSGAEVKKPGSSVKVSCKASGYAFSSDWMNWVRQA | AL2p-47 (AT.2V) - |
| | PGQGLEWMGRIYPGEGDTNYARKFHGRVTITADESTSTAY | Heavy chain |
| | MELSSLRSEDTAVYYCARLLRNKPGESYAMDYWGQGTLVT | variable domain |
| | VSS | |
| 29 | DVVMTQTPLSLSVTPGQPASISCRTSQSLVHSNAYTYLHW | AL2p-47 (AT.2V) - |
| | YLQKPGQSPQLLIYKVSNRVSGVPDRFSGSGSGTDFTLKI | Light chain |
| | SRVEAEDVGVYYCSQSTRVPYTFGQGTKLEIK | variable domain |
| 30 | DVVMTQSPDSLAVSLGERATINCRSSQSLVHSNRYTYLHW | AL2p-58 (AT.1V) - |
| | YQQKPGQSPKLLIYKVSNRFSGVPDRFSGSGSGTDFTLKI | Light chain |
| | SRVEAEDVGVYYCSQSTRVPYTFGQGTKLEIK | variable domain |
| 31 | ARLLRNQPGESYAMDY | AL2p-58 (AT.1V) - |
| | | HVR-H3 |
| 32 | SQSTRVPYT | AL2p-58 (AT.IV); |
| | | AL2p-47 (AT.2V) - |
| | | HVR-L3 |
| 33 | KVSNRFS | AL2p-58 (AT.1V) - |
| | | HVR-L2 |
| 34 | YAFSSQWMN | AL2p-58 (AT.1V) - |
| | | HVR-H1 |
| 35 | RIYPGGGDTNYAGKFQG | AL2p-58 (AT.1V) - |
| | | HVR-H2 |
| 36 | YAFSSDWMN | AL2p-47 (AT.2V) - |
| | | HVR-H1 |
| 37 | RIYPGEGDTNYARKFHG | AL2p-47 (AT.2V) - |
| | | HVR-H2 |
| 38 | ARLLRNKPGESYAMDY | AL2p-47 (AT.2V) - |
| | | HVR-H3 |
| 39 | RTSQSLVHSNAYTYLH | AL2p-47 (AT.2V) - |
| | | HVR-L1 |
| 40 | KVSNRVS | AL2p-47 (AT.2V) - |
| | | HVR-L2 |
| 41 | RSSQSLVHSNRYTYLH | AL2p-58 (AT.1V) - |
| | | HVR-L1 |
| 42 | ASTKGPSVFPLAPCSRSTSESTAALGCLVKDYFPEPVTVSW | IgG2 isotype heavy |
| | NSGALTSGVHTFPAVLQSSGLYSLSSVVTVPSSNFGTQTYT | chain constant |
| | CNVDHKPSNTKVDKTVERKCCVECPPCP | domain 1 (CH1) and |
| | | hinge region |
| 43 | QVQLVQSGAEVKKPGASVKVSCKASGYAFSSQWMNWVRQAP | AL2p-58 huIgG1 |
| | GQRLEWIGRIYPGGGDTNYAGKFQGRVTITADTSASTAYME | PSEG(AT.1FM) - |
| | LSSLRSEDTAVYYCARLLRNQPGESYAMDYWGQGTLVTVSS | Heavy chain |
| | ASTKGPSVFPLAPSSKSTSGGTAALGCLVKDYFPEPVTVSW | |
| | NSGALTSGVHTFPAVLQSSGLYSLSSVVTVPSSSLGTQTYI | |
| | CNVNHKPSNTKVDKKVEPKSCDKTHTCPPCPAPELLGGPSV | |
| | FLFPPKPKDTLMISRTPEVTCVVVDVSHEDPEVKFNWYVDG | |
| | VEVHNAKTKPREEQYNSTYRVVSVLTVLHQDWLNGKEYKCK | |
| | VSNKALPASIEKTISKAKGQPREPQVYTLPPSRDELTKNQV | |
| | SLTCLVKGFYPSDIAVEWESNGQPENNYKTTPPVLDSDGSF | |
| | FLYSKLTVDKSRWQQGNVFSCSVMHGALHNHYTQKSLSLSP | |
| | GK | |
| 44 | QVQLVQSGAEVKKPGASVKVSCKASGYAFSSQWMNWVRQAP | AL2p-58 huIgG1 |
| | GQRLEWIGRIYPGGGDTNYAGKFQGRVTITADTSASTAYME | PSEG(AT.1FM) - |
| | LSSLRSEDTAVYYCARLLRNQPGESYAMDYWGQGTLVTVSS | Heavy chain |
| | ASTKGPSVFPLAPSSKSTSGGTAALGCLVKDYFPEPVTVSW | |
| | NSGALTSGVHTFPAVLQSSGLYSLSSVVTVPSSSLGTQTYI | |
| | CNVNHKPSNTKVDKKVEPKSCDKTHTCPPCPAPELLGGPSV | |
| | FLFPPKPKDTLMISRTPEVTCVVVDVSHEDPEVKFNWYVDG | |
| | VEVHNAKTKPREEQYNSTYRVVSVLTVLHQDWLNGKEYKCK | |
| | VSNKALPASIEKTISKAKGQPREPQVYTLPPSRDELTKNQV | |
| | SLTCLVKGFYPSDIAVEWESNGQPENNYKTTPPVLDSDGSF | |
| | FLYSKLTVDKSRWQQGNVFSCSVMHGALHNHYTQKSLSLSP | |
| | G | |
| 45 | QVQLVQSGAEVKKPGSSVKVSCKASGYAFSSDWMNWVRQAP | AL2p-47 huIgG1 |
| | GQGLEWMGRIYPGEGDTNYARKFHGRVTITADESTSTAYME | (AT.2F) - Heavy |
| | LSSLRSEDTAVYYCARLLRNKPGESYAMDYWGQGTLVTVSS | chain |
| | ASTKGPSVFPLAPSSKSTSGGTAALGCLVKDYFPEPVTVSW | |
| | NSGALTSGVHTFPAVLQSSGLYSLSSVVTVPSSSLGTQTYI | |
| | CNVNHKPSNTKVDKKVEPKSCDKTHTCPPCPAPELLGGPSV | |
| | FLFPPKPKDTLMISRTPEVTCVVVDVSHEDPEVKFNWYVDG | |
| | VEVHNAKTKPREEQYNSTYRVVSVLTVLHQDWLNGKEYKCK | |
| | VSNKALPAPIEKTISKAKGQPREPQVYTLPPSRDELTKNQV | |
| | SLTCLVKGFYPSDIAVEWESNGQPENNYKTTPPVLDSDGSF | |
| | FLYSKLTVDKSRWQQGNVFSCSVMHEALHNHYTQKSLSLSP | |
| | GK | |
| 46 | QVQLVQSGAEVKKPGSSVKVSCKASGYAFSSDWMNWVRQAP | AL2p-47 huIgG1 |
| | GQGLEWMGRIYPGEGDTNYARKFHGRVTITADESTSTAYME | (AT.2F) - Heavy |
| | LSSLRSEDTAVYYCARLLRNKPGESYAMDYWGQGTLVTVSS | chain |
| | ASTKGPSVFPLAPSSKSTSGGTAALGCLVKDYFPEPVTVSW | |
| | NSGALTSGVHTFPAVLQSSGLYSLSSVVTVPSSSLGTQTYI | |
| | CNVNHKPSNTKVDKKVEPKSCDKTHTCPPCPAPELLGGPSV | |
| | FLFPPKPKDTLMISRTPEVTCVVVDVSHEDPEVKFNWYVDG | |
| | VEVHNAKTKPREEQYNSTYRVVSVLTVLHQDWLNGKEYKCK | |
| | VSNKALPAPIEKTISKAKGQPREPQVYTLPPSRDELTKNQV | |
| | SLTCLVKGFYPSDIAVEWESNGQPENNYKTTPPVLDSDGSF | |
| | FLYSKLTVDKSRWQQGNVFSCSVMHEALHNHYTQKSLSLSP | |
| | G | |
| 47 | DVVMTQSPDSLAVSLGERATINCRSSQSLVHSNRYTYLHWY | AL2p-58 huIgG1 |
| | QQKPGQSPKLLIYKVSNRFSGVPDRFSGSGSGTDFTLKISR | PSEG(AT.1FM) - |
| | VEAEDVGVYYCSQSTRVPYTFGQGTKLEIKRTVAAPSVFIF | Light chain |
| | PPSDEQLKSGTASVVCLLNNFYPREAKVQWKVDNALQSGNS | |
| | QESVTEQDSKDSTYSLSSTLTLSKADYEKHKVYACEVTHQG | |
| | LSSPVTKSFNRGEC | |
| 48 | DVVMTQTPLSLSVTPGQPASISCRTSQSLVHSNAYTYLHWY | AL2p-47 huIgG1 |
| | LQKPGQSPQLLIYKVSNRVSGVPDRFSGSGSGTDFTLKISR | (AT.2F) - Light chain |
| | VEAEDVGVYYCSQSTRVPYTFGQGTKLEIKRTVAAPSVFIF | |
| | PPSDEQLKSGTASVVCLLNNFYPREAKVQWKVDNALQSGNS | |
| | QESVTEQDSKDSTYSLSSTLTLSKADYEKHKVYACEVTHQG | |
| | LSSPVTKSFNRGEC | |
| 49 | D/Ex0-2YxxL/IX6-8YxxL/I | Receptor motif |
| 50 | GYSITSDYAWN | 42E8.H1 CDR-H1 |
| 51 | YINYSGRTIYNPSLKS | 42E8.H1 CDR-H2 |
| 52 | ARWNGNYGFAY | 42E8.H1 CDR-H3 |
| 53 | RSSQSLVHINGNTYLH | 42E8.H1 CDR-L1 |
| 54 | KVSNRFS | 42E8.H1 CDR-L2 |
| 55 | SQTTHALFT | 42E8.H1 CDR-L3 |
| 56 | DVQLQESGPGLVKPSQSLSLTCTVTGYSITSDYAWNWIRQF | 42E8.H1 |
| | PGNKLEWMGYINYSGRTIYNPSLKSRISITRDTSKNHFFLQ | Heavy Chain |
| | LISVTTEDTATYYCARWNGNYGFAYWGQGTLVTVSA | Variable Region |
| 57 | DWMTQNPLSLPVSLGDQASISCRSSQSLVHINGNTYLHWYL | 42E8.H1 |
| | QKPGQSPKLLIYKVSNRFSGVPDRFSGSGSGTDFTLKISRV | Light Chain Variable |
| | EAEDLGVYFCSQTTHALFTFGSGTKLEIK | Region |
| 58 | GYTFTSY | RS9.F6 CDR-H1 |
| 59 | IGRSDPTTGGTNYNE | RS9.F6 CDR-H2 |
| 60 | VRTSGTGDY | RS9.F6 CDR-H3 |
| 61 | RSSQSLVHNNGNTFLH | RS9.F6 CDR-L1 |
| 62 | VSNRFS | RS9.F6 CDR-L2 |
| 63 | SQTTHVPPT | RS9.F6 CDR-L3 |
| 64 | QVQLQQPGAELVKPGASVKLSCKASGYTFTSYWMHWVKQSP | RS9.F6 |
| | GRGLEWIGRSDPTTGGTNYNEKFKTKATLTVDKPSSTAYMQ | Heavy Chain |
| | LSSLTSDDSAVYYCVRTSGTGDYWGQGTSLTVSSAKTTAPS | Variable Region |
| | VYPLAPVCGGTTGSSVT | |
| 65 | DVVMTQTPLSLPVSLGDQASISCRSSQSLVHNNGNTFLHWY | RS9.F6 |
| | LQKPGQSPKLLIYKVSNRFSGVPDRFSGSGSGTDFTLKISR | Light Chain Variable |
| | VEAEDLGVYFCSQTTHVPPTFGGGTKLEIKRADAAPTVSIF | Region |
| | PPSSEQLTSGGASVVCF | |
| 66 | GFTFTDFY | WO2018/015573 |
| | | Consensus |
| | | CDR-H1 |
| 67 | IRNKANGYTT | WO2018/015573 |
| | | Consensus |
| | | CDR-H2 |
| 68 | ARIGINNGGSLDYWG | WO2018/015573 |
| | | Consensus |
| | | CDR-H3 |
| 69 | QSLLYSENNQDY | WO2018/015573 |
| | | Consensus |
| | | CDR-L1 |
| 70 | GAS | WO2018/015573 |
| | | Consensus |
| | | CDR-L2 |
| 71 | EQTYSYPYT | WO2018/015573 |
| | | Consensus |
| | | CDR-L3 |
| 72 | EVKLLESGGGLVQPGGSMRLSCAASGFTFTDFYMNWIRQPA | WO2018/015573 |
| | GKAPEWLGLIRNKANGYTTEYNPSVKGRFTISRDNTQNMLY | Consensus |
| | LQMNTLR*EDTATYYCARIGINNGGSLDYWGQGVMVTVSS | Heavy Chain |
| | | Variable Region |
| | | The asterisk (*) in |
| | | the sequence can be |
| | | any amino acid. |
| 73 | DILINQSPASLTVSAGEKVTMSCKSSQSLLYSENNQDYLAW | WO2018/015573 |
| | YQQKPGQFPKLLIYGASNRHTGVPDRFTGSGSGTDFTLTIS | Consensus |
| | SVQAEDLADYYCEQTYSYPYTFGAGTKLELK | Light Chain Variable |
| | | Region |
| 74 | GFTFTDFY | WO2018/015573 |
| | | 14D3 |
| | | CDR-H1 |
| 75 | IRNKTKGYTT | WO2018/015573 |
| | | 14D3 |
| | | CDR-H2 |
| 76 | ARIGVNNGGSLDYWG | WO2018/015573 |
| | | 14D3 |
| | | CDR-H3 |
| 77 | QSLLYSENNQDY | WO2018/015573 |
| | | 14D3 |
| | | CDR-L1 |
| 78 | GAS | WO2018/015573 |
| | | 14D3 |
| | | CDR-L2 |
| 79 | EQTYSYPYT | WO2018/015573 |
| | | 14D3 |
| | | CDR-L3 |
| 80 | EVKLLEFGGGLVQPGGSMRLSCAASGFTFTDFYMNWIRQPA | WO2018/015573 |
| | GRAPEWLGLIRNKTKGYTTEYNRSVKGRFTISRDNTQNMLY | 14D3 |
| | LQMNSLRPEDTATYYCARIGVNNGGSLDYWGQGVMVTVSS | Heavy Chain |
| | | Variable Region |
| 81 | DILIIQSPASLTVSAGARVTMSCKSSQSLLYSENNQDYLAW | WO2018/015573 |
| | YQQKPGQFPKLLIYGASNRHTGVPDRFTGSGSGTDFTLTIS | 14D3 |
| | SVQAEDLADYYCEQTYSYPYTFGAGTKLELK | Light Chain Variable |
| | | Region |
| 82 | GFTFTDFY | WO2018/015573 |
| | | 14D8 |
| | | CDR-H1 |
| 83 | IRNKANGYTT | WO2018/015573 |
| | | 14D8 |
| | | CDR-H2 |
| 84 | ARIGINNGGSLDYWG | WO2018/015573 |
| | | 14D8 |
| | | CDR-H3 |
| 85 | QSLLYSEKNQDY | WO2018/015573 |
| | | 14D8 |
| | | CDR-L1 |
| 86 | GAS | WO2018/015573 |
| | | 14D8 |
| | | CDR-L2 |
| 87 | EQTYSYPYT | WO2018/015573 |
| | | 14D8 |
| | | CDR-L3 |
| 88 | EVKLLESGGGLVQPGGSMRLSCAASGFTFTDFYMNWIRQPA | WO2018/015573 |
| | GKAPEWLGLIRNKANGYTTVYNPSVKGRFTISRDNTQNMLY | 14D8 |
| | LQMNTLRGEDTATYYCARIGINNGGSLDYWGQGVMVTVSS | Heavy Chain |
| | | Variable Region |
| 89 | DILINQSPASLTVSTGEKVTMSCRSSQSLLYSEKNQDYLAW | WO2018/015573 |
| | YQQKPGQFPKLLIYGASYRHTGVPDRFTGSGSGTDFTLTIS | 14D8 |
| | SVQAEDLADYYCEQTYSYPYTFGAGTKLELK | Light Chain Variable |
| | | Region |
| 90 | GFTFTDFY | WO2018/015573 |
| | | 7A12 |
| | | CDR-H1 |
| 91 | IRNKANGYTT | WO2018/015573 |
| | | 7A12 |
| | | CDR-H2 |
| 92 | ARIGINNGGSLDYWG | WO2018/015573 |
| | | 7A12 |
| | | CDR-H3 |
| 93 | QSLLYSEKNQDY | WO2018/015573 |
| | | 7A12 |
| | | CDR-L1 |
| 94 | GAS | WO2018/015573 |
| | | 7A12 |
| | | CDR-L2 |
| 95 | EQTYSYPYT | WO2018/015573 |
| | | 7A12 |
| | | CDR-L3 |
| 96 | EVKLLESGGGLVQPGGSMRLSCAASGFTFTDFYMNWIRQPA | WO2018/015573 |
| | GKAPEWLGLIRNKANGYTTQYNPSVKGRFTISRDNTQNMLY | 7A12 |
| | LQMNTLRGEDTATYYCARIGINNGGSLDYWGQGVMVTVSS | Heavy Chain |
| | | Variable Region |
| 97 | DILINQSPASLTVSAGEKVTMSCKSSQSLLYSEKNQDYLAW | WO2018/015573 |
| | YQQKPGQSPKLLMYGASYRHTGVPDRFTGSGSGTDFTLTIS | 7A12 |
| | SVQAEDLADYYCEQTYSYPYTFGAGTKLELK | Light Chain Variable |
| | | Region |
| 98 | GFTFTDFY | WO2018/015573 |
| | | 8A11 |
| | | CDR-H1 |
| 99 | IRNKTKGYTT | WO2018/015573 |
| | | 8A11 |
| | | CDR-H2 |
| 100 | ARIGVNNGGSLDYWG | WO2018/015573 |
| | | 8A11 |
| | | CDR-H3 |
| 101 | QSLLYSENNQDY | WO2018/015573 |
| | | 8A11 |
| | | CDR-L1 |
| 102 | GAS | WO2018/015573 |
| | | 8A11 |
| | | CDR-L2 |
| 103 | EQTYSYPYT | WO2018/015573 |
| | | 8A11 |
| | | CDR-L3 |
| 104 | EVKLLESGGGLVQPGGSMRLSCAASGFTFTDFYMNWIRQPA | WO2018/015573 |
| | GKAPEWLGLIRNKTKGYTTEYNTSVKGRFTISRDNTQNMLY | 8A11 |
| | LQMNSLRPEDTATYYCARIGVNNGGSLDYWGQGVMVTVSS | Heavy Chain |
| | | Variable Region |
| 105 | DILIIQSPASLTVSAGARVTMSCKSSQSLLYSENNQDYLAW | WO2018/015573 |
| | YQQKPGQFPKLLIYGASNRHTGVPDRFTGSGSGTDFTLTIS | 8A11 |
| | SVQAEDLADYYCEQTYSYPYTFGAGTKLELK | Light Chain Variable |
| | | Region |
| 106 | GFTFTDFY | WO2018/015573 |
| | | 21A3 |
| | | CDR-H1 |
| 107 | IRNKANGYTT | WO2018/015573 |
| | | 21A3 |
| | | CDR-H2 |
| 108 | ARIGINNGGSLDYWG | WO2018/015573 |
| | | 21A3 |
| | | CDR-H3 |
| 109 | QSLLYSEKNQDY | WO2018/015573 |
| | | 21A3 |
| | | CDR-L1 |
| 110 | GAS | WO2018/015573 |
| | | 21A3 |
| | | CDR-L2 |
| ill | EQTYSYPYT | WO2018/015573 |
| | | 21A3 |
| | | CDR-L3 |
| 112 | EVKLLESGGGLVQPGGSMRLSCAASGFTFTDFYMNWIRQPA | WO2018/015573 |
| | GKAPEWLGLIRNKANGYTTQYNPSVKGRFTISRDNTQNMLY | 21A3 |
| | LQMNTLRGEDTATYYCARIGINNGGSLDYWGQGVMVTVSS | Heavy Chain |
| | | Variable Region |
| 113 | DILINQSPASLTVSAGEKVTMSCKSSQSLLYSEKNQDYLAW | WO2018/015573 |
| | YQQKPGQSPKLLMYGASYRHTGVPDRFTGSGSGTDFTLTIS | 21A3 |
| | SVQAEDLADYYCEQTYSYPYTFGAGTKLELK | Light Chain Variable |
| | | Region |
| 114 | GFTFTDFY | WO2018/015573 |
| | | 10C3 |
| | | CDR-H1 |
| 115 | IRNKTKGYTT | WO2018/015573 |
| | | 10C3 |
| | | CDR-H2 |
| 116 | ARIGTNNGGSLDYWG | WO2018/015573 |
| | | 10C3 |
| | | CDR-H3 |
| 117 | QSLLYSENNQDY | WO2018/015573 |
| | | 10C3 |
| | | CDR-L1 |
| 118 | GAS | WO2018/015573 |
| | | 10C3 |
| | | CDR-L2 |
| 119 | EQTYSYPYT | WO2018/015573 |
| | | 10C3 |
| | | CDR-L3 |
| 120 | EVKLLESGGGLVQPGGSMRLSCAASGFTFTDFYMNWIRQPA | WO2018/015573 |
| | GETPEWLGLIRNKTKGYTTEYNPSVKGRFTISRDNTQNMLY | 10C3 |
| | LQMNSLRPEDTATYYCARIGTNNGGSLDYWGQGVMVTVSS | Heavy Chain |
| | | Variable Region |
| 121 | DILIIQSPASLTVSAGARVTMSCKSSQSLLYSENNQDYLAW | WO2018/015573 |
| | YQQKPGQFPKLLIYGASNRHTGVPDRFTGSGSGTDFTLTIS | 10C3 |
| | SVQAEDLADYYCEQTYSYPYTFGAGTKLELK | Light Chain Variable |
| | | Region |
| 122 | GFTFTDFY | WO2018/015573 |
| | | 18F9 |
| | | CDR-H1 |
| 123 | IRNKVNGYRT | WO2018/015573 |
| | | 18F9 |
| | | CDR-H2 |
| 124 | ARIGINNGGSLDYWG | WO2018/015573 |
| | | 18F9 |
| | | CDR-H3 |
| 125 | QSLLYSENNQDY | WO2018/015573 |
| | | 18F9 |
| | | CDR-L1 |
| 126 | GAS | WO2018/015573 |
| | | 18F9 |
| | | CDR-L2 |
| 127 | EQTYSYPYT | WO2018/015573 |
| | | 18F9 |
| | | CDR-L3 |
| 128 | EVKLLESGGGLVQPGGSMRLSCVVSGFTFTDFYMNWIRQAA | WO2018/015573 |
| | GKAPEWLGLIRNKVNGYRTEYNPSVKGRFTISRDNIQNMLY | 18F9 |
| | LQMNTLRAEDTATYYCARIGINNGGSLDYWGQGVMVTVSS | Heavy Chain |
| | | Variable Region |
| 129 | DILINQSPASLTVSAGEKVTMSCKSSQSLLYSENNQDYLAW | WO2018/015573 |
| | YQQKPGQFPKLLIYGASNRHTGVPDRFTGSGSGTDFTLTIS | 18F9 |
| | SVQAEDLADYYCEQTYSYPYTFGAGTKLELK | Light Chain Variable |
| | | Region |
| 130 | GFTFTDFY | WO2018/015573 |
| | | 15C5 |
| | | CDR-H1 |
| 131 | IRNKAYGYTT | WO2018/015573 |
| | | 15C5 |
| | | CDR-H2 |
| 132 | ARIGINYGGSLDYWG | WO2018/015573 |
| | | 15C5 |
| | | CDR-H3 |
| 133 | QSLLYSESNQDY | WO2018/015573 |
| | | 15C5 |
| | | CDR-L1 |
| 134 | GAS | WO2018/015573 |
| | | 15C5 |
| | | CDR-L2 |
| 135 | EQTYSYPYT | WO2018/015573 |
| | | 15C5 |
| | | CDR-L3 |
| 136 | EVKLLESGGGLVQPGGSMRLSCAASGFTFTDFYMNWIRQPA | WO2018/015573 |
| | GKAPEWLGLIRNKAYGYTTEYNPSVKGRFTISRDNTQDMLY | 15C5 |
| | LQMNTLRAEDTATYYCARIGINYGGSLDYWGQGVMVTVSS | Heavy Chain |
| | | Variable Region |
| 137 | DILINQSPASLTVSAGEKVTVSCKSSQSLLYSESNQDYLAW | WO2018/015573 |
| | YQQKPGQFPKLLIYGASYRHTGVPDRFTGSGSGTDFTLTIS | 15C5 |
| | SVQAEDLAHYYCEQTYSYPYTFGAGTKLELK | Light Chain Variable |
| | | Region |
| 138 | GFTFTDFY | WO2018/015573 |
| | | 1G6 |
| | | CDR-H1 |
| 139 | IRNKANGFTT | WO2018/015573 |
| | | 1G6 |
| | | CDR-H2 |
| 140 | ARIGINNGGSLDYWG | WO2018/015573 |
| | | 1G6 |
| | | CDR-H3 |
| 141 | QSLLYSENKQDY | WO2018/015573 |
| | | 1G6 |
| | | CDR-L1 |
| 142 | GAS | WO2018/015573 |
| | | 1G6 |
| | | CDR-L2 |
| 143 | EQTYSYPYT | WO2018/015573 |
| | | 1G6 |
| | | CDR-L3 |
| 144 | EVKLLESGGGLVQPGGSLRLSCVASGFTFTDFYMNWIRQPA | WO2018/015573 |
| | GKAPEWLGLIRNKANGFTTEYNPSVKGRFTISRDNTQHMLY | 1G6 |
| | LQMNTLRAEDTATYYCARIGINNGGSLDYWGQGVMVTVSS | Heavy Chain |
| | | Variable Region |
| 145 | DILINQSPASLTVSTGEKVTMSCKSSQSLLYSENKQDYLAW | WO2018/015573 |
| | YQQKPGQFPKLLIYGASNRHTGVPDRFTGSGSGTDFTLTIN | 1G6 |
| | IVQAEDLADYYCEQTYSYPYTFGAGTKLELK | Light Chain Variable |
| | | Region |

Any of the antibodies of the present disclosure may be produced by a cell line. In some embodiments, the cell line may be a mammalian cell line. In certain embodiments, the cell line may be a hybridoma cell line. In other embodiments, the cell line may be a yeast cell line. Any cell line known in the art suitable for antibody production may be used to produce an antibody of the present disclosure. Exemplary cell lines for antibody production are described throughout the present disclosure.

Antibody Fragments

Certain aspects of the present disclosure relate to antibody fragments that bind to one or more of human TREM2, a naturally occurring variant of human TREM2, and a disease variant of human TREM2. In some embodiments, the antibody fragment is an Fab, Fab′, Fab′-SH, F(ab′)2, Fv or scFv fragment.

Antibody Frameworks

Any of the antibodies described herein further include a framework. In some embodiments, the framework is a human immunoglobulin framework. For example, in some embodiments, an antibody (e.g., an anti-TREM2 antibody) comprises HVRs as in any of the above embodiments and further comprises an acceptor human framework, e.g., a human immunoglobulin framework or a human consensus framework. Human immunoglobulin frameworks may be part of the human antibody, or a non-human antibody may be humanized by replacing one or more endogenous frameworks with human framework region(s). Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Antibody Preparation

Anti-TREM2 antibodies of the present disclosure can encompass polyclonal antibodies, monoclonal antibodies, humanized and chimeric antibodies, human antibodies, antibody fragments (e.g., Fab, Fab′-SH, Fv, scFv, and F(ab′)₂), bispecific and polyspecific antibodies, multivalent antibodies, library derived antibodies, antibodies having modified effector functions, fusion proteins containing an antibody portion, and any other modified configuration of the immunoglobulin molecule that includes an antigen recognition site, such as an epitope having amino acid residues of a TREM2 protein of the present disclosure, including glycosylation variants of antibodies, amino acid sequence variants of antibodies, and covalently modified antibodies. The anti-TREM2 antibodies may be human, murine, rat, or of any other origin (including chimeric or humanized antibodies).

(1) Polyclonal Antibodies

Polyclonal antibodies, such as anti-TREM2 polyclonal antibodies, are generally raised in animals by multiple subcutaneous (sc) or intraperitoneal (ip) injections of the relevant antigen and an adjuvant. It may be useful to conjugate the relevant antigen (e.g., a purified or recombinant TREM2 protein of the present disclosure) to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The animals are immunized against the desired antigen, immunogenic conjugates, or derivatives by combining, e.g., 100 μg (for rabbits) or 5 μg (for mice) of the protein or conjugate with 3 volumes of Freund's complete adjuvant and injecting the solution intradermally at multiple sites. One month later, the animals are boosted with 1/5 to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant-cell culture as protein fusions. Also, aggregating agents such as alum are suitable to enhance the immune response.

(2) Monoclonal Antibodies

Monoclonal antibodies, such as anti-TREM2 monoclonal antibodies, are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the anti-TREM2 monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization (e.g., a purified or recombinant TREM2 protein of the present disclosure). Alternatively, lymphocytes may be immunized in vitro. Lymphocytes then are fused with an immortal cell line, such as myeloma cells, using a suitable fusing agent, such as polyethylene glycol, to form a hybridoma cell (Goding, Monoclonal Antibodies: Principles and Practice, pp. 59-103 (Academic Press, 1986)).

The culture medium in which the hybridoma cells are cultured can be assayed for the presence of monoclonal antibodies directed against the desired antigen (e.g., a TREM2 protein of the present disclosure), e.g., as determined by immunoprecipitation or by an in vitro binding assay, such as radioimmunoassay (RIA) or enzyme-linked assay (ELISA). Such techniques and assays are known in the in art.

After hybridoma cells are identified that produce antibodies of the desired specificity, affinity, and/or activity, the clones may be subcloned, and monoclonal antibodies secreted by the subclones may be separated from the culture medium, ascites fluid, or serum by conventional immunoglobulin purification procedures such as, for example, protein A-Sepharose chromatography, hydroxylapatite chromatography, gel electrophoresis, dialysis, affinity chromatography, and other methods as described above.

Anti-TREM2 monoclonal antibodies may also be made by recombinant DNA methods, e.g., as described above. DNA encoding the monoclonal antibodies is readily isolated and sequenced using conventional procedures (e.g., by using oligonucleotide probes that specifically bind to genes encoding the heavy and light chains of murine antibodies). The hybridoma cells serve as a preferred source of such DNA. Once isolated, the DNA may be placed into expression vectors, which are then transfected into host-cells such as E. coli cells, simian COS cells, Chinese hamster ovary (CHO) cells, or myeloma cells that do not otherwise produce immunoglobulin protein, in order to synthesize monoclonal antibodies in such recombinant host-cells. Review articles on recombinant expression in bacteria of DNA encoding the antibody include Skerra et al., Curr. Opin. Immunol., 5:256-262 (1993) and Plückthun, Immunol. Rev. 130:151-188 (1992).

In certain embodiments, anti-TREM2 antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) described the isolation of murine and human antibodies, respectively, from phage libraries. Subsequent publications describe the production of high affinity (nanomolar (“nM”) range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)).

The DNA encoding antibodies or fragments thereof may also be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(3) Humanized Antibodies

Anti-TREM2 antibodies of the present disclosure or antibody fragments thereof may further include humanized or human antibodies. Humanized forms of non-human (e.g., murine) antibodies are chimeric immunoglobulins, immunoglobulin chains or fragments thereof (such as Fab, Fab′-SH, Fv, scFv, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally will also comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Certain methods for humanizing non-human anti-TREM2 antibodies are known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies may impact immunogenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Nat'l Acad. Sci. USA 89:4285 (1992); Presta et al., J. Immunol. 151:2623 (1993).

Humanized antibodies preferably retain high affinity for the antigen and other favorable biological properties. To achieve this goal, according to a preferred method, humanized antibodies are prepared by a process of analyzing the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen or antigens (e.g., TREM2 proteins of the present disclosure), is achieved. In general, the CDR residues are directly and most substantially involved in influencing antigen binding.

Various forms of the humanized anti-TREM2 antibody are contemplated. For example, the humanized anti-TREM2 antibody may be an antibody fragment, such as an Fab, or an intact antibody, such as an intact IgG1 antibody.

(4) Antibody Fragments

In certain embodiments, there are advantages to using anti-TREM2 antibody fragments, rather than whole anti-TREM2 antibodies. In some embodiments, smaller fragment sizes allow for rapid clearance and better brain penetration.

Various techniques have been developed for the production of antibody fragments. Traditionally, these fragments were derived via proteolytic digestion of intact antibodies (see, e.g., Morimoto et al., J. Biochem. Biophys. Method. 24:107-117 (1992); and Brennan et al., Science 229:81 (1985)). However, these fragments can now be produced directly by recombinant host-cells, for example, using nucleic acids encoding anti-TREM2 antibodies of the present disclosure. Fab, Fv and scFv antibody fragments can all be expressed in and secreted from E. coli, thus allowing the straightforward production of large amounts of these fragments. Anti-TREM2 antibody fragments can also be isolated from the antibody phage libraries as discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host-cell culture. Production of Fab and F(ab′)₂ antibody fragments with increased in vivo half-lives are described in U.S. Pat. No. 5,869,046. In other embodiments, the antibody of choice is a single chain Fv fragment (scFv). See WO 93/16185; U.S. Pat. Nos. 5,571,894 and 5,587,458. The anti-TREM2 antibody fragment may also be a “linear antibody,” e.g., as described in U.S. Pat. No. 5,641,870. Such linear antibody fragments may be monospecific or bispecific.

(5) Bispecific and Polyspecific Antibodies

Bispecific antibodies (BsAbs) are antibodies that have binding specificities for at least two different epitopes, including those on the same or another protein (e.g., one or more TREM2 proteins of the present disclosure). Alternatively, one part of a BsAb can be armed to bind to the target TREM2 antigen, and another can be combined with an arm that binds to a second protein. Such antibodies can be derived from full-length antibodies or antibody fragments (e.g., F(ab′)₂ bispecific antibodies).

(6) Effector Function Engineering

It may also be desirable to modify an anti-TREM2 antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some embodiments, the effector function is impaired by removing N-glycosylation of the Fc region (e.g., in the CH 2 domain of IgG) of the antibody. In some embodiments, the effector function is impaired by modifying regions such as 233-236, 297, and/or 327-331 of human IgG as described in PCT WO 99/58572 and Armour et al., Molecular Immunology 40: 585-593 (2003); Reddy et al., J. Immunology 164:1925-1933 (2000). In other embodiments, it may also be desirable to modify an anti-TREM2 antibody of the present disclosure to modify effector function to increase finding selectivity toward the ITIM-containing FcgRIIb (CD32b) to increase clustering of TREM2 antibodies on adjacent cells without activating humoral responses including antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

(7) Other Amino Acid Sequence Modifications

Amino acid sequence modifications of anti-TREM2 antibodies of the present disclosure, or antibody fragments thereof, are also contemplated. For example, it may be desirable to improve the binding affinity and/or other biological properties of the antibodies or antibody fragments. Amino acid sequence variants of the antibodies or antibody fragments are prepared by introducing appropriate nucleotide changes into the nucleic acid encoding the antibodies or antibody fragments, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics (i.e., the ability to bind or physically interact with a TREM2 protein of the present disclosure). The amino acid changes also may alter post-translational processes of the antibody, such as changing the number or position of glycosylation sites.

A useful method for identification of certain residues or regions of the anti-TREM2 antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with the target antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed antibody variants are screened for the desired activity.

Amino acid sequence insertions include amino- (“N”) and/or carboxy- (“C”) terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table C below under the heading of “preferred substitutions”. If such substitutions result in a change in biological activity, then more substantial changes, denominated “exemplary substitutions” in Table C, or as further described below in reference to amino acid classes, may be introduced and the products screened.

TABLE C
Amino Acid Substitutions

| Original | | Preferred |
|---|---|---|
| Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

A particularly preferred type of substitutional variant involves substituting one or more hypervariable region residues of a parent antibody (e.g. a humanized or human anti-TREM2 antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen (e.g., a TREM2 protein of the present disclosure). Such contact residues and neighboring residues are candidates for substitution according to the techniques elaborated herein. Once such variants are generated, the panel of variants is subjected to screening as described herein and antibodies with superior properties in one or more relevant assays may be selected for further development. Affinity maturation may also be performed by employing a yeast presentation technology such as that disclosed in, for example, WO2009/036379A2; WO2010105256; WO2012009568; and Xu et al., Protein Eng. Des. Sel., 26(10): 663-70 (2013).

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

(8) Other Antibody Modifications

Anti-TREM2 antibodies of the present disclosure, or antibody fragments thereof, can be further modified to contain additional non-proteinaceous moieties that are known in the art and readily available, or to contain different types of drug conjugates that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice of Pharmacy, 20th Ed., Alfonso Gennaro, Ed., Philadelphia College of Pharmacy and Science (2000).

Drug conjugation involves coupling of a biological active cytotoxic (anticancer) payload or drug to an antibody that specifically targets a certain tumor marker (e.g. a protein that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue, OneLive Jul. 5, 2012; ADC Review on antibody-drug conjugates; and Ducry et al., (2010). Bioconjugate Chemistry 21 (1): 5-13).

(9) Binding Assays and Other Assays

Anti-TREM2 antibodies of the present disclosure may be tested for antigen binding activity, e.g., by known methods such as ELISA, Western blot, etc.

Detailed exemplary methods for mapping an epitope to which an antibody binds are provided in Morris (1996) “Epitope Mapping Protocols,” in Methods in Molecular Biology vol. 66 (Humana Press, Totowa, N.J.).

### Nucleic Acids, Vectors, and Host Cells

Anti-TREM2 antibodies of the present disclosure may be produced using recombinant methods and compositions, e.g., as described in U.S. Pat. No. 4,816,567. In some embodiments, isolated nucleic acids having a nucleotide sequence encoding any of the anti-TREM2 antibodies of the present disclosure are provided. Such nucleic acids may encode an amino acid sequence containing the VL and/or an amino acid sequence containing the VH of the anti-TREM2 antibody (e.g., the light and/or heavy chains of the antibody). In some embodiments, one or more vectors (e.g., expression vectors) containing such nucleic acids are provided. In some embodiments, a host cell containing such nucleic acid is also provided. In some embodiments, the host cell contains (e.g., has been transduced with): (1) a vector containing a nucleic acid that encodes an amino acid sequence containing the VL of the antibody and an amino acid sequence containing the VH of the antibody, or (2) a first vector containing a nucleic acid that encodes an amino acid sequence containing the VL of the antibody and a second vector containing a nucleic acid that encodes an amino acid sequence containing the VH of the antibody. In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-TREM2 antibody of the present disclosure are provided. In some embodiments, the method includes culturing a host cell of the present disclosure containing a nucleic acid encoding the anti-TREM2 antibody, under conditions suitable for expression of the antibody. In some embodiments, the antibody is subsequently recovered from the host cell (or host cell culture medium).

For recombinant production of an anti-TREM2 antibody of the present disclosure, a nucleic acid encoding the anti-TREM2 antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the anti-TREM2 antibodies of the present disclosure, or fragments thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-TREM2 antibody of the present disclosure.

Suitable host cells for cloning or expression of antibody-encoding vectors include prokaryotic or eukaryotic cells. For example, anti-TREM2 antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross, Nat. Biotech. 22:1409-1414 (2004); and Li et al., Nat. Biotech. 24:210-215 (2006)).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

### Pharmaceutical Compositions

Provided herein are pharmaceutical compositions and/or pharmaceutical formulations comprising the anti-TREM2 antibodies of the present disclosure and a pharmaceutically acceptable carrier.

In some embodiments, pharmaceutically acceptable carrier preferably are nontoxic to recipients at the dosages and concentrations employed. The antibodies described herein may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols. Pharmaceutically acceptable carriers can include, depending on the formulation desired, pharmaceutically-acceptable, non-toxic carriers of diluents, which are vehicles commonly used to formulate pharmaceutical compositions for animal or human administration. In certain embodiments, the pharmaceutical composition can comprise formulation materials for modifying, maintaining or preserving, for example, the pH, osmolarity, viscosity, clarity, color, isotonicity, odor, sterility, stability, rate of dissolution or release, adsorption or penetration of the composition.

In certain embodiments, pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. Further examples of formulations that are suitable for various types of administration can be found in Remington: The Science and Practice of Pharmacy, Pharmaceutical Press 22nd ed. (2013). For a brief review of methods for drug delivery, see, Langer, Science 249:1527-1533 (1990).

Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Formulations may be optimized for retention and stabilization in the brain or central nervous system. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the antibody, such as an anti-TREM2 antibody of the present disclosure, in a biodegradable or bioerodible implant. The rate of release of the therapeutically active agent is controlled by the rate of transport through the polymeric matrix, and the biodegradation of the implant. Implants may be particles, sheets, patches, plaques, fibers, microcapsules and the like and may be of any size or shape compatible with the selected site of insertion. Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the subject invention. Hydrogels are typically a copolymer material, characterized by the ability to imbibe a liquid.

### Kits/Articles of Manufacture

Provided herein are articles of manufacture (e.g., kit) comprising an anti-TREM2 antibody described herein. Article of manufacture may include one or more containers comprising an antibody described herein. Containers may be any suitable packaging including, but is not limited to, vials, bottles, jars, flexible packaging (e.g., sealed Mylar or plastic bags), and the like. The containers may be unit doses, bulk packages (e.g., multi-dose packages) or sub-unit doses.

In some embodiments, the kits may further include a second agent. In some embodiments, the second agent is a pharmaceutically-acceptable buffer or diluting agent including, but not limited to, such as bacteriostatic water for injection (BWFI), phosphate-buffered saline, Ringer's solution and dextrose solution. In some embodiments, the second agent is a pharmaceutically active agent.

In some embodiments of any of the articles of manufacture, the article of manufactures further include instructions for use in accordance with the methods of this disclosure. The instructions generally include information as to dosage, dosing schedule, and route of administration for the intended treatment. In some embodiments, these instructions comprise a description of administration of the antibody of the present disclosure (e.g., an anti-TREM2 antibody described herein) to prevent, reduce risk, or treat an individual having a disease, disorder, or injury selected from dementia, frontotemporal dementia, Alzheimer's disease, Nasu-Hakola disease, cognitive deficit, memory loss, spinal cord injury, traumatic brain injury, a demyelination disorder, multiple sclerosis, Parkinson's disease, amyotrophic lateral sclerosis (ALS), Huntington's disease, adult-onset leukoencephalopathy with axonal spheroids and pigmented glia (ALSP), and a tauopathy disease, according to any methods of this disclosure. In some embodiments, the disease, disorder, or injury is Alzheimer's disease. In some embodiments, the instructions include instructions for use of the anti-TREM2 antibody and a second agent (e.g., second pharmaceutically active agent).

### Biomarkers and Methods of Monitoring Treatment

In some embodiments of the methods of treatment provided herein, the method comprises measuring the levels of soluble TREM2 in a sample of blood, plasma, and/or cerebrospinal fluid from the individual before and after the individual has received one or more doses of the anti-TREM2 antibody. In certain embodiments, the levels of soluble TREM2 are measured in a sample of blood or plasma from the individual before and after the individual has received one or more doses of the anti-TREM2 antibody. In certain embodiments, the levels of soluble TREM2 are measured in a sample of cerebrospinal fluid from the individual before and after the individual has received one or more doses of the anti-TREM2 antibody. The levels of sTREM2 in the sample of blood, plasma, or cerebrospinal fluid from the individual may be measured using any method described herein or known in the art, such as ELISA, immunoassays, immunoblotting, and mass spectrometry.

As used herein, “CSF1R”, “CSF1R protein”, or “CSF1R polypeptide” refer to any native CSF1R from any mammalian source, including primates (e.g., humans and cynomolgus monkeys) and rodents (e.g., mice and rats), unless otherwise indicated. In some embodiments, the term encompasses both wild-type sequences and naturally occurring variant sequences, e.g., splice variants or allelic variants. In some embodiments, the term encompasses “full-length,” unprocessed CSF1R, as well as any form of CSF1R that results from processing in the cell (e.g., soluble CSF1R or sCSF1R). In some embodiments, the CSF1R is human CSF1R. As used herein, “soluble CSF1R” or “sCSF1R” refer to any form of CSF1R that results from processing, e.g., cleavage, of a CSF1R protein, resulting in a soluble, processed form of CSF1R, e.g., as described herein in Example 2.

In some embodiments of the methods of treatment provided herein, the method comprises measuring the levels of soluble CSF1R in a sample of blood, plasma, and/or cerebrospinal fluid from the individual before and after the individual has received one or more doses of the anti-TREM2 antibody. In certain embodiments, the levels of soluble CSF1R are measured in a sample of blood or plasma from the individual before and after the individual has received one or more doses of the anti-TREM2 antibody. In certain embodiments, the levels of soluble CSF1R are measured in a sample of cerebrospinal fluid from the individual before and after the individual has received one or more doses of the anti-TREM2 antibody. The levels of soluble CSF1R in the sample of blood, plasma, or cerebrospinal fluid from the individual may be measured using any method described herein or known in the art, such as ELISA (e.g., an ELISA assay from R&D Systems), immunoassays, immunoblotting, and mass spectrometry.

In some embodiments of the methods of treatment provided herein, the method comprises measuring the levels of YKL40 in a sample of blood, plasma, and/or cerebrospinal fluid from the individual before and after the individual has received one or more doses of the anti-TREM2 antibody. In certain embodiments, the levels of YKL40 are measured in a sample of blood or plasma from the individual before and after the individual has received one or more doses of the anti-TREM2 antibody. In certain embodiments, the levels of YKL40 are measured in a sample of cerebrospinal fluid from the individual before and after the individual has received one or more doses of the anti-TREM2 antibody. The levels of YKL40 in the sample of blood, plasma, or cerebrospinal fluid from the individual may be measured using any method described herein or known in the art, such as ELISA, immunoassays, immunoblotting, and mass spectrometry.

In some embodiments of the methods of treatment provided herein, the method comprises measuring the levels of IL-1RA in a sample of blood, plasma, and/or cerebrospinal fluid from the individual before and after the individual has received one or more doses of the anti-TREM2 antibody. In certain embodiments, the levels of IL-1RA are measured in a sample of blood or plasma from the individual before and after the individual has received one or more doses of the anti-TREM2 antibody. In certain embodiments, the levels of IL-1RA are measured in a sample of cerebrospinal fluid from the individual before and after the individual has received one or more doses of the anti-TREM2 antibody. The levels of IL-1RA in the sample of blood, plasma, or cerebrospinal fluid from the individual may be measured using any method described herein or known in the art, such as ELISA, immunoassays, immunoblotting, and mass spectrometry.

In some embodiments of the methods of treatment provided herein, the method comprises measuring the levels of osteopontin in a sample of blood, plasma, and/or cerebrospinal fluid from the individual before and after the individual has received one or more doses of the anti-TREM2 antibody. In certain embodiments, the levels of osteopontin are measured in a sample of blood or plasma from the individual before and after the individual has received one or more doses of the anti-TREM2 antibody. In certain embodiments, the levels of osteopontin are measured in a sample of cerebrospinal fluid from the individual before and after the individual has received one or more doses of the anti-TREM2 antibody. The levels of osteopontin in the sample of blood, plasma, or cerebrospinal fluid from the individual may be measured using any method described herein or known in the art, such as ELISA, immunoassays, immunoblotting, and mass spectrometry.

In some embodiments of the methods of treatment provided herein, the method comprises measuring the levels of brain amyloid burden in the brain of the individual before and after the individual has received one or more doses of the anti-TREM2 antibody. In certain embodiments, the levels of brain amyloid burden in the brain of the individual are measured using any method provided herein or known in the art, such as amyloid-positron emission tomography (PET), such as longitudinal amyloid-PET, e.g., using [¹⁸F]florbetaben (Neuraceq), [¹⁸F]florbetapir (Amyvid), [¹⁸F]flutametamol (Vizamyl), or any other suitable radiotracer.

In some embodiments of the methods of treatment provided herein, the method comprises measuring tau burden in the brain of the individual, assessed by measuring the levels of tau in the brain of the individual, before and after the individual has received one or more doses of the anti-TREM2 antibody. In certain embodiments, the levels of tau in the brain of the individual are measured using any method provided herein or known in the art, such as Tau-positron emission tomography (PET), e.g., using [¹⁸F]MK-6240 or any other suitable radiotracer.

In some embodiments of the methods of treatment provided herein, the method comprises measuring one or more brain abnormalities (e.g., cerebral vasogenic edema, superficial siderosis of the central nervous system, or cerebral micro- or macro-hemorrhages) in the brain of the individual before and after the individual has received one or more doses of the anti-TREM2 antibody. In certain embodiments, the one or more brain abnormalities are measured using any method provided herein or known in the art, such as magnetic resonance imaging.

In some embodiments of the methods of treatment provided herein, the method comprises measuring brain volume of the individual before and after the individual has received one or more doses of the anti-TREM2 antibody. In certain embodiments, brain volume is measured using any method provided herein or known in the art, such as magnetic resonance imaging (MRI), e.g., volumetric MRI.

In some embodiments of the methods of treatment provided herein, the method comprises detecting the presence of an alteration in one or more genes in the individual selected from APOE, ApoE4, TREM2, CSF1R, CD33, TMEM106b, or CLUSTERIN. In certain embodiments, the presence of an alteration in the one or more genes in the individual is detected using any method provided herein or known in the art, such as targeted sequencing, whole genome sequencing, next-generation sequencing, Sanger sequencing, or polymerase chain reaction (e.g., PCR or qPCR).

In some embodiments of the methods of treatment provided herein, the method comprises measuring the levels of one or more biomarkers of neuroinflammation in a sample of blood, plasma, and/or cerebrospinal fluid from the individual before and after the individual has received one or more doses of the anti-TREM2 antibody. In certain embodiments, the levels of the one or more biomarkers of neuroinflammation are measured in a sample of blood or plasma from the individual before and after the individual has received one or more doses of the anti-TREM2 antibody. In certain embodiments, the levels of the one or more biomarkers of neuroinflammation are measured in a sample of cerebrospinal fluid from the individual before and after the individual has received one or more doses of the anti-TREM2 antibody. Examples of markers of neuroinflammation include, without limitation, IL-6, SPP1, IFI2712A and TOP2A. The levels of markers of neuroinflammation may be measured using any method provided herein or known in the art, such as ELISA, immunoassays, immunoblotting, and mass spectrometry.

In some embodiments of the methods of treatment provided herein, the method comprises measuring the levels of one or more biomarkers of neurodegeneration in a sample of blood, plasma, and/or cerebrospinal fluid from the individual before and after the individual has received one or more doses of the anti-TREM2 antibody. In certain embodiments, the levels of the one or more biomarkers of neurodegeneration are measured in a sample of blood or plasma from the individual before and after the individual has received one or more doses of the anti-TREM2 antibody. In certain embodiments, the levels of the one or more biomarkers of neurodegeneration are measured in a sample of cerebrospinal fluid from the individual before and after the individual has received one or more doses of the anti-TREM2 antibody. Examples of markers of neurodegeneration include, without limitation, NfL. The levels of markers of neurodegeneration may be measured using any method provided herein or known in the art, such as ELISA, immunoassays, immunoblotting, and mass spectrometry.

In some embodiments of the methods of treatment provided herein, the method comprises measuring the expression levels of TREM2, CSF1R, YKL40, IL-1RA, and/or osteopontin in a sample of blood, plasma, and/or cerebrospinal fluid from the individual before and after the individual has received one or more doses of the anti-TREM2 antibody. In certain embodiments, the expression levels of TREM2, CSF1R, YKL40, IL-1RA, and/or osteopontin are measured in a sample of blood or plasma from the individual before and after the individual has received one or more doses of the anti-TREM2 antibody. In certain embodiments, the expression levels of TREM2, CSF1R, YKL40, IL-1RA, and/or osteopontin are measured in a sample of cerebrospinal from the individual before and after the individual has received one or more doses of the anti-TREM2 antibody. In some embodiments, the expression levels of TREM2, CSF1R, YKL40, IL-1RA, or osteopontin refer to protein expression levels. In some embodiments, the expression levels of TREM2, CSF1R, YKL40, IL-1RA, or osteopontin refer to mRNA expression levels. The expression levels of TREM2, CSF1R, YKL40, IL-1RA, and/or osteopontin may be measured using any method provided herein or known in the art, such as RNA-sequencing, polymerase chain reaction (e.g., qPCR), immunoblotting, immunoassays (e.g., ELISA), mass spectrometry, and gene expression microarray methods.

In some embodiments of the methods of treatment provided herein, the method comprises measuring the levels of one or more biomarkers of Alzheimer's disease in a sample of cerebrospinal fluid from the individual before and after the individual has received one or more doses of the anti-TREM2 antibody. Examples of biomarkers of Alzheimer's disease include, without limitation, sTREM2, sCSF1R, Abeta, Aβ42, Aβ40, Tau, p-Tau, total Tau, neurofilament light chain, neurogranin, and YKL40. In some embodiments, the levels of one or more biomarkers of Alzheimer's disease may be measured in a sample of blood or plasma from the individual before and after the individual has received one or more doses of the anti-TREM2 antibody. The levels of the one or more biomarkers of Alzheimer's disease may be measured using any method provided herein or known in the art, such as immunoblotting, immunoassays (e.g., ELISA), and mass spectrometry.

In some embodiments of the methods of treatment provided herein, the method comprises measuring the levels of one or more biomarkers of microglia function in a sample of cerebrospinal fluid from the individual before and after the individual has received one or more doses of the anti-TREM2 antibody. Examples of biomarkers of microglia function include, without limitation, CSF1R, IL1RN, YKL40 and osteopontin. In some embodiments, the levels of one or more biomarkers of microglia function may be measured in a sample of blood or plasma from the individual before and after the individual has received one or more doses of the anti-TREM2 antibody. The levels of the one or more biomarkers of microglia function may be measured using any method provided herein or known in the art, such as immunoblotting, immunoassays (e.g., ELISA), and mass spectrometry.

Also provided herein are methods of monitoring the treatment of an individual being administered an anti-TREM2 antibody.

In some embodiments of the methods of monitoring treatment provided herein, the method comprises measuring the levels of soluble TREM2 in a sample of cerebrospinal fluid, blood or plasma from the individual before and after the individual has received one or more doses of an anti-TREM2 antibody. In some embodiments, the method includes a step of assessing the activity of the anti-TREM2 antibody in the individual based on the levels of soluble TREM2 in the sample of cerebrospinal fluid, blood, or plasma. In some embodiments, the activity of an anti-TREM2 antibody of the present disclosure refers to the engagement of the target (i.e., a TREM2 protein) by the anti-TREM2 antibody (i.e., target engagement). In some embodiments, the anti-TREM2 antibody is determined to be active in the individual if the levels of soluble TREM2 in the sample of cerebrospinal fluid, blood, or plasma are decreased, e.g., by 5%, 10%, 20%, 25%, 30%, 35%, 40%, 45%, 50%, 60%, 70%, 80, 90%, or 100% after the individual has received one or more doses of the anti-TREM2 antibody, compared to the levels of soluble TREM2 in the sample of cerebrospinal fluid, blood, or plasma before the individual received a dose of the anti-TREM2 antibody.

In some embodiments, the levels of soluble TREM2 in a sample of cerebrospinal fluid, blood, or plasma from the individual after the individual has received one or more doses of the anti-TREM2 antibody are compared to the levels of soluble TREM2 in a sample of cerebrospinal fluid, blood, or plasma from the individual at between about 42 days to less than 1 day (e.g., any of 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less than 1 day) before the individual received a dose of the anti-TREM2 antibody. In some embodiments, the levels of soluble TREM2 in a sample of cerebrospinal fluid, blood, or plasma from the individual after the individual has received one or more doses of the anti-TREM2 antibody are compared to the levels of soluble TREM2 in a sample of cerebrospinal fluid, blood, or plasma from the individual at least about 4 days before the individual received a dose of the anti-TREM2 antibody.

The levels of sTREM2 in the sample of cerebrospinal fluid, blood, or plasma from the individual may be measured using any method described herein or known in the art, such as ELISA, immunoassays, immunoblotting, and mass spectrometry.

In some embodiments of the methods of monitoring treatment provided herein, the method comprises measuring the levels of soluble CSF1R in a sample of cerebrospinal fluid, blood, or plasma from the individual before and after the individual has received one or more doses of an anti-TREM2 antibody. In some embodiments, the method includes a step of assessing the activity of the anti-TREM2 antibody in the individual based on the levels of soluble CSF1R in the sample of cerebrospinal fluid, blood, or plasma. In some embodiments, the activity of an anti-TREM2 antibody of the present disclosure refers to the engagement of the target (i.e., a TREM2 protein) by the anti-TREM2 antibody (i.e., target engagement). In some embodiments, the anti-TREM2 antibody is determined to be active in the individual if the levels of soluble CSF1R in the sample of cerebrospinal fluid, blood, or plasma are increased, e.g., by 5%, 10%, 20%, 25%, 30%, 35%, 40%, 45%, 50%, 60%, 70%, 80, 90%, 100%, or more after the individual has received one or more doses of the anti-TREM2 antibody, compared to the levels of soluble CSF1R in the sample of cerebrospinal fluid, blood, or plasma before the individual received a dose of the anti-TREM2 antibody.

In some embodiments, the levels of soluble CSF1R in a sample of cerebrospinal fluid, blood, or plasma from the individual after the individual has received one or more doses of the anti-TREM2 antibody are compared to the levels of soluble CSF1R in a sample of cerebrospinal fluid, blood, or plasma from the individual at between about 42 days to less than 1 day (e.g., any of 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less than 1 day) before the individual received a dose of the anti-TREM2 antibody. In some embodiments, the levels of soluble CSF1R in a sample of cerebrospinal fluid, blood, or plasma from the individual after the individual has received one or more doses of the anti-TREM2 antibody are compared to the levels of soluble CSF1R in a sample of cerebrospinal fluid, blood, or plasma from the individual at least about 4 days before the individual received a dose of the anti-TREM2 antibody.

The levels of soluble CSF1R in the sample of cerebrospinal fluid, blood, or plasma from the individual may be measured using any method described herein or known in the art, such as ELISA, immunoassays, immunoblotting, and mass spectrometry.

In some embodiments of the methods of monitoring treatment provided herein, the method comprises measuring the levels of YKL40 in a sample of cerebrospinal fluid, blood, or plasma from the individual before and after the individual has received one or more doses of an anti-TREM2 antibody. In some embodiments, the method includes a step of assessing the activity of the anti-TREM2 antibody in the individual based on the levels of YKL40 in the sample of cerebrospinal fluid, blood, or plasma. In some embodiments, the activity of an anti-TREM2 antibody of the present disclosure refers to the engagement of the target (i.e., a TREM2 protein) by the anti-TREM2 antibody (i.e., target engagement). In some embodiments, the anti-TREM2 antibody is determined to be active in the individual if the levels of YKL40 in the sample of cerebrospinal fluid, blood, or plasma are increased, e.g., by 5%, 10%, 20%, 25%, 30%, 35%, 40%, 45%, 50%, 60%, 70%, 80, 90%, 100%, or more after the individual has received one or more doses of the anti-TREM2 antibody, compared to the levels of YKL40 in the sample of cerebrospinal fluid, blood, or plasma before the individual received a dose of the anti-TREM2 antibody.

In some embodiments, the levels of YKL40 in a sample of cerebrospinal fluid, blood, or plasma from the individual after the individual has received one or more doses of the anti-TREM2 antibody are compared to the levels of YKL40 in a sample of cerebrospinal fluid, blood, or plasma from the individual at between about 42 days to less than 1 day (e.g., any of 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less than 1 day) before the individual received a dose of the anti-TREM2 antibody. In some embodiments, the levels of YKL40 in a sample of cerebrospinal fluid, blood, or plasma from the individual after the individual has received one or more doses of the anti-TREM2 antibody are compared to the levels of YKL40 in a sample of cerebrospinal fluid, blood, or plasma from the individual at least about 4 days before the individual received a dose of the anti-TREM2 antibody.

The levels of YKL40 in the sample of cerebrospinal fluid, blood, or plasma from the individual may be measured using any method described herein or known in the art, such as ELISA, immunoassays, immunoblotting, and mass spectrometry.

In some embodiments of the methods of monitoring treatment provided herein, the method comprises measuring the levels of IL-1RA in a sample of cerebrospinal fluid, blood, or plasma from the individual before and after the individual has received one or more doses of an anti-TREM2 antibody. In some embodiments, the method includes a step of assessing the activity of the anti-TREM2 antibody in the individual based on the levels of IL-1RA in the sample of cerebrospinal fluid, blood, or plasma. In some embodiments, the activity of an anti-TREM2 antibody of the present disclosure refers to the engagement of the target (i.e., a TREM2 protein) by the anti-TREM2 antibody (i.e., target engagement). In some embodiments, the anti-TREM2 antibody is determined to be active in the individual if the levels of IL-1RA in the sample of cerebrospinal fluid, blood, or plasma are increased, e.g., by 5%, 10%, 20%, 25%, 30%, 35%, 40%, 45%, 50%, 60%, 70%, 80, 90%, 100%, or more after the individual has received one or more doses of the anti-TREM2 antibody, compared to the levels of IL-1RA in the sample of cerebrospinal fluid, blood, or plasma before the individual received a dose of the anti-TREM2 antibody.

In some embodiments, the levels of IL-1RA in a sample of cerebrospinal fluid, blood, or plasma from the individual after the individual has received one or more doses of the anti-TREM2 antibody are compared to the levels of IL-1RA in a sample of cerebrospinal fluid, blood, or plasma from the individual at between about 42 days to less than 1 day (e.g., any of 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less than 1 day) before the individual received a dose of the anti-TREM2 antibody. In some embodiments, the levels of IL-1RA in a sample of cerebrospinal fluid, blood, or plasma from the individual after the individual has received one or more doses of the anti-TREM2 antibody are compared to the levels of IL-1RA in a sample of cerebrospinal fluid, blood, or plasma from the individual at least about 4 days before the individual received a dose of the anti-TREM2 antibody.

The levels of IL-1RA in the sample of cerebrospinal fluid, blood, or plasma from the individual may be measured using any method described herein or known in the art, such as ELISA, immunoassays, immunoblotting, and mass spectrometry.

In some embodiments of the methods of monitoring treatment provided herein, the method comprises measuring the levels of osteopontin in a sample of cerebrospinal fluid, blood, or plasma from the individual before and after the individual has received one or more doses of an anti-TREM2 antibody. In some embodiments, the method includes a step of assessing the activity of the anti-TREM2 antibody in the individual based on the levels of osteopontin in the sample of cerebrospinal fluid, blood, or plasma. In some embodiments, the activity of an anti-TREM2 antibody of the present disclosure refers to the engagement of the target (i.e., a TREM2 protein) by the anti-TREM2 antibody (i.e., target engagement). In some embodiments, the anti-TREM2 antibody is determined to be active in the individual if the levels of osteopontin in the sample of cerebrospinal fluid, blood, or plasma are increased, e.g., by 5%, 10%, 20%, 25%, 30%, 35%, 40%, 45%, 50%, 60%, 70%, 80, 90%, 100%, or more after the individual has received one or more doses of the anti-TREM2 antibody, compared to the levels of osteopontin in the sample of cerebrospinal fluid, blood, or plasma before the individual received a dose of the anti-TREM2 antibody.

In some embodiments, the levels of osteopontin in a sample of cerebrospinal fluid, blood, or plasma from the individual after the individual has received one or more doses of the anti-TREM2 antibody are compared to the levels of osteopontin in a sample of cerebrospinal fluid, blood, or plasma from the individual at between about 42 days to less than 1 day (e.g., any of 42 days, 41 days, 40 days, 39 days, 38 days, 37 days, 36 days, 35 days, 34 days, 33 days, 32 days, 31 days, 30 days, 29 days, 28 days, 27 days, 26 days, 25 days, 24 days, 23 days, 22 days, 21 days, 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days, 1 day, or less than 1 day) before the individual received a dose of the anti-TREM2 antibody. In some embodiments, the levels of osteopontin in a sample of cerebrospinal fluid, blood, or plasma from the individual after the individual has received one or more doses of the anti-TREM2 antibody are compared to the levels of osteopontin in a sample of cerebrospinal fluid, blood, or plasma from the individual at least about 4 days before the individual received a dose of the anti-TREM2 antibody.

The levels of osteopontin in the sample of cerebrospinal fluid, blood, or plasma from the individual may be measured using any method described herein or known in the art, such as ELISA, immunoassays, immunoblotting, and mass spectrometry.

In some embodiments of the methods of monitoring treatment provided herein, the method comprises measuring the levels of one or more biomarkers of Alzheimer's disease in a sample of cerebrospinal fluid, blood, or plasma from the individual before and after the individual has received one or more doses of an anti-TREM2 antibody. In some embodiments, the method includes a step of assessing the activity of the anti-TREM2 antibody in the individual based on the levels of the one or more biomarkers of Alzheimer's disease in the sample of cerebrospinal fluid, blood, or plasma. In some embodiments, the activity of an anti-TREM2 antibody of the present disclosure refers to the engagement of the target (i.e., a TREM2 protein) by the anti-TREM2 antibody (i.e., target engagement). In some embodiments, the one or more biomarkers of Alzheimer's disease comprise Aβ42, Aβ40, total tau, pTau, or neurofilament light. The levels of the one or more biomarkers of Alzheimer's disease in the sample of cerebrospinal fluid, blood, or plasma from the individual may be measured using any method described herein or known in the art, such as ELISA, immunoassays, immunoblotting, and mass spectrometry.

In some embodiments of the methods of monitoring treatment provided herein, the method comprises measuring the levels of one or more biomarkers of microglia function in a sample of cerebrospinal fluid, blood, or plasma from the individual before and after the individual has received one or more doses of an anti-TREM2 antibody. In some embodiments, the method includes a step of assessing the activity of the anti-TREM2 antibody in the individual based on the levels of the one or more biomarkers of microglia function in the sample of cerebrospinal fluid, blood, or plasma. In some embodiments, the activity of an anti-TREM2 antibody of the present disclosure refers to the engagement of the target (i.e., a TREM2 protein) by the anti-TREM2 antibody (i.e., target engagement). In some embodiments, the one or more biomarkers of microglia function comprise CSF1R, IL1RN, YKL40 or osteopontin. The levels of the one or more biomarkers of microglia function in the sample of cerebrospinal fluid, blood, or plasma from the individual may be measured using any method described herein or known in the art, such as ELISA, immunoassays, immunoblotting, and mass spectrometry.

In some embodiments of the methods of monitoring treatment provided herein, the method comprises measuring the levels of amyloid burden in the brain of the individual before and after the individual has received one or more doses of an anti-TREM2 antibody. In some embodiments, the method includes a step of assessing the activity of the anti-TREM2 antibody in the individual based on the levels of amyloid burden in the brain of the individual. In some embodiments, the activity of an anti-TREM2 antibody of the present disclosure refers to the engagement of the target (i.e., a TREM2 protein) by the anti-TREM2 antibody (i.e., target engagement). The levels of brain amyloid burden in the brain of the individual may be measured using any method provided herein or known in the art, such as amyloid-positron emission tomography (PET), such as longitudinal amyloid-PET, e.g., using [¹⁸F]florbetaben (Neuraceq), [¹⁸F]florbetapir (Amyvid), [¹⁸F]flutametamol (Vizamyl), or any other suitable radiotracer.

In some embodiments of the methods of monitoring treatment provided herein, the method comprises measuring tau burden in the brain of the individual, assessed by measuring the levels of tau in the brain of the individual, before and after the individual has received one or more doses of an anti-TREM2 antibody. In some embodiments, the method includes a step of assessing the activity of the anti-TREM2 antibody in the individual based on the levels of tau in the brain of the individual. In some embodiments, the activity of an anti-TREM2 antibody of the present disclosure refers to the engagement of the target (i.e., a TREM2 protein) by the anti-TREM2 antibody (i.e., target engagement). The levels of tau in the brain of the individual may be measured using any method provided herein or known in the art, such as Tau-positron emission tomography (PET), e.g., using [¹⁸F]MK-6240 or any other suitable radiotracer.

In some embodiments of the methods of monitoring treatment provided herein, the method comprises measuring brain volume of the individual before and after the individual has received one or more doses of an anti-TREM2 antibody. In some embodiments, the method includes a step of assessing the activity of the anti-TREM2 antibody in the individual based on the brain volume of the individual. In some embodiments, the activity of an anti-TREM2 antibody of the present disclosure refers to the engagement of the target (i.e., a TREM2 protein) by the anti-TREM2 antibody (i.e., target engagement). In certain embodiments, brain volume is measured using any method provided herein or known in the art, such as magnetic resonance imaging (MRI), e.g., volumetric MRI.

In some embodiments of the methods of monitoring treatment provided herein, the method comprises measuring the expression levels of TREM2, CSF1R, YKL40, IL-1RA, and/or osteopontin in a sample of blood, plasma, and/or cerebrospinal fluid from the individual before and after the individual has received one or more doses of an anti-TREM2 antibody. In some embodiments, the method includes a step of assessing the activity of the anti-TREM2 antibody in the individual based on the expression levels of TREM2, CSF1R, YKL40, IL-1RA, and/or osteopontin in a sample of blood, plasma, and/or cerebrospinal fluid. In some embodiments, the activity of an anti-TREM2 antibody of the present disclosure refers to the engagement of the target (i.e., a TREM2 protein) by the anti-TREM2 antibody (i.e., target engagement). In some embodiments, the expression levels of TREM2, CSF1R, YKL40, IL-1RA, or osteopontin refer to protein expression levels. In some embodiments, the expression levels of TREM2, CSF1R, YKL40, IL-1RA, or osteopontin refer to mRNA expression levels. The expression levels of TREM2, CSF1R, YKL40, IL-1RA, and/or osteopontin may be measured using any method provided herein or known in the art, such as RNA-sequencing, polymerase chain reaction (e.g., qPCR), immunoblotting, immunoassays (e.g., ELISA), mass spectrometry, and gene expression microarray methods.

In some embodiments of the methods of monitoring treatment provided herein, the method comprises measuring the levels of one or more biomarkers of neurodegeneration in a sample of cerebrospinal fluid, blood, or plasma from the individual before and after the individual has received one or more doses of an anti-TREM2 antibody. In some embodiments, the method includes a step of assessing the activity of the anti-TREM2 antibody in the individual based on the levels of the one or more biomarkers of neurodegeneration in the sample of cerebrospinal fluid, blood, or plasma. In some embodiments, the activity of an anti-TREM2 antibody of the present disclosure refers to the engagement of the target (i.e., a TREM2 protein) by the anti-TREM2 antibody (i.e., target engagement). In some embodiments, the one or more biomarkers of neurodegeneration include, without limitation, NfL. The levels of the one or more biomarkers of neurodegeneration in the sample of cerebrospinal fluid, blood, or plasma from the individual may be measured using any method described herein or known in the art, such as ELISA, immunoassays, immunoblotting, and mass spectrometry.

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: A Phase I Study Evaluating the Safety, Tolerability, Pharmacokinetics, Pharmacodynamics, and Immunogenicity of Single and Multiple Doses of AT.1FM in Healthy Participants and in Participants with Mild to Moderate Alzheimer's Disease

This Example describes a multi-center, randomized, double-blind, placebo-controlled, dose escalation, first in human (FIH) study in healthy adults and in participants with mild to moderate Alzheimer's disease (AD). The study was designed to systematically assess the safety (including immunogenicity), tolerability, pharmacokinetics (PK), and pharmacodynamics (PD) of AT.1FM when administered as single ascending doses in healthy participants and as multiple doses in participants with mild to moderate AD.

### I. Study Objectives

The primary objective of this study is to evaluate the safety, tolerability, PK, and PD of AT.1FM administered in single ascending doses in healthy participants and multiple doses in participants with mild to moderate AD.

### II. Study Participants

A. Inclusion Criteria

Participants meeting all of the following Inclusion Criteria are included in the Single Ascending Dose (SAD) phase of this study:
Adults aged 18-65 years.
In good health, determined by no clinically significant findings from medical history, physical examination, ophthalmological examination, 12-lead ECG, laboratory tests, and vital signs.

Participants meeting all of the following Inclusion Criteria are included in the Multiple Dose (MD) phase of this study:
Adults aged 50-85 years.
Clinical diagnosis of probable AD dementia based on the National Institute on Aging Alzheimer's Association criteria.
Screening Mini-Mental State Examination (MMSE) score of 16-28 points.
Screening Clinical Dementia Rating-global Score (CDR-GS) of 0.5, 1.0, or 2.0.
Positive amyloid-PET scan by qualitative read using ¹⁸F-Florbeta PET/CT imaging.
If already taking cholinesterase inhibitor and/or memantine therapy for AD, on a stable dose for at least 4 weeks prior to screening.

In addition, participants that carry at least one of the TREM2 mutations R47H or R62H are enrolled in Cohort M of this study.

B. Exclusion Criteria

Participants meeting any of the following Exclusion Criteria are not included in this study:
History or presence of central nervous system or systemic autoimmune disorders, including but not limited to rheumatoid arthritis, multiple sclerosis, lupus erythematosus, anti-phospholipid antibody syndrome, and Behçet disease.
Known history of severe allergic, anaphylactic, or other hypersensitivity reactions to chimeric, human, or humanized antibodies or fusion proteins.
Current treatment with medications that are well known to prolong the QT interval.
QT interval corrected using Fridericia's formula (QTcF)>450 msec demonstrated by at least 2 ECGs>30 minutes apart.
Present or past history of uveitis requiring medical intervention, chronic inflammatory or degenerative condition of the eye, current eye infection, any ongoing eye disorder requiring injectable medical therapy (e.g. ranibizumab or aflibercept for macular degeneration), or planned invasive eye procedure during the study period.
Past history of seizures, with the exception of childhood febrile seizures.
Immunosuppression caused by disease (such as HIV) or medications; immunosuppressive therapy (such as long-term systemic corticosteroid therapy) within 12 months before screening through the entire study period.
History of major depression (within the past 5 years), unless effectively treated at enrollment and for the duration of the study.
History of schizophrenia, schizoaffective disorder, or bipolar disorder.
At risk of suicide.
Contraindication to lumbar dural puncture, including coagulopathy, concomitant anticoagulation (except for platelet inhibitor such as aspirin or clopidogrel), thrombocytopenia, or other factor that precludes safe lumbar puncture.

In addition, participants that meet any of the following Exclusion Criteria are not included in the Multiple Dose (MD) phase of this study:
Dementia due to a condition other than AD, including, but not limited to, Frontotemporal Dementia, Parkinson's disease, dementia with Lewy bodies, Huntington's disease, or vascular dementia.
History or presence of clinically evident vascular disease potentially affecting the brain (e.g., clinically significant carotid, vertebral stenosis or plaque; aortic aneurysm; intracranial aneurysm; cerebral hemorrhage; arteriovenous malformation) that has the potential to affect cognitive function.
History or presence of stroke within the past 2 years or documented history of transient ischemic attack within the last 12 months.
History of severe, clinically significant (persistent neurologic deficit or structural brain damage) central nervous system trauma (e.g. cerebral contusion).
MRI evidence of:
   More than 2 lacunar infarcts;
   Any territorial infarct>1 cm³; or
   Significant FLAIR hyperintense lesions in the cerebral white matter that may contribute to cognitive dysfunction.
The following medications are prohibited as daily treatment from 1 month prior to screening until the end of the study. They are, however, permitted on an intermittent, as needed basis at any point during the study, provided that no dose is taken within 2 days before any neurocognitive assessment:
   Typical anti-psychotic or neuroleptic medication.
   Narcotic analgesics.
   Sedative, hypnotic, or benzodiazepine medication.
   Tricyclic antidepressant medications.
   Any sedating antihistamine medication (diphenhydramine or other similar over the counter antihistamine therapy).
QT interval corrected using Fridericia's formula (QTcF)>470 msec demonstrated by at least 2 ECGs>30 minutes apart for male participants; QT interval corrected using Fridericia's formula (QTcF)>480 msec demonstrated by at least 2 ECGs>30 minutes apart for female participants.

### III. Study Design

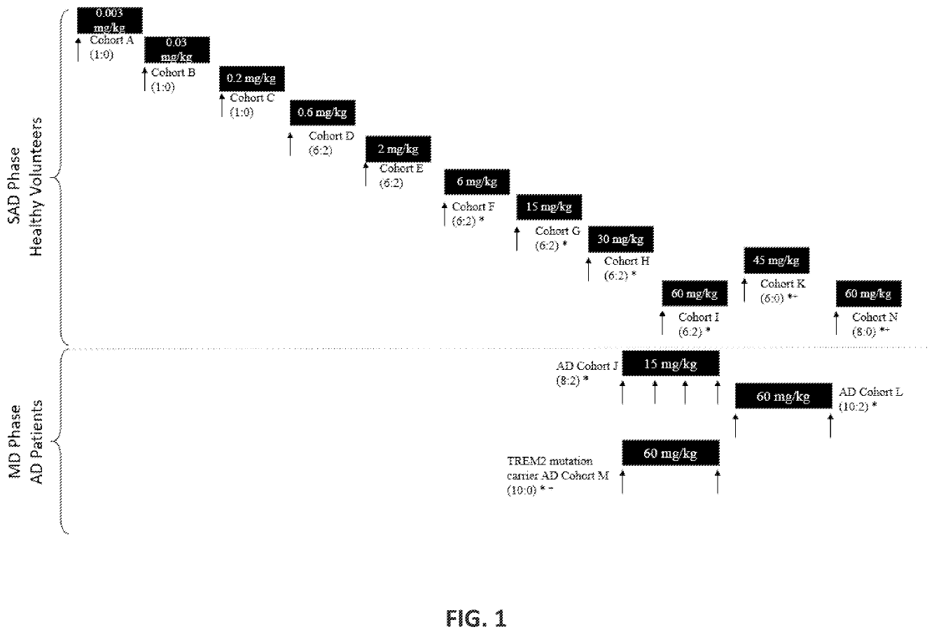

This study is conducted in two phases: a single ascending dose (SAD) phase and a multiple dose (MD) phase. FIG. 1 provides a summary of the design of this study.

A total of approximately 101 participants are enrolled in the study. Of these, approximately 65 healthy adult participants are enrolled in up to 11 predefined single dose, dose-escalating cohorts, and up to 32 participants with AD (28 active drug:4 placebo) are enrolled in up to 3 predefined MD cohorts.

A. Single Ascending Dose Phase

In the SAD phase, up to approximately 65 healthy adult participants are sequentially enrolled in up to 11 cohorts predefined as Cohorts A through I, Cohort K and Cohort N. SAD cohorts A through C include 1 to 3 participants on active drug (AT.1FM) per cohort, and SAD Cohorts D through I include 8 participants per cohort (6 active drug:2 placebo). Open-label SAD Cohort K includes 6 participants treated at a dose of 45 mg/kg. Open-label SAD Cohort N includes 8 participants treated at a dose of 60 mg/kg.

The single dose healthy volunteer phase of the study consists of a screening period, study (treatment) period, follow-up visits and a final follow-up/end of study (EOS) safety assessment visit. The duration of study participation for each participant in the SAD cohorts is approximately 16 weeks.

(i) Screening (Day −28 to Day −2)

Screening occurs within 4 weeks prior to enrollment and prior to the first administered dose of study drug on Day 1. Screening evaluations include a review of the study inclusion/exclusion criteria, complete physical examination, neurological examination, safety assessments (including safety laboratory investigations, measurement of vital signs), and 12-lead triplicate ECG. Lumbar punctures to obtain CSF baseline samples are performed in designated CSF cohorts only (SAD cohorts F, G, H and I).

(ii) Admission and Treatment (Day −1 and Day 1)

Study participants are randomized (as applicable) per cohort to receive AT.1FM or placebo by intravenous (IV) infusion. All participants in Cohorts A through C receive AT.1FM. In Cohorts D through I, a total of 6 participants per cohort receive AT.1FM and 2 participants per cohort receive placebo.

On the day of treatment (Day 1), pre-infusion assessments include review of adverse events (AEs) and concomitant medications, vital signs, 12-lead triplicate ECG, and neurological examination. Collection of baseline samples for serum PK, anti-drug antibodies (ADAs), and assessment of plasma PD biomarkers occurs prior to dosing.

On Day 1, participants receive an IV infusion of AT.1FM or placebo at the relevant dose level for their assigned cohort.

A summary of the treatment schedule for the SAD cohorts is provided in Table 1.

TABLE 1
Treatment Schedule for the SAD Phase.

Number of Participants

| SAD Cohort | Dose (mg/kg) | Active | Placebo |
|---|---|---|---|
| A | 0.003 | 1 to 3 | 0 |
| B | 0.03 | 1 to 3 | 0 |
| C | 0.2 | 1 to 3 | 0 |
| D | 0.6 | 6 | 2 |
| E | 2 | 6 | 2 |
| F | 6 | 6 | 2 |
| G | 15 | 6 | 2 |
| H | 30 | 6 | 2 |
| I | 60 | 6 | 2 |
| K | 45 | 6 | 0 |
| N | 60 | 8 | 0 |

After infusion on Day 1, assessments include review of AEs and concomitant medications, vital signs, and 12-lead triplicate ECG. Collection of samples for serum PK and plasma PD occurs at the end of infusion (within 15 minutes), and at 4, 8 and 12 hours (±15 minutes) post end of infusion. Samples for ADA assessments are collected in participants with signs and symptoms of infusion-related reactions. In such cases, a corresponding additional PK sample is obtained at the same time point as the observed infusion-related reaction. After initiation of study drug infusion, all AEs are reported until 12 weeks after the last infusion.

(iii) Dose Escalation

All participants in Cohorts A through C receive AT.1FM. Cohorts A through C initially include 1 participant per cohort. In the absence of clinically significant safety signals in the first Cohort A participant over the 48-hour safety observation period, Cohort B is initiated. In the absence of clinically significant safety signals in the Cohort B participant following infusion over the 48-hour safety observation period, Cohort C is initiated.

If the first participants in Cohorts A through C experience a clinically significant safety signal, whether it is necessary to enroll 2 more participants in the same cohort (separated by 48 hours between participants) or if it is safe to proceed to the next cohort is evaluated. In the absence of clinically significant safety signals in the Cohort C participant over a 48-hour period, Cohort D is initiated. The first 2 participants in single dose cohorts D through I are sentinels (1 active, 1 placebo). Sentinel participants receive study drug approximately 48 hours before the remaining participants in the cohort. In the absence of clinically significant safety signals in sentinel participants over this period, the remaining participants in the cohort are dosed, with a sufficient minimum interval between participants (≥1 hour) to allow monitoring of any acute post-dose safety events.

(iv) Follow-Up on Days 2 to 3

Following the IV infusion of study drug or placebo on Day 1, participants are monitored, including review of AEs, concomitant medications, and a 12-lead triplicate ECG (on Day 3 at 48 hours±60 minutes post end of infusion). Collection of blood samples for PK and PD biomarker analyses occurs on Day 2 (24 hours±60 minutes) and Day 3 (48 hours±60 minutes) post end of infusion. Lumbar punctures to obtain CSF are performed on Day 3, or on a day determined by preliminary PK and PD data from previous single dose cohorts, where applicable, for all participants within a CSF cohort (i.e., SAD cohorts F, G, H and I).

(v) Follow-Up on Days 5, 8, 13, 30, 43, and 57

Participants are assessed for safety on Day 5, 8 and 13 (±1 day), on Days 30 and 43 (±2 days), and on Day 57 (±3 days). Sampling for PK and PD biomarker measurements occurs at each visit. Sampling for immunogenicity assessments occurs on Day 30 (±2 days) and Day 57 (±3 days).

Participants in a designated CSF cohort (i.e., SAD Cohorts F, G, H and I) undergo lumbar punctures to obtain CSF on Day 13 (±1 day), or on a day determined by preliminary PK and PD data from previous single dose cohorts where applicable.

(vi) End of Study (Day 85)

Participants are evaluated at end of study (EOS) on Day 85 (±5 days). In addition to a review of AEs, concomitant medications, and all safety procedures, participants undergo a 12-lead triplicate ECG and provide samples for PK, PD biomarkers, immunogenicity.

(vii) Open Label Single Dose Cohorts K and N

Cohort K is administered AT.1FM as an open-label cohort of 6 participants at a dose of 45 mg/kg. Cohort N is administered AT.1FM as an open-label cohort of 8 participants at a dose of 60 mg/kg.

Participants in Cohort K undergo lumbar punctures at screening (at least 4 days prior to study drug infusion), on Day 3, and on Day 13 (±1 day), or on a day determined by preliminary PK and PD data from previous single dose cohorts, where applicable.

Participants in Cohort N undergo a lumbar puncture at screening (at least 4 days prior to study drug infusion) and an additional 2 lumbar punctures on either Day 18, Day 30 or Day 43 (±1 day), or a day determined by preliminary PK and PD data from previous cohorts. Each participant in Cohort N undergoes no more than 3 lumbar punctures in total.

B. Multiple Dose Phase

In the MD phase, up to 32 participants with mild to moderate AD are enrolled in up to 3 cohorts predefined as Cohorts J, L, and M. Cohort J includes up to 10 participants (8 active drug:2 placebo). Cohort L includes 12 participants (10 active drug:2 placebo). Cohort M includes 10 participants, all carrying a TREM2 mutation of either R47H or R62H, and all are treated with active drug (open-label).

The MD phase of the study consists of a screening period, study (treatment) period, follow-up visits and a final follow-up/EOS safety assessment visit. For Cohort J, the duration of study participation for each participant is approximately 25 weeks. For Cohorts L and M, the duration of study participation for each participant is approximately 26 weeks.

Participants in MD Cohort J receive AT.1FM or placebo once per week over 4 weeks (Days 1, 8, 15 and 22).

Participants in open-label MD Cohorts L and M receive 2 doses of AT.1FM 4 weeks apart (Days 1 and 29).

Cohort J is initiated once an acceptable safe and tolerable dose level has been identified in the SAD cohorts based on safety and tolerability data up to and including the Day 13 visit. Preliminary PK data from the SAD cohorts are used for predictions of MD PK to inform final selection of the dose level and dosing frequency.

(i) Pre-Screening (Before Day −1)

Pre-screening procedures occur in potential AD participants for Cohort M (TREM2 mutation cohort). Pre-screening takes place prior to Screening, or anytime during the Screening period. Pre-screening consists of a saliva-based screening for TREM2 mutations (R47H and R62H).

(ii) Screening (Day −42 to Day −1)

Screening procedures for all MD cohorts occur within 6 weeks prior to enrollment and prior to the first administered dose of study drug on Day 1.

Full screening evaluations of AD participants for all MD cohorts include a review of the study inclusion/exclusion criteria, complete physical examination, neurological examination, ophthalmological examination, safety assessments including safety laboratory investigations, measurement of vital signs, and 12-lead triplicate ECG.

Participants undergo Mini-Mental State Examination (MMSE), Repeatable Battery for the Assessment of Neuropsychological Status (RBANS), Clinical Dementia Rating (CDR), and brain magnetic resonance imaging (MRI) (including but not limited to FLAIR and T2* weighted GRE sequences) assessments. The screening MRI occurs as close to the beginning of the screening window as possible and at least 10 days prior to randomization on Day 1. A lumbar puncture to obtain a CSF baseline sample is performed. Amyloid-PET imaging is performed in all participants in the MD cohorts.

(iii) Treatment (Day 1)

Study participants are randomized (as applicable) to receive AT.1FM or placebo by IV infusion as follows: Cohort J: 8 active drug and 2 placebo; Cohort L: 10 active drug and 2 placebo; Cohort M: 10 active drug. A summary of the treatment schedule for the multiple dose phase of this study is provided in Table 2.

TABLE 2
Treatment Schedule for Multiple Dose Cohorts.

| | | Number of Participants | |
|---|---|---|---|
| MD Cohort | Dose (mg/kg) (Treatment days) | Active | Placebo |
| J | 15 mg/kg (Day 1, 8, 15 and 22) | 8 | 2 |
| L | 60 mg/kg (Day 1 and 29) | 10 | 2 |
| M* | 60 mg/kg (Day 1 and 29) | 10 | 0 |

*MD Cohort M is open-label and only includes AD participants who carry at least 1 of the 2 TREM2 mutations: R47H or R62H.

Pre-infusion assessments on Day 1 include review of AEs and concomitant medications, assessment of weight, vital signs, safety laboratory investigations, 12-lead triplicate ECG, limited and symptom-directed physical examination, and neurological examination. Participants complete a Sheehan-STS assessment. Collection of baseline samples for assessments of serum PK, ADA, plasma PD biomarkers, and whole blood for WGS occurs prior to dosing. Whole blood collection for mRNA expression and other biomarkers is performed pre-infusion on Day 1.

Safety assessments after end of infusion on Day 1 include review of AEs and concomitant medications, vital signs, and 12-lead triplicate ECG. After initiation of study drug infusion, all AEs are reported until 16 weeks after the last infusion.

Collection of samples for serum PK and plasma PD biomarkers occurs at the end of infusion (within 15 minutes), at 4, 8 and 12 hours (±15 minutes), and at 24 hours (±60 minutes) after infusion. Samples for ADA assessments are collected in participants with signs and symptoms of infusion-related reactions. In such cases, a corresponding additional PK sample is obtained at the same time point as the observed infusion-related reaction. Whole blood collection for mRNA expression and other biomarkers is performed at 24 hours (±60 minutes) after infusion.

(iv) Treatment on Days 8, 15, and 22 (Cohort J) or on Day 29 (Cohorts L and M)

Cohort J: Following the first IV infusion of study drug on Day 1, participants are administered study drug on Days 8, 15, and 22 (±1 day).

Cohorts L and M: Following the first IV infusion of study drug on Day 1, participants are administered a second dose of study drug on Day 29 (±1 day).

Safety assessments include the assessment of AEs, review of concomitant medications, assessment of weight, and a 12-lead triplicate ECG.

Participants complete a Sheehan-STS assessment prior to infusion on Day 8, Day 15 and Day 22 for Cohort J, and on Day 29 for Cohorts L and M. Whole blood collection for analyses of mRNA expression and other biomarkers is performed pre-infusion on Day 8 for Cohort J. For Cohorts L and M, whole blood collection for analyses of mRNA expression and other biomarkers is performed pre-infusion on Day 29 (±2 days).

Collection of blood samples for PK and PD biomarker analyses occurs on Day 8, Day 15 and Day 22 (prior to infusion and again at the end of infusion [within 15 minutes] and at 4 hours post end of infusion [±15 minutes]) for Cohort J. For Cohorts L and M, blood samples for PK and PD biomarker analysis are collected on Day 8, Day 15, Day 22 and Day 29 (prior to infusion and again at the end of infusion [within 15 minutes] and at 4 hours post end of infusion [±15 minutes]).

Sampling for ADA assessments occurs prior to infusion on Day 22 (Cohort J) and Day 29 (Cohorts L and M). Additionally, ADA samples are collected in participants with signs and symptoms of infusion-related reactions. In such cases, a corresponding additional PK sample is obtained at the same time point as the observed infusion-related reaction.

Post-dose lumbar punctures to obtain CSF are performed on Day 29 (±2 days) and Day 50 (±2 days) for Cohort J. Post-dose lumbar punctures to obtain CSF are performed on Day 31 (±2 days) and Day 57 (±2 days) for Cohorts L and M, or on a day determined by preliminary PK and PD data from previous single dose cohorts.

Post-dose amyloid-PET imaging is performed in on Day 106 (−2/+14 days) for Cohort J and Day 113 (−2/+14 days) for Cohorts L and M. A brain MRI is performed on Day 36 (±2 days) for Cohort J and Day 43 (±2 days) for Cohorts L and M. Participants are followed for 16 weeks after the last infusion day.

(v) Follow-Up

After completion of the treatment period, follow-up safety monitoring assessments are performed on Days 29, 36, 50, 64, 78 and 106 (±2 days) for Cohort J, and on Days 31, 36, 43, 57, 71, 85, and 113 (±2 days) for Cohorts L and M. Safety assessments include the assessment of AEs, review of concomitant medications, and a 12-lead triplicate ECG for all participants. Participants complete a Sheehan-STS assessment at each follow-up visit.

Amyloid-PET imaging is performed on Day 106 (−2/+14 days) for Cohort J, and on Day 113 (−2/+14 days) for Cohorts L and M.

A brain MRI is performed on Day 36 (±2 days) for Cohort J, and on Day 43 (±2 days) for Cohorts L and M.

An ophthalmological examination is performed on Day 57 (±6 days) for Cohorts L and M. In the event of clinically significant findings, follow up ophthalmological examinations are performed on a monthly basis, or as clinically indicated, until resolution.

Sampling for PK and PD biomarker measurements occurs at each follow-up visit for all MD cohorts. Sampling for ADA occurs on Days 50, 78 and 106 (±2 days) for Cohort J, and on Days 57, 85 and 113 (±2 days) for Cohorts L and M.

Whole blood collection for analyses of mRNA expression and other biomarkers is performed Day 29 (±2 days) and Day 50 (±2 days) for Cohort J, and on Day 57 for Cohorts L and M.

Lumbar punctures to obtain CSF are performed on Days 29 and 50 (±2 days) for Cohort J, and on Days 31 and 57 (±2 days) for Cohorts L and M, or on a day determined by preliminary PK and PD data from previous single dose cohorts. The Day 31 lumbar puncture for Cohorts L and M occurs between 24 and 48 hours after the end infusion on Day 29.

(vi) End of Study (Day 134 for Cohort J and Day 141 for Cohorts L and M)

End of study assessments occur on Day 134 (±5 days) for Cohort J, and on Day 141 (±5 days) for Cohorts L and M. In addition to a review of AEs, concomitant medications, and safety procedures, participants undergo a 12-lead triplicate ECG and provide samples for analyses of PK, PD biomarkers, and immunogenicity. Participants also complete a Sheehan-STS assessment and undergo MMSE, RBANS, CDR, and brain MRI assessments.

### IV. Study Drug and Placebo

AT.1FM is a recombinant humanized agonistic anti-TREM2 monoclonal antibody. Placebo for IV infusion is normal saline. Study drug or placebo are administered as an IV infusion over approximately 60 minutes.

### V. Study Endpoints

A. Safety Endpoints

The safety endpoints of this study include:
Incidence, nature, and severity of serious adverse events (SAEs) and adverse events of special interest (AESI). AESIs include Amyloid Related Imaging Abnormality-Edema (ARIA-E); vasogenic brain edema; Amyloid Related Imaging Abnormality-Hemosiderin (ARIA-H); new cerebral micro-hemorrhage; and an AE Grade 2 or higher of uveitis.
Incidence of dose limiting adverse events (DLAE).
Incidence of treatment discontinuations due to AEs.
Incidence of dose reductions due to AEs.
Mean changes in clinical laboratory tests from baseline over time; incidence of treatment-emergent abnormal laboratory values and abnormal laboratory values reported as AEs.
Physical and neurologic examination abnormalities.
Ophthalmological examination abnormalities.
Mean change in vital signs from baseline over time and incidence of abnormal vital sign measurements.
Suicidal ideation, suicidal behavior, and self-injurious behavior without suicidal intent, as determined using the Sheehan-STS assessment (for the MD cohorts only).
Incidence of ADAs during the study relative to the prevalence of ADAs at baseline (in SAD and MD cohorts).

B. Pharmacokinetics, Pharmacodynamics, and Biomarker Endpoints

Pharmacokinetic endpoints for this study include:
Serum concentration of AT.1FM.
Relationship between serum concentration or PK parameters for AT.1FM and safety endpoints.
Relationship between serum concentration, CSF concentration, or PK parameters for AT.1FM and activity or PD endpoints (relationship with activity is an endpoint only for the MD cohorts).

In addition, exploratory PD biomarkers for this study include:
Blood-based biomarkers: sTREM2 in plasma, markers of neuroinflammation in blood, and cell surface expression of relevant biomarkers and antigens.
CSF-based biomarkers: sTREM2, CSF biomarkers relevant to AD, and other relevant markers of neuroinflammation.
Genetic markers relevant to the disease indication: apolipoprotein E4 (ApoE4); TREM2 variants, CD33 variants, TMEM106b variants, and CLUSTERIN variants.
Imaging biomarkers (for MD cohorts): MRI and amyloid-PET.

Analyses of exploratory biomarker endpoints include:
Changes in levels of sTREM2 in plasma and CSF after dosing relative to baseline.
Relationship between biomarkers at baseline, including common and rare genetic variants, identified through whole genome sequencing (WGS) performed on deoxyribonucleic acid extracted from blood, and safety, PK, activity, immunogenicity, or other biomarker endpoints (relationship with activity is an endpoint only for the MD cohorts).
Changes in brain amyloid burden as assessed by Amyloid-positron emission tomography (PET) in the MD cohorts only.
Changes in markers of neuroinflammation and disease process in CSF and plasma.
Changes in expression of cell surface antigens.

C. Exploratory Clinical Outcome Endpoints

Exploratory clinical outcome endpoints for this study include (for the MD cohorts only):
Clinical Dementia Rating Sum of Boxes (CDR-SB) score (changes after dosing relative to baseline).
Mini-Mental State Examination (MMSE) score (changes after dosing relative to baseline).
Repeatable Battery for the Assessment of Neuropsychological Status (RBANS) score (changes after dosing relative to baseline).

### VI. Study Assessments

A. Safety Assessments

Safety is determined by evaluating vital signs, 12-lead ECGs in triplicate, monitoring of participant weight, clinical laboratory tests, physical examinations, neurological examinations, ophthalmological examinations, assessment of AEs, and review of concomitant medications. Samples to assess the development of ADAs are collected prior to and throughout the treatment and follow-up periods. In AD participants, the Sheehan-STS is used for prospective suicidality assessments. Brain MRI assessments are performed to detect non-symptomatic brain abnormalities (including but not limited to FLAIR and T2* weighted GRE sequences).

(i) Complete Neurologic Examinations

Complete neurologic examinations include evaluations of consciousness, orientation, cranial nerves, motor and sensory system, coordination and gait, and reflexes.

(ii) Ophthalmological Assessments

Ophthalmological assessments include a visual acuity exam (e.g., using a Snellen chart), slit-lamp examination before and after dilation, dilated exam of the fundus by indirect ophthalmoscopy, and Optical Coherence Tomography (OCT) exam, including Enhanced Depth Imaging OCT for examination of the choroid.

(iii) Sheehan-STS

In AD patients in the MD cohorts, prospective suicidality is assessed regularly throughout the study using Sheehan-STS. The Sheehan-STS is a prospective scale that assesses treatment emergent suicidal thoughts and behaviors. Each item of the Sheehan-STS is scored on a 5-point Likert scale (0=not at all; 1=a little; 2=moderate; 3=very; 4=extremely). For the initial visit, the reference timeframe is in the ‘last 1 year.’ For all subsequent visits, the timeframe is ‘since last evaluation.’

(iv) Magnetic Resonance Imaging

Brain MRI assessments, including but not limited to FLAIR and T2* weighted GRE sequences, are undertaken in AD study participants in the MD cohorts at screening, in the follow-up period, and at the end of study visit to detect non-symptomatic brain abnormalities, such as cerebral vasogenic edema, superficial siderosis of the central nervous system, and cerebral micro or macrohemorrhages. The screening MRI occurs as close to the beginning of the screening window as possible and at least 10 days prior to randomization on Day 1.

(v) Amyloid-Positron Emission Tomography

Amyloid-PET imaging is performed in all participants with AD.

(vi) Screening for TREM2 Mutations

A saliva sample is collected from all potential participants in MD Cohort M (TREM2 mutation cohort) at pre-screening to determine if they are carriers of the R47H or R62H TREM2 mutations. Participants who are determined to be carriers of at least 1 of these 2 mutations are included in Cohort M on the study.

(vii) Anti-Drug Antibodies

Blood samples are collected and analyzed for the presence of AT.1FM anti-drug antibodies (ADA) using a validated bridging immunoassay. Additional samples for ADA assessments are collected in participants with signs and symptoms of infusion-related reactions. In such cases, a corresponding additional PK sample is obtained at the same time point as the observed infusion-related reaction.

B. Clinical Assessments

Alzheimer's disease participants in the MD cohorts undergo MMSE, RBANS, and CDR assessments. Results are summarized by time point and treatment group (active or placebo).

(i) Mini-Mental State Examination (MMSE)

The MMSE is a brief test used to screen for cognitive impairment. It is routinely used for estimating the severity of cognitive impairment and tracking cognitive changes in an individual over time. The MMSE assesses orientation (time and place), registration, attention and calculation, recent memory, language (naming, comprehension and repetition), and constructional praxis (copying a figure). The maximum total score is 30, with a higher score indicating better cognitive performance.

(ii) Repeatable Battery for the Assessment of Neuropsychological Status (RBANS)

The RBANS is a collection of 12 subtests representing 5 neurocognitive domains: Immediate Memory, Visuospatial/Constructional, Language, Attention, and Delayed Memory. The raw scores from each subtest within a domain are converted to a summary score or an Index Score for the domain by consulting normative data tables. The RBANS also provides an overall Index Score that summarizes the patient's overall level of performance on this measure.

(iii) Clinical Dementia Rating (CDR)

Washington University's CDR is a global assessment instrument that yields global scores (i.e., CDR-GS). The sum of boxes (i.e. CDR-SB) score is a detailed quantitative general index that provides more information than the CDR-GS in patients with mild dementia (O'Bryant et al (2010) Arch Neurol, 67(6):746-49). The CDR characterizes 6 domains of cognitive and functional performance applicable to AD and related dementias: memory, orientation, judgment and problem solving, community affairs, home and hobbies, and personal care. The necessary information to make each rating is obtained through a semi-structured interview of the patient and a reliable informant or collateral source (e.g., a caregiver).

C. Pharmacokinetics Assessments

Blood samples for serum PK analyses are obtained during the following times:
Prior to dosing, within 60 minutes of actual dose.
End of infusion (±15 minutes).
Between 4-12 hours post end of infusion (±15 minutes).
Between 24-48 hours post end of infusion (±60 minutes).
After 48 hours to 12 days post end of infusion (Day 13) (±1 day).
Beyond Day 13 (>12 days post end of infusion) (±2-5 days).

Serum PK analyses are performed using validated procedures and methods.

Individual and mean serum AT.1FM concentration-time data are tabulated and plotted by cohort/dose level. PK parameters are computed from the individual serum AT.1FM concentrations using a non-compartmental approach. The following PK parameters are estimated:
Maximum drug concentration (Cₘₐₓ).
Time to reach Cₘₐₓ (Tₘₐₓ).
Area under the drug concentration-time curve from time zero to the last quantifiable concentration (AUC₍₀₋ₗₐₛₜ₎).
Area under the drug concentration-time curve from time zero to infinity (AUC_{(0-inf)}), calculated as the sum of AUC₍₀₋ₗₐₛₜ₎ plus the last measurable plasma concentration divided by elimination rate constant [kₑₗ].
Area under the drug concentration-time curve over the inter-dosing interval (AUCₜₐᵤ), where tau is the time over the inter-dosing interval (calculated for the MD cohorts only).
Apparent terminal elimination rate constant (kₑₗ) calculated by linear regression of the terminal linear portion of the log concentration vs. time curve.
Apparent terminal half-life (t_{1/2}).
Apparent total body clearance after extravascular administration (SAD cohorts: apparent total body clearance after extravascular administration [CL]; MD cohorts CLss), calculated as Dose/AUC_{0-inf} for single/first dose, and Dose/AUCₜₐᵤ after MD administrations.
Apparent total volume of distribution at the terminal phase after extravascular administration (SAD cohorts: Vz; MD cohorts: Vzss), calculated as Dose/(Kₑₗ×AUC_{0-inf}) after single/first dose, and Dose/(kₑₗ×AUCₜₐᵤ) after MD administrations.

Values for kₑₗ, t_{1/2}, AUC_{0-inf}, CL, or Vz are reported for cases that fail to exhibit a terminal log-linear phase in the concentration versus time profile.

Estimates for PK parameters are tabulated and summarized by descriptive statistics (mean, standard deviation, median, minimum, maximum, coefficient of variation (CV %), geometric mean, 90% confidence interval, and geometric CV %). Individual and mean AT.1FM CSF concentration-time data are tabulated by cohort/dose level.

Potential correlations of relevant PK parameters with dose, demographics, safety (including QT changes), and PD measures are explored. Additional modeling, including population PK analysis, to characterize these correlations are performed.

D. Pharmacodynamics Assessments

Samples for PD exploratory biomarker assessments are collected and analyzed using validated assay methods.

All PD biomarker data are summarized by time point, treatment group, and cohort with descriptive statistics (e.g., number of non-missing observations, arithmetic mean, standard deviation, median, minimum, maximum and CV %). The number of values below the limit of quantitation is also presented. Observed changes from baseline and percent changes from baseline for PD biomarker parameters are summarized separately for single dosing cohorts and multiple dosing cohorts, as applicable.

Exploratory analyses are conducted to evaluate the effect of AT.1FM on exploratory biomarkers. In addition, exploratory biomarkers are analyzed before and after dosing with AT.1FM to determine the relationship between PK exposure and biomarker levels.

(i) Blood Biomarkers

Blood samples for PD measures are collected at the same collection times as samples for PK analyses. Blood-based biomarkers include but are not limited to soluble TREM2 (sTREM2) in plasma, markers of neuroinflammation in blood, mRNA, and other biomarkers.

For the MD cohorts only, whole blood samples for the study of mRNA expression and other biomarkers are also collected.

(ii) Lumbar Punctures

Lumbar punctures to obtain CSF samples are performed for selected cohorts as described above.

The following CSF biomarkers are assessed:
Soluble TREM2.
Soluble CSF1R.
CSF biomarkers relevant to AD (this includes but is not limited to Abeta, Tau, p-Tau, neurofilament light chain, neurogranin, and YKL40).
Other relevant markers of neuroinflammation.

(iii) Whole Genome Sequencing

For the MD cohorts only, collection of a baseline blood sample for whole genome sequencing (WGS) occurs prior to dosing on Day 1. Genetic markers relevant to the disease indication are assessed, including ApoE4, TREM2 variants, CD33 variants, TMEM106b variants, and CLUSTERIN variants.

E. Statistics

Data are analysed and presented separately for the SAD and MD cohorts. All continuous data are summarized using descriptive summary statistics (number of non-missing observations, mean, standard deviation, and minimum and maximum). Categorical data are summarized as frequency counts and percentages. Baseline refers to the last available, non-missing observation prior to first study drug administration. Missing data is not imputed, unless otherwise specified.

The following analysis populations are defined for the study:

Treatment Received-Population: The treatment-received population includes all randomized participants and is based on the treatment/dose level received.

Safety Population: The safety population includes all randomized participants who receive any amount of AT.1FM or placebo and is based on the actual treatment/dose level received, if this differs to what the participant is randomized to.

PK Population: The PK population includes all randomized participants who receive any amount of active study drug (AT.1FM) with sufficient plasma concentration-time data to determine at least 1 PK parameter. Participants who receive only placebo are excluded from the PK population. For the MD cohorts, only participants that receive all doses of AT.1FM are included in the PK population.

PD Population: The PD population includes all randomized participants who receive any amount of AT.1FM or placebo and have results from baseline and from ≥1 post-baseline PD assessment. The PD population is based on the actual treatment/dose level received, if this differs from what the participant was randomized to. For the MD cohorts, only participants that receive all doses of AT.1FM are included in the PD population.

### Example 2: Results of a Phase I Study Evaluating the Safety, Tolerability, Pharmacokinetics, Pharmacodynamics, and Immunogenicity of Single Doses of AT.1FM in Healthy Participants

This Example describes results of the single ascending dose (SAD) phase of the study described in Example 1.

### Materials and Methods

Phase 1 Study (Single Ascending Dose Phase)

As described in detail in Example 1, 56 healthy adult participants were sequentially enrolled in to 10 cohorts (A-H, K and I) and received a single intravenous (IV) dose of AT.1FM, ranging from 0.003 mg/kg to 60 mg/kg 1 (see Table 1). SAD Cohorts A through C included 1 participant on active drug per cohort; Cohorts D through H included of 8 participants per cohort (6 active:2 placebo); and Cohort I included of 7 participants (6 active:1 placebo). In open-label Cohort K, 6 participants were treated with active drug at a dose of 45 mg/kg. For Cohorts F through K, lumbar punctures were performed pre-dose, 2 days post-dose, and 12 days post-dose to obtain cerebrospinal fluid (CSF) samples. All subjects were followed until Day 85.

sTREM2 Assay in Human CSF

An immunoassay method was qualified for the determination of sTREM2 in human CSF using an electrochemiluminescent methodology. A first anti-human TREM2 antibody was diluted in coating buffer and immobilized onto a 96-well microtiter sample plate. After blocking and washing the plate, endogenous quality control and study samples were diluted with Assay Buffer, dispensed onto the sample plate, and incubated. A second anti-human TREM2 antibody that binds to a different epitope than the first antibody was added as capture antibody. The plate was subsequently washed, and Sulfo-Tag streptavidin was added and incubated, followed by addition of MSD Read Buffer T. Concentrations were determined on a standard curve obtained by relative light units versus concentration. The calibration curve was generated using a four-parameter curve fit with 1/y² weighting. The qualified range for this method in human CSF is from 0.400 ng/mL to 50.0 ng/mL.

sCSF1R Assay in Human CSF

A commercial ELISA assay by R&D Systems was qualified for the determination of CSF1R in human CSF. A human M-CSF R capture antibody was diluted in coating buffer and immobilized onto a 96-well microtiter sample plate. After blocking and washing the plate, endogenous quality control and study samples were diluted, dispensed onto the sample plate, and incubated. A human M-CSF R detection antibody was added and incubated. The plate was washed and a Streptavidin-HRP reagent was subsequently added, followed by a working substrate solution. The plate was incubated at ambient temperature and stopped with the addition of Sulfuric Acid Stop Solution. The plate was read on a plate reader using two filters: 450 nm for detection and 570 nm for background. Concentrations were determined on a standard curve obtained by plotting optical density versus concentration. The calibration curve was generated using a four-parameter curve fit with 1/y² weighting. The qualified range for this method in 100% human CSF is from 125 pg/mL to 4000 pg/mL.

### Results

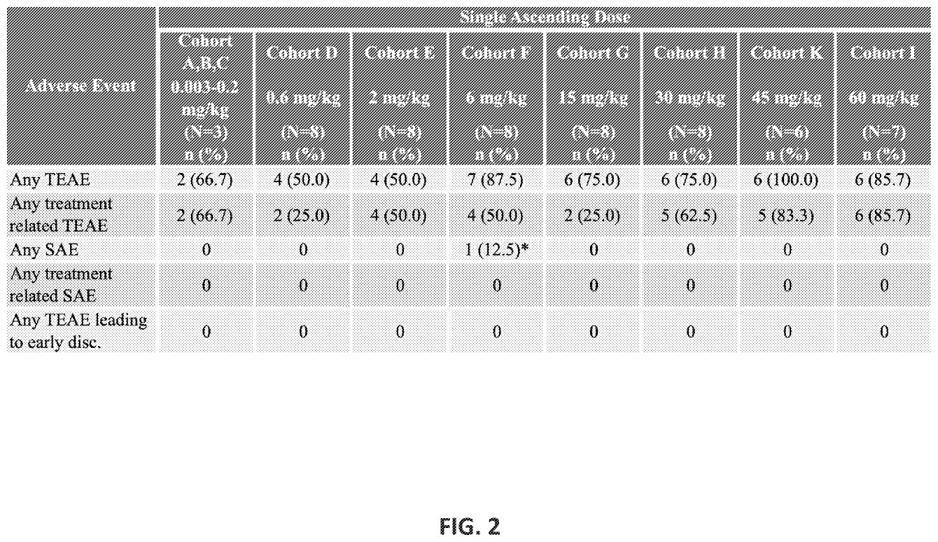

As shown in FIG. 2, AT.1FM was generally safe and well tolerated. No drug related serious adverse events or dose-limiting toxicities were observed up to the highest dose of antibody.

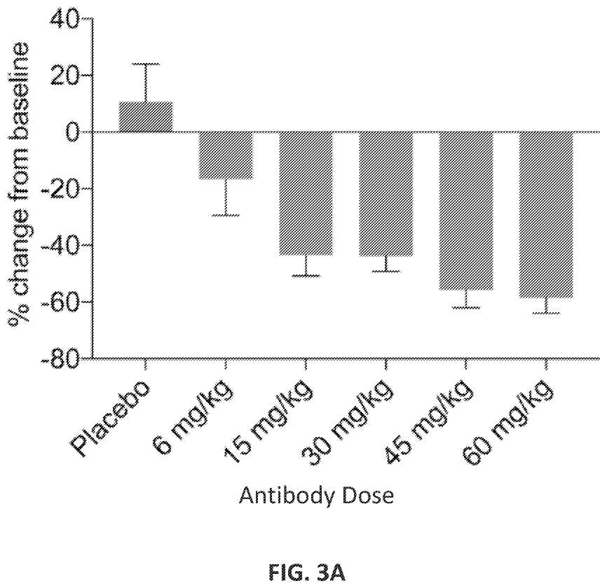

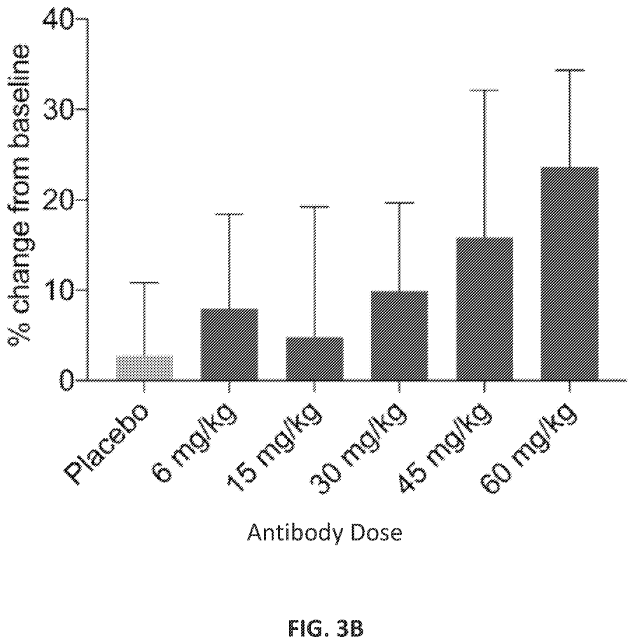

Next, the effect of AT.1FM on CSF biomarkers was assessed. As shown in FIG. 3A, administration of a single dose of AT.1FM caused a dose-dependent decrease in soluble TREM2 (sTREM2) from baseline when assessed at two days post antibody administration. sTREM2 is the product of the cleavage of cell surface TREM2 by metalloproteases (Feuerbach et al (2017) Neurosci Lett, 660:109-114.)

As shown in FIG. 3B, the decrease in sTREM2 levels was paralleled by an increase in soluble CSF1R (sCSF1R) levels when assessed at two days post antibody administration. sCSF1R is the cleavage product of transmembrane protein CSF1R, which is only expressed by microglia in the brain.

Changes in concentrations of additional biomarkers in the CSF were determined at pre-dose, and at day 2 and day 12 post dose in healthy human volunteers administered anti-TREM2 antibody AT.1FM at 6 mg/kg, 15 mg/kg, 30 mg/kg, 45 mg/kg, and 60 mg/kg.

### Example 3: Effects of Anti-TREM2 Antibody on Various Pharmacodynamic Markers in Healthy Human Volunteers

This Example describes results of experiments that evaluated the concentrations of biomarkers in the CSF of healthy human volunteers administered anti-TREM2 antibody AT.1FM at doses of 6 mg/kg, 15 mg/kg, 30 mg/kg, 45 mg/kg, and 60 mg/kg, as described in Example 2. CSF samples were obtained from healthy human volunteers pre-dose and on day 2 and day 12 following administration of anti-TREM2 antibody AT.1FM. Changes in the concentrations of the following biomarkers in the CSF were determined: sTREM2, sCSF1R, YKL40, IL-1RA, and osteopontin.

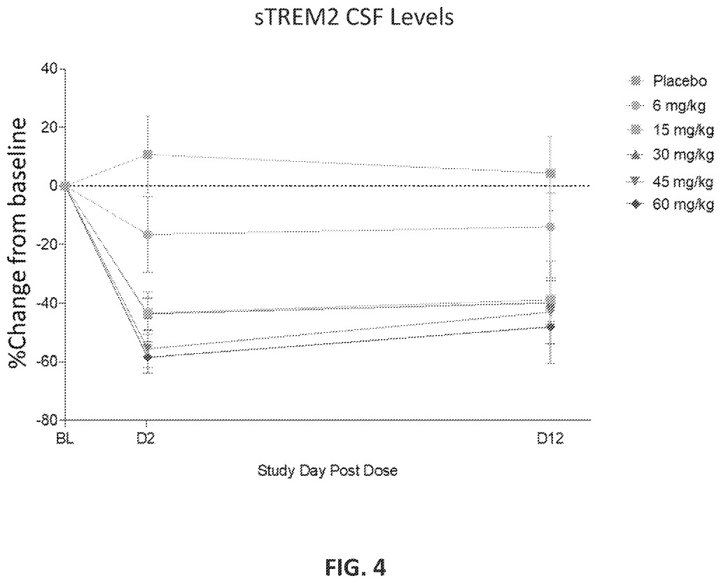

As shown in FIG. 4, anti-TREM2 antibody AT.1FM reduced sTREM2 levels in the CSF of healthy human volunteers compared to baseline in a dose-dependent manner, indicating target engagement of the antibody. The reductions in sTREM2 levels in CSF were largely maintained up to 12 days post-dose.

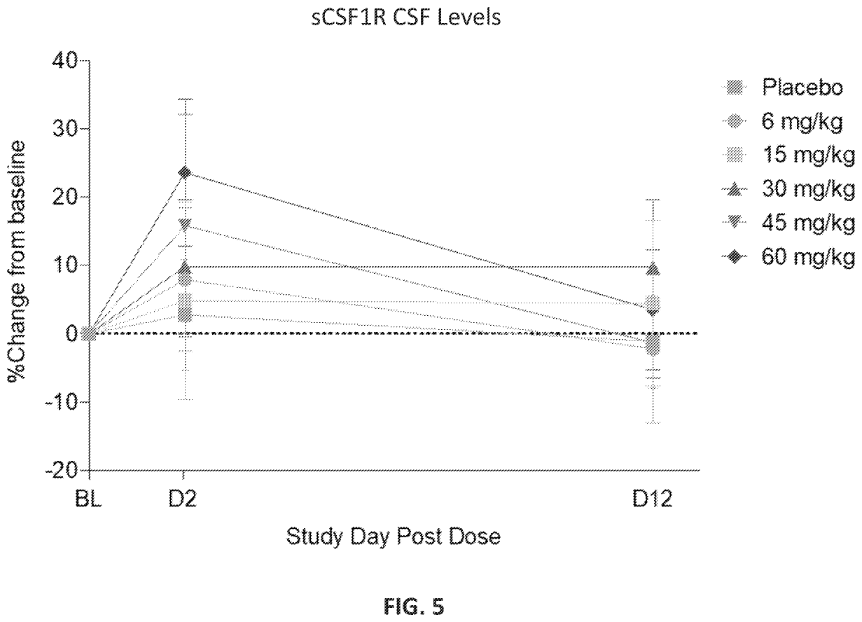

As shown in FIG. 5, anti-TREM2 antibody AT.1FM increased levels of sCSF1R in the CSF of healthy human volunteers compared to baseline.

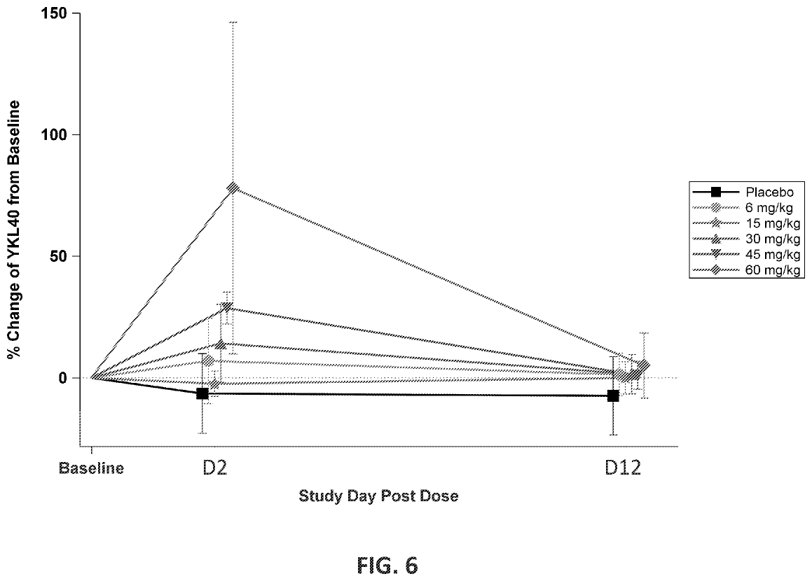

As shown in FIG. 6, anti-TREM2 antibody AT.1FM increased levels of YKL40 in the CSF of healthy human volunteers compared to baseline. YKL40 levels were determined using an immunoassay from Roche.

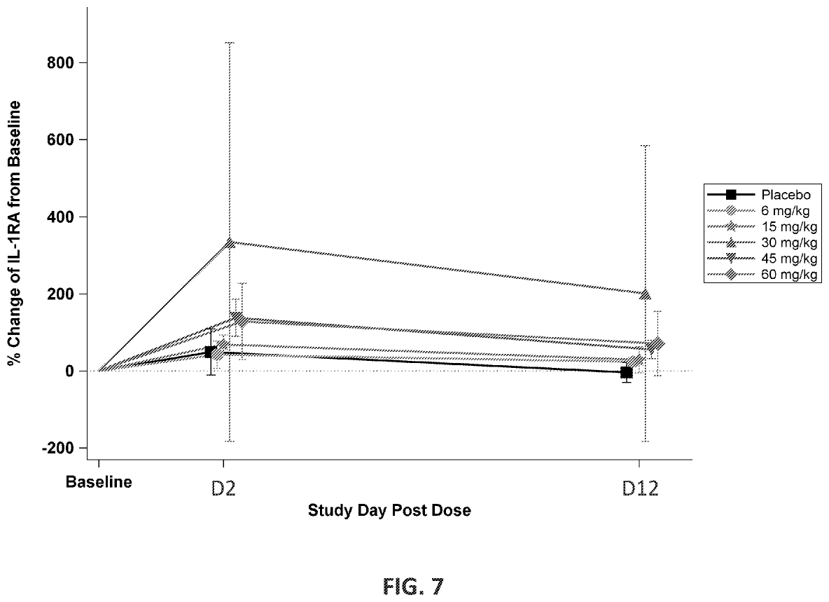

As shown in FIG. 7, anti-TREM2 antibody AT.1FM increased levels of IL-1RA (IL1RN) in the CSF of healthy human volunteers compared to baseline. IL-1RA levels were determined by ECL immunoassay using the Meso Scale Discovery system.

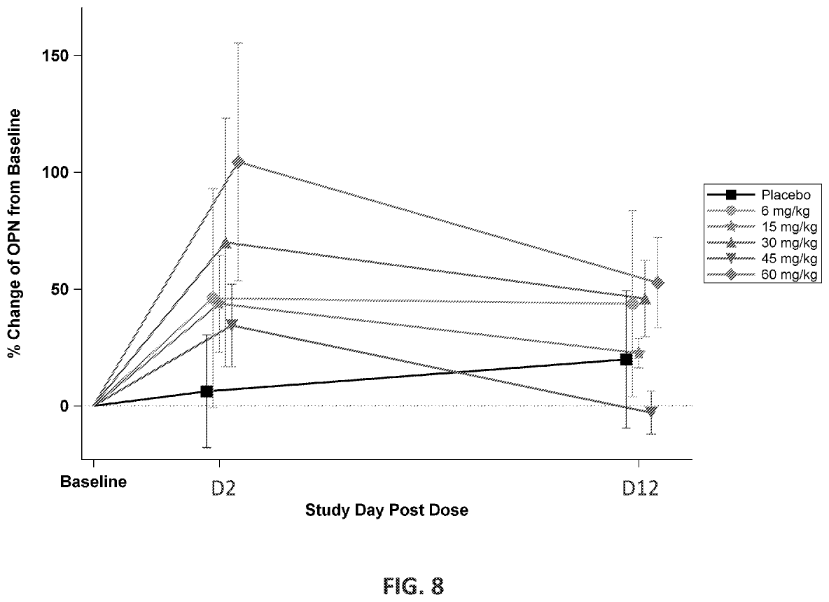

As shown in FIG. 8, anti-TREM2 antibody AT.1FM increased levels of osteopontin (OPN) in the CSF of healthy human volunteers compared to baseline. Osteopontin levels were determined by ECL immunoassay using the Meso Scale Discovery system.

The observed modulation in the CSF levels of sCSF1R, YKL40 (CHI3L1), IL-1RA (IL1RN), and osteopontin (SPP1) by anti-TREM2 antibody AT.1FM indicated activation of microglia subsequent to target engagement.

Preliminary data were available for two AD participants from the MD cohorts. Both of those AD participants had decreased CSF sTREM2 in response to AT.1FM treatment, suggesting target engagement in line with the trend seen in healthy volunteer participants.

### Example 4: Pharmacokinetics of Anti-TREM2 Antibody in Serum of Healthy Human Volunteers

This Example describes a Phase 1 study according to the protocol described in Example 1 that examined the peripheral pharmacokinetics (PK) of intravenously administered anti-TREM2 antibody AT.1FM in healthy humans.

Healthy human volunteer subjects were administered a single dose of antibody AT.1FM (or placebo control) as an intravenous infusion over approximately one hour. The anti-TREM2 antibody AT.1FM doses used in this study were 0.003 mg/kg, 0.03 mg/kg, 0.2 mg/kg, 0.6 mg/kg, 2 mg/kg, 6 mg/kg, 15 mg/kg, 30 mg/kg, 45 mg/kg, and 60 mg/kg. The 0.003 mg/kg, 0.03 mg/kg, and 0.2 mg/kg cohorts each included a single subject dosed with antibody AT.1FM. The 0.6 mg/kg, 2 mg/kg, 6 mg/kg, 15 mg/kg, 30 mg/kg and 45 mg/kg cohorts each included 8 subjects, 6 of whom were dosed with antibody AT.1FM and 2 of whom were dosed with placebo control. The 60 mg/kg cohort included 7 subjects, 6 of whom were dosed with antibody AT.1FM and 1 of whom was dosed with placebo control.

Blood was drawn from the human subjects at multiple timepoints to obtain anti-TREM2 antibody concentrations in serum for the measurement of pharmacokinetics. Anti-TREM2 antibody concentrations were available up to 84 days post-dose for all cohorts. Anti-TREM2 antibody serum concentrations were assayed using an ELISA assay.

Serum PK data for anti-TREM2 antibody AT.1FM in healthy volunteers from each of the dose cohorts are provided in Table 3.

TABLE 3
Serum pharmacokinetics measurements of
AT.1FM in healthy human volunteers.

| | | | |
|---|---|---|---|
| | | AUC_{inf} | |
| | Cₘₐₓ (μg/mL) | (hr*μg/mL) | T_{1/2} (hr) |
| Dose Level | (CV %) | (CV %) | (CV %) |

| | | | | |
|---|---|---|---|---|
| 0.003 | mg/kg | 0.06 | 3.85 | 43.5 |
| | | (N/A) | (N/A) | (N/A) |
| 0.03 | mg/kg | 0.88 | 94.3 | 101.3 |
| | | (N/A) | (N/A) | (N/A) |
| 0.2 | mg/kg | 4.32 | 432 | 116.1 |
| | | (N/A) | (N/A) | (N/A) |
| 0.6 | mg/kg | 15.4 | 1280 | 123.9 |
| | | (22.9) | (27.6) | (13.8) |
| 2 | mg/kg | 62.2 | 5369 | 188.1 |
| | | (23.0) | (17.9) | (40.5) |
| 6 | mg/kg | 147.7 | 15,990 | 196.7 |
| | | (10.9) | (10.3) | (11.0) |
| 15 | mg/kg | 439.6 | 48,090 | 207.1 |
| | | (22.2) | (6.7) | (30.1) |
| 30 | mg/kg | 725.0 | 81,860 | 178.5 |
| | | (10.6) | (20.4) | (29.2) |
| 45 | mg/kg | 1087.0 | 116,000 | 201.5 |
| | | (49.8) | (48.1) | (37.4) |
| 60 | mg/kg | 1413.4 | 129,800 | 238.2 |
| | | (21.9) | (28.1) | (21.5) |

Cₘₐₓ = maximum antibody concentration; AUC_{inf} = area under the drug concentration-time curve from time zero to infinity; T_{1/2} = terminal half-life; hr = hours; CV % = coefficient of variation; N/A = not available.

As shown in Table 3, anti-TREM2 antibody AT.1FM administered to healthy human volunteers displayed an approximate dose proportional Cₘₐₓ. The data also showed that plasma terminal half-life of anti-TREM2 antibody AT.1FM was short at all doses tested, ranging from 123.9 hours (5.16 days) at the 0.6 mg/kg dose to 238.2 hours (9.93 days) at the 60 mg/kg dose.

Overall, the results presented in this Example indicated that at the doses tested, anti-TREM2 antibody AT.1FM was cleared more rapidly than other therapeutic antibodies of a similar class. For example, anti-TREM2 antibody AT.1FM unexpectedly showed a short terminal half-life in serum compared to other antibodies of a similar class (Ovacik, M and Lin, L, (2018) Clin Transl Sci 11, 540-552). The relatively short terminal half-life of anti-TREM2 antibody AT.1FM suggested that the antibody may not have a sufficiently robust therapeutic efficacy. However, as shown in the above Examples, administration of single doses of anti-TREM2 antibody AT.1FM to healthy human volunteers resulted in changes in the protein levels in CSF of certain biomarkers of target engagement and/or microglial activation (e.g., CSF1R, YKL40, IL-1RA, osteopontin, or TREM2) that were present at 2 days after administration of the antibody, and in some cases up to 12 days after administration of the antibody (see, e.g., Examples 2 and 3). Similarly, as described in the subsequent Examples, administration of multiple doses of anti-TREM2 antibody AT.1FM (or of a variant of antibody AT.1FM) to non-human primates also resulted in sustained modulation of certain biomarkers of target engagement and/or microglial activation (e.g., TREM2, osteopontin, or CSF1R) in tissues such as the frontal cortex or hippocampus, and/or in CSF (see, e.g., Examples 6, 7 and 8). Thus, notwithstanding the relatively short terminal half-life of anti-TREM2 antibody AT.1FM, the results described herein show that anti-TREM2 antibody AT.1FM has pharmacodynamic effects that are indicative of therapeutic activity of the antibody.

### Example 5: Pharmacokinetics of Anti-TREM2 Antibody in Cerebrospinal Fluid (CSF) of Healthy Human Volunteers

Cerebrospinal fluid (CSF) was obtained by lumbar puncture from healthy human volunteers administered a single dose of anti-TREM2 antibody AT.1FM (or placebo control) as an intravenous infusion as described above in Example 1. Anti-TREM2 antibody CSF concentrations were tested at day 2 and day 12 post-dose for the 6 mg/kg, 15 mg/kg, 30 mg/kg, 45 mg/kg, and 60 mg/kg cohorts. Anti-TREM2 antibody CSF concentrations were assayed using an ELISA assay.

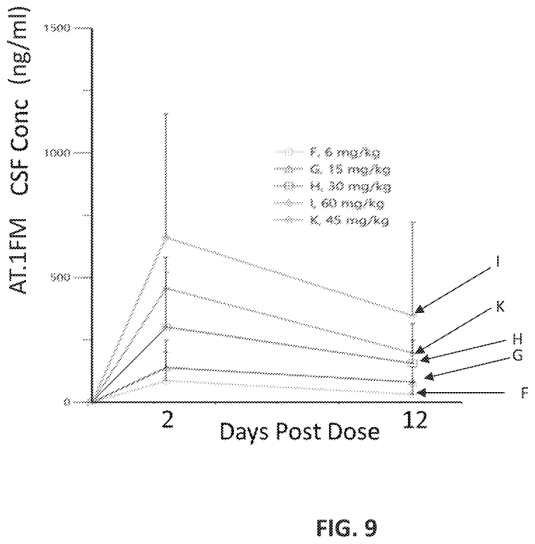

As shown in FIG. 9, anti-TREM2 antibody AT.1FM concentrations in CSF displayed a dose-dependent increase at day 2 and day 12 post-dose. In FIG. 9, data are presented as the mean (+standard deviation) of CSF concentrations of anti-TREM2 antibody AT.1FM (ng/ml). At 12 days post-dose, the ratio of CSF to serum concentrations of anti-TREM2 antibody AT.1FM was about 0.2%-0.3%.

### Example 6: Anti-TREM2 Antibody Reduces Parenchymal TREM2 Levels in Non-Human Primates

This Example describes the results of a study evaluating the pharmacodynamics (PD) of intravenously-administered anti-TREM2 antibody AT.1FM in non-human primates (cynomolgus monkeys).

For this study, the non-human primates were administered anti-TREM2 antibody AT.1FM by intravenous infusion at doses of 20 mg/kg, 80 mg/kg, or 250 mg/kg once weekly for a total of 5 doses (N=6 per dose group). Forty-eight hours after the fifth dose was administered, tissue was harvested from the animals and the amount of TREM2 protein was determined in the frontal cortex and in the hippocampus. The levels of TREM2 protein (ng) measured in the tissue samples were normalized to the total protein (mg) in each sample.

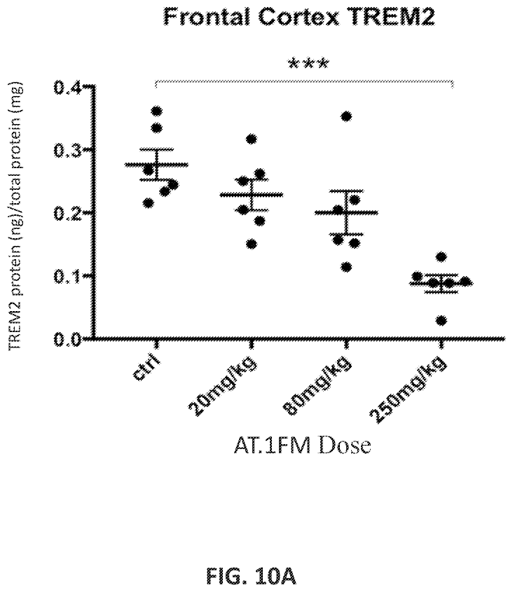

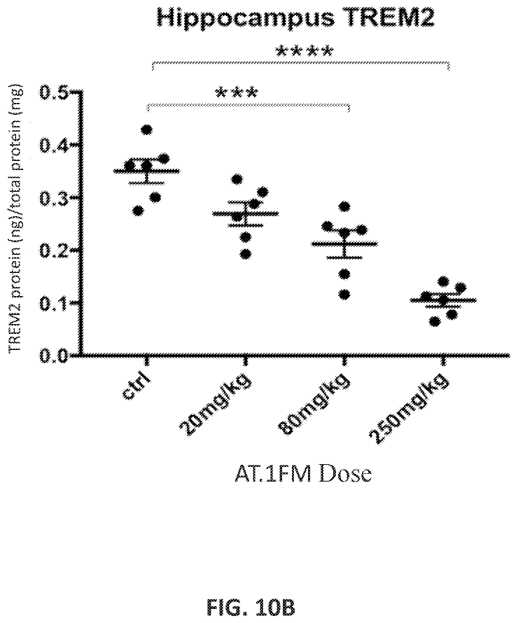

As shown in FIGS. 10A-10B, anti-TREM2 antibody AT.1FM reduced parenchymal TREM2 levels in non-human primates in a dose-dependent manner. In particular, anti-TREM2 antibody AT.1FM administered at doses of 20 mg/kg, 80 mg/kg, and 250 mg/kg reduced TREM2 protein levels in the frontal cortex of non-human primates compared to placebo-control treated animals (FIG. 10A). Additionally, anti-TREM2 antibody AT.1FM administered at doses of 20 mg/kg, 80 mg/kg, and 250 mg/kg reduced TREM2 protein levels in the hippocampus of non-human primates compared to placebo-control treated animals (FIG. 10B).

### Example 7: Anti-TREM2 Antibody Reduces sTREM2 in Cerebrospinal Fluid in Non-Human Primates

This Example describes the results of a study that evaluated soluble TREM2 (sTREM2) levels in cerebrospinal fluid (CSF) of non-human primates (cynomolgus monkeys) administered anti-TREM2 antibody AT.1FM.

Non-human primates were administered anti-TREM2 antibody AT.1FM by intravenous injection at doses of 20 mg/kg, 80 mg/kg, or 250 mg/kg once weekly (q1w) for 3 weeks (3×q1w; N=4 per dose group). CSF was obtained from each animal at various times following each antibody administration. sTREM2 levels were measured in the CSF.

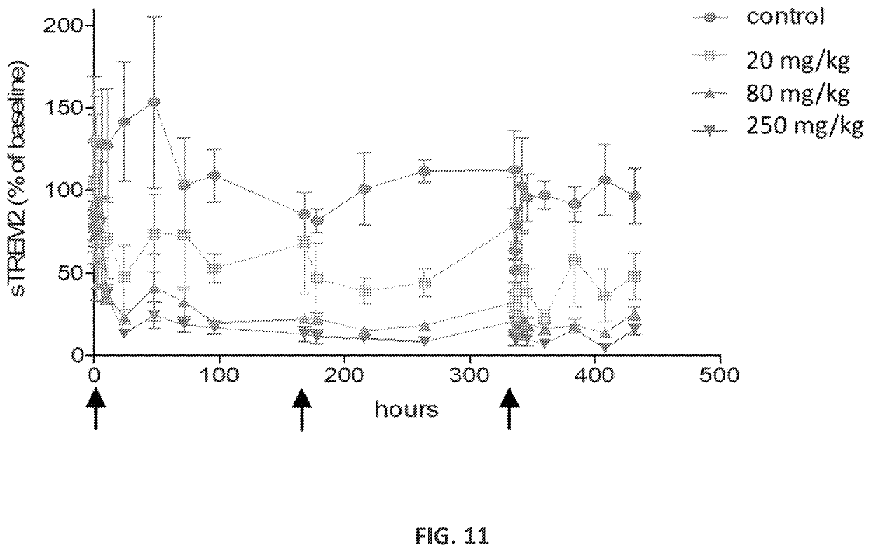

As shown in FIG. 11, the anti-TREM2 antibody reduced sTREM2 levels in the CSF in a dose-dependent manner in non-human primates compared to baseline. The arrows in FIG. 11 indicate the time of anti-TREM2 antibody dose administration according to the 3× q1w dosing regimen. CSF sTREM2 levels decreased following the initial dosing. In particular, sTREM2 levels in CSF decreased to approximately 50%-75% of baseline sTREM2 levels in animals dosed at 20 mg/kg; and decreased to approximately 20%-30% of baseline sTREM2 levels in animals dosed at either 80 mg/kg or 250 mg/kg.

### Example 8: Anti-TREM2 Antibody Increases Markers of Microglial Activity in Non-Human Primates

This Example describes the results of a study that evaluated the levels of biomarkers of microglial activity in the cerebrospinal fluid (CSF) of non-human primates (cynomolgus monkeys) administered anti-TREM2 antibody AT.1FM.

Non-human primates were administered anti-TREM2 antibody AT.1FM by intravenous injection at doses of 20 mg/kg, 80 mg/kg, or 250 mg/kg using a 3× q1w dosing regimen (N=4 per dose group). CSF was obtained from each animal at various times following each antibody administration, and the levels of osteopontin, a marker for activated microglia, were determined.

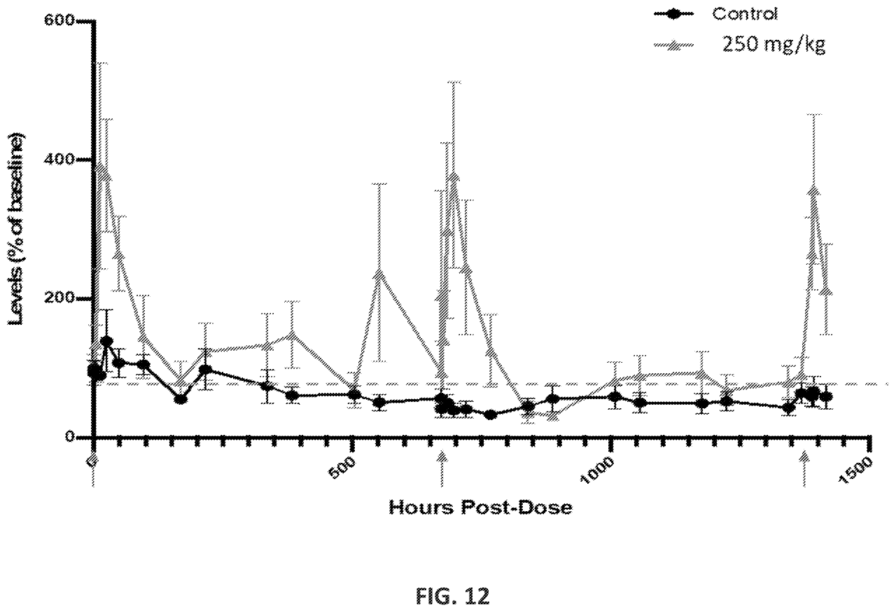

In a different study, non-human primates (cynomolgus monkeys) were administered 3 monthly IV injections of control or anti-TREM2 antibody AT.1FM at a dose of 250 mg/kg (N=4 per group). As shown in FIG. 12, osteopontin levels compared to baseline were significantly elevated in the CSF of animals administered AT.1FM when compared to the control group.

In a different study, non-human primates (cynomolgus monkeys) were administered weekly doses of control or anti-TREM2 antibody AL2p-58 huIgG1 (referred to herein as “AT.1F”) by intravenous injection at a dose of 80 mg/kg for a total of five doses (N=5 per dose group). AT.1F is a variant of anti-TREM2 antibody AT.1FM having an Fc comprising wild-type IgG1. Forty-eight hours after the 5^{th} dose, brain tissue was harvested and corresponding lysates were analyzed for CSF1R protein expression.

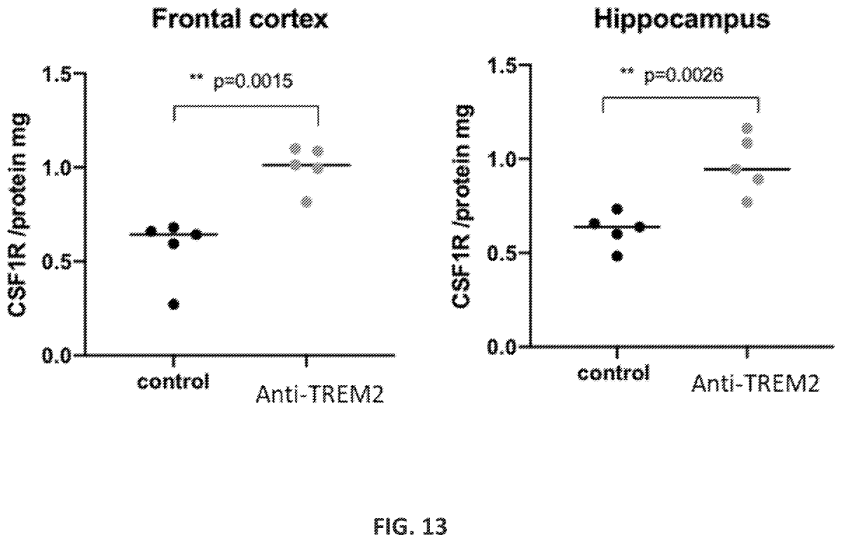

As shown in FIG. 13, CSF1R protein levels in the frontal cortex and in the hippocampus of non-human primates significantly increased following administration of anti-TREM2 antibody AT.1F as compared to control-treated animals.

### Example 9: A Phase 2 Study to Evaluate Efficacy and Safety of AT.1FM in Participants with Early Alzheimer's Disease

This Example describes a Phase 2 randomized, double-blind, placebo-controlled, multicenter study evaluating the efficacy and safety of anti-TREM2 antibody AT.1FM administered intravenously to participants with early Alzheimer's disease (AD).

### Study Design

Participant Inclusion and Exclusion Criteria

Adults aged 50 to 85 years that meet the following inclusion criteria are included in this study:
Diagnosis of Early AD, including evidence of brain amyloidosis by CSF or PET, based on the Alzheimer's disease continuum by the 2018 National Institute on Aging and Alzheimer's Association (NIA-AA) Research Framework (Jack et al., Alzheimers Dement (2018) 14(4):535-562).
Evidence of cerebral amyloidosis (A+) is required, as detailed in the following:
   The participant must be positive by the PrecivityAD™-Aβ blood test (i.e., have a high Amyloid Probability Score [APS]) prior to proceeding with either Amyloid PET or CSF studies for confirmation of amyloid beta (Aβ) pathology. The PrecivityAD™-Aβ blood test combines the blood concentration of Aβ isoforms amyloid beta (1-42) (Aβ42), amyloid beta (1-40) (Aβ40) and APOE isoforms, as measured by mass spectrometry, along with age (see, e.g., Schindler et al., Neurology (2019) 93(17):e1647-e1659).
   Demonstration of amyloid pathology by Amyloid PET or CSF phosphorylated tau (pTau)/amyloid beta (1-42) (Aβ42) ratio as outlined below is required for all participants.
   Participants with a positive historical Amyloid PET scan that has been collected≤24 months prior to the start of screening and that meets the acceptable criteria for a historical Amyloid PET scan as outlined below are not tested by PrecivityAD™-Aβ blood test.
   Participants with a validated, positive historical Amyloid PET scan are considered positive for cerebral Aβ pathology without further testing.
   Participants with an intermediate APS proceed to confirmation by Amyloid PET or CSF pTau/Aβ42 ratio. Participants with a low APS are not eligible.
Evidence of AD amyloid pathology, as demonstrated either by positive Amyloid PET scan by visual read conducted by a PET laboratory, or by a CSF pTau/Aβ42 ratio of greater than 0.024 as measured by the Roche Elecsys assay. Historical Amyloid PET collected≤24 months prior to the start of screening can fulfill this criterion; historical CSF measurements are not allowed to fulfill this criterion.
Clinical severity consistent with Stages 2, 3 or early Stage 4 as defined in the 2018 NIA-AA Research Framework, also described as mild cognitive impairment and mild dementia in the 2018 NIA-AA Research Framework.
The participant has mild symptomatology as defined by a screening Mini-Mental State Examination (MMSE) score≥22 points.
The participant has a CDR-Global Score (CDR-GS) of 0.5-1.0.
The participant has evidence of episodic memory impairment as defined by a Repeatable Battery for the Assessment of Neuropsychological Status (RBANS) score on the Delayed Memory Index (DMI)≤85.
If the participant is receiving symptomatic AD medications (for memory and/or behavioral symptoms), the dosing regimen must be stable for 90 days prior to screening start and not expected to change during study participation. Symptomatic AD medications are not initiated, modified, or stopped within 90 days prior to screening start.

Individuals that meet any of the following criteria are excluded from this study:
Any evidence of a condition other than AD that may affect cognition, including but not limited to, frontotemporal dementia, dementia with Lewy bodies, vascular dementia, Parkinson's disease, corticobasal degeneration, Creutzfeldt-Jakob disease, progressive supranuclear palsy, frontotemporal degeneration, Huntington disease, normal pressure hydrocephalus, hypoxic injury, seizure disorder, static encephalopathy, closed brain injury, or developmental disability.
Dementia due to a condition other than AD including, but not limited to, frontotemporal dementia (FTD), Parkinson's disease, dementia with Lewy bodies, Huntington disease, or vascular dementia.
Known history of severe allergic, anaphylactic, or other hypersensitivity reactions to chimeric, human, or humanized antibodies or fusion proteins.
Current uncontrolled hypertension, diabetes mellitus or thyroid disease.
Clinically significant heart disease, cardiovascular disease or disorder, liver disease or disorder, or kidney disease or disorder.
History or evidence of clinically significant brain disease other than AD.
History of unresolved cancer.
Current use of anticoagulant medications.
History or presence of vascular disease that has the potential to affect cognitive function (e.g., clinically significant carotid, vertebral stenosis, or plaque; aortic aneurysm; intracranial aneurysm; macro-hemorrhage; arteriovenous malformation).
History or presence of clinical stroke within the past 2 years, documented history within the last 180 days before screening of an acute event consistent with a transient ischemic attack, or presence on MRI of any cortical stroke regardless of age.
History of severe, clinically significant (e.g., persistent neurologic deficit or structural brain damage) CNS trauma (e.g., cerebral contusion).
History or presence of intracranial tumor (e.g., glioma, with the exception of benign brain tumors which do not cause cognitive symptoms).
Presence of infections that affect brain function, or history of infections that resulted in neurologic sequelae (e.g., human immunodeficiency virus, syphilis, neuroborreliosis, viral or bacterial meningitis/encephalitis).
Participant currently has or has had an acute illness that requires or required IV antibiotics within 30 days prior to first study treatment administration.
History or presence of systemic autoimmune disorders that potentially cause progressive neurologic disease with associated cognitive deficits (e.g., multiple sclerosis, lupus erythematosus, antiphospholipid antibody syndrome, Behçet disease).
History or presence of uveitis requiring medical intervention, chronic inflammatory or degenerative condition of the eye, current eye infection, any ongoing eye disorder (e.g., degeneration, cataract, or diabetic retinopathy) requiring injectable medical therapy (e.g., ranibizumab or aflibercept for macular degeneration), or planned invasive eye procedure during the study period.
Any history of schizophrenia, schizoaffective disorder, major depression, or bipolar disorder.
At risk of suicide.
History of alcohol and/or moderate to severe substance use disorder (according to the Diagnostic and Statistical Manual of Mental Disorders, 5th Edition) within the past 2 years.
MRI evidence of >2 lacunar infarcts, any territorial infarct>1 cm³, or white matter hyperintense lesions on the FLAIR sequence that correspond to an overall Fazekas score of 3.
Presence on MRI of >5 microbleeds and/or areas of leptomeningeal hemosiderosis.
Presence of significant cerebral vascular pathology as assessed by MRI.
Participant is positive for hepatitis B surface antigen, total hepatitis B core antibody, HIV-1 or -2 antibodies or antigen, or history of spirochetal infection of the CNS (e.g., syphilis, borreliosis, or Lyme disease). Participants with a positive hepatitis C virus antibody are allowed if hepatitis C ribonucleic acid (RNA) is negative.
Participants with active or latent TB disease.
Any chronic active immune disorder requiring systemic immunosuppressive therapy within 1 year prior to study enrollment. Underlying bone marrow dysfunction based upon hemoglobin<10 g/dL, absolute neutrophil count>1000/mm³, or platelet count<150000/mm³. Continuous use of prednisone≤10 mg/day or an equivalent corticosteroid is allowed if on a stable regimen for at least 90 days prior to study treatment; intermittent short-term use of prednisone or an equivalent corticosteroid is allowed to treat an acute condition.
Abnormal screening thyroid stimulating hormone (TSH) or tests that remain abnormal on retest or require a new treatment or an adjustment of current treatment.
Screening folic acid or vitamin B12 levels that are sufficiently low or remain low on retest such that deficiency may be contributing to cognitive impairment.
Screening hemoglobin A1c>8% or poorly controlled diabetes (including hypoglycemic episodes).
Any continuous use of medications known to impair consciousness or cognition (intermittent or short-term use [e.g., <1 week] of these medications is allowed if necessary for the treatment of a medical condition).
Any previous treatment with medications used to treat Parkinsonian symptoms or any other neurodegenerative disorder (with the exception of medications to treat Alzheimer's disease) within 1 year of screening Certain medications are acceptable if the participant is taking the medicine for a non-neurodegenerative disorder, such as restless leg disorder (e.g., pramipexole).
Typical antipsychotic or neuroleptic medication within 180 days of screening except as brief treatment for a nonpsychiatric indication (e.g., emesis).
Atypical antipsychotics except with intermittent short-term use (<1 week), which is permitted except within 2 days or 5 half-lives (whichever is longer) prior to any neurocognitive assessment.
Anticoagulation medications within 90 days of screening; antiplatelet treatments (e.g., aspirin, clopidogrel, dipyridamole) are permitted.
Systemic immunosuppressive therapy use or anticipated systemic immunosuppressive therapy use during the study. Continuous use of prednisone≤10 mg/day or an equivalent corticosteroid is allowed if on a stable regimen for at least 90 days prior to study treatment administration; intermittent short-term use of prednisone or an equivalent corticosteroid is allowed to treat an acute condition.
Chronic use of opiates or opioids (including long-acting opioid medication) within 90 days of screening. Intermittent short-term use (<1 week) of short acting opioid medications for pain is permitted except within 2 days or 5 half-lives (whichever is longer) prior to any neurocognitive assessment.
Stimulant medications (amphetamine, methylphenidate preparations, or modafinil) within 30 days of screening and throughout the study.
Chronic use of benzodiazepines, barbiturates, or hypnotics from 90 days before screening. Intermittent short-term use (<1 week) of benzodiazepines, buspirone or short acting hypnotic medication for sleep or anxiety is allowed except within 2 days or 5 half-lives (whichever is longer) prior to any neurocognitive assessment.

Study Treatments

This study includes three experimental arms and one placebo comparator arm. Table 4 provides an overview of the study arms and treatments in this study.

TABLE 4
Study arms and treatments.

| Study Arm | Assigned Treatment |
|---|---|
| AT.1FM Dose 1 | AT.1FM administered via intravenous |
| (experimental) | infusion at 15 mg/kg every 4 weeks. |
| AT.1FM Dose 2 | AT.1FM administered via intravenous |
| (experimental) | infusion at 40 mg/kg every 4 weeks. |
| AT.1FM Dose 3 | AT.1FM administered via intravenous |
| (experimental) | infusion at 60 mg/kg every 4 weeks. |
| Placebo Comparator | Placebo administered via intravenous |
| | infusion every 4 weeks. |

AT.1FM is administered as an intravenous (IV) infusion over approximately 60 minutes.

Study Objectives

The primary objective of this study is to evaluate the efficacy of AT.1FM in delaying disease progression compared to placebo in participants with Early AD.

The secondary objective of this study is to evaluate the efficacy of AT.1FM in participants with Early AD as measured by the rate of change in clinical outcome assessments, e.g., as described below.

The pharmacokinetics objective of this study is to estimate the concentration of AT.1FM in participants with Early AD in serum and CSF.

The safety objective of this study is to evaluate the safety and tolerability of AT.1FM in participants with Early AD.

The exploratory objective of this study is to evaluate the effects of AT.1FM in participants with Early AD on exploratory pharmacodynamics biomarkers (e.g., as described below).

Outcome Measures

The primary outcome measure for this study is disease progression as measured by the Clinical Dementia Rating Sum of Boxes (CDR-SB) assessment. Disease progression is assessed from the start of the study through study completion, up to 48 or 96 weeks.

The secondary outcome measures of this study include:
Change in MMSE score, assessed from the start of the study through study completion, up to 48 or 96 weeks.
Change in RBANS score, assessed from the start of the study through study completion, up to 48 or 96 weeks.
Change in Alzheimer's Disease Assessment Scale-Cognitive Subscale-13 (ADAS-Cog13) score, assessed from the start of the study through study completion, up to 48 or 96 weeks.
Change in Alzheimer's Disease Cooperative Study-Activities of Daily Living adapted to Mild Cognitive Impairment (ADCS-ADL-MCI) score, assessed from the start of the study through study completion, up to 48 or 96 weeks.
Change in Alzheimer's Disease Composite Score (ADCOMS), assessed from the start of the study through study completion, up to 48 or 96 weeks.
Evaluation of safety and tolerability of AT.1FM, including the incidence of adverse events.
Incidence of adverse events, assessed from the start of the study through study completion, up to 48 or 96 weeks.
Rate of change in MMSE score.
Rate of change in RBANS score.
Rate of change in ADAS-Cog13 score.
Rate of change in ADCS-ADL-MCI score.
Rate of change in ADCOMS score.
Change in CDR-SB assessment from baseline to Weeks 48, 72, and 96.
Change in MMSE score from baseline to Weeks 48, 72, and 96.
Change in RBANS score from baseline to Weeks 48, 72, and 96.
Change in ADAS-Cog13 score from baseline to Weeks 48, 72, and 96.
Change in ADCS-ADL-MCI score from baseline to Weeks 48, 72, and 96.
Change in ADCOMS score from baseline to Weeks 48, 72, and 96.

Additional outcome measures of this study include assessment of pharmacodynamic biomarkers, including change in magnetic resonance imaging (MRI), blood-based biomarkers, tau and amyloid positron emission tomography (PET) imaging, speech measurements, and cerebrospinal fluid (CSF) biomarkers. Pharmacodynamic biomarkers are assessed from the start of the study through study completion, up to 48 or 96 weeks.

Pharmacokinetic (PK) outcome measures of this study include:
Serum PK concentrations of AT.1FM and other PK parameters.
CSF PK concentrations of AT.1FM.
Incidence of anti-drug antibodies (ADAs).

Safety outcome measures of this study include:
Incidence of adverse events (AEs), including adverse events of special interest (AESIs), and serious adverse events (SAEs).
Changes from baseline in vital signs, physical findings, neurological findings, ophthalmological findings, ECG, and clinical laboratory results.
Columbia Suicide Severity Rating Scale (C-SSRS).
MRI abnormalities.

Exploratory pharmacodynamics (PD) biomarker outcome measures of this study include:
Changes from baseline in levels of soluble TREM2 (sTREM2) in CSF and/or plasma.
Changes from baseline in levels of biomarkers related to microglia function in CSF and/or plasma (e.g., CSF1R, IL1RN, osteopontin, and YKL40).
Changes from baseline in levels of biomarkers related to AD pathology in CSF and/or plasma (e.g., Aβ40, Aβ42, pTau, and total tau)
Changes from baseline in levels of neurodegeneration biomarkers in plasma and CSF (e.g., NfL).
Changes from baseline in brain volume, assessed by volumetric MRI.
Changes from baseline in brain pathological tau burden as assessed by tau positron emission tomography (Tau-PET).
Changes from baseline in brain amyloid burden as assessed by longitudinal Amyloid PET scanning.
Changes from baseline in speech measurements via the Winterlight Labs Speech Assessment (WLSA).

### Study Assessments

Efficacy Assessments

The primary objective of this study is to evaluate the efficacy of AT.1FM in delaying disease progression compared to placebo in participants with Early AD.

The following neurocognitive and functional tests are performed: CDR, MMSE, RBANS, ADAS-Cog13, ADCS-ADL-MCI, and WLSA. Neurocognitive and functional tests are performed prior to study treatment administration and prior to any stressful procedures (e.g., blood collections, lumbar punctures, or imaging). If a participant is taking intermittent or short-term regimens of medications known to impair consciousness or cognition, such medication is stopped 2 days or 5 half-lives (whichever is longer) prior to any cognitive or behavioral assessment. Use of cannabinoids (other than cannabidiol [CBD]) is prohibited within 72 hours prior to any cognitive or behavioral assessment.

Safety Assessments

Safety assessments include monitoring adverse events (AEs), physical, ophthalmological, and neurological examinations, vital signs, ECGs, clinical laboratory analytes, Columbia-Suicide Severity Rating Scale, and MRIs.

PK Assessments

PK blood, CSF, and ADA sample collections are performed, and AT.1FM concentrations are measured. On the dosing visits for Study Weeks 1, 5, 9, 13, 25, and 49, serum PK samples are collected pre-dose, within 15 minutes after the end of infusion, and within 60 to 90 minutes after the end of infusion. On all other dosing visits, serum PK samples are collected pre-dose and within 15 minutes after the end of infusion. The end of infusion is defined as the end of the line flush. On non-dosing visits, serum PK samples are collected at any time during the study visit.

Blood samples collected for ADA monitoring are analyzed for the presence of AT.1FM ADAs using a validated bridging immunoassay. Additional samples for ADA assessments are collected in participants with signs and symptoms of infusion-related reactions. In such cases, a corresponding additional PK sample is obtained at the same time point as the observed infusion-related reaction.

PD Biomarker Assessments

Blood-based biomarkers assessed in this study include:
sTREM2 in plasma.
Plasma biomarkers relevant to AD (e.g., Aβ42, Aβ40, total tau, pTau, and neurofilament light [NfL]).
Additional PD biomarkers, such as transcriptional analysis of whole blood following PAX gene extraction of cellular RNA to assess TREM2 expression, as well as other genes of interest.

CSF-based biomarkers assessed in this study include:
sTREM2 in CSF.
CSF biomarkers relevant to AD (e.g., Aβ42, Aβ40, total tau, pTau, NfL), and to microglia function (e.g., YKL40 and osteopontin)
Other exploratory PD biomarkers.

Imaging biomarkers assessed in this study include:
MRI imaging measures.
Longitudinal Amyloid PET imaging measures, e.g., using [¹⁸F]florbetaben (Neuraceq), [¹⁸F]florbetapir (Amyvid), or [¹⁸F]flutametamol (Vizamyl) as radiotracers.
Tau-PET imaging measures, e.g., using the [¹⁸F]MK-6240 Tau-PET radiotracer.

Genomic Assessments

A blood sample is collected at screening for DNA extraction to genotype APOE variants. Participants are stratified during randomization based on APOE e4 status (carrier vs non-carrier).

Blood samples are collected at baseline for DNA extraction to enable analysis of targeted genomic variants and whole genome sequencing (WGS) analysis to identify common and rare genetic variants that are predictive of response to AT.1FM, are associated with progression to a more severe disease state, are associated with safety findings, or can increase the knowledge and understanding of disease biology.

Targeted genomic assessments in this study include:
APOE e4
TREM2 variants, sialic acid-binding Ig-like lectin 3 (CD33) variants, transmembrane protein 106b (TMEM106b) variants, and CLUSTERIN variants.

What is claimed is:

1. A method of treating and/or delaying the progression of a disease or injury in an individual, comprising administering to the individual an anti-TREM2 antibody at a dose of at least about 15 mg/kg intravenously, wherein the anti-TREM2 antibody is an agonist.

2. A method of treating and/or delaying the progression of a disease or injury in an individual, comprising administering to the individual an anti-TREM2 antibody at a dose of at least about 15 mg/kg intravenously, wherein the antibody comprises a heavy chain variable region comprising an HVR-H1, HVR-H2, and HVR-H3 and a light chain variable region comprising an HVR-L1, HVR-L2, and HVR-L3, and wherein:
(i) the HVR-H1 comprises the amino acid sequence YAFSSQWMN (SEQ ID NO: 34), the HVR-H2 comprises the amino acid sequence RIYPGGGDTNYAGKFQG (SEQ ID NO: 35), the HVR-H3 comprises the amino acid sequence ARLLRNQPGESYAMDY (SEQ ID NO: 31), the HVR-L1 comprises the amino acid sequence RSSQSLVHSNRYTYLH (SEQ ID NO: 41), the HVR-L2 comprises the amino acid sequence KVSNRFS (SEQ ID NO: 33), and the HVR-L3 comprises the amino acid sequence SQSTRVPYT (SEQ ID NO: 32); or
(ii) the HVR-H1 comprises the amino acid sequence YAFSSDWMN (SEQ ID NO: 36), the HVR-H2 comprises the amino acid sequence RIYPGEGDTNYARKFHG (SEQ ID NO: 37), the HVR-H3 comprises the amino acid sequence ARLLRNKPGESYAMDY (SEQ ID NO: 38), the HVR-L1 comprises the amino acid sequence RTSQSLVHSNAYTYLH (SEQ ID NO: 39), the HVR-L2 comprises the amino acid sequence KVSNRVS (SEQ ID NO: 40), and the HVR-L3 comprises the amino acid sequence SQSTRVPYT (SEQ ID NO: 32).

3. The method of claim 1 or claim 2, wherein the dose is between about 15 mg/kg to about 60 mg/kg.

4. The method of any one of claims 1-3, wherein the dose is about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, or about 60 mg/kg.

5. The method of any one of claims 1-4, wherein the anti-TREM2 antibody is administered about once every four weeks at a dose of at least about 15 mg/kg.

6. The method of any one of claims 1-4, wherein the anti-TREM2 antibody is administered about once every week at a dose of at least about 15 mg/kg.

7. The method of any one of claims 1-5, wherein the anti-TREM2 antibody is administered about once every four weeks at a dose of about 15 mg/kg.

8. The method of any one of claims 1-5, wherein the anti-TREM2 antibody is administered about once every four weeks at a dose of about 20 mg/kg.

9. The method of any one of claims 1-5, wherein the anti-TREM2 antibody is administered about once every four weeks at a dose of about 25 mg/kg.

10. The method of any one of claims 1-5, wherein the anti-TREM2 antibody is administered about once every four weeks at a dose of about 30 mg/kg.

11. The method of any one of claims 1-5, wherein the anti-TREM2 antibody is administered about once every four weeks at a dose of about 35 mg/kg.

12. The method of any one of claims 1-5, wherein the anti-TREM2 antibody is administered about once every four weeks at a dose of about 40 mg/kg.

13. The method of any one of claims 1-5, wherein the anti-TREM2 antibody is administered about once every four weeks at a dose of about 45 mg/kg.

14. The method of any one of claims 1-5, wherein the anti-TREM2 antibody is administered about once every four weeks at a dose of about 50 mg/kg.

15. The method of any one of claims 1-5, wherein the anti-TREM2 antibody is administered about once every four weeks at a dose of about 55 mg/kg.

16. The method of any one of claims 1-5, wherein the anti-TREM2 antibody is administered about once every four weeks at a dose of about 60 mg/kg.

17. The method of any one of claims 1-16, wherein the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSQWMN (SEQ ID NO: 34), the HVR-H2 comprises the amino acid sequence RIYPGGGDTNYAGKFQG (SEQ ID NO: 35), the HVR-H3 comprises the amino acid sequence ARLLRNQPGESYAMDY (SEQ ID NO: 31), the HVR-L1 comprises the amino acid sequence RSSQSLVHSNRYTYLH (SEQ ID NO: 41), the HVR-L2 comprises the amino acid sequence KVSNRFS (SEQ ID NO: 33), and the HVR-L3 comprises the amino acid sequence SQSTRVPYT (SEQ ID NO: 32).

18. The method of any one of claims 1-17, wherein the antibody comprises a heavy chain variable region comprising the amino acid sequence of SEQ ID NO: 27 and a light chain variable region comprising the amino acid sequence of SEQ ID NO: 30.

19. The method of any one of claims 1-16, wherein the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSDWMN (SEQ ID NO: 36), the HVR-H2 comprises the amino acid sequence RIYPGEGDTNYARKFHG (SEQ ID NO: 37), the HVR-H3 comprises the amino acid sequence ARLLRNKPGESYAMDY (SEQ ID NO: 38), the HVR-L1 comprises the amino acid sequence RTSQSLVHSNAYTYLH (SEQ ID NO: 39), the HVR-L2 comprises the amino acid sequence KVSNRVS (SEQ ID NO: 40), and the HVR-L3 comprises the amino acid sequence SQSTRVPYT (SEQ ID NO: 32).

20. The method of any one of claim 1-16 or 19, wherein the antibody comprises a heavy chain variable region comprising the amino acid sequence of SEQ ID NO: 28 and a light chain variable region comprising the amino acid sequence of SEQ ID NO: 29.

21. The method of any one of claims 1-20, wherein the antibody has a human IgG1 isotype.

22. The method of any one of claims 1-21, wherein the antibody has a human IgG1 isotype and comprises amino acid substitutions in the Fc region at the residue positions P331S and E430G, wherein the numbering of the residues is according to EU numbering.

23. The method of any one of claim 1-18 or 21-22, wherein the antibody comprises:
a. a heavy chain comprising the amino acid sequence of SEQ ID NO: 43, and a light chain comprising the amino acid sequence of SEQ ID NO: 47; or
b. a heavy chain comprising the amino acid sequence of SEQ ID NO: 44, and a light chain comprising the amino acid sequence of SEQ ID NO: 47.

24. The method of any one of claim 1-16 or 19-21, wherein the antibody comprises:
a. a heavy chain comprising the amino acid sequence of SEQ ID NO: 45, and a light chain comprising the amino acid sequence of SEQ ID NO: 48; or
b. a heavy chain comprising the amino acid sequence of SEQ ID NO: 46, and a light chain comprising the amino acid sequence of SEQ ID NO: 48.

25. The method of any one of claims 1-24, wherein the disease or injury is selected from the group consisting of dementia, frontotemporal dementia, Alzheimer's disease, Nasu-Hakola disease, cognitive deficit, memory loss, a demyelination disorder, multiple sclerosis, Parkinson's disease, amyotrophic lateral sclerosis (ALS), Huntington's disease, adult-onset leukoencephalopathy with axonal spheroids and pigmented glia (ALSP), and a tauopathy disease.

26. The method of any one of claims 1-25, wherein the disease or injury is Alzheimer's disease.

27. The method of claim 26, wherein the individual has a Mini-Mental State Examination (MMSE) score of between about 16 points to about 28 points prior to administration of the anti-TREM2 antibody.

28. The method of claim 26 or claim 27, wherein the individual has a Clinical Dementia Rating-Global Score (CDR-GS) of 0.5, 1.0, or 2.0 prior to administration of the anti-TREM2 antibody.

29. The method of any one of claims 26-28, wherein the individual has a positive amyloid-PET scan prior to administration of the anti-TREM2 antibody.

30. The method of any one of claims 26-29, wherein the individual is being administered a cholinesterase inhibitor and/or memantine therapy.

31. The method of any one of claims 26-30, wherein the individual has symptoms of Alzheimer's disease prior to administration of the anti-TREM2 antibody.

32. The method of claim 31, wherein the symptoms are mild cognitive impairment and/or mild dementia.

33. The method of any one of claims 26-30, wherein the individual does not have symptoms of Alzheimer's disease prior to administration of the anti-TREM2 antibody.

34. The method of any one of claims 1-33, wherein the individual is heterozygous or homozygous for a mutation in TREM2.

35. The method of any one of claims 1-34, wherein the individual comprises an amino acid substitution in a human TREM2 protein at residue position R47H, R62H, or both.

36. The method of any one of claims 1-35, wherein the individual has a positive amyloid or tau blood test prior to administration of the anti-TREM2 antibody.

37. The method of any one of claims 1-36, wherein administration of the anti-TREM2 antibody results in at least about a 30% decrease in the levels of soluble TREM2 in the cerebrospinal fluid of the individual, compared to the levels of soluble TREM2 in the cerebrospinal fluid of the individual prior to administration of the anti-TREM2 antibody.

38. The method of any one of claims 1-37, wherein administration of the anti-TREM2 antibody results in at least about a 40% decrease in the levels of soluble TREM2 in the cerebrospinal fluid of the individual, compared to the levels of soluble TREM2 in the cerebrospinal fluid of the individual prior to administration of the anti-TREM2 antibody.

39. The method of claim 37 or claim 38, wherein the decrease in the levels of soluble TREM2 in the cerebrospinal fluid of the individual is present at about 2 days after administration of the anti-TREM2 antibody.

40. The method of any one of claims 37-39, wherein the levels of soluble TREM2 in the cerebrospinal fluid of the individual are measured in a sample of cerebrospinal fluid obtained from the individual using an electrochemiluminescent assay.

41. The method of any one of claims 1-40, wherein administration of the anti-TREM2 antibody results in at least about a 5% increase in the levels of soluble CSF1R in the cerebrospinal fluid of the individual, compared to the levels of soluble CSF1R in the cerebrospinal fluid of the individual prior to administration of the anti-TREM2 antibody.

42. The method of claim 41, wherein the increase in the levels of soluble CSF1R in the cerebrospinal fluid of the individual is present at about 2 days after administration of the anti-TREM2 antibody.

43. The method of claim 41 or claim 42, wherein the levels of soluble CSF1R in the cerebrospinal fluid of the individual are measured in a sample of cerebrospinal fluid obtained from the individual using an ELISA assay.

44. The method of any one of claims 1-43, further comprising measuring the levels of soluble TREM2 in a sample of blood, plasma, and/or cerebrospinal fluid from the individual before and after the individual has received one or more doses of the anti-TREM2 antibody.

45. The method of any one of claims 1-44, further comprising measuring the levels of soluble CSF1R in a sample of blood, plasma, and/or cerebrospinal fluid from the individual before and after the individual has received one or more doses of the anti-TREM2 antibody.

46. The method of any one of claims 1-45, further comprising measuring the levels of brain amyloid burden in the brain of the individual before and after the individual has received one or more doses of the anti-TREM2 antibody.

47. The method of claim 46, wherein the levels of brain amyloid burden in the brain of the individual are measured using amyloid-positron emission tomography.

48. The method of any one of claims 1-47, further comprising measuring one or more brain abnormalities in the brain of the individual before and after the individual has received one or more doses of the anti-TREM2 antibody.

49. The method of claim 48, wherein the one or more brain abnormalities are measured using magnetic resonance imaging.

50. The method of claim 48 or claim 49, wherein the one or more brain abnormalities is brain volume.

51. The method of any one of claims 1-50, further comprising detecting the presence of an alteration in one or more genes in the individual selected from the group consisting of APOE, TREM2, CD33, TMEM106b, and CLUSTERIN.

52. The method of any one of claims 1-51, further comprising measuring the levels of one or more biomarkers of neurodegeneration in a sample of blood, plasma, and/or cerebrospinal fluid from the individual before and after the individual has received one or more doses of the anti-TREM2 antibody.

53. The method of claim 52, wherein the one or more biomarkers of neurodegeneration is neurofilament light.

54. The method of any one of claims 1-53, further comprising measuring the expression levels of TREM2, CSF1R, YKL40, IL-1RA, or osteopontin in a sample of blood, plasma, and/or cerebrospinal fluid from the individual before and after the individual has received one or more doses of the anti-TREM2 antibody.

55. The method of any one of claims 1-54, further comprising measuring the levels of one or more biomarkers of Alzheimer's disease in a sample of blood, plasma, and/or cerebrospinal fluid from the individual before and after the individual has received one or more doses of the anti-TREM2 antibody.

56. The method of claim 55, wherein the one or more biomarkers of Alzheimer's disease are Aβ40, Aβ42, pTau, and/or total tau.

57. The method of any one of claims 1-56, further comprising determining a score of one or more clinical assessments of the individual before and after the individual has received one or more doses of the anti-TREM2 antibody, wherein the one or more clinical assessments are selected from the group consisting of the Mini-Mental State Examination (MMSE) score, the Clinical Dementia Rating-Global Score (CDR-GS), the Clinical Dementia Rating Sum of Boxes (CDR-SB), and the Repeatable Battery for the Assessment of Neuropsychological Status (RBANS).

58. The method of any one of claims 1-57, further comprising performing tau or amyloid positron emission tomography (PET) imaging assessments in the individual before and after the individual has received one or more doses of the anti-TREM2 antibody.

59. The method of any one of claims 1-58, wherein the disease or injury is Alzheimer's disease, and wherein the Alzheimer's disease is early Alzheimer's disease.

60. The method of claim 59, wherein the individual has brain amyloidosis prior to administration of the anti-TREM2 antibody, wherein brain amyloidosis is assessed in a sample of cerebrospinal fluid obtained from the individual, or by positron emission tomography (PET).

61. The method of claim 59 or claim 60, wherein the individual has a Mini-Mental State Examination (MMSE) score of at least about 22 points prior to administration of the anti-TREM2 antibody.

62. The method of any one of claims 59-61, wherein the individual has a Clinical Dementia Rating-Global Score (CDR-GS) of between about 0.5 and about 1.0 prior to administration of the anti-TREM2 antibody.

63. The method of any one of claims 59-62, wherein the individual has a Repeatable Battery for the Assessment of Neuropsychological Status on the Delayed Memory Index (RBANS DMI) score of 85 or less prior to administration of the anti-TREM2 antibody.

64. The method of any one of claims 59-63, wherein the individual has a positive amyloid or tau blood test prior to administration of the anti-TREM2 antibody.

65. The method of any one of claims 59-64, further comprising determining a score of one or more clinical assessments of the individual before and after the individual has received one or more doses of the anti-TREM2 antibody, wherein the one or more clinical assessments are selected from the group consisting of the Clinical Dementia Rating Sum of Boxes (CDR-SB), the Mini-Mental State Examination (MMSE), the Repeatable Battery for the Assessment of Neuropsychological Status (RBANS), the Alzheimer's Disease Assessment Scale-Cognitive Subscale-13 (ADAS-Cog13), the Alzheimer's Disease Cooperative Study-Activities of Daily Living adapted to Mild Cognitive Impairment (ADCS-ADL-MCI), and the Alzheimer's Disease Composite Score (ADCOMS).

66. The method of any one of claims 59-65, further comprising measuring the levels of one or more biomarkers of Alzheimer's disease before and after the individual has received one or more doses of the anti-TREM2 antibody, wherein the one or more biomarkers of Alzheimer's disease are measured by magnetic resonance imaging (MRI), or in a sample of blood, plasma or cerebrospinal fluid obtained from the individual.

67. The method of any one of claims 59-66, further comprising performing tau or amyloid positron emission tomography (PET) imaging assessments in the individual before and after the individual has received one or more doses of the anti-TREM2 antibody.

68. The method of any one of claims 59-67, further comprising performing one or more speech assessments in the individual before and after the individual has received one or more doses of the anti-TREM2 antibody.

69. The method of any one of claims 1-6 and 17-68, wherein (a) the dose is about 15 mg/kg and the terminal half-life of the antibody in the plasma of the individual is about 8.63 days; (b) the dose is about 30 mg/kg and the terminal half-life of the antibody in the plasma of the individual is about 7.44 days; (c) the dose is about 45 mg/kg and the terminal half-life of the antibody in the plasma of the individual is about 8.40 days; or (d) the dose is about 60 mg/kg and the terminal half-life of the antibody in the plasma of the individual is about 9.93 days.

70. A method of monitoring the treatment of an individual being administered an anti-TREM2 antibody, comprising measuring the levels of soluble TREM2 in a sample of cerebrospinal fluid, blood, or plasma from the individual before and after the individual has received one or more doses of an anti-TREM2 antibody.

71. The method of claim 70, further comprising a step of assessing the activity of the anti-TREM2 antibody in the individual based on the levels of soluble TREM2 in the sample of cerebrospinal fluid, blood, or plasma.

72. The method of claim 71, wherein the anti-TREM2 antibody is determined to be active in the individual if the levels of soluble TREM2 in the sample of cerebrospinal fluid, blood, or plasma are decreased after the individual has received one or more doses of the anti-TREM2 antibody, compared to the levels of soluble TREM2 in the sample of cerebrospinal fluid, blood, or plasma before the individual received a dose of the anti-TREM2 antibody.

73. A method of monitoring the treatment of an individual being administered an anti-TREM2 antibody, comprising measuring the levels of soluble CSF1R in a sample of cerebrospinal fluid, blood, or plasma from the individual before and after the individual has received one or more doses of an anti-TREM2 antibody.

74. The method of claim 73, further comprising a step of assessing the activity of the anti-TREM2 antibody in the individual based on the levels of soluble CSF1R in the sample of cerebrospinal fluid, blood, or plasma.

75. The method of claim 74, wherein the anti-TREM2 antibody is determined to be active in the individual if the levels of soluble CSF1R in the sample of cerebrospinal fluid, blood, or plasma are increased after the individual has received one or more doses of the anti-TREM2 antibody, compared to the levels of soluble CSF1R in the sample of cerebrospinal fluid, blood, or plasma before the individual received a dose of the anti-TREM2 antibody.

76. A method of monitoring the treatment of an individual being administered an anti-TREM2 antibody, comprising measuring the levels of YKL40 in a sample of cerebrospinal fluid, blood, or plasma from the individual before and after the individual has received one or more doses of an anti-TREM2 antibody.

77. The method of claim 76, further comprising a step of assessing the activity of the anti-TREM2 antibody in the individual based on the levels of YKL40 in the sample of cerebrospinal fluid, blood, or plasma.

78. The method of claim 77, wherein the anti-TREM2 antibody is determined to be active in the individual if the levels of YKL40 in the sample of cerebrospinal fluid, blood, or plasma are increased after the individual has received one or more doses of the anti-TREM2 antibody, compared to the levels of YKL40 in the sample of cerebrospinal fluid, blood, or plasma before the individual received a dose of the anti-TREM2 antibody.

79. A method of monitoring the treatment of an individual being administered an anti-TREM2 antibody, comprising measuring the levels of IL-1RA in a sample of cerebrospinal fluid, blood, or plasma from the individual before and after the individual has received one or more doses of an anti-TREM2 antibody.

80. The method of claim 79, further comprising a step of assessing the activity of the anti-TREM2 antibody in the individual based on the levels of IL-1RA in the sample of cerebrospinal fluid, blood, or plasma.

81. The method of claim 80, wherein the anti-TREM2 antibody is determined to be active in the individual if the levels of IL-1RA in the sample of cerebrospinal fluid, blood, or plasma are increased after the individual has received one or more doses of the anti-TREM2 antibody, compared to the levels of IL-1RA in the sample of cerebrospinal fluid, blood, or plasma before the individual received a dose of the anti-TREM2 antibody.

82. A method of monitoring the treatment of an individual being administered an anti-TREM2 antibody, comprising measuring the levels of osteopontin in a sample of cerebrospinal fluid, blood, or plasma from the individual before and after the individual has received one or more doses of an anti-TREM2 antibody.

83. The method of claim 82, further comprising a step of assessing the activity of the anti-TREM2 antibody in the individual based on the levels of osteopontin in the sample of cerebrospinal fluid, blood, or plasma.

84. The method of claim 83, wherein the anti-TREM2 antibody is determined to be active in the individual if the levels of osteopontin in the sample of cerebrospinal fluid, blood, or plasma are increased after the individual has received one or more doses of the anti-TREM2 antibody, compared to the levels of osteopontin in the sample of cerebrospinal fluid, blood, or plasma before the individual received a dose of the anti-TREM2 antibody.

85. The method of any one of claims 70-84, wherein the anti-TREM2 antibody is an agonist.
